(19) United States
(12) Patent Application Publication
(10) Pub. No.: US 2024/0359180 A1
(43) Pub. Date: Oct. 31, 2024

(54) DEVICES, SYSTEMS, AND METHODS FOR ANALYSIS OF NUCLEIC ACIDS

(71) Applicant: Mammoth Biosciences, Inc., Brisbane, CA (US)

(72) Inventors: Timothy James PATNO, Barrington, IL (US); Phillip You Fai LEE, South San Francisco, CA (US); Benjamin Andrew BLIZARD, Oakland, CA (US); Xin MIAO, Mountain View, CA (US); Jacob LESINSKI, Oakland, CA (US); Ryan A. BROWN, Oakland, CA (US); Daniel Thomas DRZAL, Pacifica, CA (US); Sarah Jane SHAPIRO, Pacifica, CA (US); Joshua BAIK, San Francisco, CA (US); Janice Sha CHEN, San Francisco, CA (US); James Paul BROUGHTON, South San Francisco, CA (US); Clare Louise FASCHING, Redwood City, CA (US); Jesus CHING, Saratoga, CA (US); Sonal JAIN, San Mateo, CA (US); Deepika VERMA, Belmont, CA (US); Matthew VEROSLOFF, South San Francisco, CA (US); Devin SPRATT, Mountain View, CA (US); Nicholas John FANTIN, San Bruno, CA (US)

(21) Appl. No.: 18/541,650

(22) Filed: Dec. 15, 2023

Related U.S. Application Data
(63) Continuation of application No. PCT/US2022/034110, filed on Jun. 17, 2022.
(60) Provisional application No. 63/211,555, filed on Jun. 17, 2021.
(60) Provisional application No. 63/293,564, filed on Dec. 23, 2021.
(60) Provisional application No. 63/336,959, filed on Apr. 29, 2022.
(60) Provisional application No. 63/348,672, filed on Jun. 3, 2022.

(51) Int. Cl. B01L 3/00

(52) U.S. Cl. CPC B01L 3/502761; B01L 2200/0647; B01L 2200/0684; B01L 2200/10; B01L 2200/16; B01L 2300/0663; B01L 2300/161; B01L 2300/1805; B01L 2400/06

(57) ABSTRACT

The present disclosure provides various systems, diagnostic devices, and methods for nucleic acid analysis. The systems, devices, and methods allow for the analysis of nucleic acids, in a sample, via programmable nuclease-based assays. Provided are systems comprising an instrument and a cartridge allowing for point of care use. The systems, devices, and methods described herein can be configured for multiplexed detection of nucleic acids in a single sample.

### CROSS-REFERENCE

This application claims priority to U.S. Provisional Application Ser. No. 63/348,672 filed on Jun. 3, 2022; U.S. Provisional Application Ser. No. 63/336,959 filed on Apr. 29, 2022; U.S. Provisional Application Ser. No. 63/293,564 filed on Dec. 23, 2021; and U.S. Provisional Application Ser. No. 63/211,555 filed on Jun. 17, 2021, each of which is incorporated herein by reference in its entirety for all purposes.

### SEQUENCE LISTING

The instant application contains a Sequence Listing which has been submitted electronically in XML format and is hereby incorporated by reference in its entirety. Said XML copy, created on Jul. 17, 2024, is named 203477-746601_SL.xml and is 201,751 bytes in size.

### BACKGROUND

Nucleic acid tests can be used to detect and identify nucleic acid sequences associated with disease-causing organisms, such as influenza. Diagnostic tests that detect the presence or absence of nucleic acids to identify pathogenic organisms often have an advantage in sensitivity and specificity over other test methods, such as those based on antigen detection of the pathogenic organism. In some cases, while antibody/antigen tests may have the ability to directly assay a subject's immune response to a pathogenic organism, these systems can have sensitivity and specificity challenges related to the response time and the variabilities of antibodies/antigens produced by the subject's body.

Although nucleic acid-based technologies have been demonstrated in decentralized testing environments, with some showing sensitivity near to that of lab-based PCR, such technologies lack the capability of detecting higher levels of multiple analytes, or targets, in a single patient sample (i.e., multiplexing). Currently, existing lower cost, easy-to-use nucleic acid-based test systems are limited in their ability to perform multiplexing due at least in part to difficulties in signal differentiation while trying to maintain a high reliability at lower cost and greater ease of use.

Additionally, nucleic acid test systems have demonstrated that they can specifically differentiate the subtype of the bacterial or viral infection. In at least some instances, pathogenic organism genotype to phenotype linking has shown that nucleic acid signatures can reliably identify a bacteria's resistance to certain antibiotics. or identify a sub-type of a disease-causing agent (e.g., influenza or coronaviruses) that is more difficult to treat with available anti-viral treatments. Sub-typing of viral infections may also be important for informing patient isolation procedures (such as to reduce the chance of infection spread in schools, workplaces, or healthcare facilities). Unfortunately, there is currently a lack of low cost and easy to use systems capable of such differentiation. Those systems which currently exist that can differentiate between infectious organism subtypes typically require several thousands of dollars to procure and, in the United States, must be used in a Clinical Laboratory Improvement Amendments (CLIA)-certified laboratory. Existing systems, due at least in part to the cost and/or ease of use hurdles described above, may be less than ideal as a candidate for widespread decentralization.

### SUMMARY

It would therefore be desirable to provide nucleic acid-based test systems, devices, and methods combining the sensitivity and specificity benefits of a lab-based PCR method with the utility of syndromic testing via multiplexing, and also combined with the ease of use and low cost of a CLIA-waived test, such systems, devices, and methods of which have not yet been demonstrated by existing systems in the in vitro diagnostic medical device industry. The specificity advantage of a nucleic acid test system over other methods lends itself to pathogenic differentiation via multiplexing. The effective diagnosis and treatment of respiratory infections, gastrointestinal infections, sexually transmitted diseases such as HPV, and/or bacterial blood infections, to name a few, would be greatly improved around the world with such a system.

Further, a decentralized, low-cost nucleic acid-based test system with multiplexing capability can also be used for diagnosing genetic diseases by facilitating the interrogation of any one of several genetic mutations that can lead to disease. Protein abnormality diseases, cystic fibrosis, and hypercoagulation disorders are non-limiting examples of genetic abnormalities that can be caused by any one of several different known genetic variants and which could be analyzed using the nucleic acid-based test systems, devices, and methods described herein.

Pharmacogenomics is also an area that could be served by a low-cost nucleic acid-based test system with a multiplexing capability. Such an instrument may allow for testing of combinations of genetic variations, thereby enabling drug dosage and drug selection optimization on a larger scale and/or at lower cost than is currently available.

It would therefore be desirable to provide an easy to use, low cost, decentralized nucleic acid test system capable of multiplexing samples and/or detected targets. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure generally relates to medical devices, particularly relates to decentralized point of care devices, systems, and methods, and more particularly relates to decentralized nucleic acid in vitro diagnostic medical devices, systems, and methods.

In various aspects, the present disclosure provides cartridges and devices for detecting nucleic acids, systems comprising the same, and methods of use thereof. The systems or components thereof may be configured for one or more of initial sample processing (e.g., nucleic acid extraction), nucleic acid amplification, and/or nucleic acid detection. In some embodiments, the system or a component thereof (e.g., a cartridge) is configured for nucleic acid amplification. In some embodiments, the system or a component thereof is configured for nucleic acid amplification and detection. A non-limiting example of a nucleic acid detection comprises methodologies using a programmable nuclease.

In one aspect, the present disclosure provides a system for detecting a target nucleic acid, the system comprising a cartridge. In some embodiments, the cartridge comprises (a) a reagent reservoir; (b) a sample interface in fluid communication with the reagent reservoir, wherein the sample interface is configured to receive a sample; and (c) an amplification region in fluid communication with one or more of the sample interface or the reagent reservoir, and configured to amplify one or more nucleic acids in the sample. In some embodiments, the cartridge further comprises a detection region in fluid communication with the amplification region. In some embodiments, the system further comprises an instrument configured to interface with the cartridge.

An aspect of the present disclosure provides a system for detecting a target nucleic acid, the system comprising: an instrument; a cartridge configured to interface with the instrument, the cartridge comprising: a reagent reservoir; a sample interface in fluid communication with the reagent reservoir, the sample interface configured to receive a sample; an amplification region in fluid communication with one or more of the sample interface or the reagent reservoir and configured to amplify one or more nucleic acids in the sample; and a detection region in fluid communication with the amplification region, the detection region comprising; a programmable nuclease disposed within the detection region that is complexed with a guide nucleic acid, wherein the guide nucleic acid is complementary to a target nucleic acid, or a portion thereof, of the one or more nucleic acids, wherein the programmable nuclease is configured to be activated through binding of the guide nucleic acid to the target nucleic acid; and a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid; and wherein the detection region is configured to enable detection of the signal. In some embodiments, the reagent reservoir contains one or more sample preparation reagents and one or more beads stored therein. In some embodiments, the one or more sample preparation reagents comprises liquid reagents, dried reagents, lyophilized reagents, or a combination thereof. In some embodiments, the one or more sample preparation reagents comprise a protein digestion reagent, a cellular digestion reagent, one or more solvents, one or more lysis reagents, or a combination thereof. In some embodiments, the one or more beads are configured to bind with the target nucleic acid. In some embodiments, the one or more beads comprise a silica coating. In some embodiments, the one or more beads is disposed within the reagent reservoir as i) a dry powder, ii) a mixture with a liquid, or iii) a combination thereof. In some embodiments, the liquid is a sample preparation reagent. In some embodiments, the one or more beads are magnetic. In some embodiments, the one or more beads are disposed within the reagent reservoir. In some embodiments, the reagent reservoir further comprises one or more capsules configured to contain a sample preparation reagent of the one or more sample preparation reagents and the one or more beads. In some embodiments, each capsule in the reagent reservoir comprises a storage volume from about 50 μL to about 500 μL. In some embodiments, the reagent reservoir comprises from about 1 to about 10 capsules. In some embodiments, the reagent reservoir further comprises a silo for holding each capsule. In some embodiments, each capsule within the reagent reservoir is slidably disposed within a corresponding silo. In some embodiments, each capsule within the reagent reservoir comprises a capsule chamber for holding the sample preparation reagent and/or the one or more beads therein. In some embodiments, each capsule chamber within the reagent reservoir further comprises a pierceable cover disposed at an end of the capsule chamber. In some embodiments, each silo comprises a piercing mechanism configured to pierce through the pierceable cover of a capsule of the one or more capsules in the reagent reservoir, wherein the capsule is translated from a closed configuration to an open configuration, so as to release the sample preparation reagent and/or one or more beads therefrom. In some embodiments, the piercing mechanism comprises a piercer core disposed within the silo, wherein the capsule chamber is configured to slide towards the piercer core. In some embodiments, the instrument comprises an actuator platform configured to translate the piercing mechanism. In some embodiments, the actuator platform is configured to release the sample reagent and/or one or more beads from each capsule simultaneously or according to any sequence of capsules. In some embodiments, the cartridge and/or instrument is configured to transfer the sample reagent and/or one or more beads to the sample interface via one or more valves. In some embodiments, the one or more valves are configured to regulate flow between the reagent reservoir and the sample interface. In some embodiments, the one or more valves comprise a rotary valve, a jumper, or any combination thereof. In some embodiments, at least one of the valves comprises the jumper, wherein the jumper defines a jumper channel disposed within a housing thereof and enables fluid communication between reagent reservoir and the sample interface. In some embodiments, the jumper comprises 1) an initial closed configuration, wherein fluid flow between the reagent reservoir and the sample interface is prevented, and 2) an open configuration, wherein fluid flow between the reagent reservoir and the sample interface is permitted. In some embodiments, a first end of the jumper is located within a first jumper silo of the reagent reservoir and a second end of the jumper is located within a second jumper silo of the sample interface. In some embodiments, the first end of the jumper is slidably disposed within the first jumper silo and wherein the second end of the jumper is slidable disposed within the second jumper silo. In some embodiments, translating the jumper relative to the first jumper silo and the second jumper silo from a first position to a second position moves the jumper from the initial closed configuration to the open configuration. In some embodiments, translating the jumper relative to the first jumper silo and the second jumper silo from a second position to a third position moves the jumper from the open configuration to a final closed position, thereby preventing fluid flow between the reagent reservoir and the sample interface. In some embodiments, the instrument comprises an actuator platform configured to translate the jumper from the first position to the second position, and from the second position to the third position. In some embodiments, the jumper channel is configured to contain any number of the one or more sample preparation reagents therein. In some embodiments, the sample interface is configured to receive the sample as a liquid, extract the sample from a swab, or both. In some embodiments, the sample interface comprises a scraper to extract the sample from the swab. In some embodiments, the sample interface comprises a sample reservoir into which the sample is configured to be transferred. In some embodiments, the sample interface is configured to mix the sample with the one or more sample preparation reagents and/or the one or more beads to form a mixed sample solution. In some embodiments, the sample interface is configured to mix the sample via a cartridge heater, direct mechanical actuation, generating bubbles, passive mixing via fluid introduced into the sample interface, or a combination thereof. In some embodiments, the cartridge heater is further configured to heat the sample to facilitate lysing therein. In some embodiments, the instrument and/or cartridge further comprises a magnet configured to immobilize the one or more beads when adjacent the one or more beads. In some embodiments, the instrument is configured to move the magnet, thereby enabling movement of the one or more beads and nucleic acid bound thereto. In some embodiments, the reagent reservoir further comprises one or more concentration reagents and/or one or more elution reagents stored therein. In some embodiments, the cartridge further comprises a sample concentration region in fluid communication with the sample interface. In some embodiments, the sample concentration region comprises one or more concentration reagents and/or one or more elution reagents stored therein. In some embodiments, the cartridge and/or instrument is configured to transfer the mixed sample solution to the sample concentration region via one or more valves. In some embodiments, the one or more valves are configured to regulate flow between the sample interface and the sample concentration region. In some embodiments, the one or more valves comprise the rotary valve or another rotary valve, a second jumper, or any combination thereof. In some embodiments, at least one concentration reagent and/or at least one elution reagent is stored within a channel defined by the second jumper. In some embodiments, the one or more concentration reagents and/or one or more elution reagents comprises liquid reagents, dried reagents, lyophilized reagents, or a combination thereof. In some embodiments, the one or more concentration reagents comprises wash reagents of one or more ionic strength, an alcohol, or a combination thereof. In some embodiments, the one or more elution reagents comprises a low to no salt reagent. In some embodiments, the one or more elution reagents comprises a prescribed pH that enables releasing the target nucleic acid from the one or more beads. In some embodiments, the sample concentration region further comprises one or more capsules. In some embodiments, each capsule in the sample concentration region is configured to contain a concentration reagent of the one or more concentration reagents and/or an elution reagent of the one or more elution reagents. In some embodiments, each capsule in the sample concentration region comprises a storage volume from about 100 μL to about 1 mL. In some embodiments, each capsule in the sample concentration region comprises a storage volume from about 250 μL to about 750 μL. In some embodiments, the sample concentration region comprises 3-10 capsules. In some embodiments, the sample concentration region comprises 5-7 capsules. In some embodiments, the sample interface, the second jumper, and/or the sample concentration region further comprises a filter mesh configured to capture the one or more beads bound to the target nucleic acid. In some embodiments, the sample interface and/or the sample concentration region further comprises a waste region configured to receive excess fluid from the mixed sample solution and at least one concentration reagent mixed therewith, wherein the target nucleic acid is immobilized via the filter mesh and/or via a magnet located on the instrument. In some embodiments, the sample interface, the reagent reservoir, and/or the sample concentration region is further configured to elute the target nucleic acid from the one or more beads by contacting the one or more elution reagents thereto, thereby forming a concentrated nucleic acid solution. In some embodiments, the amplification region is configured to amplify the target nucleic acid via an isothermal reaction, thermocycling, reverse transcription, or any combination thereof. In some embodiments, isothermal reaction is Loop-mediated isothermal amplification (LAMP). In some embodiments, amplification via thermal cycling comprises polymerase chain-reaction (PCR). In some embodiments, the sample interface, reagent reservoir, and/or sample concentration region is configured to transfer the concentrated nucleic acid solution to the amplification region. In some embodiments, the concentrated nucleic acid solution is transferred to the amplification region via one or more valves. In some embodiments, the one or more valves are configured to regulate flow between the sample interface, reagent reservoir, and/or sample concentration region and the amplification region. In some embodiments, the one or more valves comprise the rotary valve or another rotary valve, a third jumper, or any combination thereof. In some embodiments, the amplification region and/or the third jumper comprises one or more amplification elution reagents stored therein. In some embodiments, the one or more amplification reagents comprise liquid amplification reagents, dried amplification reagents, lyophilized amplification reagents, or a combination thereof. In some embodiments, the liquid amplification reagents comprise one or more activator salts. In some embodiments, the amount of liquid amplification reagents stored is about 5 μL to about 30 μL. In some embodiments, the lyophilized amplification reagents comprise assay-specific primers, dNTPs, reverse transcriptase enzymes, thermostable polymerase enzymes, additional additives, or any combination thereof. In some embodiments, the additional additives comprise i) excipients such trehalose and/or raffinose, ii) BSA, iii) fish gelatin, iv) magnesium, v) other salts, or vi) any combination thereof. In some embodiments, the lyophilized amplification reagents are in the form of one or more pellets. In some embodiments, the amplification region comprises one or more chambers. In some embodiments, the concentrated nucleic acid solution is configured to mix with the one or more amplification reagents prior to entering the one or more chambers. In some embodiments, the amplification region comprises one or more chambers. In some embodiments, instrument and/or cartridge further comprises a thermal system to provide heat to the one or more chambers. In some embodiments, the thermal system comprises a heating element, a cooling element, a controller in operative communication with the heating element or cooling element, and/or a feedback monitor in operative communication with the controller, wherein the feedback monitor is configured to detect the temperature of the one or more chambers or a fluid therein. In some embodiments, the thermal system is configured to control the temperature within each chamber of the one or more chambers individually. In some embodiments, the heating element and/or cooling element comprises a Peltier heating and/or cooling system. In some embodiments, the heating element is configured to optically heat the one or more chambers. In some embodiments, at least one chamber of the one or more chambers comprises an optically transparent material. In some embodiments, at least one chamber of the one or more chambers comprises an optically transparent window. In some embodiments, the one or more chambers comprises a plastic comprising polyethylene polyimide, or any thermally conductive plastic known in the art so as to promote nucleic acid amplification. In some embodiments, the one or more chambers comprises a plastic having a thermal conductivity of about 0.1 Watts/meter*Kelvin to about 100 Watts/meter*Kelvin. In some embodiments, the one or more chambers comprises a plastic and a metallic layer to maximize heat conductivity therein. In some embodiments, the thermal system is configured to control the temperature of at least one chamber of the one or more chambers to a prescribed temperature. In some embodiments, the prescribed temperature is different for at least two chambers of the one or more chambers. In some embodiments, the prescribed temperature for a chamber of the one or more chambers is from about 45.0° C. to about 70° C., from about 65° C. to about 90° C., or from about 80° C. to about 100° C. In some embodiments, the thermal system is configured to control the temperature in at least one chamber of the one or more chambers to within about 0.5° C. of the prescribed temperature or within about 2° C. of the prescribed temperature. In some embodiments, the feedback monitor comprises a temperature sensor for measuring the temperature in a chamber of the one or more chambers. In some embodiments, the temperature sensor comprises an infrared sensor, an integrated circuit sensor, a resistance temperature detector, and/or a thermocouple. In some embodiments, the temperature sensor comprises temperature sensitive component. In some embodiments, the temperature sensitive component comprises a thermistor. In some embodiments, the temperature sensor comprises a surface coating and/or packing element so as to minimize or prevent interference with an amplification reaction. In some embodiments, each chamber of the one or more chambers has an internal volume of about 10 μL to about 20 μL. In some embodiments, the amplification reagents stored on the amplification region is pre-aliquoted into separate amounts for each chamber of the one or more chambers. In some embodiments, the detection region is configured for spatially multiplexed detection of a plurality of target nucleic acids in the sample. In some embodiments, the programmable nuclease comprises a Cas protein. In some embodiments, the Cas protein comprises Cas12, Cas13, Cas14, CasPhi, a thermostable Cas, or any combination thereof. In some embodiments, the detection region is configured to perform a liquid-based reaction or an immobilized array reaction. In some embodiments, the detection region comprises an array having a plurality of detection spots thereon to perform an immobilized array reaction. In some embodiments, each detection spot comprises a specific guide nucleic acid corresponding to a particular target nucleic acid for detection, the specific guide nucleic acid being immobilized to a surface of the detection region. In some embodiments, each detection spot further comprises a reporter immobilized to the surface. In some embodiments, wherein each programmable nuclease, guide nucleic acid, and/or reporter are immobilized on a detection spot using NHS-amine chemistry, streptavidin-biotin chemistry, or a combination thereof. In some embodiments, wherein the array comprises a microwell array, such that each detection spot corresponds to a microwell. In some embodiments, each microwell is from about 150 μm to about 500 μm in diameter and from about 150 μm to about 500 μm in depth. In some embodiments, each microwell is comprises a hydrophilic material or coating on an inside surface, and/or a hydrophobic material or coating on the outside and/or surrounding the microwell. In some embodiments, the plurality of detection spots are from about 10 to about 200 detection spots. In some embodiments, the detection region comprises one or more liquid detection chambers for performing a liquid-based reaction. In some embodiments, the cartridge further comprises a mixing chamber between the amplification region and the detection region. In some embodiments, each chamber of the one or more chambers within the amplification region is mapped to a corresponding liquid detection chamber of the one or more liquid detection chambers. In some embodiments, the thermal system or another thermal system is configured to heat each liquid detection chamber. In some embodiments, the liquid detection chamber is heated from about 35° C. to about 40° C. In some embodiments, the programmable nucleic acid, guide nucleic acid, and/or reporter are immobilized within the detection region. In some embodiments, the amplification region is configured to transfer the amplified target nucleic acid to the detection region. In some embodiments, the amplified target nucleic acid is transferred to the detection region via one or more valves. In some embodiments, the one or more valves are configured to regulate flow between the amplification region and the detection region. In some embodiments, the one or more valves comprise the rotary valve or another rotary valve, a fourth jumper, or any combination thereof. In some embodiments, the detection region and/or the fourth jumper comprises one or more detection reagents stored therein. In some embodiments, the programmable nucleic acid, guide nucleic acid, and/or reporter are provided as lyophilized detection reagents. In some embodiments, the instrument further comprises an optical sensor for detecting the presence of the target nucleic acid. In some embodiments, the optical sensor comprises an image sensor or an array of discrete optical detectors. In some embodiments, the detection of the presence of the target nucleic acid is via detecting 1) fluorescence, 2) a color change, 3) a brightness change, 4) a wavelength change of a light, or 5) a combination thereof. In some embodiments, the one or more capsules are aligned linearly or radially. In some embodiments, the interface between the instrument and cartridge enables operative communication therebetween. In some embodiments, the instrument comprises an opening to receive the cartridge. In some embodiments, the interface between the cartridge and the instrument enables alignment with 1) ports on the cartridge that facilitate movement of the sample therein, 2) the one or more capsules in the reagent reservoir, the first jumper, the second jumper, the one or more capsules in the sample concentration region, or a combination thereof, to facilitate release of contents therein, 3) the amplification region for provision of heat, 4) the detection region, for detecting the presence of the target nucleic acid, or 5) a combination thereof. In some embodiments, the instrument comprises an XYZ motorized gantry configured to operatively communicate with the cartridge. In some embodiments, the instrument further comprises a pump. In some embodiments, the cartridge and/or instrument is configured to move fluid within the cartridge and different regions via positive and/or negative pressure. In some embodiments, the cartridge and/or instrument further comprises a syringe to supply the positive and/or negative pressure. In some embodiments, the reagent reservoir, the sample interface, the sample concentration region, the waste region, the amplification region, and/or the detection region comprises separate modules that are coupled together and in fluid communication with each other. In some embodiments, the reagent reservoir, the sample interface, the sample concentration region, the waste region, the amplification region, and/or the detection region comprises separate detachably coupled modules. In some embodiments, the sample interface is configured to be in fluid communication with a serpentine channel to enable amplification of the target nucleic acid and/or lysis of the sample. In some embodiments, the instrument is configured to control fluid, temperature, and detection parameters of reactions occurring within the cartridge. In some embodiments, the programmable nuclease and the reporter are immobilized to a surface of the detection region. In some embodiments, the programmable nuclease and the reporter are contained within a chamber of the detection region, wherein the programmable nuclease and the reporter are configured to react in liquid phase. In some embodiments, the instrument comprises an optical sensor configured to detect a detection moiety released upon cleaving of the reporter by an activated programmable nuclease. In some embodiments, the cartridge comprises two separate components coupled together.

Another aspect of the present disclosure provides a system for detecting a target nucleic acid, the system comprising: an instrument; a cartridge configured to interface with the instrument, the cartridge comprising: a sample interface configured to receive a sample comprising one or more nucleic acids; one or more reagent capsules; and a detection region; a programmable nuclease disposed within the cartridge and that is complexed with a guide nucleic acid that is complementary to the target nucleic acid, or a portion thereof, of the one or more nucleic acids, wherein the programmable nuclease is configured to be activated through binding of the guide nucleic acid to the target nucleic acid; a reporter disposed within the cartridge, the reporter comprising a cleavable nucleic acid and a detection moiety, wherein cleavage of the cleavable nucleic acid by the activated programmable nuclease releases the detection moiety from the cleavable nucleic acid, wherein the released detection moiety is configured to generate a signal; wherein the detection region is configured to detect the signal indicating the presence of the target nucleic acid. In some embodiments, the sample interface comprises a scraper to extract the sample from a swab. In some embodiments, a sample preparation region is configured to purify and concentrate the one or more nucleic acids. In some embodiments, the sample preparation region further comprises a subset of the one or more reagent capsules, wherein the subset comprises a protein digestion reagent, a cellular digestion reagent, one or more solvents, or a combination thereof. In some embodiments, the liquid capacity of each of the reagent capsules ranges from 50 μL to 500 μL in volume. In some embodiments, the subset contains 4 to 6 reagent capsules. In some embodiments, the sample preparation region comprises one or more beads having a silica coating, wherein the silica coating is configured to bind at least one nucleic acid of the one or more nucleic acids. In some embodiments, the silica beads are magnetic silica beads. In some embodiments, the instrument comprises a magnet configured to immobilize and release the magnetic silica beads. In some embodiments, the system comprises an amplification region. In some embodiments, the system further comprises amplification reagents. In some embodiments, the amplification reagents are present as liquid amplification reagents and lyophilized amplification reagents. In some embodiments, the liquid amplification reagents comprise one or more activator salts. In some embodiments, the lyophilized amplification reagents comprise assay-specific primers, probes, dNTPs, reverse transcriptase enzymes, thermostable polymerase enzymes, additional additives, or any combination thereof. In some embodiments, the lyophilized amplification reagents are in the form of one or more pellets. In some embodiments, the instrument is configured to control fluid, temperature and detection parameters of reactions occurring within the cartridge. In some embodiments, the detection region is configured for spatially multiplexed detection of a plurality of target nucleic acids in the sample. In some embodiments, the programmable nuclease and the reporter are immobilized to a surface of the detection region. In some embodiments, the programmable nuclease and the reporter are contained within a chamber of the detection region, wherein the programmable nuclease and the reporter are configured to react in liquid phase. In some embodiments, the instrument comprises an optical sensor configured to detect a detection moiety released upon cleaving of the reporter by an activated programmable nuclease.

Another aspect of the present disclosure provides a system for multiplexed detection of a plurality of target nucleic acids comprising: an instrument; a cartridge comprising: a sample interface; one or more reagent capsules; a sample preparation region; an amplification region; and a detection region; the detection region comprising a plurality of detection locations, each detection location of the plurality of detection locations comprising a reporter and a programmable nuclease complexed with a guide nucleic acid that is complementary to a different target nucleic acid of a plurality of target nucleic acids, wherein, at each detection location, the corresponding reporter and the corresponding guide nucleic acid are immobilized to a surface of the detection region, wherein, at each detection location, the corresponding programmable nuclease is configured to cleave the reporter and generate a different signal of a plurality of signals, and wherein each different signal of the plurality of signals indicates a presence or absence of each different target nucleic acid at its respective detection spot. In some embodiments, the plurality of different locations are arranged in an array configuration. In some embodiments, the plurality of different locations comprises a plurality of chambers. In some embodiments, each detection location comprises a different reporter.

Another aspect of the present disclosure provides a method for detecting a target nucleic acid, the method comprising the steps of: receiving a sample containing a target nucleic acid via a sample interface; concentrating the target nucleic acid; transferring the target nucleic acid to a detection region; and detecting the target nucleic acid, wherein a programmable nuclease is activated by the target nucleic acid, the activated programmable nuclease cleaving a reporter and releasing a detection moiety, thereby generating a signal indicating the presence or absence of the target nucleic acid. In some embodiments, the method further comprises transferring the target nucleic acid to an amplification region. In some embodiments, the method further comprises amplifying the target nucleic acid. In some embodiments, the method further comprises outputting results.

Another aspect of the present disclosure provides a method for detecting a target nucleic acid, the method comprising the steps of: combining a first cartridge component containing liquid reagents with a second cartridge component containing lyophilized reagents, thereby reconstituting the lyophilized reagents, wherein the combining results in the first cartridge component and second cartridge component comprising an assembled cartridge; receiving a sample containing a target nucleic acid via a sample interface; lysing the sample; purifying and concentrating the target nucleic acid; transferring the target nucleic acid to an amplification region; amplifying the target nucleic acid; transferring the target nucleic acid to a detection region comprising a programmable nuclease complexed to a guide nucleic acid, wherein the detection region further comprises a reporter comprising a detection moiety; and detecting the target nucleic acid, wherein the programmable nuclease is activated by binding of the target nucleic acid to the guide nucleic acid, wherein activation of the programmable nuclease cleaves a reporter, thereby releasing a detection moiety from the reporter and generating a signal indicative of a presence or absence of the target nucleic acid.

Another aspect of the present disclosure provides a method for detecting a target nucleic acid, the method comprising the steps of: receiving a sample containing a plurality of target nucleic acids via a sample interface; loading the cartridge into an instrument; transferring the sample to a sample preparation region located within the cartridge; lysing the sample; purifying the plurality of target nucleic acids from the sample; concentrating the plurality of target nucleic acids; transferring the plurality of target nucleic acids to an amplification region; amplifying the plurality of target nucleic acids; transferring the plurality of target nucleic acids to a detection region, the detection region comprising a plurality of detection locations, each detection location comprising a reporter and a programmable nuclease of a plurality of programmable nucleases, wherein each programmable nuclease comprises a different guide nucleic acid complementary to a different target nucleic acid of the plurality of target nucleic acids, and wherein the corresponding reporter and the corresponding programmable nuclease of each detection location are immobilized to a surface of the detection region, and wherein at each detection location, each programmable nuclease is configured to cleave the corresponding reporter, thereby generating a different signal of a plurality of signals, each different signal of the plurality of signals indicating the presence or absence of each different complementary target nucleic acid of the plurality of target nucleic acids.

Another aspect of the present disclosure provides a system for detecting a target nucleic acid, the system comprising: a detection region comprising: a guide nucleic acid complementary to the target nucleic acid, or a portion thereof; a reporter immobilized to a surface of the detection region, the reporter comprising a nucleic acid and a detection moiety, wherein the nucleic acid is at least 40 nucleotides in length; the nucleic acid comprises a double-stranded region; or a combination thereof, and wherein cleavage of the reporter by a programmable nuclease, activated upon hybridization to the target nucleic acid, releases the detection moiety from the nucleic acid, and wherein the release of the detection moiety is configured to generate a signal indicative of a presence of the target nucleic acid.

Another aspect of the present disclosure provides a system for detecting a target nucleic acid, the system comprising: a detection region comprising at least one detection location comprising: a programmable nuclease disposed within the detection region that is complexed with a guide nucleic acid, wherein the guide nucleic acid is complementary to a target nucleic acid, or a portion thereof, wherein the programmable nuclease is configured to be activated through binding of the guide nucleic acid to the target nucleic acid; a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid; and a surface comprising a hydrophobic membrane.

In one aspect, the present disclosure provides a system comprising a microfluidic device comprising a plurality of chambers fluidically connected in sequence. In some embodiments, (a) each chamber of the plurality of chambers comprises a well, an inlet channel, an outlet, and a capillary valve; (b) the capillary valve of each chamber (i) has a cross-sectional area that is smaller than a cross-sectional area of the inlet channel of the respective chamber, and (ii) forms an entrance of the inlet channel of the next chamber in the sequence; and (c) each outlet is air-permeable and configured to retain liquid within the respective chamber. In some embodiments, each chamber further comprises detection reagents comprising a guide nucleic acid and a reporter, wherein (a) each guide nucleic acid (i) comprises a targeting sequence that hybridizes with a target nucleic acid of a plurality of different target nucleic acids or an amplicon thereof, and (ii) is effective to form a complex with a programmable nuclease that is activated upon binding the corresponding target nucleic acid or amplicon thereof; (b) the guide nucleic acid of a first chamber in the plurality of chambers comprises a different targeting sequence from the guide nucleic acid of a second chamber in the plurality of chambers; and (c) each reporter (i) comprises a cleavable nucleic acid and a detection moiety, and (ii) is configured to be cleaved to form a detectable cleavage product in response to activation of the complex in the well of the respective chamber. In some embodiments, the capillary valve has a cross-sectional area that is 75%, 50%, or less than a cross-sectional area of the inlet channel of the respective chamber. In some embodiments, the capillary valve is oriented at an angle of 90° or greater with respect to the inlet channel of the respective chamber. In some embodiments, the capillary valve forms a junction with the inlet channel of the respective chamber. In some embodiments, the capillary valve and the inlet channel intersect the well of a respective chamber at separate points along a perimeter of the well. In some embodiments, the inlet channels comprise (a) a width of about 0.3 mm to about 0.6 mm and a depth of about 0.25 mm to about 0.45 mm; (b) a width of about 0.4 mm and a depth of about 0.35 mm; or (c) a width of about 0.5 mm and a depth of about 0.35 mm. In some embodiments, the capillary valves comprise (a) a width of about 0.2 mm to about 0.4 mm and a depth of about 0.1 mm to about 0.3 mm; or (b) a width of about 0.3 mm and a depth of about 0.2 mm. In some embodiments, each of the wells has an internal volume of (a) about 0.1 μL to about 50 μL, (b) about 0.5 μL to about 20 μL, (c) about 0.75 μL, or (d) about 10 μL. In some embodiments, the outlet comprises (a) an opening sized to permit displacement of air therethrough but to retain liquid within the well under an operating pressure of the microfluidic device, (b) a surface comprising a hydrophobic coating, or (c) a surface comprising an air-permeable hydrophobic membrane. In some embodiments, the outlet comprises the surface comprising the air-permeable hydrophobic membrane, and further wherein the hydrophobic membrane (a) comprises woven polypropylene or woven polyethylene, (b) the hydrophobic membrane comprises pores of about 0.1 microns to about 2 microns in size, and/or (c) forms a bottom surface of the respective well. In some embodiments, the system further comprises a sample interface configured to receive a sample, wherein the sample interface is in fluid communication with the plurality of chambers. In some embodiments, the sample interface is fluidically connected to the plurality of chambers via one or more sample preparation regions. In some embodiments, the one or more sample preparation regions comprise a lysis region configured to lyse one or more components of the sample, optionally wherein the lysis region comprises lysis reagents. In some embodiments, (a) the sample interface is fluidically connected to the plurality of chambers via a bubble purge channel, (b) the bubble purge channel is connected to a sample inlet channel at an upstream end and a sample exit channel at a downstream end; and (c) the bubble purge channel is configured to purge gas bubbles from the sample fluid. In some embodiments, (a) a surface of the bubble purge channel comprises a gas-permeable membrane that is hydrophobic and/or oleophobic; and (b) the bubble purge channel is dimensioned to provide a pressure drop downstream from the bubble purge channel. In some embodiments, the one or more sample preparation regions comprise an amplification region, optionally wherein the amplification region comprises amplification reagents. In some embodiments, (a) the outlets vent through a first surface of the microfluidic device, (b) the system further comprises a heater in thermal communication with a second surface of the microfluidic device, and (c) the first surface is opposite the second surface. In some embodiments, the plurality of chambers comprises at least 10, 25, 50, or 100 chambers fluidically connected in sequence. In some embodiments, the detection reagents further comprise a programmable nuclease. In some embodiments, the programmable nuclease comprises a Cas protein, optionally wherein the Cas protein is selected from a Cas12, a Cas13, a Cas14, a CasPhi, and a thermostable Cas. In some embodiments, the detection reagents further comprise amplification reagents. In some embodiments, the detection reagents are in a lyophilized form. In some embodiments, the guide nucleic acid and/or the reporter in each chamber are immobilized to or otherwise disposed on a surface of the respective chamber.

In one aspect, the present disclosure provides a method for detecting one or more of a plurality of different target nucleic acids in a system described herein. In some embodiments, the method comprises (a) flowing a liquid comprising one or more of the different target nucleic acids or amplicons thereof into the plurality of chambers; (b) in one or more of the wells, forming the activated complex and cleaving the reporters; and (c) detecting the detectable cleavage products in one or more of the wells, wherein the location of a well comprising a detectable cleavage product identifies the target nucleic acid or amplicon thereof present in the well.

In one aspect, the present disclosure provides a microfluidic device comprising: a loading channel comprising a first capillary valve disposed upstream of a second capillary valve disposed therein; a first chamber fluidically coupled to the loading channel upstream of the first capillary valve; a second chamber fluidically coupled to the loading channel between the first capillary valve and the second capillary valve; and a third chamber fluidically coupled to the loading channel downstream of the second capillary valve; wherein (a) each chamber of the first, second, and third chambers comprises an outlet; (b) each of the first and second capillary valves have a cross-sectional area that is smaller than a cross-sectional area of the loading channel; and (c) each outlet is gas-permeable and configured to retain liquid within the respective chamber.

In one aspect, the present disclosure provides a cartridge for use in a system for detecting a target nucleic acid, the cartridge being configured to interface with an instrument of the system. In some embodiments, the cartridge comprises: a reagent reservoir; a sample interface in fluid communication with the reagent reservoir, the sample interface configured to receive a sample; an amplification region in fluid communication with one or more of the sample interface or the reagent reservoir and configured to amplify one or more nucleic acids in the sample; and a detection region in fluid communication with the amplification region. In some embodiments, the detection region comprises: (a) a programmable nuclease disposed within the detection region that is complexed with a guide nucleic acid, wherein the guide nucleic acid is complementary to a target nucleic acid, or a portion thereof, of the one or more nucleic acids, wherein the programmable nuclease is configured to be activated through binding of the guide nucleic acid to the target nucleic acid; and (b) a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid. In some embodiments, the detection region is configured to enable detection of the signal. The present disclosure also provides an instrument for use in a system for detecting a target nucleic acid, wherein the instrument is configured to interface with the cartridge.

In one aspect, the present disclosure provides a cartridge for use in a system for detecting a target nucleic acid, the cartridge being configured to interface with an instrument of the system. In some embodiments, the cartridge comprises: a reagent reservoir; a sample interface in fluid communication with the reagent reservoir, the sample interface configured to receive a sample; an amplification region in fluid communication with one or more of the sample interface or the reagent reservoir and configured to amplify one or more nucleoid acids in the sample; and a detection region in fluid communication with the amplification region, the detection region configured to generate a signal indicative of a presence of the target nucleic acid; and wherein the detection region is configured to enable detection of the signal.

In one aspect, the present disclosure provides a cartridge for detecting a target nucleic acid. In some embodiments, the cartridge comprises: (a) a reagent reservoir; (b) a programmable nuclease, wherein the programmable nuclease is configured to be activated through binding of a guide nucleic acid to a target nucleic acid; (c) a reporter, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid; (d) a sample interface in fluid communication with the reagent reservoir, the sample interface configured to receive a sample; (e) a detection region in fluid communication with the sample interface, the detection region comprising: (i) a guide nucleic acid disposed within the detection region, wherein the guide nucleic acid is complementary to the target nucleic acid, or a portion thereof, of the one or more nucleic acids; and (ii) a primer disposed within the detection region, wherein the primer is designed to amplify one or more nucleic acids in the sample; and wherein the detection region is configured to amplify the one or more nucleic acids in the sample and to enable detection of the signal. In some embodiments, the programmable nuclease and/or the reporter is disposed within the detection region. In some embodiments, the programmable nuclease and/or the reporter is disposed between the sample interface and the detection region. In some embodiments, movement of at least a portion of the sample from the sample interface to the detection region moves the programmable nuclease and/or reporter into the detection region. In some embodiments, one or more amplification reagents are disposed within the detection region. In some embodiments, one or more amplification reagents are disposed upstream of the detection region. In some embodiments, movement of at least a portion of the sample from the sample interface to the detection region moves the one or more amplification reagents into the detection region. In some embodiments, the one or more amplification reagents comprise a reverse transcriptase and/or a polymerase. In some embodiments, the programmable nuclease, reporter, and/or one or more amplification reagents are dried or lyophilized prior to mixing with the sample (or the portion thereof).

These and other embodiments are described in further detail in the following description related to the appended drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure may be obtained by reference to the following detailed description that sets forth illustrative embodiments, in which the principles of the present disclosure are utilized, and the accompanying drawings of which:

FIGS. 1A-1B depict an embodiment of a system for nucleic acid analysis, in accordance with embodiments. FIG. 1A shows a perspective view of an exemplary system including an instrument for user input and output and process control with cartridge inserted therein. FIG. 1B shows a perspective view of an exemplary cartridge for use with the system of FIG. 1A.

FIG. 2 shows a schematic diagram highlighting exemplary functions of a system for nucleic acid analysis, in accordance with embodiments.

FIG. 3 depicts a schematic diagram illustrating exemplary functions which may be performed by a system for nucleic acid analysis, in accordance with embodiments.

FIGS. 4A-4E depict various components of an exemplary cartridge device, in accordance with embodiments. FIG. 4A shows a perspective view of an exemplary cartridge including a sample interface and electrical contacts configured to interface with an instrument.

FIG. 4B shows a perspective view of a custom swab with a cap which is configured for insertion into the sample interface of the cartridge. FIG. 4C shows a perspective cross-sectional view of the sample interface with swab inserted therein, including a scraper configured to facilitate collection of the sample from the swab. FIG. 4D shows a perspective view of the cartridge with housing and sample interface removed. FIG. 4E shows a plan view of the cartridge highlighting the internal components of the rotary valve in the cartridge.

FIG. 4F shows valve positioning of the device of FIGS. 4A-4E at various stages during nucleic acid analysis, in accordance with embodiments.

FIG. 5 depicts an exemplary arrangement of reagent reservoirs and a sample reservoir of a cartridge, in accordance with embodiments.

FIG. 6A depicts a perspective view of a plurality of reservoirs optionally comprising reagent capsules, in accordance with embodiments.

FIG. 6B depicts a top view of a plurality of reservoirs optionally comprising reagent capsules, in accordance with embodiments.

FIGS. 7A-7C depict an exemplary reagent reservoir/capsule, in accordance with embodiments. FIG. 7A shows a perspective view of an unfilled reagent capsule. FIG. 7B shows a perspective cross-sectional view of a filled and sealed reagent capsule. FIG. 7C shows a cross-sectional view of a reagent capsule disposed within a silo above a piercer core.

FIGS. 8A-8C show an exemplary sample reservoir and a plurality of jumper silos, in accordance with embodiments. FIG. 8A shows a cross-sectional view of a pair of jumper silos and a sample reservoir with vent open. FIG. 8B shows a perspective view of the sample reservoir with vent open and jumper silos therearound. FIG. 8C shows a cross-sectional view of the jumper silos and the sample reservoir with vent closed.

FIGS. 9A-9E show an exemplary fluid jumper, in accordance with embodiments. FIG. 9A shows a perspective view of a jumper comprising two cylindrical channels joined to one another at their respective proximal ends by a horizontal channel. FIG. 9B shows a perspective view of the jumper of FIG. 9A further comprising a proximal surface coupled the horizontal surface to complete the jumper. FIG. 9C shows a perspective cross-sectional view of the jumper of FIG. 9B. FIG. 9D shows a perspective view of a jumper comprising elastomer seals around the distal ends of each cylinder. FIG. 9E shows a perspective view of an alternative sealing embodiment utilizing flexure seal rings around the distal ends of each cylinder.

FIGS. 10A-10C show cross-sectional views of a jumper in use, in accordance with embodiments. FIG. 10A shows the jumper in an initial closed configuration prior to actuation.

FIG. 10B shows the jumper after actuation to an open configuration which links previously discontinuous fluid pathways together and enables fluid flow there between via the jumper channel. FIG. 10C shows the jumper after further actuation to a final closed configuration which shuts off fluid flow therethrough.

FIG. 11A shows an exemplary cartridge arrangement comprising a sample receiver module, two reagent modules, and an amplification module, in accordance with embodiments.

FIG. 11B shows another exemplary cartridge arrangement comprising a sample receiver module, two reagent modules, and an amplification module, in accordance with embodiments.

FIG. 12A shows an exemplary thermocycling system comprising a plurality of moveable thermal reservoirs, in accordance with embodiments.

FIG. 12B shows a cross-sectional view of a thermal reservoir comprising a flat contact surface, in accordance with embodiments.

FIG. 12C shows a cross-sectional view of a thermal reservoir comprising a curved contact surface, in accordance with embodiments.

FIG. 13 depicts a surface comprising an immobilized programmable nuclease-guide nucleic acid complex and a plurality of reporters, where one reporter has been cleaved by an activated programmable nuclease, in accordance with embodiments.

FIG. 14 shows an exemplary detection region, in accordance with embodiments.

FIG. 15 depicts an exemplary workflow of a system for nucleic acid analysis, in accordance with embodiments.

FIGS. 16A-16F depict various steps of an exemplary process for using a system for nucleic acid analysis, in accordance with embodiments.

FIG. 17 depicts an exemplary workflow for a patient use case of a system for nucleic acid analysis, in accordance with embodiments.

FIG. 18A shows plots of temperature over time (top) and the fluid temperature slope (bottom) generated in a fluid channel during thermocycling with the thermal reservoir setup of FIG. 11B, in accordance with embodiments.

FIG. 18B shows a plot of asymptotic temperature over time generated in a fluid channel with the thermal reservoir setup of FIG. 11B, in accordance with embodiments.

FIG. 19 shows an exemplary cartridge arrangement comprising a sample receiver module, two reagent modules, and an amplification/detection module, in accordance with embodiments.

FIGS. 20A-20C show an exemplary sample reservoir and a plurality of jumper silos, in accordance with embodiments. FIG. 20A shows a perspective view of a sample receiver comprising a sample receiver silo with a scraper disposed therein. FIG. 20B shows a cross-sectional view of the assembled sample receiver including swab cap disposed on the sample receiver silo to seal the chamber therewithin. FIG. 20C shows a perspective underside view of a swab cap that is configured to hold a sample collecting swab (e.g., a nasopharyngeal swab) and couple to the sample receiver silo after the sample has been collected by the swab.

FIG. 21 shows an exemplary reagent reservoir/capsule, in accordance with embodiments.

FIGS. 22A-22C show cross-sectional views of a jumper in use, in accordance with embodiments. FIG. 22A shows the jumper in an initial closed configuration prior to actuation.

FIG. 22B shows the jumper after actuation to an open configuration which links previously discontinuous fluid pathways together and enables fluid flow there between via the jumper channel. FIG. 22C shows the jumper after further actuation to a final closed configuration which shuts off fluid flow therethrough.

FIG. 23A shows a plan view of an exemplary amplification/detection module, in accordance with embodiments.

FIG. 23B shows a cross-sectional side view of a bubble trap, in accordance with embodiments.

FIGS. 24A-24C show various stages of fluid movement during amplification chamber filling of an exemplary amplification/detection module, in accordance with embodiments.

FIGS. 25A-25B shows an exemplary thermocycling system comprising a plurality of thermal reservoirs and serpentine channels for interzone cycling, in accordance with embodiments.

FIG. 25C shows plots of temperature over time generated in a fluid channel during thermocycling with the thermal reservoir setup of FIGS. 25A-25B, in accordance with embodiments.

FIGS. 26A-26F show exemplary low mass thermocycling systems, in accordance with embodiments.

FIG. 26G shows top and bottom views of the six low mass thermocycling chip configurations of FIGS. 26A-26F, in accordance with embodiments.

FIG. 27A shows an exemplary photonic thermocycling system, in accordance with embodiments.

FIG. 27B shows plots of temperature over time generated in a fluid channel during thermocycling with the photonic heater setup of FIG. 27A, in accordance with embodiments.

FIGS. 28A-28B show an exemplary thermoelectric thermocycling system, in accordance with embodiments.

FIGS. 28C-28D show plots of temperature over time generated in a fluid chamber (FIG. 28A) or at the surface of the heater (FIG. 28B) during thermocycling with the thermoelectric heater setup of FIGS. 28A-28B, in accordance with embodiments.

FIGS. 29A-29E depict exemplary reporters immobilized on a substrate, in accordance with embodiments. Arrows indicate signal (e.g., fluorescence) change when the reporter is cleaved by a programmable nuclease.

FIG. 30 depicts an exemplary workflow of detecting nucleic acids using immobilized reporters and/or guide nucleic acids, in accordance with embodiments.

FIGS. 31A-31B depict images showing exemplary visually-detectable signals resulting from the cleavage of immobilized reporters comprising a fluorophore releasable by a trans-cleavage reaction in an assay having both reporters and guide nucleic acids immobilized on a substrate, in accordance with embodiments. FIG. 31A shows the signals from the reporter immobilized (shown as replicates on the bottom) versus a negative control (shown as replicates on the top) prior to the cleavage reaction commencing (i.e., the trans-cleavage of the reporter by the programmable nuclease being activated by the binding of the guide nucleic acid to the target nucleic acid). FIG. 31B shows the signals twenty minutes subsequent to the cleavage reaction commencing. As can be seen from FIG. 31B, cleavage of the reporter decreased the detectable signal as expected, thereby indicating the presence of the target nucleic acid.

FIG. 32 depicts an image of an exemplary substrate immobilized with guide nucleic acids and reporters, in accordance with embodiments. Each spot represents a discrete detection location on the substrate immobilized with at least one guide nucleic acid and at least one reporter.

FIG. 33 depicts the average percentage increase of the detectable signal resulting from the cleavage of a reporter immobilized at a discrete detection location on a substrate versus the negative control using the workflow shown in FIG. 30, in accordance with embodiments. A target-complementary guide nucleic acid and reporter rep203 (comprising a double-stranded region, a fluorophore, a cleavable single-stranded region, and a releasable quencher as shown in FIG. 29D) were bound to the substrate using NHS-amine chemistry. The Y-axis shows the increase in signal intensity at twenty minutes after the addition of the Cas protein and the target nucleic acid (T20) relative to before they were added (TO). Error bars depict 3 standard deviations (s.d.) of the percentage signal increase. The data represents the average of 10⁻¹² discrete detection locations of each of 30 substrates.

FIGS. 34A-34D depict changes in detectable fluorescent signal resulting from the cleavage of various sets of exemplary improved reporters (rep 136, rep204, and rep200) and 4 different guide nucleic acids (G1-G4) immobilized at a discrete detection locations on a substrate before and 60 minutes subsequent to a trans cleavage reaction commencing, in accordance with embodiments. The amounts of the reporters and guide nucleic acids immobilized are listed on the X-axis. Percentage change (Y-axis) represents the change of the fluorescent signal intensity at 60 minutes after the addition of the Cas protein and the target nucleic acid. Error bars depict 1 s.d. The data represents the average of 4 discrete detection locations for each immobilized reporter and guide nucleic acid. Arrows indicate expected fluorescence change when the reporter is cleaved by a programmable nuclease.

FIG. 34E depicts changes in detectable fluorescent signal resulting from the cleavage reaction of other sets of exemplary reporters (rep136, rep112, and rep135) and guide nucleic acids (G4) immobilized at a discrete detection location on a substrate in various conditions prior to the cleavage reaction has commenced versus 60 minutes subsequent to the cleavage reaction commencing, in accordance with embodiments. The amounts of the reporters and guide nucleic acids immobilized are listed on the X-axis. Percentage change (Y-axis) represents the change of the fluorescent signal intensity at 60 minutes after the addition of the Cas protein and the target nucleic acid. Error bars depict 1 s.d. The data represents the average of 12 discrete detection locations for each immobilized reporter and guide nucleic acid. Arrows indicate expected fluorescence change when the reporter is cleaved by a programmable nuclease.

FIG. 35 depicts average percentage increase of a detectable fluorescent signal on various substrate replicates (X-axis) having an exemplary reporter rep203 and guide nucleic acid immobilized thereon, in accordance with embodiments. A negative control (rep203 without target present) is shown for comparison. The average of all thirty replicates is shown in FIG. 33. Percentage increase (Y-axis) represents the average increase of the signal intensity of the reporters on each substrate at 20 minutes after the addition of the Cas protein and the target nucleic acid (if present). Each substrate contained 10⁻¹² discrete detection locations. Data is summarized in box and whisker plots.

FIG. 36 depicts a percentage decrease of the detectable fluorescent signal on various substrate replicates (X-axis) of another exemplary reporter rep204 and guide nucleic acid immobilized thereon, in accordance with embodiments. A negative control (rep204 without target) and an off-target control (rep204 with off-target guide) are shown for comparison. Percentage increase (Y-axis) represents the average decrease of the signal intensity of the reporters on each substrate at 20 minutes after the addition of the Cas protein and the target nucleic acid (if present). Each substrate contained 10⁻¹² discrete detection locations. The data is summarized in box and whisker plots.

FIG. 37 depicts the percentage decrease (Y-axis) of the detectable signal of an exemplary reporter rep204 over time (X-axis), in accordance with embodiments. Each data point represents the average decrease of signal intensity of the reporter of 20 discrete detection locations after the cleavage reaction started. Error bars depict 1 s.d.

FIG. 38 depicts a substrate comprising a surface with an immobilized programmable nuclease-guide nucleic acid complex and a plurality of reporters, highlighting reporter cleavage by an activated programmable nuclease, in accordance with embodiments.

FIGS. 39A-39B depict representations of illustrative microfluidic devices comprising a plurality of chambers fluidically connected in sequence, in accordance with some embodiments. Each of the illustrated chambers comprises a well, an inlet channel, an outlet, and a capillary valve. The locations of wells, capillary valves, and outlets are labeled. FIGS. 39A and 39B present alternative arrangements of the capillary valves and outlets relative to the respective wells. In FIG. 39A, an outlet defines a volume of the respective chamber that is separate from but fluidically connected to the respective well. In FIG. 39B, an outlet corresponds to an opening of the respective well, which may optionally be sealed by an air-permeable membrane (not shown).

FIGS. 40A-40C depict different views of a microfluidic device in accordance with some embodiments. FIG. 40A shows a perspective view of a top surface of a detection region of the microfluidic device. FIG. 40B shows a perspective cross-sectional view of the top surface of the detection region. FIG. 40C shows a plan view of the top surface of the detection region. The illustrated microfluidic device comprises a plurality of chambers fluidically connected in sequence, in which each chamber of the plurality of chambers comprises a well, an inlet channel, an outlet, and a capillary valve. The arrangement of features in the device and fluid flow within the device is similar to those illustrated in FIG. 39A.

FIG. 41 depicts a perspective view of a bottom surface of the detection region of the microfluidic device shown in FIGS. 40A-40C. Optional features added to the bottom of the device around the outlets to help ultrasonically weld an air-permeable membrane to the device are shown.

FIG. 42 depicts a perspective view of a top surface of a microfluidic device in accordance with some embodiments. The illustrated device is similar to that illustrated in FIGS. 40A-40C, except a film is used instead of a molded lid.

FIG. 43 depicts a perspective cross-sectional view of a microfluidic device in accordance with some embodiments. The illustrated device is similar to that illustrated in FIGS. 40A-40C, except the membrane can be compounded with an adhesive backing to adhere to the device instead of using laser welding or ultrasonic welding or heat staking.

FIGS. 44A-44D depict an exemplary detection region of a microfluidic device in accordance with some embodiments. FIGS. 44A-44B show perspective and plan views of the top of the device, respectively. FIG. 44C-44D show perspective and plan views of the bottom of the device, respectively. The illustrated device includes a plurality of chambers fluidically connected in sequence, and connected to an upstream first inlet channel by way of a bubble purge channel (or a bubble trap) disposed therebetween. In operation, bubbles in the liquid of the first inlet channel enter the bubble purge channel and escape through a hydrophobic or oleophobic membrane covering the bottom surface of the bubble purge channel. The liquid itself is substantially prevented from passing through the membrane and instead continues to flow along the bubble purge channel towards its exit into a second inlet channel, reduced in or substantially free of bubbles. The second inlet channel feeds into the plurality of chambers. Also illustrated is the location of the hydrophobic and/or oleophobic membrane that covers at least a portion of a bottom surface of the device.

FIG. 45 depicts a system in accordance with some embodiments. The illustrated system comprises a microfluidic device in thermal communication with a heater or Peltier via an optional heat spreader (e.g., copper or aluminum). As illustrated, the heat spreader forms a ring shape (corresponding to the annular pattern of the wells shown in FIGS. 40A-44D) that allows for control over the temperature of the wells of the detection region of the microfluidic device, without direct thermal contact with the entire device, such as a central portion of the device that may contain other elements (e.g., membrane sealed outlets and/or a bubble purge channel, as shown in FIGS. 44A-44D).

FIGS. 46A-46C depict an exemplary detection region of a microfluidic device in accordance with some embodiments. FIGS. 46A-46B show views of the top (left) and bottom (right) of the device. FIG. 46C shows a perspective view of a cross section of the device from the top. The illustrated device includes a plurality of chambers arranged in a grid. Subsets of the chambers are fluidically connected in sequence, and the subsets are all connected in parallel to branches of a common inlet channel. Also illustrated is a bubble purge between the common inlet channel and a sample fluid inlet channel.

FIGS. 47A-47D depict a microfluidic device in accordance with some embodiments.

FIGS. 47A-47B show plan and perspective views of the top of the device, respectively. FIG. 47C shows a perspective view of a cross section of the device from the top. FIG. 47D shows a view of the bottom of the device. The illustrated device includes a plurality of chambers arranged in a grid. Subsets of the chambers are fluidically connected in sequence, and the subsets are all connected in parallel to a common inlet channel. Within a subset, each chamber comprises a well, an inlet channel, an outlet, and a capillary valve. The arrangement of the capillary valve provides that fluid flows into an upstream chamber up to a certain level before any substantial amount of fluid flows into the next chamber in the sequence. Upstream chambers and downstream chambers may continue to fill in parallel, but upstream chambers receive fluid first, and are the first to be completely filled. The outlets of the chambers are the bottom openings of the chambers shown in FIG. 47D, and are sealed by a membrane (not shown).

FIG. 48 depicts an exemplary high-level workflow for using a multiplex detection system, in accordance with some embodiments. The illustrated process provides a multiplex method for detecting target nucleic acids from influenza virus A (IVA), influenza virus B (IVB), respiratory syncytial virus (RSV), SARS-COV-2 (SC2), and RNase P (RP).

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or portions of the method or process may be performed in any suitable sequence and are not necessarily limited to any particular disclosed sequence. Various operations may be described as multiple discrete operations in turn, in a manner that may be helpful to understanding certain embodiments, however, the order of the description should not be construed to imply that these operations are order dependent. Additionally, structures, systems, and/or devices described herein may be embodied as integrated components or as separate components.

For the purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages taught herein without necessarily achieving other aspects or advantages as may also be taught or suggested herein.

The present disclosure is described in relation to systems, devices, or methods for in vitro diagnostics, and in particular for detection of an ailment such as a disease, cancer, or genetic disorder. However, one of ordinary skill in the art will appreciate that this is not intended to be limiting and the devices and methods disclosed herein may be used in other nucleic acid testing including, but not limited to, detecting genetic information, such as for phenotyping, genotyping, determining ancestry, or the like.

The systems, devices, and compositions provided herein are described in relation to methods of in vitro diagnostics utilizing programmable nuclease-based detection assays. However, one of ordinary skill in the art will appreciate that this is not intended to be limiting and that the devices, systems, and compositions described herein may be used to perform other methods or assays including, but not limited to, other diagnostic assays such as (RT-)PCR-based molecular diagnostic assays, (RT-)HDA-based detection assays, (RT-)LAMP-based detection assays, probe-based detection assays, antibody-based detection assays, sequencing-based detection assays, antigen detection assays, or the like. It will also be understood by one of ordinary skill in the art that individual elements of the systems and devices described herein (such as the reagent capsules, jumpers, microwells, reporters, etc.) may be applicable to use in other fluidic systems or other assays and the description thereof is not intended to be limiting to any particular configuration or use.

### I. NUCLEIC ACID DETECTION SYSTEM

Described herein are programmable nuclease-based systems designed to match the ease of use and cost of available CLIA-waived systems, while also providing multiplexing capability for syndromic nucleic acid testing. This combination is predicted to change how infectious disease testing is conducted by allowing patients and their health care providers to specifically and rapidly diagnose the cause of the symptoms (i.e., perform syndromic testing) and thereby reduce the time it takes to initiate effective treatment. In at least some instances, an easy to use, syndromic-capable test system may also be used to aid determination of what levels of isolation would be necessary for the affected patient depending on the infection's cause.

The present disclosure provides systems and methods for nucleic acid target detection. The systems and methods of the present disclosure can be implemented using devices that are configured for programmable nuclease-based detection. In some embodiments, the devices can be configured for single reaction detection. In some embodiments, the devices can be configured for multiplexed detection. The target can comprise a target sequence or target nucleic acid. As used herein, a target can be referred to interchangeably as a target nucleic acid. Further, a target can be referred to as a target amplicon or a target nucleic acid amplicon if such target undergoes amplification (e.g., through a thermocycling or isothermal process as described elsewhere herein). The target nucleic acid or amplicon thereof can be a portion of a nucleic acid of interest, e.g., a target nucleic acid from any plant, animal, virus, or microbe of interest. The devices provided herein can be used to perform rapid tests in a single integrated system.

In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter and generation of a signal, directly or indirectly, therefrom. In some embodiments, cleavage of a reporter by a programmable nuclease may release a detection moiety which generates a detectable signal when cleaved from the reporter. In some embodiments, the signal may be an increase in a signal (e.g., an increase in fluorescence upon release of a quencher as described herein), a decrease in signal (e.g., a decrease in fluorescence upon release of a fluorophore as described herein), or any other change in signal (e.g., a color change) as will be understood by one of ordinary skill in the art. In some embodiments, the detection moiety may trigger a downstream signal amplification reaction (e.g., the detection moiety may comprise an enzyme which, upon release from the reporter, can contact its substrate and result in a detectable color change) which can increase the amount of detectable signal generated per reporter cleavage event.

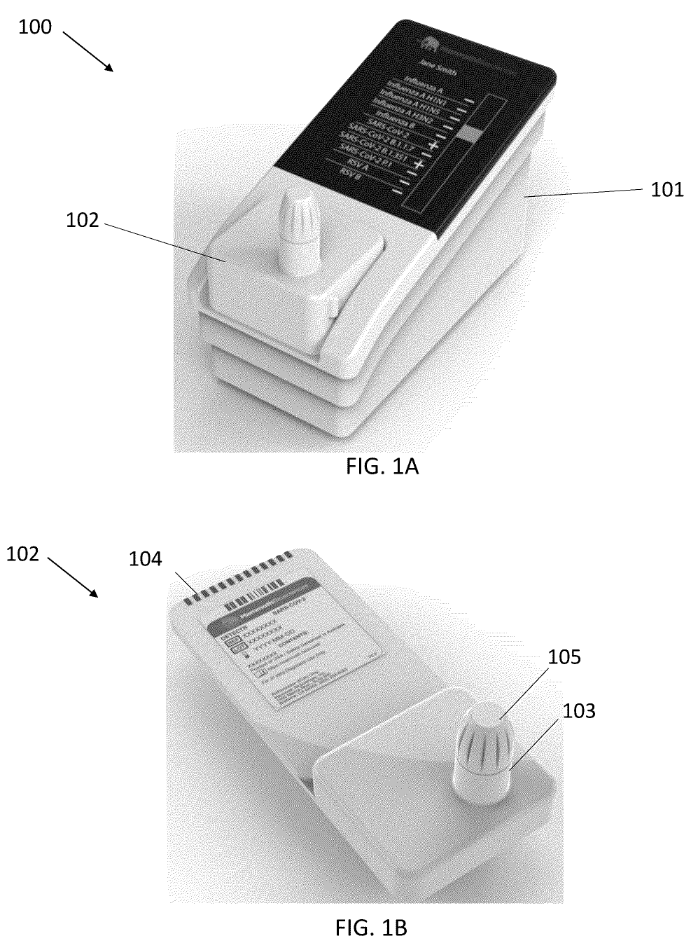

Described herein are various systems, devices, and methods for analysis of one or more different target nucleic acids. FIGS. 1A-1B show an exemplary system 100 for nucleic acid analysis. FIG. 1A shows a perspective view of the system 100 including an instrument 101 for user input, instrument output, and process control with cartridge 102 inserted therein. FIG. 1B shows a perspective view of an exemplary cartridge 102 for use with the system 101. In some embodiments, the system 100 for analysis of target nucleic acids may comprise an instrument 101 and a removable, reusable, or disposable cartridge 102. In some embodiments, the system 100 may be a single integrated unit. In some embodiments, the system 100 may be configured to provide a multiplexed nucleic acid test.

In some embodiments, the instrument 101 may provide for user input and output of results to a user. In some embodiments, the instrument 101 may provide for actuation and/or control of fluid, thermal, mechanical, electrical, optical, pneumatic, ultrasonic, and/or other processes conducted within the cartridge 102. In some embodiments, the cartridge 102 may be configured to receive a sample for testing. In some embodiments, the cartridge 102 may be configured to process the sample in order to detect the presence or absence of one or more target nucleic acids. In most embodiments, the cartridge 102 may be configured to process the sample without directly contacting the sample or any portion thereof to the instrument 101. In some embodiments, the instrument 101 may be configured to provide a user with a result indicating the presence or absence of the one or more target nucleic acids. In some embodiments, the cartridge 102 may be configured to be inserted within the instrument 101. In some embodiments, the cartridge 102 may be removably coupled with the instrument 101. In some embodiments, the cartridge 102 and instrument 101 may form an integral unit.

In some embodiments, the cartridge 102 may comprise a sample interface 103. In some embodiments, the sample interface 103 may be configured to receive the sample and/or sample collector 105 comprising a sample for testing. In some embodiments, the sample may be collected with a swab (e.g., a nasopharyngeal swab) and the swab may be placed directly into the sample interface 103 for processing. In some embodiments, the sample may be liquid and may be directly applied to the sample interface 103 for processing.

In some embodiments, the sample interface 103 may be oriented vertically relative to the rest of the cartridge 102 (as shown). In some embodiments, the sample interface 103 may be oriented horizontally relative to (e.g., within the same plane as) the rest of the cartridge 102. For example, the sample may be collected with a swab and the swab may be inserted vertically or horizontally into the vertical or horizontal sample interface 103, relatively, for processing.

In some embodiments, the cartridge may comprise one or more electrical contacts 104 for interfacing with the instrument. The contacts 104 may facilitate information transfer (e.g., date of manufacture, lifetime, lot, assay identification, and/or any assay parameters) and could carry digital, power, and/or analog signals between the instrument 101 and the cartridge 102. Alternatively, or in combination, transfer of electronic information and/or power may be accomplished through near-field communication (NFC) and/or radio-frequency identification (RFID) electronic components.

In some embodiments, the instrument is approximately 6 inches by 4 inches by 4 inches in size. In some embodiments, the cartridge may be approximately 6 inches by 4 inches by 4 inches in size.

In some embodiments, the instrument may house the electronics, thermal actuators, ultrasonic horn(s)/probe(s)/waveguide(s), optical system(s), and/or mechanical actuators needed to perform the nucleic acid test on the cartridge 102.

In some embodiments, the instrument is re-useable. In some embodiments, the instrument 101 may be single-use.

In some embodiments, the cartridge 102 may be re-usable. In some embodiments, the cartridge 102 may be single-use. In some embodiments, the cartridge 102 may be disposable. In some embodiments, the cartridge 102 may be used for one test specimen and may then be disposed at test completion. In some embodiments, the cartridge 102 may be configured to prevent contact between the sample (and/or other fluids contained therein) and the external environment (e.g., the instrument, the lab, etc.).

In some embodiments, the instrument may receive any type of assay cartridge to perform assays. In some embodiments, the user may input the type of assay cartridge into the instrument in order to identify assay protocol required for the particular cartridge in use.

In some embodiments, the system may be configured to perform a nucleic acid test with about 1 to about 100 different target nucleic acids in a single cartridge. In some embodiments, the system may be configured to perform a nucleic acid test for about 5, 10, 15, 20, 25, 30, 35, 40, 45, 50, 55, 60, 65, 70, 75, 80, 85, 90, 95, or 100 target nucleic acids in a single cartridge from a single sample. Preferably, the system may be configured to detect at least 10, 20, 30, 40, 50, or more different target nucleic acids in a single cartridge from a single sample.

In some embodiments, one or more assay reagents or components may be stored in the cartridge. In some embodiments, one or more reagents or components may be in liquid form, in gel form, dried, vitrified, or lyophilized as described herein. In some embodiments, all of the reagents or components may be stored in the cartridge 102. In some embodiments, reagents are provided to execute an assay panel specific to a particular cartridge.

In some embodiments, the instrument 101 and the cartridge 102 may be fluidly independent of one another so as to reduce or prevent contamination of the instrument with the assay reagents or components or the sample itself. In some embodiments, the cartridge 102 may be configured to maintain one or more (e.g., all) of the assay reagents or components therewithin without contacting them to the instrument at any point during the nucleic acid test. The instrument 101 may be configured to actuate fluid movement within the cartridge 102 without directly contacting the fluid itself as described herein (e.g., using capsules, jumper valves, etc.).

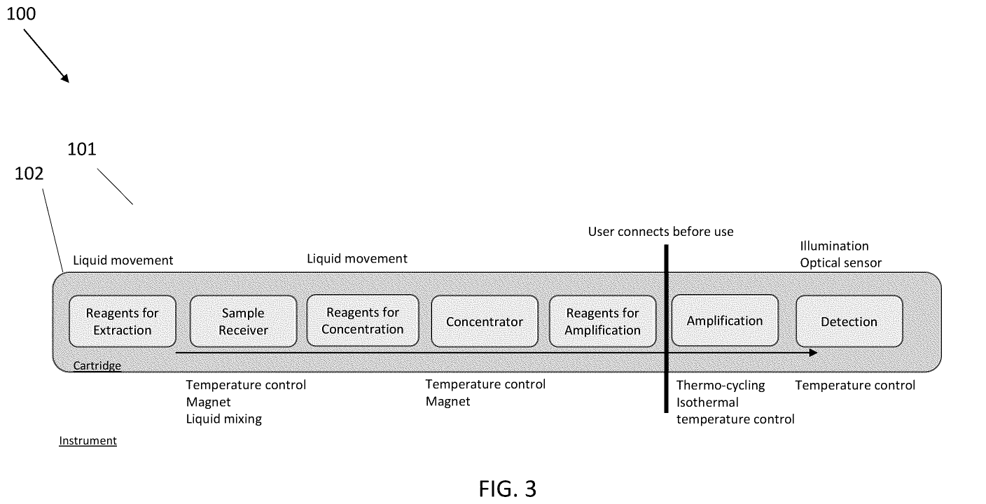

In some embodiments, the cartridge 102 is in one piece as depicted in FIG. 1B. In some embodiments, the cartridge comprises multiple different cartridge segments which may be connected to one another to form a complete cartridge (e.g., as shown schematically in FIG. 3). For example, the cartridge may comprise a first segment containing the liquid reagents and a second segment containing the amplification and/or detection zones. In some embodiments, the cartridge may comprise two or more cartridge segments. The two or more separate cartridge segments may be connected by the user prior to use or by the instrument, such as when the sample is inserted into the cartridge. In some embodiments, a multi-cartridge segment-based system 100 may comprise liquid assay reagents in a first cartridge segment and lyophilized and/or dried reagents in a second separate cartridge segment. In at least some instances, a multi-cartridge segment-based design permits long-term storage of the cartridge components before use, particularly when different reagents have different ideal storage conditions.

A segment may house one or more module described herein. In some embodiments, each segment may house a different module. In some embodiments, one or more segments may house the same module type (e.g., reagent module). In some embodiments, each segment may house multiple modules and/or types of modules. For example, a system can include a first segment including reagent storage, sample receiver, nucleic acid purification, and/or concentration modules and a second segment including amplification (e.g., thermocycling) and/or detection (e.g., array) modules. In some embodiments, the amplification/detection segment might be provided as one or two segments.

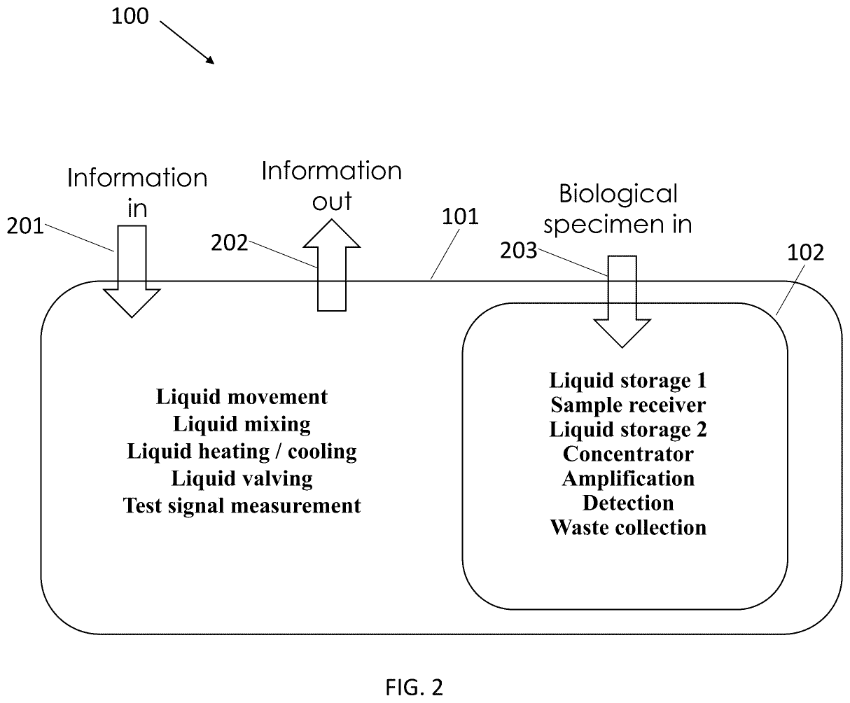

FIG. 2 shows a schematic diagram highlighting exemplary functions of a system 100 for nucleic acid analysis. In some embodiments, the instrument accepts user input 201 (e.g., patient info, sample type, assay type, cartridge type, etc.) and returns results 202 (e.g., outputs) to the user, as depicted in FIG. 2. In some embodiments, the user inputs a biological sample, patient personal information or code to identify the patient uniquely, and/or the test to be performed. In some embodiments, results returned to the user can include a status indicator to show progress/completion of tests, preliminary test results for the targets to be assayed, any error or fault information that is chosen to be exposed to the user, etc., In some embodiments, the instrument is involved in liquid movement, liquid mixing, liquid heating, liquid cooling, liquid valving, and/or test signal measurement. In some embodiments, the cartridge receives a sample 203 (e.g., a biological specimen) comprising a one or more nucleic acids. In some embodiments, the cartridge is responsible for liquid storage, receiving the sample, lysing the sample, concentrating the sample or a portion thereof (e.g., the one or more nucleic acids), amplifying the one or more nucleic acids, and/or detecting a target nucleic acid of the one or more nucleic acids (including amplicons thereof when amplification is performed), if present, as described herein.

In some embodiments, the cartridge may comprise a window. In some embodiments, the window may permit transmission of electromagnetic radiation (e.g., light) from a source within the instrument in order to irradiate a pre-determined location within the cartridge. In some embodiments, the window may permit transmission of ultrasound energy from a source within the instrument in order to vibrate and/or heat a pre-determined location within the cartridge. In some embodiments, the pre-determined location may comprise a sample preparation (e.g., lysis) region, zone, chamber, or channel, an amplification region, zone, chamber, or channel, and/or a detection region, zone, chamber, or channel. In some embodiments, the window allows for detection of reflected, emitted, refracted, polarized, scattered, diffracted, transmitted, absorbed, or otherwise sample-interacted electromagnetic radiation and/or ultrasonic energy from a sample in the cartridge to a sensor of the instrument. In some embodiments, the cartridge may be configured to accept and provide light and/or sound to/from the instrument. In some embodiments, the cartridge may comprise an optically transparent or semi-transparent window, an acoustically transparent or semi-transparent window, an opening in the cartridge that slides open, an imaging waveguide (e.g., optical or acoustic), and/or nonimaging waveguides (e.g., light pipes). In some embodiments, nonimaging waveguides may be used in cases where low number of target multiplexing is required.

In some embodiments, the cartridge may comprise one or more detection or sensing electrodes. One or more detection electrodes may be disposed within the detection region, zone, chamber, or channel. The one or more detections electrodes may be configured for electronic detection, including, but not limited to, amperometry, voltammetry, capacitance, or impedance. In some embodiments, the one or more detection electrodes may be configured for electrochemical detection. In some embodiments, the detection electrodes may comprise a working electrode, a counter electrode, and/or a reference electrode. In some embodiments, a surface (e.g., a bottom surface or portion thereof) of the detection region, zone, chamber, or channel may function as an electrode. The electrode may comprise carbon, graphene, silver, gold, platinum, boron-doped diamond, copper, bismuth, titanium, antimony, chromium, nickel, tin, aluminum, molybdenum, lead, tantalum, tungsten, steel, carbon steel, cobalt, indium tin oxide (ITO), ruthenium oxide, palladium, silver-coated copper, carbon nano-tubes, or other metals. In some embodiments, one or more capture molecules (e.g., an antibody, streptavidin, biotin, etc.) may be coupled to the detection electrode(s) and may be configured to capture the detection moiety released by the cleaved reporter, thereby resulting in a detectable signal at the detection electrode. In some embodiments, one or more nucleic acids may be coupled to the detection electrode(s). For example, reporters and/or guide nucleic acids may be coupled to the detection electrode(s). Release of a detection moiety from an electrode-bound reporter may, for example, result in a change in signal at the detection electrode (e.g., an increase or decrease in current, an increase in intensity of a potentiometric signal, etc.).

### II. PROGRAMMABLE NUCLEASE-BASED ASSAYS AND REAGENTS

### A. Reagents

### Target Nucleic Acids

The present disclosure provides systems and methods for target nucleic acid detection. The systems and methods of the present disclosure can be implemented using devices that are configured for programmable nuclease-based detection. In some embodiments, the devices can be configured for single reaction detection. In some embodiments, the devices can be disposable devices. The devices disclosed herein can be particularly well-suited for carrying out highly efficient, rapid, and accurate reactions for detecting whether a target is present in a sample. The target can comprise a target sequence or target nucleic acid. As used herein, a target can be referred to interchangeably as a target nucleic acid. Further, a target can be referred to as a target amplicon or a target nucleic acid amplicon if such target undergoes amplification (e.g., through a thermocycling process as described elsewhere herein). The target nucleic acid can be a portion of a nucleic acid of interest, e.g., a target nucleic acid from any plant, animal, virus, or microbe of interest. The devices provided herein can be used to perform rapid tests in a single integrated system.

The target nucleic acid can be a nucleic acid or a portion of a nucleic acid from a pathogen, virus, bacterium, fungi, protozoa, worm, or other agent(s) or organism(s) responsible for and/or related to a disease or condition in living organisms (e.g., humans, animals, plants, crops, and the like). The target nucleic acid can be a nucleic acid, or a portion thereof. The target nucleic acid can be a portion of a nucleic acid from a gene expressed in a cancer or genetic disorder in the sample. The target nucleic acid can be a portion of an RNA or DNA from any organism in the sample. The sample can be used for identifying a disease status or condition. For example, a sample is any sample described herein, and is obtained from a subject for use in identifying a disease status of a subject. Sometimes, a method comprises obtaining a serum sample from a subject and identifying a disease status or condition of the subject. Sometimes, a method comprises obtaining a nasal swab from a subject and identifying a disease status or condition of the subject. In certain embodiments, the sample comprises a target nucleic acid. In certain embodiments, the sample comprises a plurality of target nucleic acids.

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid may be a RNA, DNA, synthetic nucleic acids, or nucleic acids found in biological or environmental samples. The target nucleic acids include but are not limited to mRNA, IRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid population. In some cases, the sample has at least two copies of the target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acid copies. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

The systems and methods of the present disclosure can be used to detect one or more target sequences or nucleic acids in one or more samples. The one or more samples can comprise one or more target sequences or nucleic acids for detection of an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, any combination thereof. A sample can be an aspirate of a bodily fluid from an animal (e.g., human, animals, livestock, pet, etc.) or plant. A tissue sample can be from any tissue that can be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water, or soil. A sample from an environment can be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system. In some cases, the sample is contained in no more than about 200 nanoliters (nL). In some cases, the sample is contained in about 200 nL. In some cases, the sample is contained in a volume that is greater than about 200 nL and less than about 20 microliters (μL). In some cases, the sample is contained in no more than 20 μl. In some cases, the sample is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl. In some cases, the sample is contained in from 1 μL to 500 μL, from 10 μL to 500 μL, from 50 μL to 500 μL, from 100 μL to 500 μL, from 200 μL to 500 μL, from 300 μL to 500 μL, from 400 μL to 500 μL, from 1 μL to 200 μL, from 10 μL to 200 μL, from 50 μL to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL. Sometimes, the sample is contained in more than 500 μl.

In some instances, the sample is taken from a single-cell eukaryotic organism; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample may comprise nucleic acids from a cell lysate from a eukaryotic cell, a mammalian cell, a human cell, a prokaryotic cell, or a plant cell. In some cases, the sample may comprise nucleic acids expressed from a cell.

The sample used for disease testing can comprise at least one target sequence that can bind to a guide nucleic acid of the reagents described herein. In some cases, the target sequence is a portion of a nucleic acid. A nucleic acid can be from a genomic locus, a transcribed mRNA, or a reverse transcribed cDNA. A nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A nucleic acid can be from 10 to 90, from 20 to 80, from 30 to 70, or from 40 to 60 nucleotides in length. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-COV-2 (i.e., a virus that causes COVID-19), SARS, MERS, influenza, Adenovirus, Coronavirus HKUI, Coronavirus NL63, Coronavirus 229E, Coronavirus OC43, Severe Acute Respiratory Syndrome Coronavirus 2 (SARS-COV-2), Human Metapneumovirus (hMPV), Human Rhinovirus/Enterovirus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus) and respiratory bacteria (e.g. Bordetella parapertussis, Bordetella pertussis, Chlamydia pneumoniae, Mycoplasma pneumoniae). Other viruses include human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, Chlamydia pneumoniae, Chlamydia psittaci, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Bacillus anthracis, Bortadella pertussis, Burkholderia cepacia, Corynebacterium diphtheriae, Coxiella burnetii, Streptococcus agalactiae, methicillin-resistant Staphylococcus aureus, Legionella longbeachae, Legionella pneumophila, Leptospira interrogans, Moraxella catarrhalis, Streptococcus pyogenes, Escherichia coli, Neisseria gonorrhoeae, Neisseria meningitidis, Neisseria elongate, Neisseria gonorrhoeae, Parechovirus, Pneumococcus, Pneumocystis jirovecii, Cryptococcus neoformans, Histoplasma capsulatum, Haemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid may comprise a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Staphylococcus epidermidis, Legionella pneumophila, Streptococcus pyogenes, Streptococcus salivarius, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

The sample used for cancer testing or cancer risk testing can comprise at least one target sequence or target nucleic acid segment that can bind to a guide nucleic acid of the reagents described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RBI, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for genetic disorder testing can comprise at least one target sequence or target nucleic acid segment that can bind to a guide nucleic acid of the reagents described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLREIC, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGAI, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLNI, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRKI, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPTI, PROPI, PRPSI, PSAP, PTS, PUS1, PYGM, RAB23, RAG2, RAPSN, RARS2, RDH12, RMRP, RPE65, RPGRIPIL, RSI, RTELI, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCALI, SMPDI, STAR, SUMFI, TAT, TCIRGI, TECPR2, TFR2, TGM1, TH, TMEM216, TPPI, TRMU, TSFM, TTPA, TYMP, USHIC, USH2A, VPS13A, VPS13B, VPS45, VRKI, VSX2, WNT10A, XPA, XPC, and ZFYVE26.

The sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

The sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

The sample used for ancestral testing can comprise at least one target nucleic acid segment that can bind to a guide nucleic acid of the reagents described herein. The target nucleic acid segment, in some cases, is a portion of a nucleic acid from a gene associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method may comprise obtaining a serum sample from a subject; and identifying a disease status of the subject. Often, the disease status is prostate disease status. In any of the embodiments described herein, the device can be configured for asymptomatic, pre-symptomatic, and/or symptomatic diagnostic applications, irrespective of immunity. In any of the embodiments described herein, the device can be configured to perform one or more serological assays on a sample (e.g., a sample comprising blood).

In some embodiments, the sample can be used to identify a mutation in a target nucleic acid of a plant or of a bacteria, virus, or microbe associated with a plant or soil. The devices and methods of the present disclosure can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation

In some instances, the target nucleic acid is a single stranded nucleic acid.

Alternatively, or in combination, the target nucleic acid is a double stranded nucleic acid and is prepared into single stranded nucleic acids before or upon contacting the reagents. The target nucleic acid can be a RNA, DNA, synthetic nucleic acids, or nucleic acids found in biological or environmental samples. The target nucleic acids include but are not limited to mRNA, IRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the systems and methods disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250 to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the systems and methods disclosed herein. Some methods described herein can be implemented to detect two or more target nucleic acid populations present in the sample in various concentrations or amounts. In some cases, the sample has at least 2 different target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the methods of the present disclosure can be implemented to detect target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

In some embodiments, the target nucleic acid is indicative of a respiratory disorder or respiratory pathogen. In some embodiments, the respiratory disorder or respiratory pathogen selected from the group consisting of SARS-COV-2 and corresponding variants, 29E, NL63, OC43, HKUI, MERS-COV, (MERS), SARS-COV (SARS), Flu A, Flu B, RSV, Rhinovirus, Strep A, and TB. In some embodiments, the device is configured to differentiate between a viral infection and a bacterial infection. In some embodiments, the target nucleic acid is indicative of a sexually transmitted infection (STI) or infection related to a woman's health. In some embodiments, the STI or infection related to a woman's health is selected from the group consisting of CT, NG, MG, TV, HPV, Candida, B. Vaginosis Syphilis and UTI. In some embodiments, the target nucleic acid comprises a single nucleotide polymorphism (SNP). In some embodiments, the SNP is indicative of NASH disorder or Alpha-1 disorder. In some embodiments, the target nucleic acid is a blood borne pathogen selected from the group consisted of HIV, HBV, HCV and Zika. In some embodiments, the target nucleic acid is indicative of H. Pylori, C. Difficile, Norovirus, HSV and Meningitis.

### Programmable Nucleases

Disclosed herein are programmable nucleases and uses thereof, e.g., detection and editing of target nucleic acids. In some cases, a programmable nuclease is capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and can non-specifically degrade a non-target nucleic acid in its environment. The programmable nuclease has trans cleavage activity once activated. A programmable nuclease can be a Cas protein (also referred to, interchangeably, as a Cas nuclease or Cas effector protein). A guide nucleic acid (e.g., crRNA) and Cas protein can form a CRISPR enzyme (also referred to herein as a programmable nuclease complex or probe).

In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter. The programmable nuclease can be referred to as an RNA-activated programmable RNA nuclease. In some instances, the programmable nuclease as disclosed herein can bind to a target DNA to initiate trans cleavage of an RNA reporter. Such a programmable nuclease can be referred to herein as a DNA-activated programmable RNA nuclease. In some cases, a programmable nuclease as described herein can be activated by a target RNA or a target DNA. For example, a programmable nuclease, e.g., a Cas enzyme, can be activated by a target RNA nucleic acid or a target DNA nucleic acid to cleave RNA reporters. In some embodiments, the programmable nuclease can bind to a target ssDNA which initiates trans cleavage of RNA reporters. In some instances, a programmable nuclease as disclosed herein can bind to a target DNA to initiate trans cleavage of a DNA reporter, and this programmable nuclease can be referred to as a DNA-activated programmable DNA nuclease.

The nucleic acids described and referred to herein can comprise a plurality of base pairs. A base pair can be a biological unit comprising two nucleobases bound to each other by hydrogen bonds. Nucleobases can comprise adenine, guanine, cytosine, thymine, and/or uracil. In some cases, the nucleic acids described and referred to herein can comprise different base pairs. In some cases, the nucleic acids described and referred to herein can comprise one or more modified base pairs. The one or more modified base pairs can be produced when one or more base pairs undergo a chemical modification leading to new bases. The one or more modified base pairs can be, for example, Hypoxanthine, Inosine, Xanthine, Xanthosine, 7-Methylguanine, 7-Methylguanosine, 5,6-Dihydrouracil, Dihydrouridine, 5-Methylcytosine, 5-Methylcytidine, 5-hydroxymethylcytosine (5hmC), 5-formylcytosine (5fC), or 5-carboxylcytosine (5caC).

The programmable nuclease can become activated after binding of a guide nucleic acid that is complexed with the programmable nuclease with a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of reporter nucleic acids comprising a detection moiety. Once the reporter is cleaved by the activated programmable nuclease, the detection moiety can be released or separated from the reporter and can directly or indirectly generate a detectable signal. The reporter and/or the detection moiety can be immobilized, dried, or otherwise deposited on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

The systems and methods of the present disclosure can be implemented using a device that is compatible with any type of programmable nuclease that is human-engineered or naturally occurring. The programmable nuclease can comprise a nuclease that is capable of being activated when complexed with a guide nucleic acid and a target nucleic acid segment or a portion thereof. A programmable nuclease can become activated when complexed with a guide nucleic acid and a target sequence of a target gene of interest. The programmable nuclease can be activated upon binding of a guide nucleic acid to a target nucleic acid and can exhibit or enable trans cleavage activity once activated. In any instances or embodiments where a CRISPR-based programmable nuclease is described or used, it is recognized herein that any other type of programmable nuclease can be used in addition to or in substitution of such CRISPR-based programmable nuclease.

The systems and methods of the present disclosure can be implemented using a device that is compatible with a plurality of programmable nucleases. The device can comprise a plurality of programmable nuclease probes (also referred to herein as programmable nuclease complexes) comprising the plurality of programmable nucleases and one or more corresponding guide nucleic acids. The plurality of programmable nuclease probes can be the same. Alternatively, the plurality of programmable nuclease probes can be different. For example, the plurality of programmable nuclease probes can comprise different programmable nucleases and/or different guide nucleic acids associated with the programmable nucleases.

As used herein, a programmable nuclease generally refers to any enzyme that can cleave nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpf1. Programmable nucleases can also include, for example, PfAgo and/or NgAgo.

ZFNs can cut genetic material in a sequence-specific matter and can be designed, or programmed, to target specific viral targets. A ZFN is composed of two domains: a DNA-binding zinc-finger protein linked to the Fokl nuclease domain. The DNA-binding zinc-finger protein is fused with the non-specific Fokl cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) can cut genetic material in a sequence-specific matter and can be designed, or programmed, to target specific viral targets. TALENs contain the Fokl nuclease domain at their carboxyl termini and a class of DNA binding domains known as transcription activator-like effectors (TALEs). TALENs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target various nucleic acids.

The programmable nuclease can comprise any type of engineered enzyme. Alternatively, the programmable nuclease can comprise CRISPR enzymes derived from naturally occurring bacteria or phage. A programmable nuclease can be a Cas effector protein (also referred to, interchangeably, as a Cas nuclease). A guide nucleic acid (e.g., a crRNA) and Cas effector protein can form a CRISPR enzyme. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The programmable nuclease can comprise one or more amino acid modifications. The programmable nuclease can be a nuclease derived from a CRISPR-Cas system. The programmable nuclease can be a nuclease derived from recombineering. In some embodiments, the programmable nuclease further comprises a Cas enzyme. In some embodiments, the Cas enzyme is selected from the group consisting of Cas12, Cas13, Cas14, Cas14a, Cas14a1, and CasPhi.

In some cases, the programmable nuclease is Cas13. Sometimes the Cas13 is Cas13a, Cas13b, Cas13c, Cas13d, or Cas13e. In some cases, the programmable nuclease is Mad7 or Mad2. In some cases, the programmable nuclease is Cas12. Sometimes the Cas12 is Cas12a, Cas12b, Cas12c, Cas12d, or Cas12e. In some cases, the programmable nuclease is Csm1, Cas9, C2c4, C2c8, C2c5, C2c10, C2c9, or CasZ. Sometimes, the Csm1 is also called smCms1, miCms1, obCms1, or suCms1. Sometimes Cas13a is also called C2c2. Sometimes CasZ is also called Cas14a, Cas14b, Cas14c, Cas14d, Cas14e, Cas14f, Cas14g, or Cas14h. Sometimes, the programmable nuclease is a type V CRISPR-Cas system. In some cases, the programmable nuclease is a type VI CRISPR-Cas system. Sometimes the programmable nuclease is a type III CRISPR-Cas system. In some cases, the programmable nuclease is from at least one of Leptotrichia shahii (Lsh), Listeria seeligeri (Lse), Leptotrichia buccalis (Lbu), Leptotrichia wadeu (Iwa), Rhodobacter capsulatus (Rca), Herbinix hemicellulosilytica (Hhe), Paludibacter propionicigenes (Ppr), Lachnospiraceae bacterium (Lba), [Eubacterium| rectale (Ere), Listeria newyorkensis (Lny), Clostridium aminophilum (Cam), Prevotella sp. (Psm), Capnocytophaga canimorsus (Cca Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus ((ca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

Disclosed herein are programmable nucleases and uses thereof, e.g., detection and editing of target nucleic acids. In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide nucleic acids of Type V CRISPR/Cas proteins hybridize to ssDNA or dsDNA. However, the trans cleavage activity of Type V CRISPR/Cas protein is typically directed towards ssDNA. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease.

In some instances, the Type V Cas protein is a Casϕ protein. A Casϕ protein can function as an endonuclease that catalyzes cleavage at a specific sequence in a target nucleic acid. A programmable Casϕ nuclease may have a single active site in a RuvC domain that is capable of catalyzing pre-crRNA processing and nicking or cleaving of nucleic acids. This compact catalytic site may render the programmable Casϕ nuclease especially advantageous for genome engineering and new functionalities for genome manipulation.

In some instances, the programmable nuclease is a Type VI Cas protein. In some embodiments, the Type VI Cas protein is a programmable Cas13 nuclease. The general architecture of a Cas13 protein includes an N-terminal domain and two HEPN (higher eukaryotes and prokaryotes nucleotide-binding) domains separated by two helical domains. The HEPN domains each comprise aR—X₄—H motif. Shared features across Cas13 proteins include that upon binding of the crRNA of the guide nucleic acid to a target nucleic acid, the protein undergoes a conformational change to bring together the HEPN domains and form a catalytically active RNase. Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic components. In some instances, the Cas effector is a Cas 13 effector. In some instances, the Cas13 effector is a Cas13a, a Cas13b, a Cas 13c, a Cas 13d, or a Cas 13e effector protein.

In some embodiments, the programmable nuclease comprises a Cas12 protein, wherein the Cas12 enzyme binds and cleaves double stranded DNA and single stranded DNA. In some embodiments, programmable nuclease comprises a Cas13 protein, wherein the Cas13 enzyme binds and cleaves single stranded RNA. In some embodiments, programmable nuclease comprises a Cas14 protein, wherein the Cas14 enzyme binds and cleaves both double stranded DNA and single stranded DNA.

Table 1 provides illustrative amino acid sequences of programmable nucleases having trans-cleavage activity. The programmable nuclease may comprise an amino acid sequence that is at least 65%, at least 70%, at least 75%, at least 80%, at least 85%, at least 90%, at least 95%, at least 98%, at least 99%, or 100% identical to any one of SEQ ID Nos: 1-61 or 81-92. The programmable nuclease may consist of an amino acid sequence that is at least 65%, at least 70%, at least 75%, at least 80%, at least 85%, at least 90%, at least 95%, at least 98%, at least 99%, or 100% identical to any one or SEQ ID Nos: 1-61 or 81-92. The programmable nuclease may comprise at least about 50, at least about 100, at least about 150, at least about 200, at least about 250, at least about 300, at least about 350, at least about 400, at least about 450, at least about 500 consecutive amino acids of any one of SEQ ID Nos: 1-61 or 81-92.

TABLE 1
Amino Acid Sequences of Exemplary Programmable Nucleases

| SEQ ID NO: | Programmable Nuclease Amino Acid Sequence |
|---|---|
| SEQ ID | MADLSQFTHKYQVPKTLRFELIPQGKTLENLSAYGMVADDKQRSENYKKLK |
| NO: 1 | PVIDRIYKYFIEESLKNTNLDWNPLYEAIREYRKEKTTATITNLKEQQDICRRA |
| | IASRFEGKVPDKGDKSVKDFNKKQSKLFKELFGKELFTDSVLEQLPGVSLSD |
| | EDKALLKSFDKFTTYFVGFYDNRKNVFSSDDISTGIPHRLVQENFPKFIDNCD |
| | DYKRLVLVAPELKEKLEKAAEATKIFEDVSLDEIFSIKFYNRLLQQNQIDQFN |
| | QLLGGIAGAPGTPKIQGLNETLNLSMQQDKTLEQKLKSVPHRFSPLYKQILS |
| | DRSSLSFIPESFSCDAEVLLAVQEYLDNLKTEHVIEDLKEVFNRLTTLDLKHIY |
| | VNSTKVTAFSQALFGDWNLCREQLRVYKMSNGNEKITKKALGELESWLKN |
| | SDIAFTELQEALADEALPAKVNLKVQEAISGLNEQMAKSLPKELKIPEEKEEL |
| | KALLDAIQEVYHTLEWFIVSDDVETDTDFYVPLKETLQIIQPIIPLYNKVRNFA |
| | TQKPYSVEKFKLNFANPTLADGWDENKEQQNCAVLFQKGNNYYLGILNPK |
| | NKPDFDNVDTEKQGNCYQKMVYKQFPDFSKMMPKCTTQLKEVKQHFEGK |
| | DSDYILNNKNFIKPLTITREVYDLNNVLYDGKKKFQIDYLRKTKDEDGYYTA |
| | LHTWIDFAKKFVASYKSTSIYDTSTILPPEKYEKLNEFYGALDNLFYQIKFENI |
| | PEEIIDTYVEDGKLFLFQIYNKDFAAGATGAPNLHTIYWKAVFDPENVKDVV |
| | VKLNGQAELFYRPKSNMDVIRHKVGEKLVNRTLKDGSILTDELHKELYLYA |
| | NGSLKKGLSEDAKIILDKNLAVIYDVHHEIVKDRRFTTDKFFFHVPLTLNYKC |
| | DKNPVKFNAEVQEYLKENPDTYVIGIDRGERNLIYAVVIDPKGRIVEQKSEN |
| | VINGFDYHGKLDQREKERVKARQAWTAVGKIKELKQGYLSLVVHEISKMM |
| | VRYQAVVVLENLNVGFKRVRSGIAEKAVYQQFEKMLINKLNYLMFKDAGG |
| | TEPGSVLNAYQLTDRFESFAKMGLQTGFLFYIPAAFTSKIDPATGFVDPFRWG |
| | AIKTLADKREFLSGFESLKFDSTTGNFILHFDVSKNKNFQKKLEGFVPDWDII |
| | IEANKMKTGKGATYIAGKRIEFVRDNNSQGHYEDYLPCNALAETLRQCDIP |
| | YEEGKDILPLILEKNDSKLLHSVFKVVRLTLQMRNSNAETGEDYISSPVEDVS |
| | GSCFDSRMENEKLPKDADANGAYHIALKGMLALERLRKDEKMAISNNDWL |
| | NYIQEKRA* |
| SEQ ID | MAGKKKDKDVINKTLSVRIIRPRYSDDIEKEISDEKAKRKQDGKTGELDRAF |
| NO: 2 | FSELKSRNPDIITNDELFPLFTEIQKNLTEIYNKSISLLYMKLIVEEEGGSTASA |
| | LSAGPYKECKARFNSYISLGLRQKIQSNFRRKELKGFQVSLPTAKSDRFPIPF |
| | CHQVENGKGGFKVYETGDDFIFEVPLIKYTATNKKSTSGKNYTKVQLNNPP |
| | VPMNVPLLLSTMRRRQTKKGMQWNKDEGTNAELRRVMSGEYKVSYAEIIR |
| | RTRFGKHDDWFVNFSIKFKNKTDELNQNVRGGIDIGVSNPLVCAVTNGLDR |
| | YIVANNDIMAFNERAMARRRTLLRKNRFKRSGHGAKNKLEPITVLTEKNERF |
| | RKSILQRWAREVAEFFKRTSASVVNMEDLSGITEREDFFSTKLRTTWNYRLM |
| | QTTIENKLKEYGIAVNYISPKYTSQTCHSCGKRNDYFTFSYRSENNYPPFECK |
| | ECNKVKCNADFNAAKNIALKVVL |
| SEQ ID | MAKNTITKTLKLRIVRPYNSAEVEKIVADEKNNREKIALEKNKDKVKEACS |
| NO: 3 | KHLKVAAYCTTQVERNACLFCKARKLDDKFYQKLRGQFPDAVFWQEISEIF |
| | RQLQKQAAEIYNQSLIELYYEIFIKGKGIANASSVEHYLSDVCYTRAAELFKN |
| | AAIASGLRSKIKSNFRLKELKNMKSGLPTTKSDNFPIPLVKQKGGQYTGFEIS |
| | NHNSDFIIKIPFGRWQVKKEIDKYRPWEKFDFEQVQKSPKPISLLLSTQRRKR |
| | NKGWSKDEGTEAEIKKVMNGDYQTSYIEVKRGSKIGEKSAWMLNLSIDVPK |
| | IDKGVDPSIIGGIDVGVKSPLVCAINNAFSRYSISDNDLFHFNKKMFARRRILL |
| | KKNRHKRAGHGAKNKLKPITILTEKSERFRKKLIERWACEIADFFIKNKVGT |
| | VQMENLESMKRKEDSYFNIRLRGFWPYAEMQNKIEFKLKQYGIEIRKVAPN |
| | NTSKTCSKCGHLNNYFNFEYRKKNKFPHFKCEKCNFKENADYNAALNISNP |
| | KLKSTKEEP |
| SEQ ID | MATLVSFTKQYQVQKTLRFELIPQGKTQANIDAKGFINDDLKRDENYMKVK |
| NO: 4 | GVIDELHKNFIEQTLVNVDYDWRSLATAIKNYRKDRSDTNKKNLEKTQEAA |
| | RKEIIAWFEGKRGNSAFKNNQKSFYGKLFKKELFSEILRSDDLEYDEETQDAI |
| | ACFDKFTTYFVGFHENRKNMYSTEAKSTSVAYRVVNENFSKFLSNCEAFSVL |
| | EAVCPNVLVEAEQELHLHKAFSDLKLSDVFKVEAYNKYLSQTGIDYYNQIIG |
| | GISSAEGVRKIRGVNEVVNNAIQQNDELKVALRNKQFTMVQLFKQILSDRST |
| | LSFVSEQFTSDQEVITVVKQFNDDIVNNKVLAVVKTLFENFNSYDLEKIYINS |
| | KELASVSNALLKDWSKIRNAVLENKIIELGANPPKTKISAVEKEVKNKDFSIA |
| | ELASYNDKYLDKEGNDKEICSIANVVLEAVGALEIMLAESLPADLKTLENKN |
| | KVKGILDAYENLLHLLNYFKVSAVNDVDLAFYGAFEKVYVDISGVMPLYNK |
| | VRNYATKKPYSVEKFKLNFAMPTLADGWDKNKERDNGSIILLKDGQYYLG |
| | VMNPQNKPVIDNAVCNDAKGYQKMVYKMFPEISKMVTKCSTQLNAVKAH |
| | FEDNTNDFVLDDTDKFISDLTITKEIYDLNNVLYDGKKKFQIDYLRNTGDFA |
| | GYHKALETWIDFVKEFLSKYRSTAIYDLTTLLPTNYYEKLDVFYSDVNNLCY |
| | KIDYENISVEQVNEWVEEGNLYLFKIYNKDFATGSTGKPNLHTMYWNAVFA |
| | EENLHDVVVKLNGGAELFYRPKSNMPKVEHRVGEKLVNRKNVNGEPIADS |
| | VHKEIYAYANGKISKSELSENAQEELPLAIIKDVKHNITKDKRYLSDKYFFHV |
| | PITLNYKANGNPSAFNTKVQAFLKNNPDVNIIGIDRGERNLLYVVVIDQQGNI |
| | IDKKQVSYNKVNGYDYYEKLNQREKERIEARQSWGAVGKIKELKEGYLSLV |
| | VREIADMMVKYNAIVVMENLNAGFKRVRGGIAEKAVYQKFEKMLIDKLNY |
| | LVFKDVEAKEAGGVLNAYQLTDKFDSFEKMGNQSGFLFYVPAAYTSKIDPV |
| | TGFANVFSTKHITNTEAKKEFICSFNSLRYDEAKDKFVLECDLNKFKIVANSH |
| | IKNWKFIIGGKRIVYNSKNKTYMEKYPCEDLKATLNASGIDFSSSEIINLLKN |
| | VPANREYGKLFDETYWAIMNTLQMRNSNALTGEDYIISAVADDNEKVFDSRT |
| | CGAELPKDADANGAYHIALKGLYLLQRIDISEEGEKVDLSIKNEEWFKFVQQ |
| | KEYAR* |
| SEQ ID | MCMKITKIDGISHKKYKEKGKLIKNNDTAKDIIEERFNDIEKKTKELFQKTLD |
| NO: 5 | FYVKNYEKCKEQNKERREKAKNYFSKVKILVDNKKITICNENTEKMEIEDF |
| | NEYDVRSGKYFNVLNKILNGENYTEEDLEVFENDLQKRTGRIKSIKNSLEEN |
| | KAHFKKESINNNIIYDRVKGNNKKSLFYEYYRISSKHQEYVNNIFEAFDKLYS |
| | NSHEAMNNLFSEITKDSKDRNIRKIREAYHEILNKNKTEFGEELYKKIQDNRN |
| | NFDKLLEIEPEIKELTKSQIFYKYYIDKVNLDETSIKHCFCHLVEIEVNQLLKN |
| | YVYSKRNINKEKLENIFEYCKLKNLIKNKLVNKLNNYIRNCGKYNAYISNND |
| | VVVNSEKISEIRTKEAFLRSIIGVSSSAYFSLRNILNTDNTQDITNKVDKEVDK |
| | LYQENKKIELEERLKLFFGNYFDINNQQEIKVFLMNIDKIISSIRHEIIHFKMET |
| | NAQNIFDENNVNLGNTAKNIFSNEINEEKIKFKIFKQLNSANVFDYLSNKDIT |
| | EYMDKVVFSFTNRNVSFVPSFTKIYNRVQDLANSLEIKKWKIPDKSEGKDA |
| | QIYLLKNIYYGKFLDEFLNEENGIFISIKDKIIELNRNQNKRTGFYKLEKFEKIE |
| | ETNPKKYLEIIQSLYMINIEEIDSEGKNIFLDFIQKIFLKGFFEFIKNNYNYLLEL |
| | KKIQDKKNIFDSEMSEYIAGEKTLEDIGEINEIIQDIKITEIDKILNQTDKINCFY |
| | LLLKLLNYKEITELKGNLEKYQILSKTNVYEKELMLLNIVNLDNNKVKIENF |
| | KILAEEIGKFIEKINIEEINKNKKIKTFEELRNFEKGENTGEYYNIYSDDKNIKN |
| | IRNLYNIKKYGMLDLLEKISEKTNYCIKKKDLEEYSELRKQLEDEKTNFYKIQ |
| | EYLHSKYQQKPKKILLKNNKNDYEKYKKSIENIEKYVHLKNKIEFNELNLLQ |
| | SLLLKILHRLVGFTSIWERDLRFRLIGEFPDELDVEDIFDHRKRYKGTGKGIC |
| | KKYDRFINTHTEYKNNNKMENVKFADNNPVRNYIAHFNYLPNPKYSILKM |
| | MEKLRKLLDYDRKLKNAVMKSIKDILEEYGFKAEFIINSDKEIILNLVKSVEII |
| | HLGKEDLKSRRNSEDLCKLVKAMLEYSK* |
| SEQ ID | MEDKQFLERYKEFIGLNSLSKTLRNSLIPVGSTLKHIQEYGILEEDSLRAQKR |
| NO: 6 | EELKGIMDDYYRNYIEMHLRDVHDIDWNELFEALTEVKKNQTDDAKKRLE |
| | KIQEKKRKEIYQYLSDDAVFSEMFKEKMISGILPDFIRCNEGYSEEEKEEKLK |
| | TVALFHRFTSSFNDFFLNRKNVFTKEAIVTAIGYRVVHENAEIFLENMVAFQN |
| | IQKSAESQISIIERKNEHYFMEWKLSHIFTADYYMMLMTQKAIEHYNEMCG |
| | VVNQQMREYCQKEKKNWNLYRMKRLHKQILSNASTSFKIPEKYENDAEVY |
| | ESVNSFLQNVMEKTVMERIAVLKNSTDNFDLSKIYITAPYYEKISNYLCGSW |
| | NTITDCLTHYYEQQIAGKGARKDQKVKAAVKADKWKSLSEIEQLLKEYARA |
| | EEVKRKPEEYIAEIENIVSLKEAHLLEYHPEVNLIENEKYATEIKDVLDNYME |
| | LFHWMKWFYIEEAVEKEVNFYGELDDLYEEIKDIVPLYNKVRNYVTQKPYS |
| | DTKIKLNFGTPTLANGWSKSKEYDYNAILLQKDGKYYMGIFNPIQKPEKEIIE |
| | GHSQPLEGNEYKKMVYYYLPSANKMLPKVLLSKKGMEIYQPSEYIINGYKE |
| | RRHIKSEEKFDLQFCHDLIDYFKSGIERNSDWKVFGFDFSDTDTYQDISGFYR |
| | EVEDQGYKIDWTYIKEADIDRLNEEGKLYLFQIYNKDFSEKSTGRENLHTMY |
| | LKNLFSEENVREQVLKLNGEAEIFFRKSSVKKPIIHKKGTMLVNRTYMEEVN |
| | GNSVRRNIPEKEYQEIYNYKNHRLKGELSTEAKKYLEKAVCHETKKDIVKD |
| | YRYSVDKFFIHLPITINYRASGKETLNSVAQRYIAHQNDMHVIGIDRGERNLI |
| | YVSVINMQGEIKEQKSFNIINEFNYKEKLKEREQSRGAARRNWKEIGQIKDL |
| | KEGYLSGVIHEIAKMMIKYHAIIAMEDLNYGFKRGRFKVERQVYQKFENML |
| | IQKLNYLVFKDRPADEDGGVLRGYQLAYIPDSVKKMGRQCGMIFYVPAAFT |
| | SKIDPTTGFVDIFKHKVYTTEQAKREFILSFDEICYDVERQLFRFTFDYANFVT |
| | QNVTLARNNWTIYTNGTRAQKEFGNGRMRDKEDYNPKDKMVELLESEGIE |
| | FKSGKNLLPALKKVSNAKVFEELQKIVRFTVQLRNSKSEENDVDYDHVISPV |
| | LNEEGNFFDSSKYKNKEEKKESLLPVDADANGAYCIALKGLYIMQAIQKNW |
| | SEEKALSPDVLRLNNNDWFDYIQNKRYR* |
| SEQ ID | MEEKKMSKIEKFIGKYKISKTLRFRAVPVGKTQDNIEKKGILEKDKKRSEDY |
| NO: 7 | EKVKAYLDSLHRDFIENTLKKVKLNELNEYACLFFSGTKDDGDKKKMEKLE |
| | EKMRKTISNEFCNDEMYKKIFSEKILSENNEEDVSDIVSSYKGFFTSLNGYVN |
| | NRKNLYVSDAKPTSIAYRCINENLPKFLRNVECYKKVVQVIPKEQIEYMSNN |
| | LNLSPYRIEDCFNIDFFEFCLSQGGIDLYNTFIGGYSKKDGTKVQGINEIVNLY |
| | NQKNKKDKEKYKLPQFTPLFKQILSDRDTKSFSIEKLENIYEVVELVKKSYS |
| | DEMFDDIETVFSNLNYYDASGIYVKNGPAITHISMNLTKDWATIRNNWNYEY |
| | DEKHSTKKNKNIEKYEDTRNTMYKKIDSFTLEYISRLVGKDIDELVKYFENE |
| | VANFVMDIKKTYSKLTPLFDRCQKENFDISEDEVNDIKGYLDNVKLLESFMK |
| | SFTINGKENNIDYVFYGKFTDDYDKLHEFDHIYNKVRNYITTSRKPYKLDKY |
| | KLYFDNPQLLGGWDINKEKDYRTVMLTKDGKYYFAIIDKGEHPFDNIPKDYF |
| | DNNGYYKKIIYRQIPNAAKYLSSKQIVPQNPPEEVKRILDKKKADSKSLTEEE |
| | KNIFIDYIKSDFLKNYKLLFDKNNNPYFNFAFRESSTYESLNEFFEDVERQAY |
| | SVRYENLPADYIDNLVNEGKIYLFEIYSKDFSEYSKGTNNLHTMYFKALFDN |
| | DNLKNTVFKLSGNAELFIRPASIKKDELVIHPKNQLLQNKNPLNPKKQSIFDY |
| | DLVKDKRFFENQYMLHISIEINKNERDAKKIKNINEMVRKELKDSDDNYIIGI |
| | DRGERNLLYVCVINSAGKIVEQMSLNEIINEYNGIKHTVDYQGLLDKCEKER |
| | NAQRQSWKSIENIKELKDGYISQVVHKLCQLVEKYDAIIAMENLNGGFKRG |
| | RTKFEKQVYQKFENKLINKMEYMADKKRKTTENGGILRGYQLTNGCINNSY |
| | QNGFIFYVPAWLTSKIDPTTGFVDLLKPKYTNVEEAHLWINKFNSITYDKKL |
| | DMFAFNINYSQFPRADIDYRKIWTFYTNGYRIETFRNSEKNNEFDWKEVHLT |
| | SVIKKLLEEYQINYISGKNIIDDLIQIKDKPFWNSFIKYIRLTLQMRNSITGRTD |
| | VDYIISPVINNEGTFYDSRKDLDEITLPQDADANGAYNIARKALWIIEKLKESP |
| | DEELNKVKLAITQREWLEYAQINI* |
| SEQ ID | MEKIKKPSNRNSIPSIIISDYDANKIKEIKVKYLKLARLDKITIQDMEIVDNIVE |
| NO: 8 | FKKILLNGVEHTIIDNQKIEFDNYEITGCIKPSNKRRDGRISQAKYVVTITDKY |
| | LRENEKEKRFKSTERELPNNTLLSRYKQISGFDTLTSKDIYKIKRYIDFKNEM |
| | LFYFQFIEEFFNPLLPKGKNFYDLNIEQNKDKVAKFIVYRLNDDFKNKSLNSY |
| | ITDTCMIINDFKKIQKILSDFRHALAHFDFDFIQKFFDDQLDKNKFDINTISLIE |
| | TLLDQKEEKNYQEKNNYIDDNDILTIFDEKGSKFSKLHNFYTKISQKKPAFN |
| | KLINSFLSQDGVPNEEFKSYLVTKKLDFFEDIHSNKEYKKIYIQHKNLVIKKQ |
| | KEESQEKPDGQKLKNYNDELQKLKDEMNTITKQNSLNRLEVKLRLAFGFIA |
| | NEYNYNFKNFNDEFTNDVKNEQKIKAFKNSSNEKLKEYFESTFIEKRFFHFS |
| | VNFFNKKTKKEETKQKNIFNSIENETLEELVKESPLLQIITLLYLFIPRELQGEF |
| | VGFILKIYHHTKNITSDTKEDEISIEDAQNSFSLKFKILAKNLRGLQLFHYSLS |
| | HNTLYNNKQCFFYEKGNRWQSVYKSFQISHNQDEFDIHLVIPVIKYYINLNK |
| | LMGDFEIYALLKYADKNSITVKLSDITSRDDLKYNGHYNFATLLFKTFGIDTN |
| | YKQNKVSIQNIKKTRNNLAHQNIENMLKAFENSEIFAQREEIVNYLQTEHRM |
| | QEVLHYNPINDFTMKTVQYLKSLSVHSQKEGKIADIHKKESLVPNDYYLIYK |
| | LKAIELLKQKVIEVIGESEDEKKIKNAIAKEEQIKKGNN |
| SEQ ID | MENYGGFTGLYPLQKTLKFELRPQGRTMEHLVSSNFFEEDRDRAEKYKIVK |
| NO: 9 | KVIDNYHKDFINECLSKRSFDWTPLMKTSEKYYASKEKNGKKKQDLDQKIIP |
| | TIENLSEKDRKELELEQKRMRKEIVSVFKEDKRFKYLFSEKLFSILLKDEDYS |
| | KEKLTEKEILALKSFNKFSGYFIGLHKNRANFYSEGDESTAIAYRIVNENFPKF |
| | LSNLKKYREVCEKYPEIIQDAEQSLAGLNIKMDDIFPMENFNKVMTQDGIDL |
| | YNLAIGGKAQALGEKQKGLNEFLNEVNQSYKKGNDRIRMTPLFKQILSERT |
| | SYSYILDAFDDNSQLITSINGFFTEVEKDKEGNTFDRAVGLIASYMKYDLSRV |
| | YIRKADLNKVSMEIFGSWERLGGLLRIFKSELYGDVNAEKTSKKVDKWLNS |
| | GEFSLSDVINAIAGSKSAETFDEYILKMRVARGEIDNALEKIKCINGNFSEDEN |
| | SKMIIKAILDSVQRLFHLFSSFQVRADFSQDGDFYAEYNEIYEKLFAIVPLYNR |
| | VRNYLTKNNLSMKKIKLNFKNPALANGWDLNKEYDNTAVIFLREGKYYLGI |
| | MNPSKKKNIKFEEGSGTGPFYKKMAYKLLPDPNKMLPKVFFAKKNINYYNP |
| | SDEIVKGYKAGKYKKGENFDIDFCHKLIDFFKESIQKNEDWRAFNYLFSATE |
| | SYKDISDFYSEVEDQGYRMYFLNVPVANIDEYVEKGDLFLFQIYNKDFASGA |
| | KGNKDMHTIYWNAAFSDENLRNVVVKLNGEAELFYRDKSIIEPICHKKGEM |
| | LVNRTCFDKTPVPDKIHKELFDYHNGRAKTLSIEAKGYLDRVGVFQASYEII |
| | KDRRYSENKMYFHVPLKLNFKADGKKNLNKMVIEKFLSDKDVHIIGIDRGE |
| | RNLLYYSVIDRRGNIIDQDSLNIIDGFDYQKKLGQREIERREARQSWNSIGKI |
| | KDLKEGYLSKAVHKVSKMVLEYNAIVVLEDLNFGFKRGRFKVEKQVYQKF |
| | EKMLIDKLNYLVFKEVLDSRDAGGVLNAYQLTTQLESFNKLGKQSGILFYVP |
| | AAYTSKIDPTTGFVSLENTSRIESDSEKKDFLSGFDSIVYSAKDGGIFAFKFDY |
| | RNRNFQREKTDHKNIWTVYTNGDRIKYKGRMKGYEITSPTKRIKDVLSSSGI |
| | RYDDGQELRDSIIQSGNKVLINEVYNSFIDTLQMRNSDGEQDYIISPVKNRNG |
| | EFFRTDPDRRELPVDADANGAYHIALRGELLMQKIAEDFDPKSDKFTMPKM |
| | EHKDWFEFMQTRGD* |
| SEQ ID | MEVQKTVMKTLSLRILRPLYSQEIEKEIKEEKERRKQAGGTGELDGGFYKKL |
| NO: 10 | EKKHSEMFSFDRLNLLLNQLQREIAKVYNHAISELYIATIAQGNKSNKHYISS |
| | IVYNRAYGYFYNAYIALGICSKVEANFRSNELLTQQSALPTAKSDNFPIVLHK |
| | QKGAEGEDGGFRISTEGSDLIFEIPIPFYEYNGENRKEPYKWVKKGGQKPVL |
| | KLILSTFRRQRNKGWAKDEGTDAEIRKVTEGKYQVSQIEINRGKKLGEHQK |
| | WFANFSIEQPIYERKPNRSIVGGLDVGIRSPLVCAINNSFSRYSVDSNDVFKFS |
| | KQVFAFRRRLLSKNSLKRKGHGAAHKLEPITEMTEKNDKFRKKIIERWAKE |
| | VTNFFVKNQVGIVQIEDLSTMKDREDHFFNQYLRGFWPYYQMQTLIENKLK |
| | EYGIEVKRVQAKYTSQLCSNPNCRYWNNYFNFEYRKVNKFPKFKCEKCNLE |
| | ISADYNAARNLSTPDIEKFVAKATKGINLPEK* |
| SEQ ID | MIIHNCYIGGSFMKKIDSFTNCYSLSKTLRFKLIPIGATQSNFDLNKMLDEDK |
| NO: 11 | KRAENYSKAKSIIDKYHRFFIDKVLSSVTENKAFDSFLEDVRAYAELYYRSN |
| | KDDSDKASMKTLESKMRKFIALALQSDEGFKDLFGQNLIKKTLPEFLESDTD |
| | KEIIAEFDGFSTYFTGFFNNRKNMYSADDQPTAISYRCINDNLPKFLDNVRTF |
| | KNSDVASILNDNLKILNEDFDGIYGTSAEDVFNVDYFPFVLSQKGIEAYNSIL |
| | GGYTNSDGSKIKGLNEYINLYNQKNENIHRIPKMKQLFKQILSERESVSFIPE |
| | KFDSDDDVLSSINDYYLERDGGKVLSIEKTVEKIEKLFSAVTDYSTDGIFVKN |
| | AAELTAVCSGAFGYWGTVQNAWNNEYDALNGYKETEKYIDKRKKAYKSIE |
| | SFSLADIQKYADVSESSETNAEVTEWLRNEIKEKCNLAVQGYESSKDLISKPY |
| | TESKKLFNNDNAVELIKNALDSVKELENVLRLLLGTGKEESKDENFYGEFLP |
| | CYERICEVDSLYDKVRNYMTQKLYKTDKIKLNFQNPQFLGGWDRNKEADY |
| | SAVLLRRNSLYYIAIMPSGYKRVFEKIPAPKADETVYEKVIYKLLPGPNKMLP |
| | KVFFSKKGIETFNPPKEILEKYELGTHKTGDGFNLDDCHALIDYFKSALDVH |
| | SDWSNFGFRFSDTSTYKNIADFYNEVKNQGYKITFCDVPQSYINELVDEGKL |
| | YLFQLYNKDFSEHSKGTPNLHTLYFKMLFDERNLENVVFKLNGEAEMFYRE |
| | ASISKDDMIVHPKNQPIKNKNEQNSRKQSTFEYDIVKDRRYTVDQFMLHIPIT |
| | LNFTANGGTNINNEVRKALKDCDKNYVIGIDRGERNLLYICVVDSEGRIIEQ |
| | YSLNEIINEYNGNTYSTDYHALLDKKEKERLESRKAWKTVENIKELKEGYIS |
| | QVVHKICELVEKYDAVIVMEDLNLGFKQGRSGKFEKSVYQKFEKMLIDKLN |
| | YFADKKKSPEEIGSVLNAYQLTNAFESFEKMGKQNGFIFYVPAYLTSKIDPTT |
| | GFADLLHPSSKQSKESMRDFVGRFDSITENKTENYFEFELDYNKFPRCNTDY |
| | RKKWTVCTYGSRIKTFRNPEKNSEWDNKTVELTPAFMALFEKYSIDVNGDI |
| | KAQIMSVDKKDFFVELIGLLRLTLQMRNSETGKVDRDYLISPVKNSEGVFYN |
| | SDDYKGIENASLPKDADANGAYNIARKGLWIIEQIKACENDAELNKIRLAISN |
| | AEWLEYAQKK* |
| SEQ ID | MKDYIRKTLSLRILRPYYGEEIEKEIAAAKKKSQAEGGDGALDNKFWDRLK |
| NO: 12 | AEHPEIISSREFYDLLDAIQRETTLYYNRAISKLYHSLIVEREQVSTAKALSAG |
| | PYHEFREKFNAYISLGLREKIQSNFRRKELARYQVALPTAKSDTFPIPIYKGFD |
| | KNGKGGFKVREIENGDFVIDLPLMAYHRVGGKAGREYIELDRPPAVLNVPVI |
| | LSTSRRRANKTWFRDEGTDAEIRRVMAGEYKVSWVEILQRKRFGKPYGGW |
| | YVNFTIKYQPRDYGLDPKVKGGIDIGLSSPLVCAVTNSLARLTIRDNDLVAFN |
| | RKAMARRRTLLRQNRYKRSGHGSANKLKPIEALTEKNELYRKAIMRRWARE |
| | AADFFRQHRAATVNMEDLTGIKDREDYFSQMLRCYWNYSQLQTMLENKLK |
| | EYGIAVKYIEPKDTSKTCHSCGHVNEYFDFNYRSAHKFPMFKCEKCGVECG |
| | ADYNAARNIAQA |
| SEQ ID | MKEQFINRYPLSKTLRFSLIPVGETENNFNKNLLLKKDKQRAENYEKVKCYI |
| NO: 13 | DRFHKEYIESVLSKARIEKVNEYANLYWKSNKDDSDIKAMESLENDMRKQI |
| | SKQLTSTEIYKKRLFGKELICEDLPSFLTDKDERETVECFRSFTTYFKGFNTNR |
| | ENMYSSDGKSTAIAYRCINDNLPRFLDNVKSFQKVFDNLSDETITKLNTDLY |
| | NIFGRNIEDIFSVDYFEFVLTQSGIEIYNSMIGGYTCSDKTKIQGLNECINLYN |
| | QQVAKNEKSKKLPLMKPLYKQILSEKDSVSFIPEKFNSDNEVLHAIDDYYTG |
| | HIGDFDLLTELLQSLNTYNANGIFVKSGVAITDISNGAFNSWNVLRSAWNEK |
| | YEALHPVTSKTKIDKYIEKQDKIYKAIKSFSLFELQSLGNENGNEITDWYISSI |
| | NESNSKIKEAYLQAQKLLNSDYEKSYNKRLYKNEKATELVKNLLDAIKEFQK |
| | LIKPLNGTGKEENKDELFYGKFTSYYDSIADIDRLYDKVRNYITQKPYSKDKI |
| | KLNFDNPQLLGGWDKNKESDYRTVLLHKDGLYYLAVMDKSHSKAFVDAPE |
| | ITSDDKDYYEKMEYKLLPGPNKMLPKVFFASKNIDTFQPSDRILDIRKRESFK |
| | KGATFNKAECHEFIDYFKDSIKKHDDWSQFGFKFSPTESYNDISEFYREISDQ |
| | GYSVRFNKISKNYIDGLVNNGYIYLFQIYNKDFSKYSKGTPNLHTLYFKMLF |
| | DERNLSNVVYKLNGEAEMFYREASIGDKEKITHYANQPIKNKNPDNEKKES |
| | VFEYDIVKDKRFTKRQFSLHLPITINFKAHGQEFLNYDVRKAVKYKDDNYVI |
| | GIDRGERNLIYISVINSNGEIVEQMSLNEIISDNGHKVDYQKLLDTKEKERDK |
| | ARKNWTSVENIKELKEGYISQVVHKICELVIKYDAVIAMEDLNFGFKRGRFP |
| | VEKQVYQKFENMLISKLNLLIDKKAEPTEDGGLLRAYQLTNKFDGVNKAKQ |
| | NGIIFYVPAWDTSKIDPATGFVNLLKPKCNTSVPEAKKLFETIDDIKYNANTD |
| | MFEFYIDYSKFPRCNSDFKKSWTVCTNSSRILTFRNKEKNNKWDNKQIVLTD |
| | EFKSLFNEFGIDYKGNLKDSILSISNADFYRRLIKLLSLTLQMRNSITGSTLPE |
| | DDYLISPVANKSGEFYDSRNYKGTNAALPCDADANGAYNIARKALWAINVL |
| | KDTPDDMLNKAKLSITNAEWLEYTQK* |
| SEQ ID | MKEQFVNQYPISKTLRFSLIPIGKTEENFNKNLLLKEDEKKAEEYQKVKGYI |
| NO: 14 | DRYHKFFIETALCNINFEGFEEYSLLYYKCSKDDNDLKTMEDIEIKLRKQISK |
| | TMTSHKLYKDLFGENMIKTILPNFLDSDEEKNSLEMFRGFYTYFSGFNTNRK |
| | NMYTEEAKSTSIAYRCINDNLPKFLDNSKSFEKIKCALNKEELKAKNEEFYEI |
| | FQIYATDIFNIDFFNFVLTQPGIDKYNGIIGGYTCSDGTKVQGLNEIINLYNQQI |
| | AKDDKSKRLPLLKMLYKQILSDRETVSFIPEKFSSDNEVLESINNYFSKNVSN |
| | AIKSLKELFQGFEAYNMNGIFISSGVAITDLSNAVFGDWNAISTAWEKAYFET |
| | NPPKKNKSQEKYEEELKANYKKIKSFSLDEIQRLGSIAKSPDSIGSVAEYYKIT |
| | VTEKIDNITELYDGSKELLNCNYSESYDKKLIKNDTVIEKVKTLLDAVKSLEK |
| | LIKPLVGTGKEDKDELFYGTFLPLYTSLSAVDRLYDKVRNYATQKPYSKDKI |
| | KLNFNCSSFLSGWATDYSSNGGLIFEKDGLYYLGIVNKKFTTEEIDYLQQNA |
| | DENPAQRIVYDFQKPDNKNTPRLFIRSKGTNYSPSVKEYNLPVEEIVELYDKR |
| | YFTTEYRNKNPELYKASLVKLIDYFKLGFTRHESYRHYDFKWKKSEEYNDIS |
| | EFYKDVEISCYSLKQEKINYNTLLNFVAENRIYLFQIYNKDFSKYSKGTPNLH |
| | TRYFKALFDENNLSDVVFKLNGGSEMFFRKASIKDNEKVVHPANQPIDNKN |
| | PDNSKKQSTFDYELIKDKRFTKHQFSIHIPITMNFKARGRDFINNDIRKAIKSE |
| | YKPYVIGIDRGERNLIYISVINNNGEIVEQMSLNDIISDNGYKVDYQRLLDRK |
| | EKERDNARKSWGTIENIKELKEGYISQVIHKICELVIKYDAVIAMEDLNFGFK |
| | RGRFNVEKQVYQKFENMLISKLNYLCDKKSEANSEGGLLKAYQLTNKFDG |
| | VNKGKQNGIIFYVPAWLTSKIDPVTGFVDLLHPKYISVEETHSLFEKLDDIRY |
| | NFEKDMFEFDIDYSKLPKCNADFKQKWTVCTNADRIMTFRNSEKNNEWDN |
| | KRILLSDEFKRLFEEFGIDYCHNLKNKILSISNKDFCYRFIKLFALTMQMRNSI |
| | TGSTNPEDDYLISPVRDENGVFYDSRNFIGSKAGLPIDADANGAYNIARKGL |
| SEQ ID | WAINAIKSTADDMLDKVDLSISNAKWLEYVQK* |
| NO: 15 | MKITKIDGILHKKYIKEGKLVKSTSEENKTDERLSELLTIRLDTYIKNPDNASE |
| | EENRIRRETLKEFFSNKVLYLKDSILYLKDRREKNQLQNKNYSEEDISEYDLK |
| | NKNSFLVLKKILLNEDINSEELEIFRNDFEKKLDKINSLKYSLEENKANYQKI |
| | NENNIKKVEGKSKRNIFYNYYKDSAKRNDYINNIQEAFDKLYKKEDIENLFF |
| | LIENSKKHEKYKIRECYHKIIGRKNDKENFATIIYEEIQNVNNMKELIEKVPN |
| | VSELKKSQVFYKYYLNKEKLNDENIKYVFCHFVEIEMSKLLKNYVYKKPSN |
| | ISNDKVKRIFEYQSLKKLIENKLLNKLDTYVRNCGKYSFYLQDGEIATSDFIV |
| | GNRQNEAFLRNIIGVSSTAYFSLRNILETENENDITGRIKGKTVKNKKGEEKY |
| | ISGEIDKLYDNNKQNEVKKNLKMFYSYDFNMNRKKEIEDFFSNIDEAISSIRH |
| | GIVHFNLELEGKDIFTFKNIVPSQISKKMFQNEINEKKLKLKIFRQLNSANVFR |
| | YLEKYKILNYLNRTRFEFVNKNIPFVPSFTKLYSRIDDLKNSLCIYWKIPKAN |
| | DNNKTKEITDAQIYLLKNIYYGEFLNYFMSNNGNFFEIIKEIIELNKNDKRNL |
| | KTGFYKLQKFENLQEKTPKEYLANIQSFYMIDAGNKDEEEKDAYIDFIQKIFL |
| | KGFMTYLANNGRLSLMYIGNDEQINTSLAGKKQEFDKFLKKYEQNNNIEIP |
| | HEINEFVREIKLGKILKYTESLNMFYLILKLLNHKELTNLKGSLEKYQSANKE |
| | EAFSDQLELINLLNLDNNRVTEDFELEADEIGKFLDFNGNKVKDNKELKKFD |
| | TNKIYFDGENIIKHRAFYNIKKYGILNLLEKISDEAKYKISIEELKNYSNKKIEI |
| | EKNHTTQENLHRKYARPRKDEKFNDEDYKKYEKTIRNIQQYTHLKNKVEFN |
| | ELNLLQSLLLRILHRLVGYTSIWERDLRFRLKGEFPENQYIEEIFNFDNSKNV |
| | KYKNGQIVEKYISFYKELYKDDMEKISIYSDKKVKELKKEKKDLYIRNYIAH |
| | FNYIPNAEVSLLEVLENLRKLLSYDRKLKNAIMKSIVDILKEYGFVVTFKIEK |
| | DKKIRIESLKSEEVVHLKKLKLKDNDKKKEPIKTYRNSKELCKLVKVMFEY |
| | KMKEKKSEN* |
| SEQ ID | MKITKIDGISHKKYIKEGKLVKSTSEENKTDERLSELLTIRLDTYIKNPDNASE |
| NO: 16 | EENRIRRENLKEFFSNKVLYLKDGILYLKDRREKNQLQNKNYSEEDISEYDL |
| | KNKNSFLVLKKILLNEDINSEELEIFRKDVEAKLNKINSLKYSFEENKANYQK |
| | INENNVEKVGGKSKRNIIYDYYRESAKRNDYINNVQEAFDKLYKKEDIEKLF |
| | FLIENSKKHEKYKIRECYHKIIGRKNDKENFAKIIYEEIQNVNNIKELIEKVPD |
| | MSELKKSQVFYKYYLDKEELNDKNIKYAFCHFVEIEMSQLLKNYVYKRLSN |
| | ISNDKIKRIFEYQNLKKLIENKLLNKLDTYVRNCGKYNYYLQDGEIATSDFIA |
| | GNRQNEAFLRNIIGVSSVAYFSLRNILETENKDDITGKMRGKTRIDSKTGEEK |
| | YIPGEVDQIYYENKQNEVKNKLKMFYGYDFDMDNKKEIEDFFANIDEAISSI |
| | RHGIVHFNLDLDGKDIFAFKNIVPSEISKKMFQNEINEKKLKLKIFRQLNSAN |
| | VFRYLEKYKILNYLKRTRFEFVNKNIPFVPSFTKLYSRIDDLKNSLGIYWKTP |
| | KINDDNKTKEIIDAQIYLLKNIYYGEFLNYFMSNNGNFFEISREIIELNKNDK |
| | RNLKTGFYKLQKFEDIQEKTPKKYLANIQSLYMINAGNQDEEEKDTYIDFIQ |
| | KIFLKGFMTYLANNGRLSLMYIGNDEQINTSLAGKKQEFDKFLKKYEQNNN |
| | IEIPHEINEFLREIKLGKILKYTESLNMFYLILKLLNHKELTNLKGSLEKYQSA |
| | NKEETFSDELELINLLNLDNNRVTEDFELEANEIGKFLDFNGNKIKDRKELK |
| | KFDTKKIYFDGENIIKHRAFYNIKKYGMLNLLEKIADKAKYKISLKELKEYS |
| | NKKNEIEKNYTMQQNLHRKYARPKKDEKFNDEDYKEYEKAIGNIQKYTHL |
| | KNKVEFNELNLLQGLLLKILHRLVGYTSIWERDLRFRLKGEFPENQYIEEIFN |
| | FDNSKNVKYKSGQIVEKYINFYKELYKDNVEKRSIYSDKKVKKLKQEKKDL |
| | YIRNYIAHFNYIPHAEISLLEVLENLRKLLSYDRKLKNAIMKSVVDILKEYGF |
| | VATFKIGADKKIGIQTLESEKIVHLKNLKKKKLMTDRNSEELCKLVKVMFEY |
| | KMEEKNLKTKKCKVI* |
| | MKKIDNFVGCYPVSKTLRFKAIPIGKTQENIEKKRLVEEDEVRAKDYKAVK |
| SEQ ID | KLIDRYHREFIEGVLDNVKLDGLEEYYMLFNKSDREESDNKKIEIMEERFRR |
| NO: 17 | VISKSFKNNEEYKKIFSKKIIEEILPNYIKDEEEKELVKGFKGFYTAFVGYAQN |
| | RENMYSDEKKSTAISYRIVNENMPRFITNIKVFEKAKSILDVDKINEINEYILN |
| | NDYYVDDFFNIDFFNYVLNQKGIDIYNAIIGGIVTGDGRKIQGLNECINLYNQ |
| | ENKKIRLPQFKPLYKQILSESESMSFYIDEIESDDMLIDMLKESLQIDSTINNAI |
| | DDLKVLFNNIFDYDLSGIFINNGLPITTISNDVYGQWSTISDGWNERYDVLSN |
| | AKDKESEKYFEKRRKEYKKVKSFSISDLQELGGKDLSICKKINEIISEMIDDY |
| | KSKIEEIQYLFDIKELEKPLVTDLNKIELIKNSLDGLKRIERYVIPFLGTGKEQ |
| | NRDEVFYGYFIKCIDAIKEIDGVYNKTRNYLTKKPYSKDKFKLYFENPQLMG |
| | GWDRNKESDYRSTLLRKNGKYYVAIIDKSSSNCMMNIEEDENDNYEKINYK |
| | LLPGPNKMLPKVFFSKKNREYFAPSKEIERIYSTGTFKKDTNFVKKDCENLIT |
| | FYKDSLDRHEDWSKSFDFSFKESSAYRDISEFYRDVEKQGYRVSFDLLSSNA |
| | VNTLVEEGKLYLFQLYNKDFSEKSHGIPNLHTMYFRSLFDDNNKGNIRLNG |
| | GAEMFMRRASLNKQDVTVHKANQPIKNKNLLNPKKTTTLPYDVYKDKRFT |
| | EDQYEVHIPITMNKVPNNPYKINHMVREQLVKDDNPYVIGIDRGERNLIYVV |
| | VVDGQGHIVEQLSLNEIINENNGISIRTDYHTLLDAKERERDESRKQWKQIE |
| | NIKELKEGYISQVVHKICELVEKYDAVIALEDLNSGFKNSRVKVEKQVYQKF |
| | EKMLITKLNYMVDKKKDYNKPGGVLNGYQLTTQFESFSKMGTQNGIMFYI |
| | PAWLTSKMDPTTGFVDLLKPKYKNKADAQKFFSQFDSIRYDNQEDAFVFK |
| | VNYTKFPRTDADYNKEWEIYTNGERIRVFRNPKKNNEYDYETVNVSERMK |
| | ELFDSYDLLYDKGELKETICEMEESKFFEELIKLFRLTLQMRNSISGRTDVDY |
| | LISPVKNSNGYFYNSNDYKKEGAKYPKDADANGAYNIARKVLWAIEQFKM |
| | ADEDKLDKTKISIKNQEWLEYAQTHCE |
| SEQ ID | MKKIDSFVNYYPLSKTLRFSLIPVGKTEDNFNAKLLLEEDEKRAIEYEKVKR |
| NO: 18 | YIDRYHKHFIETVLANFHLDDVNEYAELYYKAGKDDKDLKYMEKLEGKMR |
| | KSISAAFTKDKKYKEIFGQEIIKNILPEFLENEDEKESVKMFQGFFTYFTGFND |
| | NRKNMYTHEAQTTAISYRCINENLPKFLDNVQSFAKIKESISSDIMNKLDEVC |
| | MDLYGVYAQDMFCTDYFSFVLSQSGIDRYNNIIGGYVDDKGVKIQGINEYIN |
| | LYNQQVDEKNKRLPLMKKLYKQILIEKESISFIPEKFESDNIVINAISDYYHNN |
| | VENLFDDFNKLFNEFSEYDDNGIFVTSGLAVTDISNAVFGSWNIISDSWNEEY |
| | KDSHPMKKTTNAEKYYEDMKKEYKKNLSFTIAELQRLGEAGCNDECKGDI |
| | KEYYKTTVAEKIENIKNAYEISKDLLASDYEKSNDKKLCKNDSAISLLKNLL |
| | DSIKDLEKTIKPLLGTGKEENKDDVFYGKFTNLYEMISEIDRLYDKVRNYVT |
| | QKPYSKDKIKLNFENPQHLGGWDKNKERDYRSVLLKKEDKYYLAIMDKSN |
| | NKAFIDFPDDGECYEKIEYKLLPGPNKMLPKVFFASSNIEYFAPSKKILEIRSR |
| | ESFKKGDMFNLKDCHEFIDFFKESIKKHEDWSQFGFEFSPTEKYNDISEFYNE |
| | VKIQGYSLKYKNVSKKYIDELIECGQLYLFQIYNKDFSVYAKGNPNLHTMYF |
| | KMLFDERNLANVVYQLNGGAEMFYRKASIKDSEKIVHHANQPIKNKNADN |
| | VKKESVFEYDIIKDKRFTKRQFSIHIPITLNFKAKGQNFINNDVRMALKKADE |
| | NYVIGIDRGERNLLYICVINSKGEIVEQKSLNEIIGDNGYRVDYHKLLDKKEA |
| | ERDEARKSWGTIENIKELKEGYLSQIVHEISKLVIKYDAVIAIEDLNSGFKKGR |
| | FKVEKQVYQKFENMLCTKLNYLVDKNADANECGGLLKAYQLINKEDGAN |
| | RGRQNGIIFSVPAWLTSKIDPVTGFADLLRPKYKSVSESVEFISKIDNIRYNSKE |
| | DYFEFDIDYSKFPNSTASYKKKWTVCTYGERIINVRNKEKNNMWDNKTIVL |
| | TDEFKKLFADFGVDVSKNIKESVLAIDSKDFYYRFINLLANTLQLRNSEVGN |
| | VDVDYLISPVKGVDGSFYDSRLVKEKTLPENADANGAYNIARKALWAIDVL |
| | KQTKDEELKNANLSIKNAEWLEYVQK* |
| SEQ ID | MKNQNTLPSNPTDILKDKPFWAAFFNLARHNVYLTVNHINKLLDLEKLYNK |
| NO: 19 | DKHKEIFEHEDIFNISDDVMNDVNSNGKKRKLDIKKIWANLDTDLTRKYQL |
| | RELILKHFPFIQPAIIGAQTKERTTIDKDKRSTSTSNDSLKPTGEGDINDPLSLS |
| | NVKSIFFRLLQMLEQLRNYYSHVKHSKSATMPNFDEGLLKSMYNIFIDSVNK |
| | VKEDYSSNSVIDPNTSFSHLISKDEQGEIKPCRYSFTSKDGSINASGLLFFVSL |
| | FLEKQDSIWMQKKIPGFKKTSENYMKMTNEVFCRNHILLPKMRLETVYDK |
| | DWMLLDMLNEVVRCPLSLYKRLAPADQNKFKVPEKSSDNANRQEDDNPFS |
| | RILVRHQNRFPYFALRFFDLNEVFTTLRFQINLGCYHFAICKKQIGDKKEVHH |
| | LTRTLYGFSRLQNFTQNTRPEEWNTLVKTTEPSSGNDGKTVQGVPLPYISYTI |
| | PHYQIENEKIGIKIFDGDTAVDTDIWPSVSTEKQLNKPDKYTLTPGFKADVFL |
| | SVHELLPMMFYYQLLLCEGMLKTDAGNAVEKVLIDTRNAIFNLYDAFVQEK |
| | INTITDLENYLQDKPILIGHLPKQMIDLLKGHQRDMLKAVEQKKAMLIKDTE |
| | RRLERLNKQPEQKPNVAAKNTGTLLRNGQIADWLVKDMMRFQPVKRDKEG |
| | NPINCSKANSTEYQMLQRAFAFYTTDSYRLPRYFEQLHLINCDNSHLFLSRFE |
| | YDKQPNLIAFYAAYLEAKLEFLNELQPQNWASDNYFLLLRAPKNDRQKLAE |
| | GWKNGFNLPRGLFTEKIKTWFNEHKTIVDISDCDIFKNRVGQVARLIPVFFD |
| | KKFKDHSQPFYTYNFNVGNVSKITEANYLSKEKRENLFKSYQNKFKNNIPAE |
| | KTKEYREYKNFSSWKKFERELRLIKNQDILTWLMCKNLFDEKIKPKKDILEP |
| | RIAVSYIKLDSLQTNTSTAGSLNALAKVVPMTLAIHIDSPKPKGKAGNNEKE |
| | NKEFTVYIKEEGTKLLKWGNFKTLLADRRIKGLFSYIEHDDINLEKYPLTKY |
| | QVDSELDLYQKYRIDIFKQTLDLEAQLLDKYSDLNTDNFNQMLSGWSEKEG |
| | IPRNIKQDVAFLIGVRNGFSHNQYPDSKRIAFSRIKKFNPKTSSLQESEGLNIA |
| | KQMYEEAQQVVNKIKNIESFD* |
| SEQ ID | MKVTKIDGISHKKFEDEGKLVKFTGHFNIKNEMKERLEKLKELKLSNYIKNP |
| NO: 20 | ENVKNKDKNKEKETKSRRENLKKYFSEIILRKKEEKYLLKKTRKFKNITEEI |
| | NYDDIKKRENQQKIFDVLKELLEQRINENDKEEILNFDSVKLKEAFEEDFIKK |
| | ELKIKAIEESLEKNRADYRKDYVELENEKYEDVKGQNKRSLVFEYYKNPEN |
| | REKFKENIKYAFENLYTEENIKNLYSEIKEIFEKVHLKSKVRYFYQNEIIGESEF |
| | SEKDEEGISILYKQIINSVEKKEKFIEFLQKVKIKDLTRSQIFYKYFLENEELND |
| | ENIKYVFSYFVEIEVNKLLKENVYKTKKFNEGNKYRVKNIFNYDKLKNLVV |
| | YKLENKLNNYVRNCGKYNYHMENGDIATSDINMKNRQTEAFLRSILGVSSF |
| | GYFSLRNILGVNDDDFYKIEKDERKNENFILKKAKEDFTSKNIFEKVVDKSF |
| | EKKGIYQIKENLKMFYGNSFDKVDKDELKKFFVNMLEAITSVRHRIVHYNIN |
| | TNSENIFDFSNIEVSKLLKNIFEKEIDTRELKLKIFRQLNSAGVFDYWESWVIK |
| | KYLENVKFEFVNKNVPFVPSFKKLYDRIDNLKGWNALKLGNNINIPKRKEA |
| | KDSQIYLLKNIYYGEFVEKFVNDNKNFEKIVKEIIEINRGAGTNKKTGFYKLE |
| | KFETLKANTPTKYLEKLQSLHKISYDKEKIEEDKDVYVDFVQKIFLKGFVNY |
| | LKKLDSLKSLNLLNLRKDETITDKKSVHDEKLKLWENSGSNLSKMPEEIYEY |
| | VKKIKISNINYNDRMSIFYLLLKLIDYRELTNLRGNLEKYESMNKNKIYSEEL |
| | TIINLVNLDNNKVRTNFSLEAEDIGKFLKSSITIKNIAQLNNFSKIFADGENVIK |
| | HRSFYNIKKYGILDLLEKIVAKADLKITKEEIKKYENLQNELKRNDFYKIQEQ |
| | IHRNYNQKPFSIKKIENKKDFEKYKKVIEKIQDYTQLKNKIEFNDLNLLQSLIF |
| | RILHRLAGYTSLWERDLQFKLKGEFPEDKYIDEIFNSDGNNNQKYKHGGIAD |
| | KYANFLIEKKEEKSGEILNKKQRKKKIKEDLEIRNYIAHFNYLPNAEKSILEIL |
| | EELRELLKHDRKLKNAVMKSIKDIFREYGFIVEFTISHTKNGKKIKVCSVKSE |
| | KIKHLKNNELITTRNSEDLCELVKIMLEHKELQK* |
| SEQ ID | MKVTKVGGISHKKYTSEGRLVKSESEENRTDERLSALLNMRLDMYIKNPSS |
| NO: 21 | TETKENQKRIGKLKKFFSNKMVYLKDNTLSLKNGKKENIDREYSETDILESD |
| | VRDKKNFAVLKKIYLNENVNSEELEVFRNDIKKKLNKINSLKYSFEKNKAN |
| | YQKINENNIEKVEGKSKRNIIYDYYRESAKRDAYVSNVKEAFDKLYKEEDIA |
| | KLVLEIENLTKLEKYKIREFYHEIIGRKNDKENFAKIIYEEIQNVNNMKELIEK |
| | VPDMSELKKSQVFYKYYLDKEELNDKNIKYAFCHFVEIEMSQLLKNYVYKR |
| | LSNISNDKIKRIFEYQNLKKLIENKLLNKLDTYVRNCGKYNYYLQDGEIATS |
| | DFIARNRQNEAFLRNIIGVSSVAYFSLRNILETENENDITGRMRGKTVKNNKG |
| | EEKYVSGEVDKIYNENKKNEVKENLKMFYSYDFNMDNKNEIEDFFANIDEA |
| | ISSIRHGIVHFNLELEGKDIFAFKNIAPSEISKKMFQNEINEKKLKLKIFRQLNS |
| | ANVFRYLEKYKILNYLKRTRFEFVNKNIPFVPSFTKLYSRIDDLKNSLGIYWK |
| | TPKTNDDNKTKEIIDAQIYLLKNIYYGEFLNYFMSNNGNFFEISKEIIELNKND |
| | KRNLKTGFYKLQKFEDIQEKIPKEYLANIQSLYMINAGNQDEEEKDTYIDFIQ |
| | KIFLKGFMTYLANNGRLSLIYIGSDEETNTSLAEKKQEFDKFLKKYEQNNNI |
| | KIPYEINEFLREIKLGNILKYTERLNMFYLILKLLNHKELTNLKGSLEKYQSA |
| | NKEEAFSDQLELINLLNLDNNRVTEDFELEADEIGKFLDFNGNKVKDNKELK |
| | KFDTNKIYFDGENIIKHRAFYNIKKYGMLNLLEKIADKAGYKISIEELKKYSN |
| | KKNEIEKNHKMQENLHRKYARPRKDEKFTDEDYESYKQAIENIEEYTHLKN |
| | KVEFNELNLLQGLLLRILHRLVGYTSIWERDLRFRLKGEFPENQYIEEIFNFE |
| | NKKNVKYKGGQIVEKYIKFYKELHQNDEVKINKYSSANIKVLKQEKKDLYI |
| | RNYIAHFNYIPHAEISLLEVLENLRKLLSYDRKLKNAVMKSVVDILKEYGFV |
| | ATFKIGADKKIGIQTLESEKIVHLKNLKKKKLMTDRNSEELCKLVKIMFEYK |
| | MEEKKSEN |
| SEQ ID | MNELVKNRCKQTKTICQKLIPIGKTRETIEKYNLMEIDRKIAANKELMNKLF |
| NO: 22 | SLIAGKHINDTLSKCTDLDFEPLLTSLSSLNNAKENDRDNLREYYDSVFEEK |
| | KTLAEEISSRLTAVKFAGKDFFTKNIPDFLETYEGDDKNEMSELVSLVIENTVT |
| | AGYVKKLEKIDRSMEYRLVSGTVVKRVLTDNADIYEKNIEKAKDFDYGVLN |
| | IDEASQFTTLVAKDYANYLTADGIAIYNVGIGKINLALNEYCQKNKEYSYNK |
| | LALLPLQKMLYGEKLSLFEKLEDFTSDEELINSYNKFAKTVNESGLAEIIKKA |
| | VPSYDEIVIKPNKISNYSNSITGHWSLVNRIMKDYLENNGIKNADKYMEKGL |
| | TLSEIGDALENKNIKHSDFISNLINDLGHTYTEIKENKESLKKDESVNALIIKK |
| | ELDMLLSILQNLKVFDIDNEMFDTGFGIEVSKAIEILGYGVPLYNKIRNYITK |
| | KPDPKKKFMTKFGSATIGTGITTSVEGSKKATFLKDGDAVFLLLYNTAGCKA |
| | NNVSVSNLADLINSSLEIENSGKCYQKMIYQTPGDIKKQIPRVFVYKSEDDDL |
| | IKDFKAGLHKTDLSFLNGRLIPYLKEAFATHETYKNYTFSYRNSYESYDEFCE |
| | HMSEQAYILEWKWIDKKLIDDLVEDGSLLMFRVWNRFMKKKEGKISKHAKI |
| | VNELFSDENASNAAIKLLSVFDIFYRDKQIDNPIVHKAGTTLYNKRTKDGEVI |
| | VDYTTMVKNKEKRPNVYTTTKKYDIIKDRRYTEEQFEIHLHVNIGKEENKE |
| | KLETSKVINEKKNTLVVTRSNEHLLYVVIFDENDNILLKKSLNTVKGMNFKS |
| | KLEVVEIQKKENMQSWKTVGSNQALMEGYLSFAIKEIADLVKEYDAILVLE |
| | QNSVGKNILNERVYTRFKEMLITNLSLDVDYENKDFYSYTELGGKVASWRD |
| | CVTNGICIQVPSAYKYKDPTTSFSTISMYAKTTAEKSKKLKQIKSFKYNRERG |
| | LFELVIAKGVGLENNIVCDSFGSRSIIENDISKEVSCTLKIEKYLIDAGIEYNDE |
| | KEVLKDLDTAAKTDAVHKAVTLLLKCFNESPDGRYYISPCGEHFTLCDAPEV |
| | LSAINYYIRSRYIREQIVEGVKKMEYKKTILLAK* |
| SEQ ID | MNGNRIIVYREFVGVTPVAKTLRNELRPIGHTQEHIIHNGLIQEDELRQEKST |
| NO: 23 | ELKNIMDDYYREYIDKSLSGVTDLDFTLLFELMNLVQSSPSKDNKKALEKE |
| | QSKMREQICTHMQSDSNYKNIFNAKFLKEILPDFIKNYNQYDAKDKAGKLE |
| | TLALFNGFSTYFTDFFEKRKNVFTKEAVSTSIAYRIVHENSLTFLANMTSYKK |
| | ISEKALDEIEVIEKNNQDKMGDWELNQIFNPDFYNMVLIQSGIDFYNEICGV |
| | VNAHMNLYCQQTKNNYNLFKMRKLHKQILAYTSTSFEVPKMFEDDMSVYN |
| | AVNAFIDETEKGNIIGKLKDIVNKYDELDEKRIYISKDFYETLSCFMSGNWNL |
| | ITGCVENFYDENIHAKGKSKEEKVKKAVKEDKYKSINDVNDLVEKYIDEKE |
| | RNEFKNSNAKQYIREISNIITDTETAHLEYDEHISLIESEEKADEMKKRLDMY |
| | MNMYHWAKAFIVDEVLDRDEMFYSDIDDIYNILENIVPLYNRVRNYVTQKP |
| | YNSKKIKLNFQSPTLANGWSQSKEFDNNAIILIRDNKYYLAIFNAKNKPDKKI |
| | IQGNSDKKNDNDYKKMVYNLLPGANKMLPKVFLSKKGIETFKPSDYIISGY |
| | NAHKHIKTSENFDISFCRDLIDYFKNSIEKHAEWRKYEFKFSATDSYNDISEF |
| | YREVEMQGYRIDWTYISEADINKLDEEGKIYLFQIYNKDFAENSTGKENLHT |
| | MYFKNIFSEENLKDIIIKLNGQAELFYRRASVKNPVKHKKDSVLVNKTYKNQ |
| | LDNGDVVRIPIPDDIYNEIYKMYNGYIKENDLSEAAKEYLDKVEVRTAQKDI |
| | VKDYRYTVDKYFIHTPITINYKVTARNNVNDMAVKYIAQNDDIHVIGIDRGE |
| | RNLIYISVIDSHGNIVKQKSYNILNNYDYKKKLVEKEKTREYARKNWKSIGN |
| | IKELKEGYISGVVHEIAMLMVEYNAIIAMEDLNYGFKRGRFKVERQVYQKF |
| | ESMLINKLNYFASKGKSVDEPGGLLKGYQLTYVPDNIKNLGKQCGVIFYVPA |
| | AFTSKIDPSTGFISAFNFKSISTNASRKQFFMQFDEIRYCAEKDMFSFGFDYN |
| | NFDTYNITMSKTQWTVYTNGERLQSEFNNARRTGKTKSINLTETIKLLLEDN |
| | EINYADGHDVRIDMEKMDEDKNSEFFAQLLSLYKLTVQMRNSYTEAEEQEK |
| | GISYDKIISPVINDEGEFFDSDNYKESDDKECKMPKDADANGAYCIALKGLY |
| | EVLKIKSEWTEDGFDRNCLKLPHAEWLDFIQNKRYE* |
| SEQ ID | MNKDIRKNFTDFVGISEIQKTLRFILIPIGKTAQNIDKYNMFEDDEIRHEYYPI |
| NO: 24 | LKEACDDFYRNHIDQQFENLELDWSKLDEALASEDRDLINETRATYRQVLF |
| | NRLKNSVDIKGDSKKNKTLSLESSDKNLGKKKTKNTFQYNFNDLFKAKLIK |
| | AILPLYIEYIYEGEKLENAKKALKMYNRFTSRLSNFWQARANIFTDDEISTGS |
| | PYRLVNDNFTIFRINNSIYTKNKPFIEEDILEFEKKLKSKKIIKDFESVDDYFTV |
| | NAFNKLCTQNGIDKYNSILGGFTTKEREKVKGLNELFNLAQQSINKGKKGE |
| | YRKNIRLGKLTKLKKQILAISDSTSFLIEQIEDDQDLYNKIKDFFELLLKEEIEN |
| | ENIFTQYANLQKLIEQADLSKIYINAKHLNKISHQVTGKWDSLNKGIALLLEN |
| | ININEESKEKSEVISNGQTKDISSEAYKRYLQIQSEEKDIERLRTQIYFSLEDLE |
| | KALDLVLIDENMDRSDKSILSYVQSPDLNVNFERDLTDLYSRIMKLEENNEK |
| | LLANHSAIDLIKEFLDLIMLRYSRWQILFCDSNYELDQTFYPIYDAVMEILSNI |
| | IRLYNLARNYLSRKPDRMKKKKINFNNPTLADGWSESKIPDNSSMLFIKDG |
| | MYYLGIIKNRAAYSELLEAESLQSSEKKKSENSSYERMNYHELPDAFRSIPKS |
| | SIAMKAVKEHFEINQKTADLLLDTDKFSKPLRITKEIFDMQYVDLHKNKKKY |
| | QVDYLRDTGDKKGYRKALNTWLNFCKDFISKYKGRNLFDYSKIKDADHYE |
| | TVNEFYNDVDKYSYHIFFTSVAETTVEKFISEGKLYLFQLYNKDFSPHSTGKP |
| | NLHTIYWRALFSEENLTSKNIKLNGQAEIFFRPKQIETPFTHKKGSILVNRFDV |
| | NGNPIPINVYQEIKGFKNNVIKWDDLNKTTQEGLENDQYLYFESEFEIIKDRR |
| | YTEDQLFFHVPISFNWDIGSNPKINDLATQYIVNSNDIHIIGIDRGENHLIYYS |
| | VIDLQGAIVEQGSLNTITEYTENKFLNNKTNNLRKIPYKDILQQREDERADA |
| | RIKWHAIDKIKDLKDGYLGQIVHFLAKLIIKYNAIVILEDLNYGFKRGRFKVE |
| | RQVYQKFEMALMKKLNVLVFKDYDIDEIGGPLKPWQLTRPIDSYERMGRQ |
| | NGILFYVPAAYTSAVDPVTGFANLFYLNNVKNSEKFHFFSKFESIKYHSDQD |
| | MFSFAFDYNNFGTTTRINDLSKSKWQVFTNHERSVWNNKEKNYVTQNLTD |
| | LIKKLLRTYNIEFKNNQNVLDSILKIENNTDKENFARELFRLFRLTIQLRNTTV |
| | NENNTEITENELDYIISPVKDKNGNFFDSRDELKNLPDNGDANGAYNIARKG |
| | LLYIEQLQESIKTGKLPTLSISTLDWFNYIMK* |
| SEQ ID | MNKGGYVIMEKMTEKNRWENQFRITKTIKEEIIPTGYTKVNLQRVNMLKRE |
| NO: 25 | MERNEDLKKMKEICDEYYRNMIDVSLRLEQVRTLGWESLIHKYRMLNKDE |
| | KEIKALEKEQEDLRKKISKGFGEKKAWTGEQFIKKILPQYLMDHYTGEELEE |
| | KLRIVKKFKGCTMFLSTFFKNRENIFTDKPIHTAVGHRITSENAMLFAANINT |
| | YEKMESNVTLEIERLQREFWRRGINISEIFTDAYYVNVLTQKQIEAYNKICGD |
| | INQHMNEYCQKQKLKFSEFRMRELKKQILAVVGEHFEIPEKIESTKEVYREL |
| | NEYYESLKELHGQFEELKSVQLKYSQIYVQKKGYDRISRYIGGQWDLIQEC |
| | MKKDCASGMKGTKKNHDAKIEEEVAKVKYQSIEHIQKLVCTYEEDRGHKV |
| | TDYVDEFIVSVCDLLGADHIITRDGERIELPLQYEPGTDLLKNDTINQRRLSDI |
| | KTILDWHMDMLEWLKTFLVNDLVIKDEEFYMAIEELNERMQCVISVYNRIR |
| | NYVTQKGYEPEKIRICFDKGTILTGWTTGDNYQYSGFLLMRNDKYYLGIINT |
| | NEKSVRKILDGNEECKDENDYIRVGYHLINDASKQLPRIFVMPKAGKKSEIL |
| | MKDEQCDYIWDGYCHNKHNESKEFMRELIDYYKRSIMNYDKWEGYCFKFS |
| | STESYDNMQDFYKEVREQSYNISFSYINENVLEQLDKDGKIYLFQVYNKDFA |
| | AGSTGTPNLHTMYLQNLFSSQNLELKRLRLGGNAELFYRPGTEKDVTHRKG |
| | SILVDRTYVREEKDGIEVRDTVPEKEYLEIYRYLNGKQKGDLSESAKQWLD |
| | KVHYREAPCDIIKDKRYAQEKYFLHFSVEINPNAEGQTALNDNVRRWLSEEE |
| | DIHVIGIDRGERNLIYVSLMDGKGRIKDQKSYNIVNSGNKEPVDYLAKLKVR |
| | EKERDEARRNWKAIGKIKDIKTGYLSYVVHEIVEMAVREKAIIVMEDLNYG |
| | FKRGRFKVERQVYQKFEEMLINKLNYVVDKQLSVDEPGGLLRGYQLAFIPK |
| | DKKSSMRQNGIVFYVPAGYTSKIDPTTGFVNIFKFPQFGKGDDDGNGKDYD |
| | KIRAFFGKFDEIRYECDEKVTADNTREVKERYRFDFDYSKFETHLVHMKKTK |
| | WTVYAEGERIKRKKVGNYWTSEVISDIALRMSNTLNIAGIEYKDGHNLVNEI |
| | CALRGKQAGIILNELLEIVRLTVQLRNSTTEGDVDERDEIISPVLNEKYGCFY |
| | HSTEYKQQNGDVLPKDADANGAYCIGLKGIYEIRQIKNKWKEDMTKGEGK |
| | ALNEGMRISHDQWFEFIQNMNKGE* |
| SEQ ID | MNNPRGAFGGFTNLYSLSKTLRFELKPYLEIPEGEKGKLFGDDKEYYKNCK |
| NO: 26 | TYTEYYLKKANKEYYDNEKVKNTDLQLVNFLHDERIEDAYQVLKPVFDTL |
| | HEEFITDSLESAEAKKIDFGNYYGLYEKQKSEQNKDEKKKIDKPLETERGKL |
| | RKAFTPIYEAEGKNLKNKAGKEKKDKDILKESGFKVLIEAGILKYIKNNIDEF |
| | ADKKLKNNEGKEITKKDIETALGAENIEGIFDGFFTYFSGFNQNRENYYSTEE |
| | KATAVASRIVDENLSKFCDNILLYRKNENDYLKIFNFLKNKGKDLKLKNSKF |
| | GKENEPEFIPAYDMKNDEKSFSVADFVNCLSQGEIEKYNAKIANANYLINLY |
| | NQNKDGNSSKLSMFKILYKQIGCGEKKDFIKTIKDNAELKQILEKACEAGKK |
| | YFIRGKSEDGGVSNIFDFTDYIQSHENYKGVYWSDKAINTISGKYFANWDTL |
| | KNKLGDAKVFNKNTGEDKADVKYKVPQAVMLSELFAVLDDNAGEDWREK |
| | GIFFKASLFEGDQNKSEIIKNANRPSQALLKMICDDMESLAKNFIDSGDKILKI |
| | SDRDYQKDENKQKIKNWLDNALWINQILKYFKVKANKIKGDSIDARIDSGL |
| | DMLVFSSDNPAEDYDMIRNYLTQKPQDEINKLKLNFENSSLAGGWDENKEK |
| | DNSCIILKDEQDKQYLAVMKYENTKVFEQKNSQLYIADNAAWKKMIYKLVP |
| | GASKTLPKVFFSKKWTANRPTPSDIVEIYQKGSFKKENVDFNDKKEKDESRK |
| | EKNREKIIAELQKTCWMDIRYNIDGKIESAKYVNKEKLAKLIDFYKENLKKY |
| | PSEEESWDRLFAFGFSDTKSYKSIDQFYIEVDKQGYKLEFVTINKARLDEYV |
| | RDGKIYLFEIRSRDNNLVNGEEKTSAKNLQTIYWNAAFGGDDNKPKLNGEA |
| | EIFYRPAIAENKLNKKKDKNGKEIIDGYRFSKEKFIFHCPITLNFCLKETKIND |
| | KLNAALAKPENGQGVYFLGIDRGEKHLAYYSLVNQKGEILEQGTLNLPFLD |
| | KNGKSRSIKVEKKSFEKDSNGGIIKDKDGNDKIKIEFVECWNYNDLLDARA |
| | GDRDYARKNWTTIGTIKELKDGYISQVVRKIVDLSIYKNTETKEFREMPAFIV |
| | LEDLNIGFKRGRQKIEKQVYQKLELALAKKLNFLVDKKADIGEIGSVTKAIQ |
| | LTPPVNNFGDMENRKQFGNMLYIRADYTSQTDPATGWRKSIYLKSGSESNV |
| | KEQIEKSFFDIRYESGDYCFEYRDRHGKMWQLYSSKNGVSLDRFHGERNNS |
| | KNVWESEKQPLNEMLDILFDEKRFDKSKSLYEQMFKGGVALTRLPKEINKK |
| | DKPAWESLRFVIILIQQIRNTGKNGDDRNGDFIQSPVRDEKTGEHFDSRIYLD |
| | KEQKGEKADLPTSGDANGAYNIARKGIVVAEHIKRGFDKLYISDEEWDTWL |
| | AGDEIWDKWLKENRESLTKTRK* |
| SEQ ID | MNYKTGLEDFIGKESLSKTLRNALIPTESTKIHMEEMGVIRDDELRAEKQQE |
| NO: 27 | LKEIMDDYYRTFIEEKLGQIQGIQWNSLFQKMEETMEDISVRKDLDKIQNEK |
| | RKEICCYFTSDKRFKDLFNAKLITDILPNFIKDNKEYTEEEKAEKEQTRVLFQ |
| | RFATAFTNYFNQRRNNFSEDNISTAISFRIVNENSEIHLQNMRAFQRIEQQYPE |
| | EVCGMEEEYKDMLQEWQMKHIYSVDFYDRELTQPGIEYYNGICGKINEHM |
| | NQFCQKNRINKNDFRMKKLHKQILCKKSSYYEIPFRFESDQEVYDALNEFIK |
| | TMKKKEIIRRCVHLGQECDDYDLGKIYISSNKYEQISNALYGSWDTIRKCIKE |
| | EYMDALPGKGEKKEEKAEAAAKKEEYRSIADIDKIISLYGSEMDRTISAKKCI |
| | TEICDMAGQISIDPLVCNSDIKLLQNKEKTTEIKTILDSFLHVYQWGQTFIVSD |
| | IIEKDSYFYSELEDVLEDFEGITTLYNHVRSYVTQKPYSTVKFKLHFGSPTLA |
| | NGWSQSKEYDNNAILLMRDQKFYLGIFNVRNKPDKQIIKGHEKEEKGDYKK |
| | MIYNLLPGPSKMLPKVFITSRSGQETYKPSKHILDGYNEKRHIKSSPKFDLGY |
| | CWDLIDYYKECIHKHPDWKNYDFHFSDTKDYEDISGFYREVEMQGYQIKW |
| | TYISADEIQKLDEKGQIFLFQIYNKDFSVHSTGKDNLHTMYLKNLFSEENLK |
| | DIVLKLNGEAELFFRKASIKTPIVHKKGSVLVNRSYTQTVGNKEIRVSIPEEY |
| | YTEIYNYLNHIGKGKLSSEAQRYLDEGKIKSFTATKDIVKNYRYCCDHYFLH |
| | LPITINFKAKSDVAVNERTLAYIAKKEDIHIIGIDRGERNLLYISVVDVHGNIRE |
| | QRSFNIVNGYDYQQKLKDREKSRDAARKNWEEIEKIKELKEGYLSMVIHYI |
| | AQLVVKYNAVVAMEDLNYGFKTGRFKVERQVYQKFETMLIEKLHYLVFKD |
| | REVCEEGGVLRGYQLTYIPESLKKVGKQCGFIFYVPAGYTSKIDPTTGFVNLF |
| | SFKNLTNRESRQDFVGKFDEIRYDRDKKMFEFSFDYNNYIKKGTILASTKWK |
| | VYTNGTRLKKIVVNGKYTSQSMEVELTDAMEKMLQRAGIEYHDGKDLKG |
| | QIVEKGIEAEIIDIFRLTVQMRNSRSESEDREYDRLISPVLNDKGEFFDTATAD |
| | KTLPQDADANGAYCIALKGLYEVKQIKENWKENEQFPRNKLVQDNKTWFD |
| | FMQKKRYL* |
| SEQ ID | MRISKTLSLRIVRPFYTPEVEAGIKAEKDKREAQGQTRSLDAKFFNELKKKH |
| NO: 28 | SEIILSSEFYSLLSEVQRQLTSIYNHAMSNLYHKIIVEGEKTSTSKALSNIGYDE |
| | CKAIFPSYMALGLRQKIQSNFRRRDLKNFRMAVPTAKSDKFPIPIYRQVDGS |
| | KGGFKISENDGKDFIVELPLVDYVAEEVKTAKGRFTKINISKPPKIKNIPVILST |
| | LRRRQSGQWFSDDGTNAEIRRVISGEYKVSWIEIVRRTRFGKHDDWFVNMV |
| | IKYDKPEEGLDSKVVGGIDVGVSSPLVCALNNSLDRYFVKSSDIIAFNKRAM |
| | ARRRTLLRQNKYKRSGHGSKNKLEPITVLTEKNERFKKSIMQRWAKEVAEFF |
| | RGKGASVVRMEELSGLKEKDNFFSSYLRMYWNYGQLQQIIENKLKEYGIKV |
| | NYVSPKDTSKKCHSCTHINEFFTFEYRQKNNFPLFKCEKCGVECSADYNAA |
| | KNMAIA |
| SEQ ID | MRTMVTFEDFTKQYQVSKTLRFELIPQGKTLENMKRDGIISVDRQRNEDYQ |
| NO: 29 | KAKGILDKLYKYILDFTMETVVIDWEALATATEEFRKSKDKKTYEKVQSKIR |
| | TALLEHVKKQKVGTEDLFKGMFSSKIITGEVLAAFPEIRLSDEENLILEKFKD |
| | FTTYFTGFFENRKNVFTDEALSTSFTYRLVNDNFIKFFDNCIVFKNVVNISPH |
| | MAKSLETCASDLGIFPGVSLEEVFSISFYNRLLTQTGIDQFNQLLGGISGKEGE |
| | HKQQGLNEIINLAMQQNLEVKEVLKNKAHRFTPLFKQILSDRSTMSFIPDAF |
| | ADDDEVLSAVDAYRKYLSEKNIGDRAFQLISDMEAYSPELMRIGGKYVSVLS |
| | QLLFYSWSEIRDGVKAYKESLITGKKTKKELENIDKEIKYGVTLQEIKEALPK |
| | KDIYEEVKKYAMSVVKDYHAGLAEPLPEKIETDDERASIKHIMDSMLGLYR |
| | FLEYFSHDSIEDTDPVFGECLDTILDDMNETVPLYNKVRNFSTRKVYSTEKF |
| | KLNFNNSSLANGWDKNKEQANGAILLRKEGEYFLGIFNSKNKPKLVSDGGA |
| | GIGYEKMIYKQFPDFKKMLPKCTISLKDTKAHFQKSDEDFTLQTDKFEKSIVI |
| | TKQIYDLGTQTVNGKKKFQVDYPRLTGDMEGYRAALKEWIDFGKEFIQAYT |
| | STAIYDTSLFRDSSDYPDLPSFYKDVDNICYKLTFEWIPDAVIDDCIDDGSLYL |
| | FKLHNKDFSSGSIGKPNLHTLYWKALFEEENLSDVVVKLNGQAELFYRPKS |
| | LTRPVVHEEGEVIINKTTSTGLPVPDDVYVELSKFVRNGKKGNLTDKAKNW |
| | LDKVTVRKMPHAITKDRRFTVDKFFFHVPITLNYKADSSPYRFNDFVRQYIK |
| | DCSDVKIIGIDRGERNLIYAVVIDGKGNIIEQRSFNTVGTYNYQEKLEQKEKE |
| | RQTARQDWATVTKIKDLKKGYLSAVVHELSKMIVKYKAIVALENLNVGFKR |
| | MRGGIAERSVYQQFEKALIDKLNYLVFKDEEQSGYGGVLNAYQLTDKFESF |
| | SKMGQQTGFLFYVPAAYTSKIDPLTGFINPFSWKHVKNREDRRNFLNLFSKL |
| | YYDVNTHDFVLAYHHSNKDSKYTIKGNWEIADWDILIQENKEVFGKTGTPY |
| | CVGKRIVYMDDSTTGHNRMCAYYPHTELKKLLSEYGIEYTSGQDLLKIIQEF |
| | DDDKLVKGLFYIIKAALQMRNSNSETGEDYISSPIEGRPGICFDSRAEADTLP |
| | YDADANGAFHIAMKGLLLTERIRNDDKLAISNEEWLNYIQEMRG* |
| SEQ ID | MSKLEKFTNCYSLSKTLRFKAIPVGKTQENIDNKRLLVEDEKRAEDYKGVK |
| NO: 30 | KLLDRYYLSFINDVLHSIKLKNLNNYISLFRKKTRTEKENKELENLEINLRKEI |
| | AKAFKGNEGYKSLFKKDIIETILPEFLDDKDEIALVNSFNGFTTAFTGFFDNRE |
| | NMFSEEAKSTSIAFRCINENLTRYISNMDIFEKVDAIFDKHEVQEIKEKILNSD |
| | YDVEDFFEGEFFNFVLTQEGIDVYNAIIGGFVTESGEKIKGLNEYINLYNQKT |
| | KQKLPKFKPLYKQVLSDRESLSFYGEGYTSDEEVLEVFRNTLNKNSEIFSSIK |
| | KLEKLFKNFDEYSSAGIFVKNGPAISTISKDIFGEWNVIRDKWNAEYDDIHLK |
| | KKAVVTEKYEDDRRKSFKKIGSFSLEQLQEYADADLSVVEKLKEIIIQKVDEI |
| | YKVYGSSEKLFDADFVLEKSLKKNDAVVAIMKDLLDSVKSFENYIKAFFGE |
| | GKETNRDESFYGDFVLAYDILLKVDHIYDAIRNYVTQKPYSKDKFKLYFQNP |
| | QFMGGWDKDKETDYRATILRYGSKYYLAIMDKKYAKCLQKIDKDDVNGN |
| | YEKINYKLLPGPNKMLPKVFFSKKWMAYYNPSEDIQKIYKNGTFKKGDMF |
| | NLNDCHKLIDFFKDSISRYPKWSNAYDFNFSETEKYKDIAGFYREVEEQGYK |
| | VSFESASKKEVDKLVEEGKLYMFQIYNKDFSDKSHGTPNLHTMYFKLLFDE |
| | NNHGQIRLSGGAELFMRRASLKKEELVVHPANSPIANKNPDNPKKTTTLSYD |
| | VYKDKRFSEDQYELHIPIAINKCPKNIFKINTEVRVLLKHDDNPYVIGIDRGE |
| | RNLLYIVVVDGKGNIVEQYSLNEIINNFNGIRIKTDYHSLLDKKEKERFEARQ |
| | NWTSIENIKELKAGYISQVVHKICELVEKYDAVIALEDLNSGFKNSRVKVEK |
| | QVYQKFEKMLIDKLNYMVDKKSNPCATGGALKGYQITNKFESFKSMSTQN |
| | GFIFYIPAWLTSKIDPSTGFVNLLKTKYTSIADSKKFISSFDRIMYVPEEDLFEF |
| | ALDYKNFSRTDADYIKKWKLYSYGNRIRIFRNPKKNNVFDWEEVCLTSAYK |
| | ELFNKYGINYQQGDIRALLCEQSDKAFYSSFMALMSLMLQMRNSITGRTDV |
| | DFLISPVKNSDGIFYDSRNYEAQENAILPKNADANGAYNIARKVLWAIGQFK |
| | KAEDEKLDKVKIAISNKEWLEYAQTSVKH |
| SEQ ID | MTNFDNFTKKYVNSKTIRLEAIPVGKTLKNIEKMGFIAADRQRDEDYQKAK |
| NO: 31 | SVIDHIYKAFMDDCLKDLFLDWDPLYEAVVACWRERSPEGRQALQIMQADY |
| | RKKIADRFRNHELYGSLFTKKIFDGSVAQRLPDLEQSAEEKSLLSNFNKFTSY |
| | FRDFFDKRKRLFSDDEKHSAIAYRLINENFLKFVANCEAFRRMTERVPELRE |
| | KLQNTGSLQVYNGLALDEVFSADFYNQLIVQKQIDLYNQLIGGIAGEPGTPN |
| | IQGLNATINLALQGDSSLHEKLAGIPHRFNPLYKQILSDVSTLSFVPSAFQSDG |
| | EMLAAVRGFKVQLESGRVLQNVRRLFNGLETEADLSRVYVNNSKLAAFSSM |
| | FFGRWNLCSDALFAWKKGKQKKITNKKLTEIKKWLKNSDIAIAEIQEAFGED |
| | FPRGKINEKIQAQADALHSQLALPIPENLKALCAKDGLKSMLDTVLGLYRM |
| | LQWFIVGDDNEKDSDFYFGLGKILGSLDPVLVLYNRVRNYITKKPYSLTKFR |
| | LNFDNSQLLNGWDENNLDTNCASIFIKDGKYYLGISNKNNRPQFDTVATSGK |
| | SGYQRMVYKQFANWGRDLPHSTTQMKKVKKHFSASDADYVLDGDKFIRP |
| | LIITKEIFDLNNVKFNGKKKLQVDYLRNTGDREGYTHALHTWINFAKDFCA |
| | CYKSTSIYDISCLRPTDQYDNLMDFYADLGNLSHRIVWQTIPEEAIDNYVEQ |
| | GQLFLFQLYNKDFAPGADGKPNLHTLYWKAVFNPENLEDVVVKLNGKAEL |
| | FYRPRSNMDVVRHKVGEKLVNRKLKNGLTLPSRLHEEIYRYVNGTLNKDLS |
| | ADARSVLPLAVVRDVQHEIIKDRRFTADKFFFHASLTFNFKSSDKPVGFNED |
| | VREYLREHPDTYVVGVDRGERNLIYIVVIDPQGNIVEQRSFNMINGIDYWSL |
| | LDQKEKERVEAKQAWETVGKIKDLKCGYLSFLIHEITKIIIKYHAVVILENLSL |
| | GFKRVRTGIAEKAVYQQFERMLVTKLGYVVFKDRAGKAPGGVLNAYQLTD |
| | NTRTAENTGIQNGFLFYVPAAFTSRVDPATGFFDFYDWGKIKTATDKKNFIAG |
| | FNSVRYERSTGDFIVHVGAKNLAVRRVAEDVRTEWDIVIEANVRKMGIDGN |
| | SYISGKRIRYRSGEQGHGQYENHLPCQELIRALQQYGIQYETGKDILPAILQQ |
| | DDAKLTDTVFDVFRLALQMRNTSAETGEDYFNSVVRDRSGRCFDTRRAEA |
| | AMPKEADANDAYHIALKGLFVLEKLRKGESIGIKNTEWLRYVQQRHS* |
| SEQ ID | MTPIFCNFVVYQIMLFNNNININVKTMNKKHLSDFTNLFPVSKTLRFRLEPQ |
| NO: 32 | GKTMENIVKAQTIETDEERSHDYEKTKEYIDDYHRQFIDDTLDKFAFKVEST |
| | GNNDSLQDYLDAYLSANDNRTKQTEEIQTNLRKAIVSAFKMQPQFNLLFKK |
| | EMVKHLLPQFVDTDDKKRIVAKFNDFTTYFTGFFTNRENMYSDEAKSTSIAY |
| | RIVNQNLIKFVENMLTFKSHILPILPQEQLATLYDDFKEYLNVASIAEMFELDH |
| | FSIVLTQRQIEVYNSVIGGRKDENNKQIKPGLNQYINQHNQAVKDKSARLPL |
| | LKPLFNQILSEKAGVSFLPKQFKSASEVVKSLNEAYAELSPVLAAIQDVVTNI |
| | TDYDCNGIFIKNDLGLTDIAQRFYGNYDAVKRGLRNQYELETPMHNGQKAE |
| | KYEEQVAKHLKSIESVSLAQINQVVTDGGDICDYFKAFGATDDGDIQRENLL |
| | ASINNAHTAISPVLNKENANDNELRKNTMLIKDLLDAIKRLQWFAKPLLGAG |
| | DETNKDQVFYGKFEPLYNQLDETISPLYDKVRSYLTKKPYSLDKFKINFEKS |
| | NLLGGWDPGADRKYQYNAVILRKDNDFYLGIMRDEATSKRKCIQVLDCND |
| | EGLDENFEKVEYKQIKPSQNMPRCAFAKKECEENADIMELKRKKNAKSYNT |
| | NKDDKNALIRHYQRYLDRTYPEFGFVYKDADEYDTVKAFTDSMDSQDYKL |
| | SFLQVSETGLNKLVDEGDLYLFKITNKDFSSYAKGRPNLHTIYWRMLFDPKN |
| | LANVVYKLEGKAEVFFRRKSLASTTTHKAKQAIKNKSRYNEAVKPQSTFDY |
| | DIIKDRRFTADKFEFHVPIKMNFKAAGWNSTRLTNEVREFIKSQGVRHIIGID |
| | RGERHLLYLTMIDMDGNIVKQCSLNAPAQDNARASEVDYHQLLDSKEADRL |
| | AARRNWGTIENIKELKQGYLSQVVHLLATMMVDNDAILVLENLNAGFMRG |
| | RQKVEKSVYQKFEKMLIDKLNYIVDKGQSPDKPTGALHAVQLTGLYSDENK |
| | SNMKRANVRQCGFVFYIPAWNTSKIDPVTGFVNLFDTHLSSMGEIKAFFSKF |
| | DSIRYNQDKGWFEFKFDYSRFTTRAEGCRTQWTVCTYGERIWTHRSKNQN |
| | NQFVNDTVNVTQQMLQLLQDCGIDPNGNLKEAIANIDSKKSLETLLHLFKLT |
| | VQMRNSVTGSEVDYMISPVADERGHFFDSRESDEHLPANADANGAFNIARK |
| | GLMVVRQIMATDDVSKIKFAVSNKDWLRFAQHIDD* |
| SEQ ID | VKISKTLSLRIIRPYYTPEVESAIKAEKDKREAQGQTRNLDAKFFNELKKKHP |
| NO: 33 | QIILSGEFYSLLFEMQRQLTSIYNRAMSSLYHKIIVEGEKTSTSKALSDIGYDE |
| | CKSVFPSYIALGLRQKIQSNFRRKELKGFRMAVPTAKSDKFPIPIYKQVDDGK |
| | GGFKISENKEGDFIVELPLVEYTAEDVKTAKGKFTKINISKPPKIKNIPVILSTL |
| | RRKQSGQWFSDEGTNAEIRRVISGEYKVSWIEVVRRTRFGKHDDWFLNIVIK |
| | YDKTEDGLDPEVVGGIDVGVSTPLVCAVNNSLDRYFVKSSDIIAFKKRAMAR |
| | RRTLLRQNRFKRSGHGSKSKLEPITILTEKNERFKKSIMQRWAKEVAEFFKGE |
| | RASVVQMEELSGLKEKDNFFGSYLRMYWNYGQLQQIIENKLKEYGIKVNY |
| | VSPKDTSKKCHSCGYINEFFTFEFRQKNNFPLFKCKKCGVECNADYNAAKNI |
| | AIA |
| SEQ ID | MLKSYDYFTKLYSLQKTLRFELKPIGKTLEHIKNSGIIESDETLEEQYAIVKNII |
| NO: 34 | DKLHRKHIDEALSLVDFTKHLDTLKTFQELYLKRGKTDKEKEELEKLSADLR |
| | KLIVSYLKGNVKEKTQHNLNPIKERFEILFGKELFTNEEFFLLAENEKEKKAI |
| | QAFKGFTTYFKGFQENRKNMYSEEGNSTSIAYRIINENLPLFIENIARFQKVM |
| | STIEKTTIKKLEQNLKTELKKHNLPGIFTIEYFNNVLTQEGISRYNTIIGGKTTH |
| | EGVKIQGLNEIINLYNQQSKDVKLPILKPLHKQILSEEYSTSFKIKAFENDNEV |
| | LKAIDTFWNEHIEKSIHPVTGNKFNILSKIENLCDQLQKYKDKDLEKLFIERK |
| | NLSTVSHQVYGQWNIIRDALRMHLEMNNKNIKEKDIDKYLDNDAFSWKEI |
| | KDSIKIYKEHVEDAKELNENGIIKYFSAMSINEEDDEKEYSISLIKNINEKYNN |
| | VKSILQEDRTGKSDLHQDKEKVGIIKEFLDSLKQLQWFLRLLYVTVPLDEKD |
| | YEFYNELEVYYEALLPLNSLYNKVRNYMTRKPYSVEKFKLNFNSPTLLDGW |
| | DKNKETANLSIILRKNGKYYLGIMNKENNTIFEYYPGTKSNDYYEKMIYKLL |
| | PGPNKMLPKVFFSKKGLEYYNPPKEILNIYEKGEFKKDKSGNFKKESLHTLI |
| | DFYKEAIAKNEDWEVFNFKFKNTKEYEDISQFYRDVEEQGYLITFEKVDAN |
| | YVDKLVKEGKLYLFQIYNKDFSENKKSKGNPNLHTIYWKGLYDSENLKNVV |
| | YKLNGEAEVFYRKKSIDYPEEIYNHGHHKEELLGKFNYPIIKDRRYTQDKFL |
| | FHVPITMNFISKEEKRVNQLACEYLSATKEDVHIIGIDRGERHLLYLSLIDKEG |
| | NIKKQLSLNTIKNENYDKEIDYRVKLDEKEKKRDEARKNWDVIENIKELKE |
| | GYMSQVIHIIAKMMVEEKAILIMEDLNIGFKRGRFKVEKQVYQKFEKMLID |
| | KLNYLVFKNKNPLEPGGSLNAYQLTSKFDSFKKLGKQSGFIFYVPSAYTSKID |
| | PTTGFYNFIQVDVPNLEKGKEFFSKFEKIIYNTKEDYFEFHCKYGKFVSEPKN |
| | KDNDRKTKESLTYYNAIKDTVWVVCSTNHERYKIVRNKAGYYESHPVDVT |
| | KNLKDIFSQANINYNEGKDIKPIIIESNNAKLLKSIAEQLKLILAMRYNNGKH |
| | GDDEKDYILSPVKNKQGKFFCTLDGNQTLPINADANGAYNIALKGLLLIEKI |
| | KKQQGKIKDLYISNLEWFMFMMSR |
| SEQ ID | MEKSLNDFIGLYSVSKTLRFELKPVSETLENIKKFHFLEEDKKKANDYKDVK |
| NO: 35 | KIIDNYHKYFIDDVLKNASFNWKKLEEAIREYNKNKSDDSALVAEQKKLGD |
| | AILKLFTSDKRYKALTAATPKELFESILPDWFGEQCNQDLNKAALKTFQKFTS |
| | YFTGFQENRKNVYSAEAIPTAVPYRIVNDNFPKFLQNVLIFKTIQEKCPQIIDE |
| | VEKELSSYLGKEKLAGIFTLESFNKYLGQGGKENQRGIDFYNQIIGGVVEKE |
| | GGINLRGVNQFLNLYWQQHPDFTKEDRRIKMVPLYKQILSDRSSLSFKIESIE |
| | NDEELKNALLECADKLELKNDEKKSIFEEVCDLFSSVKNLDLSGIYINRKDIN |
| | SVSRILTGDWSWLQSRMNVYAEEKFTTKAEKARWQKSLDDEGENKSKGFY |
| | SLTDLNEVLEYSSENVAETDIRITDYFEHRCRYYVDKETEMFVQGSELVALSL |
| | QEMCDDILKKRKAMNTVLENLSSENKLREKTDDVAVIKEYLDAVQELLHRI |
| | KPLKVNGVGDSTFYSVYDSIYSALSEVISVYNKTRNYITKKAASPEKYKLNF |
| | DNPTLADGWDLNKEQANTSVILRKDGMFYLGIMNPKNKPKFAEKYDCGNE |
| | SCYEKMIYKQFDATKQIPKCSTQKKEVQKYFLSGATEPYILNDKKSFKSELII |
| | TKDIWFMNNHVWDGEKFVPKRDNETRPKKFQIGYFKQTGDFDGYKNALSN |
| | WISFCKNFLQSYLSATVYDYNFKNSEEYEGLDEFYNYLNATCYKLNFINIPET |
| | EINKMVSEGKLYLFQIYNKDFASGSTGMPNMHTLYWKNLFSDENLKNVCLK |
| | LNGEAELFYRPAGIKEPVIHKEGSYLVNRTTEDGESIPEKIYFEIYKNANGKLE |
| | KLSDEAQNYISNHEVVIKKAGHEIIKDRHYTEPKFLFHVPLTINFKASGNSYSI |
| | NENVRKFLKNNPDVNIIGLDRGERHLIYLSLINQKGEIIKQFTFNEVERNKNG |
| | RTIKVNYHEKLDQREKERDAARKSWQAIGKIAELKEGYLSAVIHQLTKLMV |
| | EYNAVVVMEDLNFGFKRGRFHVEKQVYQKFEHILIDKSNYLVFKDRGLNEP |
| | GGVLNGYQIAGQFESFQKLGKQSGMLFYVPAGYTSKIDPKTGFVSMMNFK |
| | DLTNVHKKRDFFSKFDNIHYDEANGSFVFTFDYKKFDGKAKEEMKLTKWS |
| | VYSRDKRIVYFAKTKSYEDVLPTEKLQKIFESNGIDYKSGNNIQDSVMAIGA |
| | DLKEGAKPSKEISDFWDGLLSNFKLILQMRNSNARTGEDYIISPVMADDGTF |
| | FDSREEFKKGEDAKLPLDADANGAYHIALKGLSLINKINLSKDEELKKFDMK |
| | ISNADWFKFAQEKNYAK* |
| SEQ ID | MIKNPSNRHSLPKVIISEVDHEKILEFKIKYEKLARLDRFEVKAMHYEGKEIV |
| NO: 36 | FDEVLVNGGLIEVEYQDDNKTLFVKVGEKSYSIRGKKVGGKQRLLEDRVSK |
| | TKVQLELSDGVVDNKGNLRKSRTERELIVADNIKLYSQIVGREVTTTKEIYLV |
| | KRFLAYRSDLLFYYSFVDNFFKVAGNEKELWKINFDDATSAQFMGYIPFMV |
| | NDNLKNDNAYLKDYVRNDVQIKDDLKKVQTIFSALRHTLLHFNYEFFEKLF |
| | NGEDVGFDFDIGFLNLLIENIDKLNIDAKKEFIDNEKIRLFGENLSLAKVYRL |
| | YSDICVNRVGFNKFINSMLIKDGVENQVLKAEFNRKFGGNAYTIDIHSNQEY |
| | KRIYNEHKKLVIKVSTLKDGQAIRRGNKKISELKEQMKSMTKKNSLARLEC |
| | KMRLAFGFLYGEYNNYKAFKNNFDTNIKNSQFDVNDVEKSKAYFLSTYERR |
| | KPRTREKLEKVAKDIESLELKTVIANDTLLKFILLMFVFMPQELKGDFLGFVK |
| | KYYHDVHSIDDDTKEQEEDVVEAMSTSLKLKILGRNIRSLTLFKYALSSQVN |
| | YNSTDNIFYVEGNRYGKIYKKLGISHNQEEFDKTLVVPLLRYYSSLFKLMND |
| | FEIYSLAKANPTAVSLQELVDDETSPYKQGNYFNFNKMLRDIYGLTSDEIKSG |
| | QVVFMRNKIAHFDTEVLLSKPLLGQTKMNLQRKDIVSFIEARGDIKELLGYD |
| | AINDFRMKVIHLRTKMRVYSDKLQTMMDLLRNAKTPNDFYNVYKVKGVES |
| | INKHLLEVLAQTAEERTVEKQIRDGNEKYDL |
| SEQ ID | LNSIEKIKKPSNRNSIPSIIISDYDENKIKEIKVKYLKLARLDKITIQDMEIRDNI |
| NO: 37 | VEFKKILLNGIEHTIKDNQKIEFDNYEITAYVRASKQRRDGKITQAKYVVTIT |
| | DKYLRDNEKEKRFKSTERELPNDTLLMRYKQISGFDTLTSKDIYKIKRYIDFK |
| | NEMLFYFQFIEEFFSPLLPKGTNFYSLNIEQNKDKVVKYIVYRLNDDFKNQS |
| | LNQFIKKTDTIKYDFLKIQKILSDFRHALAHFDFDFIQKFFDDELDKNRFDIST |
| | ISLIKTMLQEKEEKYYQEKNNYIEDSDTLTLFDEKESNFSKIHNFYIKISQKKP |
| | AFNKLINSFLSKDGVPNEELKSYLATKKIDFFEDIHSNKEYKKIYIKHKNLVV |
| | EKQKEESQEKPNGQKLKNYNDELQKLKDEMNKITKQNSLNRLEVKLRLAF |
| | GFIANEYNYNFKNFNDKFTLDVKKEQKIKVFKNSSNEKLKEYFESTFIEKRFF |
| | HFCVKFFNKKTKKEETKQKNIFNLIENETLEELVKESPLLQIITLLYLFIPKELQ |
| | GEFVGFILKIYHHTKNITNDTKEDEKSIEDTQNSFSLKLKILAKNLRGLQLFN |
| | YSLSHNTLYNTKEHFFYEKGNRWQSVYKSLEISHNQDEFDIHLVIPVIKYYIN |
| | LNKLIGDFEIYALLTYADKNSITEKLSDITKRDDLKFRGYYNFSTLLFKTFMIN |
| | TNYEQNQKSTQYIKQTRNDIAHQNIENMLKAFENNEIFAQREEIVNYLQKEH |
| | KMQEILHYNPINDFTMKTVQYLKSLNIHSQKESKIADIHKKESLVPNDYYLIY |
| | KLKVIELLKQKVIEAIGETKDEEKIKNAIAKEEQIKKGYNK |
| SEQ ID | MLKHKRKNKNSLARVVLSNYDSNNIYEIKIKYEKLAKLDKINIIEMDYDAD |
| NO: 38 | NNVMFKKVLFNNKEIDLSHKDKTKINIELDNKKYNISAKKQIGKTHLVVRN |
| | KQTSKISRIKKIQDTYYRGKDVFILDNNIEILDKKQTKDKFIVTLNDITNNKTT |
| | STEAELIDDTKDIFKKISAKKDLKSSDIYKIKRFISIRSNFSFYYTFVDNYFKIF |
| | HAKKDKNKEELYKIKFKDEINIKPYLENILDNMKNKNGILYNYANDRKKVL |
| | NDLRNIQYVFKEFRHKLAHFDYNFLDNFFSNSVEEKYKQKVNEIKLLDILLD |
| | NIDSLNVVPKQNYIEDETISVFDAKDIKLKRLYTYYIKLTINYPGFKKLINSFFI |
| | QDGIENQELKEYINNKEKDTQVLKELDNKAYYMDISQYRKYKNIYNKHKEL |
| | VSEKELSSDGKKINSLNQKINKLKIDMKNITKPNALNRLIYRLRVAFGFIYKE |
| | YATINNFNKSFLQDTKTKRFENISQQDIKSYLDISYQDKGKFFVKSKKTFKNK |
| | TTVKYTFEDLDLTLNEIITQDDIFVKVIFLFSIFMPKELNGDFFGFINMYYHK |
| | MKNISYDTKDIDMLDTISQNMKLKILEQNIKKTYVFKYYLDLDSSIYSKLVQ |
| | NIKITEDIDSKKYLYAKIFKYYQHLYKLISDVEIYLLYKYNSKENLSITIDKDEL |
| | KHRGYYNFQSLLIKNNINKDDAYWSIVNMRNNLSHQNIDELVGHFCKGCLR |
| | KSTTDIAELWLRKDILTITNEIINKIESFKDIKITLGYDCVNDFTQKVKQYKQK |
| | LKASNERLAKKIEEKQNQVVDEKNKEELEKNILNMKNIQKINRYILDIL |
| SEQ ID | MLKHKRKNKNSLARVVLSNYDSNNIYEIKIKYEKLAKLDKINIIEMDYDAD |
| NO: 39 | NNVMFKKVLFNNKEIDLSHKDKTKINIELDNKKYNISAKKQIGKTHLVVRD |
| | KQTSKISRIKKIQDTYYRGKDVFILDNNIEILDKKQTKDKFIVTLNDITNDKTT |
| | STEAELIDDTKDIFKKISAKKDLKSSDIYKIKRFISIRSNFSFYYTFVDNYFKIF |
| | HAKKDKNKEELYKIKFKDEINIKPYLENILDNMKNKNGILYDYADDREKVL |
| | NDLKNIQYVFTEFRHKLAHFDYNFLDNFFSNSVTDQYKQKVNEIKLLDILLD |
| | NIDSLNVVPKQNYIEDETISVFDAKDIKLKRLYTYYIKLTINYPGFKKLINSFFI |
| | QDGIENQELKEYINNKEKDTQVLKELDNKAYYMDISQYRKYKNIYNKHKEL |
| | VSEKELSSDGQKINSLNQKINKLKIEMKNITKPNALNRLIYRLRVAFGFIYKE |
| | YATINNFNKSFLQDTKIKRFENISQQDIKNYLDISYQDKGKFFVKSKKTFKNK |
| | TTIKYTFEDLDLTLNEIITQDDIFVKVIFLFSIFMPKELNGDFFGFINMYYHKM |
| | KNISYDTKDIDMLDTISQNMKLKILEQNIKKTYVFKYYLDLDSSIYSKLVQNI |
| | KITEDIDSKKYLYAKIFKYYQHLYKLISDVEIYLLYKYNSKENLSITIDKDELK |
| | HRGYYNFQSLLIKNNINKDDAYWSIVNMRNNLSHQNIDELVGHFCKGCLRK |
| | STTDIAELWLRKDILTITNEIINKIESFKDIKITLGYDCVNDFTQKVKQYKQKL |
| | KASNERLAKKIEEKQNQVVDEKNKEELEKKILNMKNIQKINRYILDIL |
| SEQ ID | MSQLKNPSNKNSLPRIIISDFNEIKINEIKIKYHKLDRLDKIIVKEMEIINNKIFF |
| NO: 40 | KKILFNNQIKDINSENIELENYILAGEVKPSNTKIILNRDGKEKSFIVYDGFTF |
| | KYKPNDKRISETKTNAKYILTIKDKTRHRESSTQRDILKSSIIETYKQISGFENI |
| | TSKDIYTIKRYIDFKNEMMFYYTFIDDFFFPITGKNKQDKKNNFYNYKIKEN |
| | AKKFISLINYRINDDFKNKNGILYDYLSNKEEIIINDFIHIQTILKDVRHAIAHF |
| | NFDFIQKLFDNEQAFNSKFDGIEILNILFNQKQEKYFEAQTNYIEEETIKILDE |
| | KELSFKKLHSFYSQICQKKPAFNKLINSFIIQDGIENKELKDYISQKYNSKFDY |
| | YLDIHTCKIYKDIYNQHKKFVADKQFLENQKTDGQKIKKLNDQINQLKTKM |
| | NNLTKKNSLKRLEIKFRLAFGFIFTEYQTFKNFNERFIEDIKANKYSTKIELLD |
| | YGKIKEYISITHEEKRFFNYKTFNKKTNKNINKTIFQSLEKETFENLVKNDNLI |
| | KMMFLFQLLLPRELKGEFLGFILKIYHDLKNIDNDTKPDEKSLSELNISTALK |
| | LKILVKNIRQINLFNYTISNNTKYEEKEKRFYEEGNQWKDIYKKLYISHDFDI |
| | FDIHLIIPIIKYNINLYKLIGDFEVYLLLKYLERNTNYKTLDKLIEAEELKYKG |
| | YYNFTTLLSKAINIALNDKEYHNITHLRNNTSHQDIQNIISSFKNNKLLEQRE |
| | NIIELISKESLKKKLHFDPINDFTMKTLQLLKSLEVHSDKSEKIENLLKKEPLL |
| | PNDVYLLYKLKGIEFIKKELISNIGITKYEEKIQEKIAKGVEK |
| SEQ ID | MVKNPANRHALPKVIISEVDNNNILEFKIKYEKLARLDKVEVKSMHFDNNK |
| NO: 41 | QVVFDEVVINGGLIEPTYEDKHKKLVVTAGEKSYSIVGQKVGGKPRLLEDR |
| | VSKTKVQLELTNYVEDKEGKKRVSKTERELIVADNIELYSQIVGREVKTTKEI |
| | YLIKRFLEYRSDLLFYYGFVDNFFKVAGNGKELWKIDFTNSDSLHLIEYFKFS |
| | INDNLKNDENYLKNYVSDNTKIENDLVKCQNNFNSLRHALMHFDYDFFEKL |
| | FNGEDVGFDFDIEFLNIMIDKVDKLNIDTKKEFIDDEEVTLFGEALSLKKLYG |
| | LFSHIAINRVAFNKLINSFIIEDGIENKELKDFFNNKKESQAYEIDIHSNAEYKA |
| | LYVQHKKLVMATSAMTDGDEIAKKNQEISDLKEKMKVITKENSLARLEHKL |
| | RLAFGFIYTEYKDYKTFKKHFDQDIKGAKYKGLNVEKLKEYYETTLKNSKP |
| | KTDEKLEDVAKKIDKLSLKELIDDDTLLKFVLLLFIFMPQELKGDFLGFIKKY |
| | YHDKKHIDQDTKDKDTEIEELSTGLKLKVLDKNIRSLSILKHSFSFQVKYNR |
| | KDKNFYEDGNLHGKFYKKLSISHNQEEFNKSVYAPLFRYYSALYKLINDFEI |
| | YALAQHVENHETLADQVNKSQFIQKSYFNFRKLLDNTDSISQSSSYNTLIVM |
| | RNDISHLSYEPLFNYPLDERKSYKKKTQKGVKTFHVELLYISRAKIIELISLQT |
| | DMKKLLGYDAVNDFNMKVVHLRKRLSVYANKEESIRKMQADAKTPNDFY |
| | NIYKVKGVESINQHLLKVIGVTEAEKSIEKQINEGNKKHNT |
| SEQ ID | MTKKPSNRNSLPKVIINKVDESSILEFKIKYEKLARLDRFEVRSMRYDGDGRI |
| NO: 42 | IFDEVVANAGLLDVDYEDDNRTIVVKIENKAYNIYGKKVGGEKRLNGKISK |
| | AKVQLILTDSIRKNANDTHRHSLTERELINKNEVDLYSKIAEREISTTKDIYLV |
| | KRFLAYRSDLLLYYAFINHYVRVNGNKKEFWKTEIDDKIIDYFIYTINDTLKN |
| | KEGYLEKYIVDRDQIKKDLEKIKQIFSHLRHKLMHYDFRFFTDLFDGKDVDI |
| | KVDNSIQKISELLDIEFLNIVIDKLEKLNIDAKKEFIDDEKITLFGQEIELKKLY |
| | SLYAHTSINRVAFNKLINSFLIKDGVENKELKEYFNAHNQGKESYYIDIHQNQ |
| | EYKKLYIEHKNLVAKLSATTDGKEIAKINRELADKKEQMKQITKANSLKRLE |
| | YKLRLAFGFIYTEYKDYERFKNSFDTDTKKKKFDAIDNAKIIEYFEATNKAK |
| | KIEKLEEILKGIDKLSLKTLIQDDILLKFLLLFFTFLPQEIKGEFLGFIKKYYHDI |
| | TSLDEDTKDKDDEITELPRSLKLKIFSKNIRKLSILKHSLSYQIKYNKKESSYY |
| | EAGNVFNKMFKKQAISHNLEEFGKSIYLPMLKYYSALYKLINDFEIYALYKD |
| | MDTSETLSQQVDKQEYKRNEYFNFETLLRKKFGNDIEKVLVTYRNKIAHLD |
| | FNFLYDKPINKFISLYKSREKIVNYIKNHDIQAVLKYDAVNDFVMKVIQLRTK |
| | LKVYADKEQTIESMIQNTQNPNGFYNIYKVKAVENINRHLLKVIGYTESEKA |
| | VEEKIRAGNTSKS |
| SEQ ID | MIKNPSNRYALPKVIISKIDNQNILEFKIKYKKLSKLDIVKVKSMHYDDRAIIF |
| NO: 43 | DEVIVNDGLIDVEYRDNHKTIFVKVGNKSYSISGQKVGGKERLLENRVSKTK |
| | VQLELKDKATNRVSKTERELIVDDNIKIYSQIVGRDVKTTKDIYLIKRFLAYR |
| | SDLLFYYGFVNNFFHVANNRSEFWKIDFNDSNNSKLIEYFKFTINDHLKNDE |
| | NYLKDYISDNEKLKNDLIKVKNSFEKIRHALMHFDYDFFVKLFNGEDVGLE |
| | LDIEFLDIMIDKLDKLNIDTKKEFIDDEKITIFGEELSLAKLYRFYAHTAINRVA |
| | FNKLINSFIIENGVENQSLKEYFNQQAGGIAYEIDIHQNREYKNLYNEHKKLV |
| | SRVLSISDGQEIAILNQKIAKLKDQMKQITKANSIKRLEYKLRLALGFIYTEYE |
| | NYEEFKNNFDTDIKNGRFTPKDNDGNKRAFDSRELEQLKGYYEATIQTQKP |
| | KTDEKIEEVSKKIDRLSLKSLIADDILLKFILLMFTFMPQELKGEFLGFIKKYY |
| | HDTKHIDQDTISDSDDTIETLSIGLKLKILDKNIRSLSILKHSLSFQTKYNKKD |
| | RNYYEDGNIHGKFFKKLGISHNQEEFNKSVYAPLFRYYSALYKLINDFEIYTL |
| | SLHIVGSETLTDQVNKSQFLSGRYFNFRKLLTQSYHINNNSTHSTIFNAVINM |
| | RNDISHLSYEPLFDCPLNGKKSYKRKIRNQFKTINIKPLVESRKIIIDFITLQTD |
| | MQKVLGYDAVNDFTMKIVQLRTRLKAYANKEQTIQKMITEAKTPNDFYNIY |
| | KVQGVEEINKYLLEVIGETQAEKEIREKIERGNIANF |
| SEQ ID | MKKSIFDQFVNQYALSKTLRFELKPVGETGRMLEEAKVFAKDETIKKKYEA |
| NO: 44 | TKPFFNKLHREFVEEALNEVELAGLPEYFEIFKYWKRYKKKFEKDLQKKEK |
| | ELRKSVVGFFNAQAKEWAKKYETLGVKKKDVGLLFEENVFAILKERYGNEE |
| | GSQIVDESTGKDVSIFDSWKGFTGYFIKFQETRKNFYKDDGTATALATRIIDQ |
| | NLKRFCDNLLIFESIRDKIDFSEVEQTMGNSIDKVFSVIFYSSCLLQEGIDFYN |
| | CVLGGETLPNGEKRQGINELINLYRQKTSEKVPFLKLLDKQILSEKEKFMDEI |
| | ENDEALLDTLKIFRKSAEEKTTLLKNIFGDFVMNQGKYDLAQIYISRESLNTI |
| | SRKWTSETDIFEDSLYEVLKKSKIVSASVKKKDGGYAFPEFIALIYVKSALEQ |
| | IPTEKFWKERYYKNIGDVLNKGFLNGKEGVWLQFLLIFDFEFNSLFEREIIDE |
| | NGDKKVAGYNLFAKGFDDLLNNFKYDQKAKVVIKDFADEVLHIYQMGKYF |
| | AIEKKRSWLADYDIDSFYTDPEKGYLKFYENAYEEIIQVYNKLRNYLTKKPY |
| | SEDKWKLNFENPTLADGWDKNKEADNSTVILKKDGRYYLGLMARGRNKL |
| | FDDRNLPKILEGVENGKYEKVVYKYFPDQAKMFPKVCFSTKGLEFFQPSEE |
| | VITIYKNSEFKKGYTFNVRSMQRLIDFYKDCLVRYEGWQCYDFRNLRKTED |
| | YRKNIEEFFSDVAMDGYKISFQDVSESYIKEKNQNGDLYLFEIKNKDWNEGA |
| | NGKKNLHTIYFESLFSADNIAMNFPVKLNGQAEIFYRPRTEGLEKERIITKKG |
| | NVLEKGDKAFHKRRYTENKVFFHVPITLNRTKKNPFQFNAKINDFLAKNSDI |
| | NVIGVDRGEKQLAYFSVISQRGKILDRGSLNVINGVNYAEKLEEKARGREQA |
| | RKDWQQIEGIKDLKKGYISQVVRKLADLAIQYNAIIVFEDLNMRFKQIRGGI |
| | EKSVYQQLEKALIDKLTFLVEKEEKDVEKAGHLLKAYQLAAPFETFQKMGK |
| | QTGIVFYTQAAYTSRIDPVTGWRPHLYLKYSSAEKAKADLLKFKKIKFVDGR |
| | FEFTYDIKSFREQKEHPKATVWTVCSCVERFRWNRYLNSNKGGYDHYSDVT |
| | KFLVELFQEYGIDFERGDIVGQIEVLETKGNEKFFKNFVFFFNLICQIRNTNAS |
| | ELAKKDGKDDFILSPVEPFFDSRNSEKFGEDLPKNGDDNGAFNIARKGLVIM |
| | DKITKFADENGGCEKMKWGDLYVSNVEWDNFVANK |
| SEQ ID | MFNNFIKKYSLQKTLRFELKPVGETADYIEDFKSEYLKDTVLKDEQRAKDY |
| NO: 45 | QEIKTLIDDYHREYIEECLREPVDKKTGEILDFTQDLEDAFSYYQKLKENPTE |
| | NRVGWEKEQESLRKKLVTSFVGNDGLFKKEFITRDLPEWLQKKGLWGEYK |
| | DTVENFKKFTTYFSGFHENRKNMYTAEAQSTAIANRLMNDNLPKFFNNYLA |
| | YQTIKEKHPDLVFRLDDALLQAAGVEHLDEAFQPRYFSRLFAQSGITAFNELI |
| | GGRTTENGEKIQGLNEQINLYRQQNPEKAKGFPRFMPLFKQILSDRETHSFLP |
| | DAFENDKELLQALRDYVDAATSEEGMISQLNKAMNQFVTADLKRVYIKSAA |
| | LTSLSQELFHFFGVISDAIAWYAEKRLSPKKAQESFLKQEVYAIEELNQAVVG |
| | YIDQLEDQSELQQLLVDLPDPQKPVSSFILTHWQKSQEPLQAVIAKVEPLFEL |
| | EELSKNKRAPKHDKDQGGEGFQQVDAIKNMLDAFMEVSHAIKPLYLVKGR |
| | KAIDMPDVDTGFYADFAEAYSAYEQVTVSLYNKTRNHLSKKPFSKDKIKINF |
| | DAPTLLNGWDLNKESDNKSIILRKDGNFYLAIMHPKHTKVFDCYSASEAAG |
| | KCYEKMNYKLLSGANKMLPKVFFSKKGIETFSPPQEILDLYKNNEHKKGAT |
| | FKLESCHKLIDFFKRNIPKYKVHPTDNFGWDVFGFHFSPTSSYGDLSGFYRE |
| | VEAQGYKLWFSDVSEAYINKCVEEGKLFLFQIYNKDFSPNSTGKPNLHTLY |
| | WKGLFEPENLKDVVLKLNGEAEIFYRKHSIKHEDKTIHRAKDPIANKNADN |
| | PKKQSVFDYDIIKDKRYTQDKFFFHVPISLNFKSQGVVRENDKINGLLAAQD |
| | DVHVIGIDRGERHLLYYTVVNGKGEVVEQGSLNQVATDQGYVVDYQQKLH |
| | AKEKERDQARKNWSTIENIKELKAGYLSQVVHKLAQLIVKHNAIVCLEDLN |
| | FGFKRGRFKVEKQVYQKFEKALIDKLNYLVFKERGATQAGGYLNAYQLAA |
| | PFESFEKLGKQTGILYYVRSDYTSKIDPATGFVDFLKPKYESMAKSKVFFESF |
| | ERIQWNQAKGYFEFEFDYKKMCPSRKFGDYRTRWVVCTFGDTRYQNRRNK |
| | SSGQWETETIDVTAQLKALFAAYGITYNQEDNIKDAIAAVKYTKFYKQLYW |
| | LLRLTLSLRHSVTGTDEDFILSPVADENGVFFDSRKATDKQPKDADANGAYH |
| | IALKGLWNLQQIRQHDWNVEKPKKLNLAMKNEEWFGFAQKKKFRA |
| SEQ ID | MIKNPSNRYALPKVIISKIDNQNILEFKIKYKKLSKLDIVKVKSMHYDDRAIIF |
| NO: 46 | DEVIVNDGLIDVEYRDNHKTIFVKVGNKSYSISGQKVGGKERLLENRVSKTK |
| | VQLELKDKATNRVSKTERELIVDDNIKIYSQIVGRDVKTTKDIYLIKRFLAYR |
| | SDLLFYYGFVNNFFHVANNRSEFWKIDFNDSNNSKLIEYFKFTINDHLKNDE |
| | NYLKDYISDNEKLKNDLIKVKNSFEKIRHALMHFDYDFFVKLFNGEDVGLE |
| | LDIEFLDIMIDKLDKLNIDTKKEFIDDEKITIFGEELSLAKLYRFYAHTAINRVA |
| | FNKLINSFIIENGVENQSLKEYFNQQAGGIAYEIDIHQNREYKNLYNEHKKLV |
| | SRVLSISDGQEIAILNQKIAKLKDQMKQITKANSIKRLEYKLRLALGFIYTEYE |
| | NYEEFKNNFDTDIKNGRFTPKDNDGNKRAFDSRELEQLKGYYEATIQTQKP |
| | KTDEKIEEVSKKIDRLSLKSLIADDILLKFILLMFTFMPQELKGEFLGFIKKYY |
| | HDTKHIDQDTISDSDDTIETLSIGLKLKILDKNIRSLSILKHSLSFQTKYNKKD |
| | RNYYEDGNIHGKFFKKLGISHNQEEFNKSVYAPLFRYYSALYKLINDFEIYTL |
| | SLHIVGSETLTDQVNKSQFLSGRYFNFRKLLTQSYHINNNSTHSTIFNAVINM |
| | RNDISHLSYEPLFDCPLNGKKSYKRKIRNQFKTINIKPLVESRKIIIDFITLQTD |
| | MQKVLGYDAVNDFTMKIVQLRTRLKAYANKEQTIQKMITEAKTPNDFYNIY |
| | KVQGVEEINKYLLEVIGETQAEKEIREKIERGNIANF |
| SEQ ID | MIKNPSNRHSLPKVIISEVDHEKILEFKIKYEKLARLDRFEVKAMHYEGKEIV |
| NO: 47 | FDEVLVNGGLIEVEYQDDNKTLFVKVGEKSYSIRGKKVGGKQRLLEDRVSK |
| | TKVQLELSDGVVDNKGNLRKSRTERELIVADNIKLYSQIVGREVTTTKEIYLV |
| | KRFLAYRSDLLFYYSFVDNFFKVAGNEKELWKINFDDATSAQFMGYIPFMV |
| | NDNLKNDNAYLKDYVRNDVQIKDDLKKVQTIFSALRHTLLHFNYEFFEKLF |
| | NGEDVGFDFDIGFLNLLIENIDKLNIDAKKEFIDNEKIRLFGENLSLAKVYRL |
| | YSDICVNRVGFNKFINSMLIKDGVENQVLKAEFNRKFGGNAYTIDIHSNQEY |
| | KRIYNEHKKLVIKVSTLKDGQAIRRGNKKISELKEQMKSMTKKNSLARLEC |
| | KMRLAFGFLYGEYNNYKAFKNNFDTNIKNSQFDVNDVEKSKAYFLSTYERR |
| | KPRTREKLEKVAKDIESLELKTVIANDTLLKFILLMFVFMPQELKGDFLGFVK |
| | KYYHDVHSIDDDTKEQEEDVVEAMSTSLKLKILGRNIRSLTLFKYALSSQVN |
| | YNSTDNIFYVEGNRYGKIYKKLGISHNQEEFDKTLVVPLLRYYSSLFKLMND |
| | FEIYSLAKANPTAVSLQELVDDETSPYKQGNYFNFNKMLRDIYGLTSDEIKSG |
| | QVVFMRNKIAHFDTEVLLSKPLLGQTKMNLQRKDIVSFIEARGDIKELLGYD |
| | AINDFRMKVIHLRTKMRVYSDKLQTMMDLLRNAKTPNDFYNVYKVKGVES |
| | INKHLLEVLAQTAEERTVEKQIRDGNEKYDL |
| SEQ ID | MEEKMLKSYDYFTKLYSLQKTLRFELKPIGKTLEHIKNSGIIESDETLEEQYAI |
| NO: 48 | VKNIIDKLHRKHIDEALSLVDFTKHLDTLKTFQELYLKRGKTDKEKEELEKL |
| | SADLRKLIVSYLKGNVKEKTQHNLNPIKERFEILFGKELFTNEEFFLLAENEK |
| | EKKAIQAFKGFTTYFKGFQENRKNMYSEEGNSTSIAYRIINENLPLFIENIARF |
| | QKVMSTIEKTTIKKLEQNLKTELKKHNLPGIFTIEYFNNVLTQEGISRYNTIIG |
| | GKTTHEGVKIQGLNEIINLYNQQSKDVKLPILKPLHKQILSEEYSTSFKIKAFE |
| | NDNEVLKAIDTFWNEHIEKSIHPVTGNKFNILSKIENLCDQLQKYKDKDLEK |
| | LFIERKNLSTVSHQVYGQWNIIRDALRMHLEMNNKNIKEKDIDKYLDNDAF |
| | SWKEIKDSIKIYKEHVEDAKELNENGIIKYFSAMSINEEDDEKEYSISLIKNIN |
| | EKYNNVKSILQEDRTGKSDLHQDKEKVGIIKEFLDSLKQLQWFLRLLYVTVP |
| | LDEKDYEFYNELEVYYEALLPLNSLYNKVRNYMTRKPYSVEKFKLNFNSPT |
| | LLDGWDKNKETANLSIILRKNGKYYLGIMNKENNTIFEYYPGTKSNDYYEK |
| | MIYKLLPGPNKMLPKVFFSKKGLEYYNPPKEILNIYEKGEFKKDKSGNFKKE |
| | SLHTLIDFYKEAIAKNEDWEVFNFKFKNTKEYEDISQFYRDVEEQGYLITFE |
| | KVDANYVDKLVKEGKLYLFQIYNKDFSENKKSKGNPNLHTIYWKGLYDSE |
| | NLKNVVYKLNGEAEVFYRKKSIDYPEEIYNHGHHKEELLGKFNYPIIKDRRY |
| | TQDKFLFHVPITMNFISKEEKRVNQLACEYLSATKEDVHIIGIDRGERHLLYL |
| | SLIDKEGNIKKQLSLNTIKNENYDKEIDYRVKLDEKEKKRDEARKNWDVIEN |
| | IKELKEGYMSQVIHIIAKMMVEEKAILIMEDLNIGFKRGRFKVEKQVYQKFE |
| | KMLIDKLNYLVFKNKNPLEPGGSLNAYQLTSKFDSFKKLGKQSGFIFYVPSA |
| | YTSKIDPTTGFYNFIQVDVPNLEKGKEFFSKFEKIIYNTKEDYFEFHCKYGKF |
| | VSEPKNKDNDRKTKESLTYYNAIKDTVWVVCSTNHERYKIVRNKAGYYES |
| | HPVDVTKNLKDIFSQANINYNEGKDIKPIIIESNNAKLLKSIAEQLKLILAMRY |
| | NNGKHGDDEKDYILSPVKNKQGKFFCTLDGNQTLPINADANGAYNIALKGL |
| | LLIEKIKKQQGKIKDLYISNLEWFMFMMSR |
| SEQ ID | MEKSLNDFIGLYSVSKTLRFELKPVSETLENIKKFHFLEEDKKKANDYKDVK |
| NO: 49 | KIIDNYHKYFIDDVLKNASFNWKKLEEAIREYNKNKSDDSALVAEQKKLGD |
| | AILKLFTSDKRYKALTAATPKELFESILPDWFGEQCNQDLNKAALKTFQKFTS |
| | YFTGFQENRKNVYSAEAIPTAVPYRIVNDNFPKFLQNVLIFKTIQEKCPQIIDE |
| | VEKELSSYLGKEKLAGIFTLESFNKYLGQGGKENQRGIDFYNQIIGGVVEKE |
| | GGINLRGVNQFLNLYWQQHPDFTKEDRRIKMVPLYKQILSDRSSLSFKIESIE |
| | NDEELKNALLECADKLELKNDEKKSIFEEVCDLFSSVKNLDLSGIYINRKDIN |
| | SVSRILTGDWSWLQSRMNVYAEEKFTTKAEKARWQKSLDDEGENKSKGFY |
| | SLTDLNEVLEYSSENVAETDIRITDYFEHRCRYYVDKETEMFVQGSELVALSL |
| | QEMCDDILKKRKAMNTVLENLSSENKLREKTDDVAVIKEYLDAVQELLHRI |
| | KPLKVNGVGDSTFYSVYDSIYSALSEVISVYNKTRNYITKKAASPEKYKLNF |
| | DNPTLADGWDLNKEQANTSVILRKDGMFYLGIMNPKNKPKFAEKYDCGNE |
| | SCYEKMIYKQFDATKQIPKCSTQKKEVQKYFLSGATEPYILNDKKSFKSELII |
| | TKDIWFMNNHVWDGEKFVPKRDNETRPKKFQIGYFKQTGDFDGYKNALSN |
| | WISFCKNFLQSYLSATVYDYNFKNSEEYEGLDEFYNYLNATCYKLNFINIPET |
| | EINKMVSEGKLYLFQIYNKDFASGSTGMPNMHTLYWKNLFSDENLKNVCLK |
| | LNGEAELFYRPAGIKEPVIHKEGSYLVNRTTEDGESIPEKIYFEIYKNANGKLE |
| | KLSDEAQNYISNHEVVIKKAGHEIIKDRHYTEPKFLFHVPLTINFKASGNSYSI |
| | NENVRKFLKNNPDVNIIGLDRGERHLIYLSLINQKGEIIKQFTFNEVERNKNG |
| | RTIKVNYHEKLDQREKERDAARKSWQAIGKIAELKEGYLSAVIHQLTKLMV |
| | EYNAVVVMEDLNFGFKRGRFHVEKQVYQKFEHILIDKSNYLVFKDRGLNEP |
| | GGVLNGYQIAGQFESFQKLGKQSGMLFYVPAGYTSKIDPKTGFVSMMNFK |
| | DLTNVHKKRDFFSKFDNIHYDEANGSFVFTFDYKKFDGKAKEEMKLTKWS |
| | VYSRDKRIVYFAKTKSYEDVLPTEKLQKIFESNGIDYKSGNNIQDSVMAIGA |
| | DLKEGAKPSKEISDFWDGLLSNFKLILQMRNSNARTGEDYIISPVMADDGTF |
| | FDSREEFKKGEDAKLPLDADANGAYHIALKGLSLINKINLSKDEELKKFDMK |
| | ISNADWFKFAQEKNYAK |
| SEQ ID | MNTQKKEFNPKSFKDFTNLYSLNKTLRFSLTPNKKTAEILEFNKQKEVKCFS |
| NO: 50 | NDRKIAGAYQEIKKYLNKLHQEFIQEAMKFFAFSEEELKGFEKEYLNLLNFT |
| | DKDNFKKKNKIRNEYEQERKILTIKIATYFSKFKSEKYQSFNLANITGKKVFSI |
| | LEQKYKEDKKTLKIIHIFKYKPTKDEKKEGEAVNFSTYLTGFNENRKNFYKS |
| | EDKAGQFATRTIDNLAQFIKNKKLFEDKYQKNYSKIGILDEQIKIFNLDYENN |
| | LFLQEGLDEYNGILGNNKGEENKSNEGINQKINIFKQKEKARLKKEKENFNK |
| | SDFPLFKELYKQIGSIRKENDVYVEIKTDKELVEELNNFPKNVENYLKDIQSF |
| | YKTFFEKLQNEEYELDKIYLPKSVGTYFSYIAFSDWNKLAFIYNKRYKNEKI |
| | KIVEGGDVNVQYRSLEVLKNRIDELKDEDNLNFNKFFIDKLKFNEAKKENN |
| | WQNFWFCIEYYINSQFIGGEKNILNKEKNEYEILPFGSLKELKEKYFEAVKKY |
| | KEKMVDTESGLTDDEEKEIKETLKNYLDRIKEIERIAKYFDLKKSFEEIKQED |
| | LDSNFYGEYQKVVDKTNELKIYQYYSEFRNYLTQNNSVEEKIKLNFNSGLLL |
| | DGWDLNKEKVKFSIIFQENGKYYLGIINKEKDKTILDKDKHPEIFTKNSDFRK |
| | MEYKLFPSPSKMLPKISFSETAKKGDEDVGWSEEIQKIKDEFAEFQEYKKKS |
| | KDNWKDEFNRGKLNKLIDYYKQVLEKHSEGYMNTYNFELKDSSKYKNLG |
| | EFNDDIARQNYKVKFVGIDKNYIDEKVANGELFLFQIYNKDFSEDKKEGSTN |
| | NLETIYFKELFSKENLENPVFKLSGGAEMFFRNKIEKKKEKKKLDKDGKPMI |
| | SKKGEKVVDKRRFSENKILFHLPIEINYGKGKMPNFNKKINEYISKNPENIKII |
| | GIDRGEKHLLYYSIIDQNGNNIESMSLNAVDEFGNFVNPEKLEEYEIDNNGK |
| | KERRWKYIVNDKEIKVTNYQRKLDELEKERQKSRQSWQNINKIKNLKKGYI |
| | SFVVKKIVDLAIENNAIIILEDLNFGFKSFRQKIEKNVYQQFEKALIDKLGFVV |
| | DKQKQNQRFAPQLSAPFESFQKIGKQTGIVYYVLANNTSKVCPSCQWIKNF |
| | YLKYEKKNTIFNLQKNQKLKVFFEQEKNRFRFEYQMSKEYISVYSDVDRQR |
| | YDKTKNQNKGGYLEYKNSNQKEIIDKDGVIQKQSITLQLKELFKENHIDLEK |
| | EILKQLDNKKEKNSGYTGVYNKFIYLFNLILQIRNAISFREKDYIQCPSCHFD |
| | TRKENYLKINDGDGNGAYNIALRGLYLLKGKNGIINNLEKIKLIFSNNDYFQ |
| | WAKKLKNKK |
| SEQ ID | MQNKQSFADFTNLYSLSKTLRFELKPIGQTQAMLDENKIFEVDENRKKAYD |
| NO: 51 | KTKPYFDRLHREFINESLSNAQLKGISEYFETFKQFRSNQNNKDLKELINKQQ |
| | KFLRHQIVTLFDENGKHWATTKYAHLKIKKKNLDILFDEQVFYILKERYGSE |
| | KETQLVDKETGAVTSIFDNWKGFTGYFTKFFETRKNFYKSDGTSTALATRIID |
| | QNLNRFFDNLETFHKIKDKIDVKEVEIFFKLKADNVFSIDFYNQCLLQNGIDK |
| | YNDFLGGQTLENGEKQKGINEIINKYRQDNKDQKLPFLKKLDKQILSEKDRF |
| | INEIESKEEFFQVLTEFYQSATVKVTIIKTLLNDFVHNTDKYKLEKIYLTKEAF |
| | NTIANKWTDETQIFEDNLDLVLKNKKITAKQDFIPLAYIKEALEVIEKDRKFF |
| | KDRYYNDPQIGFFPDQSYWEQFLAILNFEFMTHFQRVAKDKITGKKIELGYF |
| | VFEKRIKELLDSDPSLNSQSKIIIKEFADEVLHIFQMAKYFALEKKREWKGDY |
| | YQLDDQFYNHIDYGFKDQFYENAYEKIVQPYNKIRNYLTKKPYSDVKWKL |
| | NFGNPTLANGWDKNKEADNTAVILKKDGNYYLGVMKKGKNKIFSDQNKE |
| | KYKAYNSAYYEKLVYKLFPDPSKMFPKVCFSKKGLNFFQPSEEILRIYKNNE |
| | FKKGNTFSISSMQKLIAFYIDCLGLYEGWKHYEFKNIKDVRQYKENIGEFYA |
| | DVAESGYKLWFEKISEEYITQKNQLGELFLFQIYNKDFAKKTTGRKNLHTIYF |
| | EELFSQTNIDNNFPFKLNGQAELFYRPKSLEKIEEKRNFKRSIVNKKRYTQNK |
| | IFFHVPITLNRTSENIGRFNVRVNNFLANNSNVNIVGVDRGEKNLAYYSIIKQ |
| | NGEVLKSGSLNIINGVDYHALLTDRAQRREQERRNWQDVESIKDLKRGYIS |
| | QVVHELVSLAIKYNAIIVMEDLNMRFKQIRGGIEKSTYQQLEKALIEKLNFLV |
| | NKEETDSNQAGNLLNAYQLTAPFKTFKDMGKQTGIIFYTQASYTSKIDPLTG |
| | WRPNIYLRYSNAKQAKADILMFTNIYFSEKKDRFEFTYDLEKIDDKRKDLPI |
| | KTEWTVCSNVERFSWEKSLNNNKGGYVHYPIQDSNGEESITSKLKKLFMDF |
| | GIDLTDIKTQIESLDTNKKDNANFFRKFIFYFQLICQIRNTQVNKSDDGNDFIF |
| | SPVEPFFDSRFADKFRKNLPKNGDENGAYNIARKGLIILHKISDYFVKEGSTD |
| | KISWKDLSISQTEWDNFTTDK |
| SEQ ID | MKKEKEFKSFGDFTNLYEISKTLRFELKPVENTQTMLDEADVFGKDKVIKD |
| NO: 52 | KYTKTKPFIDKLHREFVDESLKDVSLSGLKKYSEVLENWKKNKKDKDIVKE |
| | LKKEEERLRKEVVEFFDNTAKKWANEKYKELGLKKKDIGILFEESVFDLLKE |
| | KYGEEQDSFLKEEKGDFLKNEKGEKVSIFDEWKGFVGYFTKFQETRKNFYK |
| | NDGTETALATRIIDQNLKRFCDNIDDFKKIKNKIDFSEVEKNFNKTADVFSLD |
| | FYNQCLLQKGIDSYNEFIGGKTLENGKKLKGVNELVNEYRQKNKNEKVSFL |
| | KLLDKQILSEKEKLSFGIENDEQLLVVLNSFYETAEEKTKILRTLFGDFVEHN |
| | ENYDLDKTYISKVAFNTISHKWTNETHKFEELLYGAMKEDKPIGLNYDKKE |
| | DSYKFPDFIALGYLKKCLNNLDCDTKFWKEKYYENNADKKDKDKGFLTGG |
| | QNAWDQFLQIFIFEFNQLFNSEAFDNKGKEIKIGYDNFRKDFEEIINQKDFKN |
| | DENLKIAIKNFADSVLWIYQMAKYFAIEKKRGWDDDFELSEFYTNPSNGYSL |
| | FYDRAYEEIVQKYNDLRNYLTKKPYKEDKWKLNFENPTLANGFDKNKESD |
| | NSTVILRKKRKYYLGLMKKGNNKIFEDRNKAEFIRNIESGAYEKMAYKYLP |
| | DVAKMIPKCSTQLNEAKNHFRNSADDLEIKKSFSNPLKITKRIFDLNNIQYDK |
| | TNVSKKISGDNKGIKIFQKEYYKISGDFDVYKSALNDWIDFCKDFLSKYDST |
| | KDFDFSILRKTKDYKSLDEFYVDVAKITYKISFTPVSESYIDQKNKNGELYLF |
| | EIYNQDFAKGKMGAKNLHTLYFENVFSPENISKNFPIKLNGNAELFFRPKSIE |
| | SKKEKRNFVREIVNKKRYSEDKIFFHCPITLNRETGSIYRFNNYVNNFLSENN |
| | INIIGVDRGEKHLAYYSVIDKNGVKIGGGSFNEINKVDYAKKLEERAGEREQ |
| | SRKDWQVVEGIKDLKKGYISQVVRELADLAIKHNAIIVLEDLNMRFKQIRG |
| | GIEKSIYQQLEKALIDKLSFLVEKGEKDPNQAGHILKAYQLAAPFTSFKDMG |
| | KQTGIVFYTQASYTSKTCPNCGFRKNNNKFYFENNIGKAQDALKKLKTFEY |
| | DSENKCFGLSYCLSDFANKEEVEKNKNKKRNNAPYSDIEKKDCFELSTKDA |
| | VRYRWHDKNTERGKTFFEGESVYEEKEEKEIGQTKRGLVKEYDISKCLIGLF |
| | EKTGLDYKQNLLDKINSGKFDGTFYKNLFNYLNLLFEIRNSISGTEIDYISCPE |
| | CQFHTDKSKTIKNGDDNGSYNIARKGMIILDKIKQFKKENGSLDKMGWGEL |
| | FIDLEEWDKFAQKKNNNIIDK |
| SEQ ID | MKYTDFTGIYSVSKTLRFELIPQGSTVENMKREGILNNDMHRADSYKEMKK |
| NO: 53 | LIDEYHKAFIERCLSDFSLKYDDTGKHDSLEEYFFYYEQKRNDKTKKIFEDIQ |
| | VALRKQISKRFTGDTAFKRLFKKELIKEDLPSFVKNDPVKTELIKEFSDFTTYF |
| | QEFHKNRKNMYTSDAKSTAIAYRIINENLPKFIDNINAFDIVAKVPEMQEHFK |
| | TIADELRSHLQVGNDIDKMFNLQFFNKVLTQSQLDVYNAVIGGKSEGNKKIQ |
| | GINEYINLYNQQHKKARLPMLKLLYKQILSDRVAISWLQDEFDNDQDMLDTI |
| | EAFYNKLNSNETGVLGEGKLKQILMGLDGYNLDGVFMRNDLQLSEVSQRL |
| | CGGWNIIKDAMTSDLKRSVQKKKKETDADFEERVSKLFSAQNSFSIAYINQC |
| | LGQAGIRCKIQDYFACLGAKEGENEAETTPDIFDQIAEAYHGAAPILNARPSS |
| | HNLAQDIEKVKAIKALLDALKRLQRFVKPLLGRGDEGDKDNFFYGDFMPIW |
| | EVLDQLTPLYNKVRNRMTRKPYSQEKIKLNFENSTLLNGWDLNKEHDNTSV |
| | ILRREGLYYLGIMNKNYNKIFDANNVETIGDCYEKMIYKLLPGPNKMLPKVF |
| | FSKSRVQEFSPSKKILEIWESKSFKKGDNFNLDDCHALIDFYKDSIAKHPDW |
| | NKFNFKFSDTQSYTNISDFYRDVNQQGYSLSFTKVSVDYVNRMVDEGKLYL |
| | FQIYNKDFSPQSKGTPNMHTLYWRMLFDERNLHNVIYKLNGEAEVFYRKAS |
| | LRCDRPTHPAHQPITCKNENDSKRVCVFDYDIIKNRRYTVDKFMFHVPITINY |
| | KCTGSDNINQQVCDYLRSAGDDTHIIGIDRGERNLLYLVIIDQHGTIKEQFSL |
| | NEIVNEYKGNTYCTNYHSLLEEKEAGNKKARQDWQTIESIKELKEGYLSQVI |
| | HKISMLMQRYHAIVVLEDLNGSFMRSRQKVEKQVYQKFEHMLINKLNYLV |
| | NKQYDATEPGGLLHALQLTSRMDSFKKLGKQSGFLFYIPAWNTSKIDPVTGF |
| | VNLFDTRYCNEAKAKEFFEKFDDISYNDERDWFEFSFDYRHFTNKPTGTRTQ |
| | WTLCTQGTRVRTFRNPEKSNHWDNEEFDLTQAFKDLFNKYGIDIASGLKARI |
| | VNGQLTKETSAVKDFYESLLKLLKLTLQMRNSVTGTDIDYLVSPVADKDGIF |
| | FDSRTCGSLLPANADANGAFNIARKGLMLLRQIQQSSIDAEKIQLAPIKNED |
| | WLEFAQEKPYL |
| SEQ ID | MEKEITELTKIRREFPNKKFSSTDMKKAGKLLKAEGPDAVRDFLNSCQEIIGD |
| NO: 54 | FKPPVKTNIVSISRPFEEWPVSMVGRAIQEYYFSLTKEELESVHPGTSSEDHK |
| | SFFNITGLSNYNYTSVQGLNLIFKNAKAIYDGTLVKANNKNKKLEKKFNEIN |
| | HKRSLEGLPIITPDFEEPFDENGHLNNPPGINRNIYGYQGCAAKVFVPSKHK |
| | MVSLPKEYEGYNRDPNLSLAGFRNRLEIPEGEPGHVPWFQRMDIPEGQIGHV |
| | NKIQRFNFVHGKNSGKVKFSDKTGRVKRYHHSKYKDATKPYKFLEESKKVS |
| | ALDSILAIITIGDDWVVFDIRGLYRNVFYRELAQKGLTAVQLLDLFTGDPVID |
| | PKKGVVTFSYKEGVVPVFSQKIVPRFKSRDTLEKLTSQGPVALLSVDLGQNE |
| | PVAARVCSLKNINDKITLDNSCRISFLDDYKKQIKDYRDSLDELEIKIRLEAIN |
| | SLETNQQVEIRDLDVFSADRAKANTVDMFDIDPNLISWDSMSDARVSTQISD |
| | LYLKNGGDESRVYFEINNKRIKRSDYNISQLVRPKLSDSTRKNLNDSIWKLK |
| | RTSEEYLKLSKRKLELSRAVVNYTIRQSKLLSGINDIVIILEDLDVKKKFNGR |
| | GIRDIGWDNFFSSRKENRWFIPAFHKAFSELSSNRGLCVIEVNPAWTSATCPD |
| | CGFCSKENRDGINFTCRKCGVSYHADIDVATLNIARVAVLGKPMSGPADRER |
| | LGDTKKPRVARSRKTMKRKDISNSTVEAMVTA |
| SEQ ID | MIKPTVSQFLTPGFKLIRNHSRTAGLKLKNEGEEACKKFVRENEIPKDECPNF |
| NO: 55 | QGGPAIANIIAKSREFTEWEIYQSSLAIQEVIFTLPKDKLPEPILKEEWRAQWL |
| | SEHGLDTVPYKEAAGLNLIIKNAVNTYKGVQVKVDNKNKNNLAKINRKNEI |
| | AKLNGEQEISFEEIKAFDDKGYLLQKPSPNKSIYCYQSVSPKPFITSKYHNVN |
| | LPEEYIGYYRKSNEPIVSPYQFDRLRIPIGEPGYVPKWQYTFLSKKENKRRKL |
| | SKRIKNVSPILGIICIKKDWCVFDMRGLLRTNHWKKYHKPTDSINDLFDYFT |
| | GDPVIDTKANVVRFRYKMENGIVNYKPVREKKGKELLENICDQNGSCKLAT |
| | VDVGQNNPVAIGLFELKKVNGELTKTLISRHPTPIDFCNKITAYRERYDKLES |
| | SIKLDAIKQLTSEQKIEVDNYNNNFTPQNTKQIVCSKLNINPNDLPWDKMISG |
| | THFISEKAQVSNKSEIYFTSTDKGKTKDVMKSDYKWFQDYKPKLSKEVRDA |
| | LSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTS |
| | ITCPKCKYCDSKNRNGEKFNCLKCGIELNADIDVATENLATVAITAQSMPKPT |
| | CERSGDAKKPVRARKAKAPEFHDKLAPSYTVVLREAV |
| SEQ ID | MEKEITELTKIRREFPNKKFSSTDMKKAGKLLKAEGPDAVRDFLNSCQEIIGD |
| NO: 56 | FKPPVKTNIVSISRPFEEWPVSMVGRAIQEYYFSLTKEELESVHPGTSSEDHK |
| | SFFNITGLSNYNYTSVQGLNLIFKNAKAIYDGTLVKANNKNKKLEKKFNEIN |
| | HKRSLEGLPIITPDFEEPFDENGHLNNPPGINRNIYGYQGCAAKVFVPSKHK |
| | MVSLPKEYEGYNRDPNLSLAGFRNRLEIPEGEPGHVPWFQRMDIPEGQIGHV |
| | NKIQRFNFVHGKNSGKVKFSDKTGRVKRYHHSKYKDATKPYKFLEESKKVS |
| | ALDSILAHITIGDDWVVFDIRGLYRNVFYRELAQKGLTAVQLLDLFTGDPVID |
| | PKKGVVTFSYKEGVVPVFSQKIVPRFKSRDTLEKLTSQGPVALLSVDLGQNE |
| | PVAARVCSLKNINDKITLDNSCRISFLDDYKKQIKDYRDSLDELEIKIRLEAIN |
| | SLETNQQVEIRDLDVFSADRAKANTVDMFDIDPNLISWDSMSDARVSTQISD |
| | LYLKNGGDESRVYFEINNKRIKRSDYNISQLVRPKLSDSTRKNLNDSIWKLK |
| | RTSEEYLKLSKRKLELSRAVVNYTIRQSKLLSGINDIVIILEDLDVKKKFNGR |
| | GIRDIGWDNFFSSRKENRWFIPAFHKTFSELSSNRGLCVIEVNPAWTSATCPD |
| | CGFCSKENRDGINFTCRKCGVSYHADIDVATLNIARVAVLGKPMSGPADRER |
| | LGDTKKPRVARSRKTMKRKDISNSTVEAMVTA |
| SEQ ID | VPDKKETPLVALCKKSFPGLRFKKHDSRQAGRILKSKGEGAAVAFLEGKGGT |
| NO: 57 | TQPNFKPPVKCNIVAMSRPLEEWPIYKASVVIQKYVYAQSYEEFKATDPGKS |
| | EAGLRAWLKATRVDTDGYFNVQGLNLIFQNARATYEGVLKKVENRNSKKV |
| | AKIEQRNEHRAERGLPLLTLDEPETALDETGHLRHRPGINCSVFGYQHMKLK |
| | PYVPGSIPGVTGYSRDPSTPIAACGVDRLEIPEGQPGYVPPWDRENLSVKKH |
| | RRKRASWARSRGGAIDDNMLLAVVRVADDWALLDLRGLLRNTQYRKLLDR |
| | SVPVTIESLLNLVTNDPTLSVVKKPGKPVRYTATLIYKQGVVPVVKAKVVKG |
| | SYVSKMLDDTTETFSLVGVDLGVNNLIAANALRIRPGKCVERLQAFTLPEQT |
| | VEDFFRFRKAYDKHQENLRLAAVRSLTAEQQAEVLALDTFGPEQAKMQVC |
| | GHLGLSVDEVPWDKVNSRSSILSDLAKERGVDDTLYMFPFFKGKGKKRKTE |
| | IRKRWDVNWAQHFRPQLTSETRKALNEAKWEAERNSSKYHQLSIRKKELSR |
| | HCVNYVIRTAEKRAQCGKVIVAVEDLHHSFRRGGKGSRKSGWGGFFAAKQE |
| | GRWLMDALFGAFCDLAVHRGYRVIKVDPYNTSRTCPECGHCDKANRDRVN |
| | REAFICVCCGYRGNADIDVAAYNIAMVAITGVSLRKAARASVASTPLESLAA |
| | E |
| SEQ ID | MPKPAVESEFSKVLKKHFPGERFRSSYMKRGGKILAAQGEEAVVAYLQGKSE |
| NO: 58 | EEPPNFQPPAKCHVVTKSRDFAEWPIMKASEAIQRYIYALSTTERAACKPGKS |
| | SESHAAWFAATGVSNHGYSHVQGLNLIFDHTLGRYDGVLKKVQLRNEKAR |
| | ARLESINASRADEGLPEIKAEEEEVATNETGHLLQPPGINPSFYVYQTISPQAY |
| | RPRDEIVLPPEYAGYVRDPNAPIPLGVVRNRCDIQKGCPGYIPEWQREAGTAI |
| | SPKTGKAVTVPGLSPKKNKRMRRYWRSEKEKAQDALLVTVRIGTDWVVID |
| | VRGLLRNARWRTIAPKDISLNALLDLFTGDPVIDVRRNIVTFTYTLDACGTY |
| | ARKWTLKGKQTKATLDKLTATQTVALVAIDLGQTNPISAGISRVTQENGALQ |
| | CEPLDRFTLPDDLLKDISAYRIAWDRNEEELRARSVEALPEAQQAEVRALDG |
| | VSKETARTQLCADFGLDPKRLPWDKMSSNTTFISEALLSNSVSRDQVFFTPA |
| | PKKGAKKKAPVEVMRKDRTWARAYKPRLSVEAQKLKNEALWALKRTSPEY |
| | LKLSRRKEELCRRSINYVIEKTRRRTQCQIVIPVIEDLNVRFFHGSGKRLPGW |
| | DNFFTAKKENRWFIQGLHKAFSDLRTHRSFYVFEVRPERTSITCPKCGHCEV |
| | GNRDGEAFQCLSCGKTCNADLDVATHNLTQVALTGKTMPKREEPRDAQGTA |
| | PARKTKKASKSKAPPAEREDQTPAQEPSQTS |
| SEQ ID | MSNKTTPPSPLSLLLRAHFPGLKFESQDYKIAGKKLRDGGPEAVISYLTGKG |
| NO: 59 | QAKLKDVKPPAKAFVIAQSRPFIEWDLVRVSRQIQEKIFGIPATKGRPKQDGL |
| | SETAFNEAVASLEVDGKSKLNEETRAAFYEVLGLDAPSLHAQAQNALIKSAI |
| | SIREGVLKKVENRNEKNLSKTKRRKEAGEEATFVEEKAHDERGYLIHPPGV |
| | NQTIPGYQAVVIKSCPSDFIGLPSGCLAKESAEALTDYLPHDRMTIPKGQPGY |
| | VPEWQHPLLNRRKNRRRRDWYSASLNKPKATCSKRSGTPNRKNSRTDQIQS |
| | GRFKGAIPVLMRFQDEWVIIDIRGLLRNARYRKLLKEKSTIPDLLSLFTGDPSI |
| | DMRQGVCTFIYKAGQACSAKMVKTKNAPEILSELTKSGPVVLVSIDLGQTNP |
| | IAAKVSRVTQLSDGQLSHETLLRELLSNDSSDGKEIARYRVASDRLRDKLAN |
| | LAVERLSPEHKSEILRAKNDTPALCKARVCAALGLNPEMIAWDKMTPYTEFL |
| | ATAYLEKGGDRKVATLKPKNRPEMLRRDIKFKGTEGVRIEVSPEAAEAYREA |
| | QWDLQRTSPEYLRLSTWKQELTKRILNQLRHKAAKSSQCEVVVMAFEDLNI |
| | KMMHGNGKWADGGWDAFFIKKRENRWFMQAFHKSLTELGAHKGVPTIEV |
| | TPHRTSITCTKCGHCDKANRDGERFACQKCGFVAHADLEIATDNIERVALTG |
| | KPMPKPESERSGDAKKSVGARKAAFKPEEDAEAAE |
| SEQ ID | MSKTKELNDYQEALARRLPGVRHQKSVRRAARLVYDRQGEDAMVAFLDG |
| NO: 60 | KEVDEPYTLQPPAKCHILAVSRPIEEWPIARVTMAVQEHVYALPVHEVEKSRP |
| | ETTEGSRSAWFKNSGVSNHGVTHAQTLNAILKNAYNVYNGVIKKVENRNA |
| | KKRDSLAAKNKSRERKGLPHFKADPPELATDEQGYLLQPPSPNSSVYLVQQ |
| | HLRTPQIDLPSGYTGPVVDPRSPIPSLIPIDRLAIPPGQPGYVPLHDREKLTSNK |
| | HRRMKLPKSLRAQGALPVCFRVFDDWAVVDGRGLLRHAQYRRLAPKNVSI |
| | AELLELYTGDPVIDIKRNLMTFRFAEAVVEVTARKIVEKYHNKYLLKLTEPK |
| | GKPVREIGLVSIDLNVQRLIALAIYRVHQTGESQLALSPCLHREILPAKGLGDF |
| | DKYKSKFNQLTEEILTAAVQTLTSAQQEEYQRYVEESSHEAKADLCLKYSITP |
| | HELAWDKMTSSTQYISRWLRDHGWNASDFTQITKGRKKVERLWSDSRWAQ |
| | ELKPKLSNETRRKLEDAKHDLQRANPEWQRLAKRKQEYSRHLANTVLSMA |
| | REYTACETVVIAIENLPMKGGFVDGNGSRESGWDNFFTHKKENRWMIKDIH |
| | KALSDLAPNRGVHVLEVNPQYTSQTCPECGHRDKANRDPIQRERFCCTHCG |
| | AQRHADLEVATHNIAMVATTGKSLTGKSLAPQRLQEAAE |
| SEQ ID | VAFLDGKEVDEPYTLQPPAKCHILAVSRPIEEWPIARVTMAVQEHVYALPVH |
| NO: 61 | EVEKSRPETTEGSRSAWFKNSGVSNHGVTHAQTLNAILKNAYNVYNGVIKK |
| | VENRNAKKRDSLAAKNKSRERKGLPHFKADPPELATDEQGYLLQPPSPNSS |
| | VYLVQQHLRTPQIDLPSGYTGPVVDPRSPIPSLIPIDRLAIPPGQPGYVPLHDR |
| | EKLTSNKHRRMKLPKSLRAQGALPVCFRVFDDWAVVDGRGLLRHAQYRRL |
| | APKNVSIAELLELYTGDPVIDIKRNLMTFRFAEAVVEVTARKIVEKYHNKYLL |
| | KLTEPKGKPVREIGLVSIDLNVQRLIALAIYRVHQTGESQLALSPCLHREILPA |
| | KGLGDFDKYKSKFNQLTEEILTAAVQTLTSAQQEEYQRYVEESSHEAKADLC |
| | LKYSITPHELAWDKMTSSTQYISRWLRDHGWNASDFTQITKGRKKVERLWS |
| | DSRWAQELKPKLSNETRRKLEDAKHDLQRANPEWQRLAKRKQEYSRHLAN |
| | TVLSMAREYTACETVVIAIENLPMKGGFVDGNGSRESGWDNFFTHKKENR |
| | WMIKDIHKALSDLAPNRGVHVLEVNPQYTSQTCPECGHRDKANRDPIQRER |
| | FCCTHCGAQRHADLEVATHNIAMVATTGKSLTGKSLAPQRLQ |
| SEQ ID | MIKKPSNRHALPKVIISKVDNQNILEFKIKYKKLSRLDRVEIKTMHYDDRAIV |
| NO: 81 | FDEVIINGGLIDVEYRDNHKTIFVKVGDKSYSISGQKVGGKERLLENRISQTK |
| | VQLELKDEATNRVSKTERELIVDDNIKLYSQIVGRDVKTTKDIYLIKRFLGYR |
| | SDLLFYYGFVNNFFHVANNRPEFWKIDFNDNRNSKLIEYFIFTINDHLKNDE |
| | NYLKDYISDRGQIVDDLENIKHIFSALRHGLMHFDYDFFEALFNGEDIDIKM |
| | DNQGNTQPLSSLNIKFLDIMIDKLDKLNIDTKKEFIDAEKITIFGEELSLAKLY |
| | RFYAHTAINRVAFNKLINSFIIENGVENQSLKEYFNQQAGGIAYEIDIHQNREY |
| | KNLYNEHKKLVSRVLSISDGQEIATLNQKIVELKEQMKQITKINSIKRLEYKL |
| | RLAFGFIYTEYKNYEEFKNSFDTDIKNGRFTPKDEDGNKRAFDSRELEHLKG |
| | YYKATLQTQKPQTDEKMEEVSKRVDRLSLKSLIGDDTLLKFILLMFTFMPQE |
| | LKGEFLGFIKKYYHDTKHIDQDTISDSDDTIEEGLSIGLKLKILDKNIRSLSILK |
| | HSLSFQTKYNKKDRSYYEDGNIHGKFFKKLGISHNQEEFNKSVYAPLFRYYS |
| | ALYKLINDFEIYTLSLHIVGNETLSDQVNKPQFLSGRYFNFRKLLTQSYNISN |
| | NSTHSVIFNAVINMRNDISHLSYEPLLDCPLNGKKSYKRKIRNQFRTINIKPLV |
| | ESRKMIIDFITLQTDMQKVLGCDAVNDFTMKIVQLRTRLKAYANKEQTIEKM |
| | ITEAKTPNDFYNIYKVKGVEAINKYLLEVIGETQVEKEIREEIERGNIANS |
| SEQ ID | MLKKPSNRYALPKVILSTVDHEKILEFKVKYEKLARLDRLVVERMHEDGES |
| NO: 82 | VVFDEVIANSGDLEIAYQDDHRKLLIQAAGKSYTITGKKVGGKKRKLEERIS |
| | RAKIQLTLTDGQEDQHRRIRATVTEKALLEPKEDRDIYSKISDRKIKTSKEIYL |
| | VKRFLSYRSDLLFYYFFVDNFFKVGNNKQELWKIKFQNQPELIEYFRFIINDR |
| | FKNAKNDKFDNYLKNDKAIQEDLEKIQKVFEKLRHALMHYDYGFFEKLFG |
| | GEDQGFDLDIAFLDNFVKKIDKLNIDTKKEFVDDEKIKIFGEDLNLADLYKL |
| | YASISINRVGFNRVVNEMIIKDGIEKSELKRAFEKKLDKTYALDIHSDPSYKKL |
| | YNEHKRLVTEVSTYTDGNKIKEGNQKIAKLKYEMKEITKKNALVRLECKMR |
| | LAFGLIYGRYDTHEAFKNGFDTDLKRGEFAQIGSEEAIGYFNTTFEKSKPKSK |
| | EEIKKIARQIDNLSLSTLIEDDPLMKFIVLMFLFVPRELKGEFLGFWRKYYHD |
| | IHSIDSDAKSDEMPDEVSLSLKLKILTRNIRRLNLFEYSLSEKIKYSPKNTQFY |
| | TDKSPYQKVYKRLKISHNKEEFDKTLLVPLFRYYSILFKLINDFEIYSLAKAN |
| | PDASSLSELTKTKHGFRGHYNFTTLMMDAHKVSQGDSKKHFGIRGEIAHINT |
| | KDLIYDPLFRKSKMAQQRNDVIDFVLKYEKEIKAVLGYDAINDFRMKVVQL |
| | RTKLKVYSDKTQTIEKLLNEVEAPDDFYVLYKVKGVEAINKYLLEIVSVTQA |
| | EEEIERKIITGNKRYNT |
| SEQ ID | MIKSYDDFTKLYSLQKTLRFELKPIGKTLEHIKKSEIIESDETLEEQYAIVKNII |
| NO: 83 | DRFHRKHIDEALSLVDFTKHLDTFKTIQELYLKRGKTDREKKELEELSADLR |
| | KLIVSYLKGNVKQKTQHNLNPIKERFEILFGKELFTNEEFFTLAENKEEKKAI |
| | QAFKGFTTYFKGFQENRKNMYSEEDKSTAIAYRIINENMPLFIENIARFQKVL |
| | DVIEKTKLTELKQNLKTELKGHSVSDIFRIEYFNNVLTQEGISRYNTIIGGKTT |
| | TEGVKIQGLNEIINLHNQQSKDVKLPILKPLHKQILSEEYSTSFKIKAFENDNE |
| | VLKAIDTFWNEHIEKSIHPVTGKRFNILLKIENLCKKLEKYKDKEIEKLFIERK |
| | NLSTVSHQVYGQWNIIRDALRMHLEMNNKNIKEKDIDKYLDNDAFAWKEI |
| | KDSIKIYKEHVEDAKELDENGIVKYFSSMSINEEDDEKEYSISLIKNINEKYN |
| | NVKSILEEDRTGKSDLHQDKEKVAIIKEFLDSLKQLQWFLKLLYVTVPLDEK |
| | DYEFYNELEVYYEALLPLNSLYNKVRNYMTRKPYSVEKFKLNFYSPTLLDG |
| | WDKNKETANLSIILKKNGKYYLGIMNKENNTIFENFPKSKSNDYYEKMIYK |
| | LLPGPNKMLPKVFFSKKGLEYYKPSKEILRIYEKGEFKKDKSGNFKKESLHT |
| | LIDFYKEAIAKNEDWKIFKFKFKNTREYEDISQFYRDVEEQGYLIIFEKVDAN |
| | YVDKLVEEGELYLFQIYNKDFSENKKSKGNPNLHTIYWESLFDNQNLKDVV |
| | YKLNGEAEVFYRKKSIDYPEEIYNNGHHKEELNGKFNYPIIKDRRYTQDKFL |
| | FHVPITMNFISKEEKRVNQLACEYLSTTKEDVHIIGIDRGERHLLYLSLIDKEG |
| | NIKKQLSLNTIKNENYDKEIDYRVKLDEKEKKRDEARKNWDVIENIKELKE |
| | GYMSQVIHIIAKMMAEEKAILIMEDLNIGFKRGRFKVEKQVYQKFEKMMID |
| | KLNYLVFKNKEPLEPGGSLNAYQLTSKFDSFKKLGKQSGFIFYVPSAYTSKID |
| | PTTGFYNFIRVDVPNLEKGKEFFSKFEKIIYNTKEDYFEFHCKYGKFVPEPKN |
| | KDNDRKTKESLTYYNAIKDTVWVVCSTHHERYKIVRNKAGYYESQPVDVT |
| | KNLRDIFSEANINYSDGKDIKPIIIESNNAKLLKSIAEQLKLILAMRYNNGKHD |
| | DDEKDYILSPVKNKQGKFFCSLDGDQSLPINADANGAYNIALKGLLLIEKIK |
| | KQQGKAKDLYISNLEWFMFMMSR |
| SEQ ID | MIKSYDDFTKLYSLQKTLRFELKPIGKTLEHIKKSEIIESDETLEEQYAIVKNII |
| NO: 84 | DRFHRKHIDEALSLVDFTKHLDTFKTIQELYLKRGKTDREKKELEELSADLR |
| | KLIVSYLKGNVKQKTQHNLNPIKERFEILFGKELFTNEEFFTLAENKEEKKAI |
| | QAFKGFTTYFKGFQENRKNMYSEEDKSTAIAYRIINENMPLFIENIARFQKVL |
| | DVIEKTKLTELKQNLKTELKGHSVSDIFRIEYFNNVLTQEGISRYNTIIGGKTT |
| | TEGVKIQGLNEIINLHNQQSKDVKLPILKPLHKQILSEEYSTSFKIKAFENDNE |
| | VLKAIDTFWNEHIEKSIHPVTGKRFNILLKIENLCKKLEKYKDKEIEKLFIERK |
| | NLSTVSHQVYGQWNIIRDALRMHLEMNNKNIKEKDIDKYLDNDAFAWKEI |
| | KDSIKIYKEHVEDAKELDENGIVKYFSSMSINEEDDEKEYSISLIKNINEKYN |
| | NVKSILEEDRTGKSDLHQDKEKVAIIKEFLDSLKQLQWFLKLLYVTVPLDEK |
| | DYEFYNELEVYYEALLPLNSLYNKVRNYMTRKPYSVEKFKLNFYSPTLLDG |
| | WDKNKETANLSIILKKNGKYYLGIMNKENNTIFENFPKSKSNDYYEKMIYK |
| | LLPGPNKMLPKVFFSKKGLEYYKPSKEILRIYEKGEFKKDKSGNFKKESLHT |
| | LIDFYKEAIAKNEDWKIFKFKFKNTREYEDISQFYRDVEEQGYLIIFEKVDAN |
| | YVDKLVEEGELYLFQIYNKDFSENKKSKGNPNLHTIYWESLFDNQNLKDVV |
| | YKLNGEAEVFYRKKSIDYPEEIYNNGHHKEELNGKFNYPIIKDRRYTQDKFL |
| | FHVPITMNFISKEEKRVNQLACEYLSTTKEDVHIIGIDRGERHLLYLSLIDKEG |
| | NIKKQLSLNTIKNENYDKEIDYRVKLDEKEKKRDEARKNWDVIENIKELKE |
| | GYMSQVIHIIAKMMAEEKAILIMEDLNIGFKRGRFKVEKQVYQKFEKMMID |
| | KLNYLVFKNKEPLEPGGSLNAYQLTSKFDSFKKLGKQSGFIFYVPSAYTSKID |
| | PTTGFYNFIRVDVPNLEKGKEFFSKFEKIIYNTKEDYFEFHCKYGKFVPEPKN |
| | KDNDRKTKESLTYYNAIKDTVWVVCSTHHERYKIVRNKAGYYESQPVDVT |
| | KNLRDIFSEANINYSDGKDIKPIIIESNNAKLLKSIAEQLKLILAMRYNNGKHD |
| | DDEKDYILSPVKNKQGKFFCSLDGDQSLPINADANGAYNIALKGLLLIEKIK |
| | KQQGKAKDLYISNLEWFMFMMSR |
| SEQ ID | MKSIFDNFTGLYSLSKTLRFELRPVGQTLENIKNGHFLESDKKMADDYQDV |
| NO: 85 | KKIIDNYHKFFIDDVLKGASFDWALLEKELTDFNKNKTDDSKVEAEQKKLR |
| | EQIAKTLAGDKRFKSLTASTPNDLFNKDKDFIGWLEQSSVKEIRKDALDTFK |
| | KFSSYFKGFQENRKNVYSADDIPTAVPYRIVNDNFPKFLQNISIFKTIQEKCP |
| | QVIADVENELASYLGKEKLADIFTVQAFNKYLCQGGKENQRGIDFYNQVIG |
| | GIAEKEGGVNLRGINQFLNLYWQQHPDFAKENRRIKMVPLYKQILSDRSSLS |
| | FKIETIDTDEELKTAISEYADKLESKSNDEKKSVLDVCVELFDSIKEQNLQEIY |
| | VNRKDINNISRILTGDWSWLQSRMNLYADEVFTTKAEKTRWQKSVDGDEG |
| | ENKSKGAYSLAELNRVLEYASENVAETDIRITDYFCHRNRFYYEKESGLFKQ |
| | GEELVALSIKESCEDILSKRKAMNEAFANISESNSLRDNSEDIAKTKTYLDSV |
| | QDLLHRIKPLKVNGLGDPSFYAVFDSIYSALSEVISIYNKTRNYITRKAESPEK |
| | YKLNFDNPTLANGWDLNKEKDNTCVLLRKNGMYYLGIMNPKDKPKFAEK |
| | YDCGTESCYEKMIYKLLPGPNKMLPKVFFSTKGKKQYNPPENILHGYEQGK |
| | HKKGVAFDINFCHELIDWFKSAINQHEDWKKFGFKFSDTKSYKDISDFYREV |
| | TEQGYKLTFINIPESEISKMVSEGKLYLFQIYNMDFAPGANGMPNMHTLYW |
| | KNLFSEENLKDVVLKLNGEAELFYRPAGIKEPVVHAKGSYLVNRITKDGEPI |
| | PEKIHDEIYRNANGNLNNLSKEASEYKESHKVVIKQASHEIIKDRHYTEPKFL |
| | FHIPLTINFKAPSMKPTVCTAIQNENVRRFLKNNPDVNIIGLDRGERNLIYLSL |
| | INQKGEIIKQFSFNDVEREQNGQTVKVNYHEKLDQREKERDAARKSWQAIG |
| | KIAELKEGYLSAVIHQLTKLMVEYNAIVVMEDLNFGFKRGRFHVEKQVYQ |
| | KFEHMLIDKLNYLVFKDRGLNEPGGALNGYQLAGQFESFQKLGKQSGMLF |
| | YVPAAYTSKIDPKTGFVSMMNFKDLTNVHKKRDFFSKFEDIHFDEATCSFVF |
| | TFDYKNINGKAKEEMKQTKWAVYSREKRIVYFSKTKSSEDIMPTEKLRALF |
| | ESDGIEYKSGNNIHDSVMAVGADLKEGAKPSKEIADFWDGLLYNFKLILQM |
| | RNSNAKTGEDYIISPVMASDGTFFDSRVEAKKGKDAKLPLDADANGAYHIA |
| | LKGLSLINKINLAGEDELKKFDMKISNEDWFNFAQEKKYAE |
| SEQ ID | MTKQNKSVTQNTKRKNFGEFTNLYSLSKTLRFELKPVKATKTILEVERKDRE |
| NO: 86 | NFKKDRKIAKNYQKLKGILNELHQEFIQDVMREFSFQEKEIKEFEERYLEAL |
| | NFKEKDNYKKRTQLKNAYEKVAKKLAGKIATAFGKYNQEKYGVKFTKKNL |
| | TGENVFDILEGKYKGDKKILGIIHTFKFKPTKEEKKQGKEAVNFSTYLGGFN |
| | QNRENFYKGEMKAGQFATRTIENLIQFLKNKKLFIDKYKDNYQKIGFSQEQV |
| | EIFNLNYFNNLFLQEGLDVYNGILGAKKGEKNTENDGLNQKINLFKQKEKT |
| | RCKANGEKFNKSDYPIFKELYKQIGSIKKDNDVYVEIKSDEELVNVLQSLPE |
| | KTANTLREVQKFYENFFDKIFNDEFDLDKIYLPKSVGTHFSHLAFSDWSKLA |
| | FVFNKRWRNEKVKIKEGEDVNVQSRSLADIKKRMEEILEMDGGVSFGKTYC |
| | QKVGLEKEARTIEDVWSGFWKIIQYHINSQFIGGEKEVFDKEKKDDKTEKIQ |
| | TIDDLQEEYLQATEMYRERMVESEEGLNDGEEKEIKTKLKNYLDRIKDIERI |
| | ARYFDLRKHFDDIDEASKDGDFYFIYQELLQDISEAKINDHYNEIRNYLTKA |
| | NVVDDKFKLNFNDGQTLSGWDLNKETEKFSLIFKRKVDGGVEYYLGIINKE |
| | KNKTIFDKKKHPEIFTENSEFEKMEYKLFPSPSKMLPKIAFTKNKEGERIKPV |
| | FLDENAGKEIAQIKKEFALFQDAKKEDKNKWSDEFDRKKLNKLIDYYKLVL |
| | EKHPEKYMQTFNFVFKSSAKYKNLGEFNDDVARQNYVTKFVSVDKDYIDQ |
| | KVESGELYLFKIHNKDWNLTKAGDTKKQSKKNLHTIYFEELFSEKNIAEPVF |
| | KLSGGAEVFFRDAIEKKKQKKKKDKKGKEILEKFRFTKNKILFHVPITINYG |
| | KPSINQGQFNQKINEFIADNSRSVNILGIDRGEKHLLYYSLVDNNGKIIKSGSL |
| | NEINGVDYHEKLDKAEKERQEARKSWQKINQIKNLKAGYISQVIKKIVDLAI |
| | ENNAIIVLEDLNFGFKSFRQKIEKNVYQQFEKALIDKLGFVTDKEKLNHRQA |
| | PQLSAPFESFEKMGKQTGIVFYVLATNTSKVCPQCQWKKNIFPHYSTKKSIA |
| | ENLQKQYKMKMYWRENENRFEFEYKGDGDKEFSSIFSNVDRVRYDKRANN |
| | NQGGYVIYQIDSTTKEKDGRNIKEKSITNLLKELLLEKFEIDNLEGELLVKLS |
| | EKSPDVSKETIKDFFGLLNSILNIRNSMTDTEEDYIQCPACGFDTRKENKIGIK |
| | NGDDNGAYNIALRGRFLIERIKKAKKEDKKPNLTFSNNDYFQWVREFVK |
| SEQ ID | MRTTTSLDAFTNRYALSKTLRFELKPIGNTQMMLEQNNVFAKDRAIREKYE |
| NO: 87 | KTKPWIDLLHREFVAESLQNAQLGNLDDYYAALQNVQKITKDTNAEDKKR |
| | WKKGFEKQEKRLRKEVVALFDKAAHIWATQRYPQLKKKTKDFLFEEGVFE |
| | HVLFARYGSAPDTTVKIVTSNPETGEVIDEREESIFKGWKGFTGYFDKFFETR |
| | KNFYKDNGTATAIATRAINQNLRRFAENMQKLTDIKNNYPELLAHTDFGDFD |
| | IAHAQSLDFYARTCLLQEGIDAYNKKFVGVLKSAINEYQQKNKGVRISYPKT |
| | LDNQILGERERRLFDVIEDDRELHDVFRAFVDDGTVFAAEMRQLAQAFSAQ |
| | NGTYDYTQIYISKKGFETISRKYTHDTRAWHDALADVFKAKAKKRIATTASG |
| | EKKFPAYIPVAYITQALTLVQESEDTECTWKERYASITENKTLEEGFFAIFADEF |
| | ERLFVHMEATVQDTDYVVAEDKAKKLLSDGQITKNEQTTQIIKEYADALLRI |
| | YQMAKYFAVEKKSMWDDAVAIDDTFYETFKEIYGNTHSTIVASYNLLRNYL |
| | TKKPWEDVQKWKLNFENPTLLDGWDKNKEAANFGVILRDGDKFYLGIMR |
| | KGHNNIFANQHHSNFEGQGLQKMVYKFFPDPKKMFPKVCFSAKGMEFFAPS |
| | EEIVRIYKNAEFKSGDTFNVESMQKLIDFYKNALQKYDGWKIYDFKHLKDT |
| | AQYTSNIGEFYDDVAKGGYQLGWQNISKEYVEEKNANGELYLFQIKNKDW |
| | NDGATGRKNLHTLYFEYLFSEKNAAADFVFRLNGGAEVFYRPAAIESKTERR |
| | GNREVAAKKRYTQDKVFLHVPITLNRTAGDVKTSAFNDAVNRFLAGNPDINI |
| | MGIDRGEKHLAYYSIIDQNGNRIVSGSFNTIGSKDYHALLTERQGAREEARK |
| | NWQRVEQIKDLKKGYISLVVREIADLAIKHNAIIVLENLNMRFKQIRGGIEKS |
| | VYQQLEKALIEKLNFLVNKGEVDATKAGHLLRAYQLAAPFETFEKMGNQTG |
| | IIFYTTASYTSQVDPVTGWRPHVYLKYRNAQKTKEDILRIFDDIVENDEKQRF |
| | EFAYRHNGVSWTVCSSVERHRWNRSNNAGKGGYDVFPVEGEGSITQRLQE |
| | ACAQRGIDTTRNILAQIDELDESASATVSFLRDLCFYFRLICQIRNTDDGADDI |
| | NAQDFLMSPVEPFFDTRNAQEQYPQNGDENGAYNIARKGIIILQKITAWGRS |
| | QDTQRRYPDTFVSQDEWDTFLTQHTT |
| SEQ ID | MLKEKQFKTFGDFTNLYELSKTLRFELRPTPETKDLLDKNKIIQTDKKIAENY |
| NO: 88 | QEIKKYFDKLHKKFIKEALSNTQIDFSDFCKLWEQNSKDSGKIKDLSRKLRK |
| | SIKQAFDKKGADWHKRYLEKGIKLKKKNLDILFEERVLDILKEEFKDDVDV |
| | KLFESFKGFSTYFTNFHESRKNFYKDDGTASAIATRIIDENLKRFCDNIKVKK |
| | HSKKLISELNEREAKIFEADFYNRCLLQQGIDDYNQVIGDINKKINNLRQNKI |
| | ENTPTLKILYKQILGDVRRQETEQDAFIEIKNNEEVFDFLQDFIKHSDENNKY |
| | FKNLFYKFIEGKHSLDKIFLAKRFVNTISGKWFASWEVFGAELIKKEGNKKD |
| | LPDFIPFAAVKDVLQNCNIPANELFKEKIKNDEDKNIYDIFINLWKEEFDSNLK |
| | KVEESKKEVENMIAEDKVYSNKKEKRKNDNGEEIEIEIQKEKIKNYADAAM |
| | NIFRMMKYFLLEKNGKTVEGMGEDNNFYNELNIVFKGGEIDGKVYEGVKT |
| | YLYYNEFRNYLTKKPFNEEKTKLNFDCGQILSGWDKNKESEKLGVILRKDN |
| | KYYLAIINKKHNKIFDVKKNSYAYIVGDNFYEKMEYKLFPDAKRMIPKIAFA |
| | KNNKEKFGWTDEIQKIKNEYAEFQEGKKNDKNLWKDKFNKNKMEKLITYY |
| | QNCLEKGGYKDIYNFRWKSPDKYSGIGEFNDEIDRQSYCLKFVKVDFNYVF |
| | EKVKSGELYLFQIYNKDFSDKADRAQKENIHTEYFKLLFDQRNLDNVVLKL |
| | SGGAEIFYRPKTEGLPKKKDNKGNEVVRHRRYADDKYFLHLPIQLNFGRGN |
| | LSGGEFNSKINQYLSEQREIKIIGIDRGEKHLAYYSVINQDGKIEEIESLNTVN |
| | GIDYRKKLDELEKKREQERKSWQSISKIKDLKKGYISHVIKKICDLAIEHNAII |
| | VFEDLSGGFKNSRKKIEKQIYQNLELALATKLNYLTFKDKNFGESGHYLNAY |
| | QLAPKIDNYQDIKMQTGIVFYTPAGYTSSTCPQCGFRKTLKFDYTATISKAED |
| | LIRGSKLNIVFEKEKNRFKINYLFNPIEKKKKKIKENELFADAGAKNEFTIYSD |
| | VKRIKWHNTGTKRLEEAEGERLLENKNSRGRDKEYDINKCLTRLFRENKID |
| | VNGDIIGQITKIKSLKLYQDLFYYLFLATLIRNNVSGSDIDYIQCPSCHFHSDG |
| | GFQKQKFNGDANGAYNIARKGILILKKIKQFAAQDKDMKNFGWKHLTVDIN |
| | EWDKFTQK |
| SEQ ID | MNKNFSNFTELYTLSKTLRFELKPVAQTKENIKKGKFLESDKKKADDYKDV |
| NO: 89 | KKIIDNYHKFFIDDVLKNASFDWTVLEKEMSDFNKSKADDSKVEAEQKKLR |
| | DQIAKKLTSDKRFKALTASTPSDLFNKDKDFIDWFTQNSTKDINKEALETFK |
| | RFSSYFKGFQENRKNVYSAEPIPTAVPYRLVNDNFPKFLQNIALFKIIQEKCPQ |
| | VISDVEKELASYLGKEKLADIFTVQAFNKYLCQGGKENQRGIDFYNNILGGI |
| | AEKEGGINLRGINQFLNLYWQQHQDFAKQNKRIKMIPLFKQILSDRSSLSFKI |
| | ESINTDQELLTSITEYADKLETKSNDEKKSVLDICSDLFASIKAQNLQEIYVNR |
| | KDINSISRILTGDWSWLQSRMNVYADEVFTTKAEKTRWQKSIDGDEGENKS |
| | KGVFSLAELNSVLEYSSENVSETDVRITDFFDHRNRFYYEKESGLFKQGDEL |
| | VALSIRESCEDILAKRKAMDEAFANVSENNSLRDNSEDVAKIKIYLDCVQELL |
| | HRIKPLKVNGLGDPAFYAVFDTVYNSLSEVISLYNKTRNYITKKAANPEKYK |
| | LNFDNPTLADGWDLNKEQANTSVLMRKDGMYYLGIMNPKDKPKFAEKYE |
| | CGNEACYEKMIYKQFDATKQIPKCSTQVKEVKKHFQSGATDSIILNDKSKFK |
| | LDLVITKEIWFLNNHVWNGEKFVPKRESNETRPKKFQIGYYKQTGDLGGYK |
| | EALNIWISFCKTFLQSYISSSIYDYDFKESSNYDSLDEFYNYLNATCYKLSFINI |
| | PEATISQMVSEGKLYLFQIYNKDFAPGASGMPNMHTLYWKNLFSEENLKNV |
| | VLKLNGEAELFYRPAGIKEPVIHAKGSYLVNRITKDGEPIPEKIHDEIYRNAN |
| | GKLESLSKEATEYKASHKVVIKEAKHDIIKDRHYTEPKFLFHVPLTINFKASG |
| | NSYINENVRRFLKNNPDVNVIGLDRGERHLIYLSLINQKGEIIKQFTFNEVER |
| | NKNGQVIKVNYHEKLDQREKVRGAARKSWQAIGKIAELKEGYLSAVIHQLT |
| | KLMVEYNAIVVMEDLNFGFKRGRFHVEKQVYQKFEHMLIDKLNYLVFKDR |
| | GLTEAGGVLNGYQLAGQFESFQKLGKQSGMLFYVPAGYTSKIDPKTGFASM |
| | SNFKDLTNVHKKRAFFSKFDDIHFDDATGSFVFTFDYKNFDGKAKEEMKRT |
| | KWSVYSKDKRIVYLSKTKSYEDVQPTEKIKASLESVGIEYMSGNNLIDSIMVI |
| | GAELKDGAKPSKEIADFWDRLLYNFKLILQMRNSNAKTGEDYIISPVMADD |
| | GTFFDSREEFKKGENAKMPVDADANGAYHIALKGLSLLKRFDAASENELKK |
| | FDMKISNVDWFKFAQEKSYAE |
| SEQ ID | MKAKKSFYNQKRKFGKRGYRLHDERIAYSGGIGSMRSIKYELKDSYGIAGL |
| NO: 90 | RNRIADATISDNKWLYGNINLNDYLEWRSSKTDKQIEDGDRESSLLGFWLEA |
| | LRLGFVFSKQSHAPNDFNETALQDLFETLDDDLKHVLDRKKWCDFIKIGTPK |
| | TNDQGRLKKQIKNLLKGNKREEIEKTLNESDDELKEKINRIADVFAKNKSDK |
| | YTIFKLDKPNTEKYPRINDVQVAFFCHPDFEEITERDRTKTLDLIINRFNKRYE |
| | ITENKKDDKTSNRMALYSLNQGYIPRVLNDLFLFVKDNEDDFSQFLSDLENF |
| | FSFSNEQIKIIKERLKKLKKYAEPIPGKPQLADKWDDYASDFGGKLESWYSN |
| | RIEKLKKIPESVSDLRNNLEKIRNVLKKQNNASKILELSQKIIEYIRDYGVSFE |
| | KPEIIKFSWINKTKDGQKKVFYVAKMADREFIEKLDLWMADLRSQLNEYNQ |
| | DNKVSFKKKGKKIEELGVLDFALNKAKKNKSTKNENGWQQKLSESIQSAPL |
| | FFGEGNRVRNEEVYNLKDLLFSEIKNVENILMSSEAEDLKNIKIEYKEDGAK |
| | KGNYVLNVLARFYARFNEDGYGGWNKVKTVLENIAREAGTDFSKYGNNN |
| | NRNAGRFYLNGRERQVFTLIKFEKSITVEKILELVKLPSLLDEAYRDLVNENK |
| | NHKLRDVIQLSKTIMALVLSHSDKEKQIGGNYIHSKLSGYNALISKRDFISRY |
| | SVQTTNGTQCKLAIGKGKSKKGNEIDRYFYAFQFFKNDDSKINLKVIKNNSH |
| | KNIDENDNENKINALQVYSSNYQIQFLDWFFEKHQGKKTSLEVGGSFTIAEK |
| | SLTIDWSGSNPRVGFKRSDTEEKRVFVSQPFTLIPDDEDKERRKERMIKTKNR |
| | FIGIDIGEYGLAWSLIEVDNGDKNNRGIRQLESGFITDNQQQVLKKNVKSWR |
| | QNQIRQTFTSPDTKIARLRESLIGSYKNQLESLMVAKKANLSFEYEVSGFEVG |
| | GKRVAKIYDSIKRGSVRKKDNNSQNDQSWGKKGINEWSFETTAAGTSQFCT |
| | HCKRWSSLAIVDIEEYELKDYNDNLFKVKINDGEVRLLGKKGWRSGEKIKG |
| | KELFGPVKDAMRPNVDGLGMKIVKRKYLKLDLRDWVSRYGNMAIFICPYV |
| | DCHHISHADKQAAFNIAV |
| SEQ ID | MLRRDKEVKKLYNVFNQIQVGTKPKKWNNDEKLSPEENERRAQQKNIKM |
| NO: 91 | KNYKWREACSKYVESSQRIINDVIFYSYRKAKNKLRYMRKNEDILKKMQE |
| | AEKLSKFSGGKLEDFVAYTLRKSLVVSKYDTQEFDSLAAMVVFLECIGKNN |
| | ISDHEREIVCKLLELIRKDFSKLDPNVKGSQGANIVRSVRNQNMIVQPQGDR |
| | FLFPQVYAKENETVTNKNVEKEGLNEFLLNYANLDDEKRAESLRKLRRILD |
| | VYFSAPNHYEKDMDITLSDNIEKEKFNVWEKHECGKKETGLFVDIPDVLME |
| | AEAENIKLDAVVEKRERKVLNDRVRKQNIICYRYTRAVVEKYNSNEPLFFE |
| | NNAINQYWIHHIENAVERILKNCKAGKLFKLRKGYLAEKVWKDAINLISIKY |
| | IALGKAVYNFALDDIWKDKKNKELGIVDERIRNGITSFDYEMIKAHENLQRE |
| | LAVDIAFSVNNLARAVCDMSNLGNKESDFLLWKRNDIADKLKNKDDMASV |
| | SAVLQFFGGKSSWDINIFKDAYKGKKKYNYEVRFIDDLRKAIYCARNENFH |
| | FKTALVNDEKWNTELFGKIFERETEFCLNVEKDRFYSNNLYMFYQVSELRN |
| | MLDHLYSRSVSRAAQVPSYNSVIVRTAFPEYITNVLGYQKPSYDADTLGKW |
| | YSACYYLLKEIYYNSFLQSDRALQLFEKSVKTLSWDDKKQQRAVDNFKDH |
| | FSDIKSACTSLAQVCQIYMTEYNQQNNQIKKVRSSNDSIFDQPVYQHYKVLL |
| | KKAIANAFADYLKNNKDLFGFIGKPFKANEIREIDKEQFLPDWTSRKYEALC |
| | IEVSGSQELQKWYIVGKFLNARSLNLMVGSMRSYIQYVTDIKRRAASIGNEL |
| | HVSVHDVEKVEKWVQVIEVCSLLASRTSNQFEDYFNDKDDYARYLKSYVD |
| | FSNVDMPSEYSALVDFSNEEQSDLYVDPKNPKVNRNIVHSKLFAADHILRDI |
| | VEPVSKDNIEEFYSQKAEIAYCKIKGKEITAEEQKAVLKYQKLKNRVELRDI |
| | VEYGEIINELLGQLINWSFMRERDLLYFQLGFHYDCLRNDSKKPEGYKNIKV |
| | DENSIKDAILYQIIGMYVNGVTVYAPEKDGDKLKEQCVKGGVGVKVSAFH |
| | RYSKYLGLNEKTLYNAGLEIFEVVAEHEDIINLRNGIDHFKYYLGDYRSMLS |
| | IYSEVFDRFFTYDIKYQKNVLNLLQNILLRHNVIVEPILESGFKTIGEQTKPGA |
| | KLSIRSIKSDTFQYKVKGGTLITDAKDERYLETIRKILYYAENEEDNLKKSVV |
| | VTNADKYEKNKESDDQNKQKEKKNKDNKGKKNEETKSDAEKNNNERLSY |
| | NPFANLNFKLSN |
| SEQ ID | MKLTRRRISGNSVDQKITAAFYRDMSQGLLYYDSEDNDCTDKVIESMDFER |
| NO: 92 | SWRGRILKNGEDDKNPFYMFVKGLVGSNDKIVCEPIDVDSDPDNLDILINKN |
| | LTGFGRNLKAPDSNDTLENLIRKIQAGIPEEEVLPELKKIKEMIQKDIVNRKE |
| | QLLKSIKNNRIPFSLEGSKLVPSTKKMKWLFKLIDVPNKTFNEKMLEKYWEI |
| | YDYDKLKANITNRLDKTDKKARSISRAVSEELREYHKNLRTNYNRFVSGDR |
| | PAAGLDNGGSAKYNPDKEEFLLFLKEVEQYFKKYFPVKSKHSNKSKDKSLV |
| | DKYKNYCSYKVVKKEVNRSIINQLVAGLIQQGKLLYYFYYNDTWQEDFLN |
| | SYGLSYIQVEEAFKKSVMTSLSWGINRLTSFFIDDSNTVKFDDITTKKAKEAI |
| | ESNYFNKLRTCSRMQDHFKEKLAFFYPVYVKDKKDRPDDDIENLIVLVKNA |
| | IESVSYLRNRTFHFKESSLLELLKELDDKNSGQNKIDYSVAAEFIKRDIENLY |
| | DVFREQIRSLGIAEYYKADMISDCFKTCGLEFALYSPKNSLMPAFKNVYKRG |
| | ANLNKAYIRDKGPKETGDQGQNSYKALEEYRELTWYIEVKNNDQSYNAYK |
| | NLLQLIYYHAFLPEVRENEALITDFINRTKEWNRKETEERLNTKNNKKHKNF |
| | DENDDITVNTYRYESIPDYQGESLDDYLKVLQRKQMARAKEVNEKEEGNN |
| | NYIQFIRDVVVWAFGAYLENKLKNYKNELQPPLSKENIGLNDTLKELFPEEK |
| | VKSPFNIKCRFSISTFIDNKGKSTDNTSAEAVKTDGKEDEKDKKNIKRKDLL |
| | CFYLFLRLLDENEICKLQHQFIKYRCSLKERRFPGNRTKLEKETELLAELEEL |
| | MELVRFTMPSIPEISAKAESGYDTMIKKYFKDFIEKKVFKNPKTSNLYYHSD |
| | SKTPVTRKYMALLMRSAPLHLYKDIFKGYYLITKKECLEYIKLSNIIKDYQN |
| | SLNELHEQLERIKLKSEKQNGKDSLYLDKKDFYKVKEYVENLEQVARYKH |
| | LQHKINFESLYRIFRIHVDIAARMVGYTQDWERDMHFLFKALVYNGVLEER |
| | RFEAIFNNNDDNNDGRIVKKIQNNLNNKNRELVSMLCWNKKLNKNEFGAII |
| | WKRNPIAHLNHFTQTEQNSKSSLESLINSLRILLAYDRKRQNAVTKTINDLLL |
| | NDYHIRIKWEGRVDEGQIYFNIKEKEDIENEPIIHLKHLHKKDCYIYKNSYMF |
| | DKQKEWICNGIKEEVYDKSILKCIGNLFKFDYEDKNKSSANPKHT |

Other Exemplary protein sequences are described in the following applications: PCT/US2021/033271; PCT/US2021/035031, and PCT/US2022/028865, all of which are herein incorporated by reference in their entirety.

In some cases, the effector proteins comprise a RuvC domain (e.g., a partial RuvC domain). In some instances, the RuvC domain may be defined by a single, contiguous sequence, or a set of partial RuvC domains that are not contiguous with respect to the primary amino acid sequence of the protein. An effector protein of the present disclosure may include multiple partial RuvC domains, which may combine to generate a RuvC domain with substrate binding or catalytic activity. For example, an effector protein may include three partial RuvC domains (RuvC-I, RuvC-II, and RuvC-III, also referred to herein as subdomains) that are not contiguous with respect to the primary amino acid sequence of the effector protein, but form a RuvC domain once the protein is produced and folds. In some cases, effector proteins comprise a recognition domain with a binding affinity for a guide nucleic acid or for a guide nucleic acid-target nucleic acid heteroduplex. In some instances, the effector protein does not comprise a zinc finger domain. In some instances, the effector protein does not comprise an HNH domain.

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some instances, the target nucleic acid is single-stranded DNA. In some instances, the target nucleic acid is single-stranded RNA. The effector proteins may provide cis cleavage activity, trans cleavage activity, nickase activity, or a combination thereof. Cis cleavage activity is cleavage of a target nucleic acid that is hybridized to a guide nucleic acid (e.g., a dual gRNA or a sgRNA), wherein cleavage occurs within or directly adjacent to the region of the target nucleic acid that is hybridized to guide nucleic acid. Trans cleavage activity (also referred to as transcollateral cleavage) is cleavage of ssDNA or ssRNA that is near, but not hybridized to the guide nucleic acid. Trans cleavage activity is triggered by the hybridization of guide nucleic acid to the target nucleic acid. Nickase activity is a selective cleavage of one strand of a dsDNA.

Effector proteins of the present disclosure, dimers thereof, and multimeric complexes thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer region.

In some instances, the Type V CRISPR/Cas protein has been modified (also referred to as an engineered protein). For example, a Type V CRISPR/Cas protein disclosed herein or a variant thereof may comprise a nuclear localization signal (NLS). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

Several programmable nucleases are consistent with the methods and devices of the present disclosure. For example, Cas proteins are programmable nucleases used in the methods and systems disclosed herein. Cas proteins can include any of the known Classes and Types of CRISPR/Cas enzymes. Programmable nucleases disclosed herein include Class 1 Cas proteins, such as the Type I, Type IV, or Type III Cas proteins. Programmable nucleases disclosed herein also include the Class 2 Cas proteins, such as the Type II, Type V, and Type VI Cas proteins. Programmable nucleases included in the devices disclosed herein and methods of use thereof include a Type V or Type VI Cas proteins.

In some instances, the programmable nuclease is a Type V Cas protein. In general, a Type V Cas effector protein comprises a RuvC domain, but lacks an HNH domain. In most instances, the RuvC domain of the Type V Cas effector protein comprises three partial RuvC domains (RuvC-I, RuvC-II, and RuvC-III, also referred to herein as subdomains). In some instances, the three RuvC subdomains are located within the C-terminal half of the Type V Cas effector protein. In some instances, none of the RuvC subdomains are located at the N terminus of the protein. In some instances, the RuvC subdomains are contiguous. In some instances, the RuvC subdomains are not contiguous with respect to the primary amino acid sequence of the Type V Cas protein, but form a ruvC domain once the protein is produced and folds. In some instances, there are zero to about 50 amino acids between the first and second RuvC subdomains. In some instances, there are zero to about 50 amino acids between the second and third RuvC subdomains. In some instances, the Cas effector is a Cas14 effector. In some instances, the Cas14 effector is a Cas14a, Cas14a1, Cas14b, Cas14c, Cas14d, Cas14e, Cas14f, Cas14g, Cas14h, or Cas 14u effector. In some instances, the Cas effector is a CasPhi effector. In some instances, the Cas effector is a Cas12 effector. In some instances, the Cas12 effector is a Cas12a, Cas12b, Cas12c, Cas12d, Cas12e, or Cas12j effector.

In some instances, the Type V CRISPR/Cas protein comprises a Cas 14 protein. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between B-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas14 proteins may comprise a RuvC domain or a partial RuvC domain. The RuvC domain may be defined by a single, contiguous sequence, or a set of partial RuvC domains that are not contiguous with respect to the primary amino acid sequence of the Cas14 protein. In some instances, a partial RuvC domain does not have any substrate binding activity or catalytic activity on its own. A Cas 14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A Cas14 protein may comprise a linker loop connecting a carboxy terminal domain of the Cas14 protein with the amino terminal domain of the Cas 14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas14 protein comprises a zinc finger domain. In some instances, an amino terminal domain of a Cas 14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

Cas14 proteins may be relatively small compared to many other Cas proteins, making them suitable for nucleic acid detection or gene editing. For instance, a Cas 14 protein may be less likely to adsorb to a surface or another biological species due to its small size. The smaller nature of these proteins also allows for them to be more easily packaged as a reagent in a system or assay, and delivered with higher efficiency as compared to other larger Cas proteins. In some cases, a Cas 14 protein is 400 to 800 amino acid residues long, 400 to 600 amino acid residues long, 440 to 580 amino acid residues long, 460 to 560 amino acid residues long, 460 to 540 amino acid residues long, 460 to 500 amino acid residues long, 400 to 500 amino acid residues long, or 500 to 600 amino acid residues long. In some cases, a Cas 14 protein is less than about 550 amino acid residues long. In some cases, a Cas14 protein is less than about 500 amino acid residues long.

In some instances, a Cas14 protein may function as an endonuclease that catalyzes cleavage at a specific position within a target nucleic acid. In some instances, a Cas14 protein is capable of catalyzing non-sequence-specific cleavage of a single stranded nucleic acid. In some cases, a Cas14 protein is activated to perform trans cleavage activity after binding of a guide nucleic acid with a target nucleic acid. This trans cleavage activity is also referred to as “collateral” or “transcollateral” cleavage. Trans cleavage activity may be non-specific cleavage of nearby single-stranded nucleic acid by the activated programmable nuclease, such as trans cleavage of reporters with a detection moiety.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acid via a single catalytic RuvC domain. The RuvC domain is within a nuclease, or “NUC” lobe of the protein, and the Cas 12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In some instances, a programmable Cas12 nuclease can be a Cas12a protein, a Cas12b protein, Cas12c protein, Cas12d protein, or a Cas12e protein.

In some embodiments, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the Cas12 can be Cas12M08, which is a specific protein variant within the Cas12 protein family/classification). In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium| rectale (Ere), Listeria newyorkensis (Lny), Clostridium aminophilum ((am), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus ((ca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR/Cas enzyme (e.g., a Cas13 nuclease). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter. An RNA reporter can be an RNA-based reporter. In some embodiments, the Cas13a recognizes and detects ssDNA to initiate transcleavage of RNA reporters. Multiple Cas13a isolates can recognize, be activated by, and detect target DNA, including ssDNA, upon hybridization of a guide nucleic acid with the target DNA. For example, Lbu-Cas13a and Lwa-Cas13a can both be activated to transcollaterally cleave RNA reporters by target DNA. Thus, Type VI CRISPR/Cas enzyme (e.g., a Cas13 nuclease, such as Cas13a) can be DNA-activated programmable RNA nucleases, and therefore can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly taken from organisms or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example, target ssDNA detection by Cas13a can be employed in a detection device as disclosed herein.

Other Exemplary protein sequences are described in the following applications: PCT/US2021/033271; PCT/US2021/035031, and PCT/US2022/028865, all of which are herein incorporated by reference in their entirety.

In some embodiments a programmable nuclease is referred to as an effector protein. In some instances, an effector protein disclosed herein is an engineered protein. The engineered protein is not identical to a naturally-occurring protein. The engineered protein may provide enhanced nuclease or nickase activity as compared to a naturally occurring nuclease or nickase. By way of non-limiting example, some engineered proteins exhibit optimal activity at lower salinity and viscosity than the protoplasm of their bacterial cell of origin. Also by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise. Compositions and systems may comprise an engineered protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wildtype counterpart protein. The modified form of the wildtype counterpart may comprise an amino acid change (e.g., deletion, insertion, or substitution) that reduces the nucleic acid-cleaving activity of the programmable nuclease. For example, a nuclease domain (e.g., RuvC domain) of a Type V CRISPR/Cas protein may be deleted or mutated so that it is no longer functional or comprises reduced nuclease activity. The modified form of the programmable nuclease may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g. inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with an engineered guide nucleic acid to activate or repress transcription of a target nucleic acid sequence. In some embodiments, the enzymatically inactive protein is fused with a protein comprising recombinase activity.

In some instances, a programmable nuclease is a fusion protein, wherein the fusion protein comprises a protein comprising the amino acid sequence of any one of SEQ ID NOs: 1-61 or 81-92. In some instances, the fusion protein comprises a programmable nuclease and a fusion partner protein.

A fusion partner protein is also simply referred to herein as a fusion partner. In some cases, the fusion partner promotes the formation of a multimeric complex of the programmable nuclease. In some cases, the fusion partner is an additional programmable nuclease. In some cases, the multimeric complex comprising the programmable nuclease and the additional programmable nuclease binds a guide nucleic acid. The programmable nucleases of the multimeric complex may bind the guide nucleic acid in an asymmetric fashion. In some cases, one programmable nuclease of the multimeric complex interacts more strongly with the guide nucleic acid than the additional programmable nuclease of the multimeric complex. In some cases, a programmable nuclease interacts more strongly with a target nucleic acid when it is complexed with the guide nucleic acid relative to when the programmable nuclease or the multimeric complex is not complexed with the guide nucleic acid.

In some cases, the fusion partner has enzymatic activity in the presence of its enzyme substrate. For example, the fusion partner may comprise an enzyme such as horse radish peroxidase (HRP) which can catalyze a detectable color change reaction in the presence of its stubstrate (e.g., TMB).

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some cases, a terminus of the programmable nuclease is linked to a terminus of the fusion partner through an amide bond. In some cases, a programmable nuclease is coupled to a fusion partner via a linker protein. In some cases, a programmable nuclease is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. These linkers may be produced by using synthetic, linker-encoding oligonucleotides to couple the proteins, or may be encoded by a nucleic acid sequence encoding a fusion protein (e.g., an programmable nuclease coupled to a fusion partner). Examples of linker proteins include glycine polymers (G) n (SEQ ID NO: 70), glycine-serine polymers (including, for example, (GS) n (SEQ ID NO: 71), GSGGSn (SEQ ID NO: 72), GGSGGSn (SEQ ID NO: 73), and GGGSn (SEQ ID NO: 74), where n is an integer of at least one), glycine-alanine polymers, and alanine-serine polymers. Exemplary linkers may comprise amino acid sequences including, but not limited to, GGSG (SEQ ID NO: 75), GGSGG (SEQ ID NO: 76), GSGSG (SEQ ID NO: 77), GSGGG (SEQ ID NO: 78), GGGSG (SEQ ID NO: 79), and GSSSG (SEQ ID NO: 80).

Disclosed herein are non-naturally occurring compositions and systems comprising at least one of an engineered Cas protein and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, compositions and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid may comprise a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally-occurring sequence. In some instances, the guide nucleic acid may comprise two naturally occurring sequences arranged in an order or proximity that is not observed in nature. In some instances, compositions and systems comprise a ribonucleotide complex comprising a programmable nuclease and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

Described herein are various embodiments of thermostable programmable nucleases. In some embodiments, a programmable nuclease is referred to as a programmable nuclease. A programmable nuclease may be thermostable. In some instances, known programmable nucleases (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of a programmable nuclease in a trans cleavage assay at 40° C. may be at least 50% of that at 37° C. In some instances, the trans cleavage activity of a programmable nuclease in a trans cleavage assay at 40° C. may be at least 55% of that at 37° C. In some instances, the trans cleavage activity of a programmable nuclease in a trans cleavage assay at 40° C. may be at least 60% of that at 37° C. In some instances, the trans cleavage activity of a programmable nuclease in a trans cleavage assay at 40° C. may be at least 65% of that at 37° C. In some instances, the trans cleavage activity of a programmable nuclease in a trans cleavage assay at 40° C. may be at least 70% of that at 37° C. In some instances, the trans cleavage activity of a programmable nuclease in a trans cleavage assay at 40° C. may be at least 75% of that at 37° C. In some instances, the trans cleavage activity of a programmable nuclease in a trans cleavage assay at 40° C. may be at least 80% of that at 37° C. In some instances, the trans cleavage activity of a programmable nuclease in a trans cleavage assay at 40° C. may be at least 85% of that at 37° C. In some instances, the trans cleavage activity of a programmable nuclease in a trans cleavage assay at 40° C. may be at least 90% of that at 37° C. In some instances, the trans cleavage activity of a programmable nuclease in a trans cleavage assay at 40° C. may be at least 95% of that at 37° C. In some instances, the trans cleavage activity of a programmable nuclease in a trans cleavage assay at 40° C. may be at least 100% of that at 37° C. In some instances, the trans cleavage activity of a programmable nuclease in a trans cleavage assay at 40° C. may be at least 1-fold of that at 37° C. In some instances, the trans cleavage activity of a programmable nuclease in a trans cleavage assay at 40° C. may be at least 2-fold of that at 37° C. In some instances, the trans cleavage activity of a programmable nuclease in a trans cleavage assay at 40° C. may be at least 3-fold of that at 37° C. In some instances, the trans cleavage activity of a programmable nuclease in a trans cleavage assay at 40° C. may be at least 4-fold of that at 37° C. In some instances, the trans cleavage activity of a programmable nuclease in a trans cleavage assay at 40° C. may be at least 5-fold of that at 37° C. In some instances, the trans cleavage activity of a programmable nuclease in a trans cleavage assay at 40° C. may be at least 6-fold of that at 37° C. In some instances, the trans cleavage activity of a programmable nuclease in a trans cleavage assay at 40° C. may be at least 7-fold of that at 37° C. In some instances, the trans cleavage activity of a programmable nuclease in a trans cleavage assay at 40° C. may be at least 8-fold of that at 37° C. In some instances, the trans cleavage activity of a programmable nuclease in a trans cleavage assay at 40° C. may be at least 9-fold of that at 37° C. In some instances, the trans cleavage activity of a programmable nuclease in a trans cleavage assay at 40° C. may be at least 10-fold of that at 37° C. In some instances, the trans cleavage activity of a programmable nuclease in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 45° C. may be at least 50% of that at 37° C. In some instances, the trans cleavage activity of a programmable nuclease in a trans cleavage assay at 45° C. may be at least 55% of that at 37° C. In some instances, the trans cleavage activity of a programmable nuclease in a trans cleavage assay at 45° C. may be at least 60% of that at 37° C. In some instances, the trans cleavage activity of a programmable nuclease in a trans cleavage assay at 45° C. may be at least 65% of that at 37° C. In some instances, the trans cleavage activity of a programmable nuclease in a trans cleavage assay at 45° C. may be at least 70% of that at 37° C. In some instances, the trans cleavage activity of a programmable nuclease in a trans cleavage assay at 45° C. may be at least 75% of that at 37° C. In some instances, the trans cleavage activity of a programmable nuclease in a trans cleavage assay at 45° C. may be at least 80% of that at 37° C. In some instances, the trans cleavage activity of a programmable nuclease in a trans cleavage assay at 45° C. may be at least 85% of that at 37° C. In some instances, the trans cleavage activity of a programmable nuclease in a trans cleavage assay at 45° C. may be at least 90% of that at 37° C. In some instances, the trans cleavage activity of a programmable nuclease in a trans cleavage assay at 45° C. may be at least 95% of that at 37° C. In some instances, the trans cleavage activity of a programmable nuclease in a trans cleavage assay at 45° C. may be at least 100% of that at 37° C. In some instances, the trans cleavage activity of a programmable nuclease in a trans cleavage assay at 45° C. may be at least 1-fold of that at 37° C. In some instances, the trans cleavage activity of a programmable nuclease in a trans cleavage assay at 45° C. may be at least 2-fold of that at 37° C. In some instances, the trans cleavage activity of a programmable nuclease in a trans cleavage assay at 45° C. may be at least 3-fold of that at 37° C. In some instances, the trans cleavage activity of a programmable nuclease in a trans cleavage assay at 45° C. may be at least 4-fold of that at 37° C. In some instances, the trans cleavage activity of a programmable nuclease in a trans cleavage assay at 45° C. may be at least 5-fold of that at 37° C. In some instances, the trans cleavage activity of a programmable nuclease in a trans cleavage assay at 45° C. may be at least 6-fold of that at 37° C. In some instances, the trans cleavage activity of a programmable nuclease in a trans cleavage assay at 45° C. may be at least 7-fold of that at 37° C. In some instances, the trans cleavage activity of a programmable nuclease in a trans cleavage assay at 45° C. may be at least 8-fold of that at 37° C. In some instances, the trans cleavage activity of a programmable nuclease in a trans cleavage assay at 45° C. may be at least 9-fold of that at 37° C. In some instances, the trans cleavage activity of a programmable nuclease in a trans cleavage assay at 45° C. may be at least 10-fold of that at 37° C. In some instances, the trans cleavage activity of a programmable nuclease in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 50° C. may be at least 50% of that at 37° C. In some instances, the trans cleavage activity of a programmable nuclease in a trans cleavage assay at 50° C. may be at least 55% of that at 37° C. In some instances, the trans cleavage activity of a programmable nuclease in a trans cleavage assay at 50° C. may be at least 60% of that at 37° C. In some instances, the trans cleavage activity of a programmable nuclease in a trans cleavage assay at 50° C. may be at least 65% of that at 37° C. In some instances, the trans cleavage activity of a programmable nuclease in a trans cleavage assay at 50° C. may be at least 70% of that at 37° C. In some instances, the trans cleavage activity of a programmable nuclease in a trans cleavage assay at 50° C. may be at least 75% of that at 37° C. In some instances, the trans cleavage activity of a programmable nuclease in a trans cleavage assay at 50° C. may be at least 80% of that at 37° C. In some instances, the trans cleavage activity of a programmable nuclease in a trans cleavage assay at 50° C. may be at least 85% of that at 37° C. In some instances, the trans cleavage activity of a programmable nuclease in a trans cleavage assay at 50° C. may be at least 90% of that at 37° C. In some instances, the trans cleavage activity of a programmable nuclease in a trans cleavage assay at 50° C. may be at least 95% of that at 37° C. In some instances, the trans cleavage activity of a programmable nuclease in a trans cleavage assay at 50° C. may be at least 100% of that at 37° C. In some instances, the trans cleavage activity of a programmable nuclease in a trans cleavage assay at 50° C. may be at least 1-fold of that at 37° C. In some instances, the trans cleavage activity of a programmable nuclease in a trans cleavage assay at 50° C. may be at least 2-fold of that at 37° C. In some instances, the trans cleavage activity of a programmable nuclease in a trans cleavage assay at 50° C. may be at least 3-fold of that at 37° C. In some instances, the trans cleavage activity of a programmable nuclease in a trans cleavage assay at 50° C. may be at least 4-fold of that at 37° C. In some instances, the trans cleavage activity of a programmable nuclease in a trans cleavage assay at 50° C. may be at least 5-fold of that at 37° C. In some instances, the trans cleavage activity of a programmable nuclease in a trans cleavage assay at 50° C. may be at least 6-fold of that at 37° C. In some instances, the trans cleavage activity of a programmable nuclease in a trans cleavage assay at 50° C. may be at least 7-fold of that at 37° C. In some instances, the trans cleavage activity of a programmable nuclease in a trans cleavage assay at 50° C. may be at least 8-fold of that at 37° C. In some instances, the trans cleavage activity of a programmable nuclease in a trans cleavage assay at 50° C. may be at least 9-fold of that at 37° C. In some instances, the trans cleavage activity of a programmable nuclease in a trans cleavage assay at 50° C. may be at least 10-fold of that at 37° C. In some instances, the trans cleavage activity of a programmable nuclease in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 55° C. may be at least 50% of that at 37° C. In some instances, the trans cleavage activity of a programmable nuclease in a trans cleavage assay at 55° C. may be at least 55% of that at 37° C. In some instances, the trans cleavage activity of a programmable nuclease in a trans cleavage assay at 55° C. may be at least 60% of that at 37° C. In some instances, the trans cleavage activity of a programmable nuclease in a trans cleavage assay at 55° C. may be at least 65% of that at 37° C. In some instances, the trans cleavage activity of a programmable nuclease in a trans cleavage assay at 55° C. may be at least 70% of that at 37° C. In some instances, the trans cleavage activity of a programmable nuclease in a trans cleavage assay at 55° C. may be at least 75% of that at 37° C. In some instances, the trans cleavage activity of a programmable nuclease in a trans cleavage assay at 55° C. may be at least 80% of that at 37° C. In some instances, the trans cleavage activity of a programmable nuclease in a trans cleavage assay at 55° C. may be at least 85% of that at 37° C. In some instances, the trans cleavage activity of a programmable nuclease in a trans cleavage assay at 55° C. may be at least 90% of that at 37° C. In some instances, the trans cleavage activity of a programmable nuclease in a trans cleavage assay at 55° C. may be at least 95% of that at 37° C. In some instances, the trans cleavage activity of a programmable nuclease in a trans cleavage assay at 55° C. may be at least 100% of that at 37° C. In some instances, the trans cleavage activity of a programmable nuclease in a trans cleavage assay at 55° C. may be at least 1-fold of that at 37° C. In some instances, the trans cleavage activity of a programmable nuclease in a trans cleavage assay at 55° C. may be at least 2-fold of that at 37° C. In some instances, the trans cleavage activity of a programmable nuclease in a trans cleavage assay at 55° C. may be at least 3-fold of that at 37° C. In some instances, the trans cleavage activity of a programmable nuclease in a trans cleavage assay at 55° C. may be at least 4-fold of that at 37° C. In some instances, the trans cleavage activity of a programmable nuclease in a trans cleavage assay at 55° C. may be at least 5-fold of that at 37° C. In some instances, the trans cleavage activity of a programmable nuclease in a trans cleavage assay at 55° C. may be at least 6-fold of that at 37° C. In some instances, the trans cleavage activity of a programmable nuclease in a trans cleavage assay at 55° C. may be at least 7-fold of that at 37° C. In some instances, the trans cleavage activity of a programmable nuclease in a trans cleavage assay at 55° C. may be at least 8-fold of that at 37° C. In some instances, the trans cleavage activity of a programmable nuclease in a trans cleavage assay at 55° C. may be at least 9-fold of that at 37° C. In some instances, the trans cleavage activity of a programmable nuclease in a trans cleavage assay at 55° C. may be at least 10-fold of that at 37° C. In some instances, the trans cleavage activity of a programmable nuclease in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 60° C. may be at least 50% of that at 37° C. In some instances, the trans cleavage activity of a programmable nuclease in a trans cleavage assay at 60° C. may be at least 55% of that at 37° C. In some instances, the trans cleavage activity of a programmable nuclease in a trans cleavage assay at 60° C. may be at least 60% of that at 37° C. In some instances, the trans cleavage activity of a programmable nuclease in a trans cleavage assay at 60° C. may be at least 65% of that at 37° C. In some instances, the trans cleavage activity of a programmable nuclease in a trans cleavage assay at 60° C. may be at least 70% of that at 37° C. In some instances, the trans cleavage activity of a programmable nuclease in a trans cleavage assay at 60° C. may be at least 75% of that at 37° C. In some instances, the trans cleavage activity of a programmable nuclease in a trans cleavage assay at 60° C. may be at least 80% of that at 37° C. In some instances, the trans cleavage activity of a programmable nuclease in a trans cleavage assay at 60° C. may be at least 85% of that at 37° C. In some instances, the trans cleavage activity of a programmable nuclease in a trans cleavage assay at 60° C. may be at least 90% of that at 37° C. In some instances, the trans cleavage activity of a programmable nuclease in a trans cleavage assay at 60° C. may be at least 95% of that at 37° C. In some instances, the trans cleavage activity of a programmable nuclease in a trans cleavage assay at 60° C. may be at least 100% of that at 37° C. In some instances, the trans cleavage activity of a programmable nuclease in a trans cleavage assay at 60° C. may be at least 1-fold of that at 37° C. In some instances, the trans cleavage activity of a programmable nuclease in a trans cleavage assay at 60° C. may be at least 2-fold of that at 37° C. In some instances, the trans cleavage activity of a programmable nuclease in a trans cleavage assay at 60° C. may be at least 3-fold of that at 37° C. In some instances, the trans cleavage activity of a programmable nuclease in a trans cleavage assay at 60° C. may be at least 4-fold of that at 37° C. In some instances, the trans cleavage activity of a programmable nuclease in a trans cleavage assay at 60° C. may be at least 5-fold of that at 37° C. In some instances, the trans cleavage activity of a programmable nuclease in a trans cleavage assay at 60° C. may be at least 6-fold of that at 37° C. In some instances, the trans cleavage activity of a programmable nuclease in a trans cleavage assay at 60° C. may be at least 7-fold of that at 37° C. In some instances, the trans cleavage activity of a programmable nuclease in a trans cleavage assay at 60° C. may be at least 8-fold of that at 37° C. In some instances, the trans cleavage activity of a programmable nuclease in a trans cleavage assay at 60° C. may be at least 9-fold of that at 37° C. In some instances, the trans cleavage activity of a programmable nuclease in a trans cleavage assay at 60° C. may be at least 10-fold of that at 37° C. In some instances, the trans cleavage activity of a programmable nuclease in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 65° C. may be at least 50% of that at 37° C. In some instances, the trans cleavage activity of a programmable nuclease in a trans cleavage assay at 65° C. may be at least 55% of that at 37° C. In some instances, the trans cleavage activity of a programmable nuclease in a trans cleavage assay at 65° C. may be at least 60% of that at 37° C. In some instances, the trans cleavage activity of a programmable nuclease in a trans cleavage assay at 65° C. may be at least 65% of that at 37° C. In some instances, the trans cleavage activity of a programmable nuclease in a trans cleavage assay at 65° C. may be at least 70% of that at 37° C. In some instances, the trans cleavage activity of a programmable nuclease in a trans cleavage assay at 65° C. may be at least 75% of that at 37° C. In some instances, the trans cleavage activity of a programmable nuclease in a trans cleavage assay at 65° C. may be at least 80% of that at 37° C. In some instances, the trans cleavage activity of a programmable nuclease in a trans cleavage assay at 65° C. may be at least 85% of that at 37° C. In some instances, the trans cleavage activity of a programmable nuclease in a trans cleavage assay at 65° C. may be at least 90% of that at 37° C. In some instances, the trans cleavage activity of a programmable nuclease in a trans cleavage assay at 65° C. may be at least 95% of that at 37° C. In some instances, the trans cleavage activity of a programmable nuclease in a trans cleavage assay at 65° C. may be at least 100% of that at 37° C. In some instances, the trans cleavage activity of a programmable nuclease in a trans cleavage assay at 65° C. may be at least 1-fold of that at 37° C. In some instances, the trans cleavage activity of a programmable nuclease in a trans cleavage assay at 65° C. may be at least 2-fold of that at 37° C. In some instances, the trans cleavage activity of a programmable nuclease in a trans cleavage assay at 65° C. may be at least 3-fold of that at 37° C. In some instances, the trans cleavage activity of a programmable nuclease in a trans cleavage assay at 65° C. may be at least 4-fold of that at 37° C. In some instances, the trans cleavage activity of a programmable nuclease in a trans cleavage assay at 65° C. may be at least 5-fold of that at 37° C. In some instances, the trans cleavage activity of a programmable nuclease in a trans cleavage assay at 65° C. may be at least 6-fold of that at 37° C. In some instances, the trans cleavage activity of a programmable nuclease in a trans cleavage assay at 65° C. may be at least 7-fold of that at 37° C. In some instances, the trans cleavage activity of a programmable nuclease in a trans cleavage assay at 65° C. may be at least 8-fold of that at 37° C. In some instances, the trans cleavage activity of a programmable nuclease in a trans cleavage assay at 65° C. may be at least 9-fold of that at 37° C. In some instances, the trans cleavage activity of a programmable nuclease in a trans cleavage assay at 65° C. may be at least 10-fold of that at 37° C. In some instances, the trans cleavage activity of a programmable nuclease in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 70° C., 75° C. 80° C., or more may be at least 50, at least 60%, at least 65%, at least 70%, at least 75%, at least 80%, at least 85%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity may be measured against a negative control in a trans cleavage assay. In some instances, the trans cleavage activity of a programmable nuclease against a nucleic acid in a trans cleavage assay at 37° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 37° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 65° C. may be at least 50%, at least 55%, at least 60%, at least 65%, at least 70%, at least 75% at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 70° C., 75° C., 80° C., or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 70° C., 75° C., 80° C., or more may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid.

The reporters described herein can be RNA reporters. The RNA reporters can comprise at least one ribonucleic acid and a detectable moiety. In some embodiments, a programmable nuclease probe or a CRISPR probe comprising a programmable nuclease can recognize and detect ssDNA and, further, can specifically trans-cleave RNA reporters. The detection of the target nucleic acid in the sample can indicate the presence of the disease (or disease-causing agent) in the sample and can provide information for taking action to reduce the transmission of the disease to individuals in the disease-affected environment or near the disease-carrying individual.

Cleavage of a reporter (i.e., a protein-nucleic acid or detector nucleic acid) can produce a signal. The signal can indicate a presence of the target nucleic acid in the sample, and an absence of the signal can indicate an absence of the target nucleic acid in the sample. In some cases, cleavage of the reporter can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors and detectors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor or detector suitable for detecting calorimetric signals, potentiometric signals, amperometric signals, optical signals, or piezo-electric signals.

In an aspect, the present disclosure provides a method for target detection. The method can comprise sample collection. The method can further comprise sample preparation. The method can further comprise detection of one or more target molecules in the collected and prepared sample. In some embodiments, sample preparation can include nucleic acid amplification and the target molecules can include target amplicons.

In another aspect, the present disclosure provides a detection device for target detection. The detection device can be configured for multiplexed target detection. The detection device can be used to collect one or more samples, prepare or process the one or more samples for detection, and optionally divide the one or more samples into a plurality of droplets, aliquots, volumes, or subsamples for amplification of one or more target sequences or target nucleic acids. The target sequences may comprise, for example, a biological sequence. The biological sequence can comprise a nucleic acid sequence or an amino acid sequence. In some embodiments, the target sequences are associated with an organism of interest, a disease of interest, a disease state of interest, a phenotype of interest, a genotype of interest, or a gene of interest.

The detection device can be configured to amplify target nucleic acids contained within the plurality of droplets, aliquots, or subsamples. The detection device can be configured to amplify the target sequences or target nucleic acids contained within the plurality of droplets or volumes by individually processing each of the plurality of droplets or volumes (e.g., by using a thermocycling process or any other suitable amplification process as described in greater detail below). In some cases, the plurality of droplets or volumes can undergo separate thermocycling processes. In some cases, the thermocycling processes can occur simultaneously. In other cases, the thermocycling processes can occur at different times for each droplet or volume.

The detection device can be further configured to remix the droplets, aliquots, volumes, or subsamples after the target nucleic acids in each of the droplets undergo amplification. The detection device can be configured to provide the remixed sample comprising the droplets, aliquots, volumes, or subsamples to a detection chamber of the device. The detection chamber can be configured to direct the remixed droplets, aliquots, volumes, or subsamples to a plurality of programmable nuclease probes. The detection chamber can be configured to direct the remixed droplets, aliquots, volumes, or subsamples along one or more fluid flow paths such that the remixed droplets, aliquots, volumes, or subsamples are positioned adjacent to and/or in contact with the one or more programmable nuclease probes. In some cases, the detection chamber can be configured to recirculate or recycle the remixed droplets, aliquots, volumes, or subsamples such that the remixed droplets, aliquots, volumes, or subsamples are repeatedly placed in contact with one or more programmable nuclease probes over a predetermined period of time.

The instrument and/or detection device can comprise one or more sensors or detectors. The one or more sensors or detectors of the instrument and/or detection device can be configured to detect one or more signals that are generated after one or more programmable nucleases of the one or more programmable nuclease probes become activated due to a binding of a guide nucleic acid of the programmable nuclease probes with a target nucleic acid present in the sample or amplicon thereof. As described elsewhere herein, the activated programmable nuclease can bind or cleave the target nucleic acid, which can result in a trans cleavage activity. Trans cleavage activity can be a non-specific cleavage of nearby single-stranded nucleic acids by the activated programmable nuclease, such as trans cleavage of reporter nucleic acids with a detection moiety. Once the reporter nucleic acids are cleaved by the activated programmable nucleases, the detection moiety can be released or separated from the reporter, thereby generating one or more detectable signals. The one or more sensors or detectors of the instrument or detection device can be configured to register and/or process the one or more detectable signals to confirm a presence and/or an absence of a particular target (e.g., a target nucleic acid) in a sample.

The one or more programmable nuclease probes of the detection device can be configured for multiplexed detection. In some cases, each programmable nuclease probe can be configured to detect a particular target. In other cases, each programmable nuclease probe can be configured to detect a plurality of targets. In some cases, a first programmable nuclease probe can be configured to detect a first target or a first set of targets, and a second programmable nuclease probe can be configured to detect a second target or a second set of targets. In other cases, a first programmable nuclease probe can be configured to detect a first set of targets, and a second programmable nuclease probe can be configured to detect a second set of targets. The programmable nuclease probes of the present disclosure can be used to detect a plurality of different target sequences or target nucleic acids. In any of the embodiments described herein, the sample provided to the detection device can comprise a plurality of target sequences or target nucleic acids. In any of the embodiments described herein, the sample provided to the detection device can comprise multiple classes of target sequences or target nucleic acids. Each class of target sequences or class of target nucleic acids can comprise a plurality of target sequences or target nucleic acids associated with a particular organism, disease state, phenotype, or genotype present within the sample. In some cases, each programmable nuclease probe can be used to detect a particular class of target sequences, or a particular class of target nucleic acids associated with a particular organism, disease state, phenotype, or genotype present within the sample. In some cases, two or more programmable nuclease probes can be used to detect different classes of target sequences or different classes of target nucleic acids. In such cases, the two or more programmable nuclease probes can comprise different sets or classes of guide nucleic acids complexed to the programmable nucleases of the probes.

### Guide Nucleic Acids

Guide nucleic acids are compatible for use in the devices described herein and may be used in conjunction with compositions disclosed herein (e.g., programmable nucleases, reagents for in vitro transcription, reagents for amplification, reagents for reverse transcription, and reporters, or any combination thereof) to carry out highly efficient, rapid, and accurate reactions for detecting whether a target nucleic acid is present in a sample (e.g., DETECTR reactions). The guide nucleic acid binds to the single stranded or double stranded target nucleic acid comprising a portion of a nucleic acid from a virus or a bacterium or other agents responsible for a disease as described herein. The guide nucleic acid can bind to the single stranded or double stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded or double stranded target nucleic acid comprising a portion of a nucleic acid from a cancer gene or gene associated with a genetic disorder as described herein. The guide nucleic acid is complementary to the target nucleic acid or a portion thereof. Often the guide nucleic acid binds specifically to the target nucleic acid. The target nucleic acid may be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid may comprise a crRNA and tracrRNA. The guide nucleic acid can bind specifically to the target nucleic acid. In some cases, the guide nucleic acid is not naturally occurring. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30 nt to about 40 nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of HPV 16 or HPV 18, for example. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling may comprise gaps between the tiled guide nucleic acids along the target nucleic acid. In some instances, the tiling of the guide nucleic acids is non-sequential. Often, a method for detecting a target nucleic acid may comprise contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some reporters of a population of reporters. Pooling of guide nucleic acids can ensure broad spectrum identification, or broad coverage, of a target species within a single reaction. This can be particularly helpful in diseases or indications, like sepsis, that may be caused by multiple organisms.

### Reporters

Reporters, which can be referred to interchangeably reporter molecules, or detector nucleic acids, described herein are compatible for use in the devices described herein and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). Described herein is a reporter comprising a single stranded nucleic acid and a detection moiety, wherein the reporter is capable of being cleaved by the activated programmable nuclease, thereby generating a first detectable signal. As used herein, a detector nucleic acid is used interchangeably with reporter or reporter molecule. In some cases, the reporter comprises a single-stranded nucleic acid. In some cases, the reporter comprises a double-stranded nucleic acid. In some cases, the reporter can comprise a single-stranded nucleic acid coupled to a double-stranded nucleic acid. In some cases, the reporter comprises a single-stranded nucleic acid comprising deoxyribonucleotides. In some cases, the reporter comprises a double-stranded nucleic acid comprising deoxyribonucleotides. In some cases, the reporter comprises a single-stranded nucleic acid comprising ribonucleotides. The reporter can comprise a single-stranded nucleic acid comprising at least one deoxyribonucleotide and at least one ribonucleotide. In some cases, the reporter comprises a single-stranded nucleic acid comprising at least one ribonucleotide residue at an internal position that functions as a cleavage site. In some cases, the reporter may comprise at least 2, 3, 4, 5, 6, 7, 8, 9, or 10 ribonucleotide residues at an internal position. In some cases, the reporter may comprise from 2 to 10, from 3 to 9, from 4 to 8, or from 5 to 7 ribonucleotide residues at an internal position. In some cases, the reporter may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter has only deoxyribonucleotide residues. In some cases, the reporter may comprise nucleotides resistant to cleavage by the programmable nuclease described herein. In some cases, the reporter may comprise synthetic nucleotides. In some cases, the reporter may comprise at least one ribonucleotide residue and at least one non-ribonucleotide residue. In some cases, the reporter is 5-20, 5-15, 5-10, 7-20, 7-15, or 7-10 nucleotides in length. In some cases, the reporter is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the reporter may comprise at least one uracil ribonucleotide. In some cases, the reporter may comprise at least two uracil ribonucleotides. Sometimes the reporter has only uracil ribonucleotides. In some cases, the reporter may comprise at least one adenine ribonucleotide. In some cases, the reporter may comprise at least two adenine ribonucleotide. In some cases, the reporter has only adenine ribonucleotides. In some cases, the reporter may comprise at least one cytosine ribonucleotide. In some cases, the reporter may comprise at least two cytosine ribonucleotide. In some cases, the reporter may comprise at least one guanine ribonucleotide. In some cases, the reporter may comprise at least two guanine ribonucleotide. A reporter can comprise only unmodified ribonucleotides, only unmodified deoxyribonucleotides, or a combination thereof. In some cases, the reporter is from 5 to 12 nucleotides in length. In some cases, the reporter is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the reporter is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a reporter can be 5, 8, or 10 nucleotides in length. For cleavage by a programmable nuclease comprising Cas12, a reporter can be 10 nucleotides in length.

In some embodiments, the reporter may comprise a nucleic acid and a detection moiety. In some embodiments, a reporter is connected to a surface by a linkage. In some embodiments, a reporter may comprise at least one of a nucleic acid, a chemical functionality, a detection moiety, a quenching moiety, or a combination thereof. In some embodiments, a reporter is configured for the detection moiety to remain immobilized to the surface and the quenching moiety to be released into solution upon cleavage of the reporter. In some embodiments, a reporter is configured for the quenching moiety to remain immobilized to the surface and for the detection moiety to be released into solution, upon cleavage of the reporter. Often the detection moiety is at least one of a label, a polypeptide, a dendrimer, or a nucleic acid or a combination thereof. In some embodiments, the reporter contains a label. In some embodiments, label may be FITC, DIG, TAMRA, Cy5, AF594, or Cy3. In some embodiments, the label may comprise a dye, a nanoparticle configured to produce a signal, or the like. In some embodiments, the dye may be a fluorescent dye. In some embodiments, the at least one chemical functionality may comprise biotin. In some embodiments, the at least one chemical functionality may be configured to be captured by a capture probe. In some embodiments, the at least one chemical functionality may comprise biotin and the capture probe may comprise anti-biotin, streptavidin, avidin or other molecule configured to bind with biotin. In some embodiments, the dye is the chemical functionality. In some embodiments, a capture probe may comprise a molecule that is complementary to the chemical functionality of the reporter. In some embodiments, the capture antibodies are anti-FITC, anti-DIG, anti-TAMRA, anti-Cy5, anti-AF594, or any other appropriate capture antibody capable of binding the detection moiety or conjugate. In some embodiments, the detection moiety can be the chemical functionality.

In some embodiments, the reporter may comprise a quenching moiety. In some embodiments, a quenching moiety is any entity that decreases the fluorescence intensity of a given substance. Exemplary embodiments of reporters, labels, quenchers, chemical functionalities, detection moieties, dendrimers, quenching moieties and other reporter elements are described in: PCT/US2021/033271; PCT/US2021/035031, and PCT/US2022/028865, all of which are herein incorporated by reference in their entirety.

In some cases, the reporter comprises a detection moiety and a quenching moiety. In some instances, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some cases, the detection moiety is at the 5′ terminus of the nucleic acid of a reporter. In some cases, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, (E≤-glucuronidase, invertase, Xanthine Oxidase, firefly luciferase, and glucose oxidase (GO).

In some instances, the detection moiety comprises an invertase. The substrate of the invertase may be sucrose. A DNS reagent may be included in the system to produce a colorimetric change when the invertase converts sucrose to glucose. In some cases, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). Non-limiting examples of fluorophores are fluorescein amidite, 6-Fluorescein, IRDye 700, TYE 665, Alex Fluor 594, or ATTO™ 633 (NHS Ester). The fluorophore may be an infrared fluorophore. The fluorophore may emit fluorescence in the range of 500 nm and 720 nm. In some cases, the fluorophore emits fluorescence at a wavelength of 700 nm or higher. In other cases, the fluorophore emits fluorescence at about 665 nm. In some cases, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some cases, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO™ 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-trade name of the quenching moieties listed.

The generation of the detectable signal from the release of the detection moiety indicates that cleavage by the programmable nucleases has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter. Other methods of detection can also be used, such as optical imaging, surface plasmon resonance (SPR), and/or interferometric sensing.

The detectable signal may be a colorimetric signal or a signal visible by eye. In some instances, the detectable signal may be fluorescent, electrical, chemical, electrochemical, or magnetic. In some cases, a detectable signal (e.g., a first detectable signal) may be generated by binding of the detection moiety to the capture molecule in the detection region, where the detectable signal indicates that the sample contained the target nucleic acid. Sometimes systems are capable of detecting more than one type of target nucleic acid, wherein the system comprises more than one type of guide nucleic acid and more than one type of reporter nucleic acid. In some cases, the detectable signal may be generated directly by the cleavage event. Alternatively, or in combination, the detectable signal may be generated indirectly by the cleavage event. Sometimes the detectable signal is not a fluorescent signal. In some instances, the detectable signal may be a colorimetric or color-based signal. In some cases, the detected target nucleic acid may be identified based on its spatial location on the detection region of the support medium. In some cases, a second detectable signal may be generated in a spatially distinct location than a first detectable signal when two or more detectable signals are generated.

In some cases, the one or more detectable signals generated after cleavage can produce an index of refraction change or one or more electrochemical changes. In some cases, real-time detection of the Cas reaction can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope).

Often, the reporter is an enzyme-nucleic acid. The enzyme may be sterically hindered when present as in the enzyme-nucleic acid, but then functional upon cleavage from the nucleic acid by the programmable nuclease. Often, the enzyme is an enzyme that produces a reaction with an enzyme substrate. An enzyme can be invertase. Often, the substrate of invertase is sucrose and DNS reagent.

Sometimes the reporter is a substrate-nucleic acid. Often the substrate is a substrate that produces a reaction with an enzyme. Release of the substrate upon cleavage by the programmable nuclease may free the substrate to react with the enzyme.

A reporter may be attached to a solid support. The solid support, for example, is a surface. A surface can be an electrode. Sometimes the solid support is a bead. Often the bead is a magnetic bead. Upon cleavage, the detection moiety is liberated from the solid support and interacts with other mixtures. For example, the detection moiety is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the detection moiety is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the reporter comprises a nucleic acid conjugated to an affinity molecule which is in turn conjugated to the fluorophore (e.g., nucleic acid-affinity molecule-fluorophore) or the nucleic acid conjugated to the fluorophore which is in turn conjugated to the affinity molecule (e.g., nucleic acid-fluorophore-affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicates that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore-no intervening moiety is present between the nucleic acid and the affinity molecule and no intervening moiety is present between the affinity molecule and the fluorophore. The affinity molecule can be biotin, avidin, streptavidin, or any similar molecule.

In some cases, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

The reporter can be lyophilized or vitrified. The reporter can be suspended in solution or immobilized on a surface. For example, the reporter can be immobilized, dried, or otherwise deposited on the surface of a chamber in a device as disclosed herein. In some cases, the reporter is immobilized on beads, such as magnetic beads, in a chamber of a device as disclosed herein where they can be held in position by a magnet placed below the chamber.

In some cases, the reporter is a single-stranded nucleic acid comprising deoxyribonucleotides. In some cases, the reporter nucleic acid is a single-stranded nucleic acid sequence comprising ribonucleotides. The nucleic acid of a reporter may be a single-stranded nucleic acid sequence comprising at least one ribonucleotide. In some cases, the nucleic acid of a reporter is a single-stranded nucleic acid comprising at least one ribonucleotide residue at an internal position that functions as a cleavage site. In some cases, the nucleic acid of a reporter comprises at least 2, at least 3, at least 4, at least 5, at least 6, at least 7, at least 8, at least 9, or at least 10 ribonucleotide residues at an internal position. In some cases, the nucleic acid of a reporter comprises from 2 to 10, from 3 to 9, from 4 to 8, or from 5 to 7 ribonucleotide residues at an internal position. In some cases, the reporter may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the nucleic acid of a reporter has only ribonucleotide residues. In some cases, the nucleic acid of a reporter has only deoxyribonucleotide residues. In some cases, the nucleic acid comprises nucleotides resistant to cleavage by the programmable nuclease described herein. In some cases, the nucleic acid of a reporter comprises synthetic nucleotides. In some cases, the nucleic acid of a reporter comprises at least one ribonucleotide residue and at least one non-ribonucleotide residue.

In some cases, the nucleic acid of a reporter comprises at least one uracil ribonucleotide. In some cases, the nucleic acid of a reporter comprises at least two uracil ribonucleotides. Sometimes the nucleic acid of a reporter has only uracil ribonucleotides. In some cases, the nucleic acid of a reporter comprises at least one adenine ribonucleotide. In some cases, the nucleic acid of a reporter comprises at least two adenine ribonucleotide. In some cases, the nucleic acid of a reporter has only adenine ribonucleotides. In some cases, the nucleic acid of a reporter comprises at least one cytosine ribonucleotide. In some cases, the nucleic acid of a reporter comprises at least two cytosine ribonucleotide. In some cases, the nucleic acid of a reporter comprises at least one guanine ribonucleotide. In some cases, the nucleic acid of a reporter comprises at least two guanine ribonucleotide. In some instances, a nucleic acid of a reporter comprises a single unmodified ribonucleotide. In some instances, a nucleic acid of a reporter comprises only unmodified ribonucleotides. In some instances, a nucleic acid of a reporter comprises only unmodified deoxyribonucleotides.

In some cases, the nucleic acid of a reporter is 5 to 20, 5 to 15, 5 to 10, 7 to 20, 7 to 15, or 7 to 10 nucleotides in length. In some cases, the nucleic acid of a reporter is 3 to 20, 4 to 20, 5 to 20, 6 to 20, 7 to 20, 8 to 20, 9 to 20, 10 to 20, 13 to 20, 15 to 20, 3 to 15, 4 to 15, 5 to 15, 6 to 15, 7 to 15, 8 to 15, 9 to 15, 10 to 15, 3 to 10, 4 to 10, 5 to 10, 6 to 10, 7 to 10, 8 to 10, 9 to 10, 3 to 8, 4 to 8, 5 to 8, 6 to 8, or 7 to 8, nucleotides in length. In some cases, the nucleic acid of a reporter is 5 to 12 nucleotides in length. In some cases, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a reporter can be 5, 8, or 10 nucleotides in length. For cleavage by a programmable nuclease comprising Cas12, a reporter can be 10 nucleotides in length.

In some cases, systems comprise a plurality of reporters. The plurality of reporters may comprise a plurality of signals. In some cases, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some cases, there are 2 to 50, 3 to 40, 4 to 30, 5 to 20, or 6 to 10 different reporters.

In some instances, systems comprise a Type V CRISPR/Cas protein and a reporter nucleic acid configured to undergo transcollateral cleavage by the Type V CRISPR/Cas protein. Transcollateral cleavage of the reporter may generate a signal from the reporter or alter a signal from the reporter. In some cases, the signal is an optical signal, such as a fluorescence signal or absorbance band. Transcollateral cleavage of the reporter may alter the wavelength, intensity, or polarization of the optical signal. For example, the reporter may comprise a fluorophore and a quencher, such that transcollateral cleavage of the reporter separates the fluorophore and the quencher thereby increasing a fluorescence signal from the fluorophore. Herein, detection of reporter cleavage to determine the presence of a target nucleic acid sequence may be referred to as ‘DETECTR’. In some embodiments described herein is a method of assaying for a target nucleic acid in a sample comprising contacting the target nucleic acid with a programmable nuclease, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of a programmable nuclease (e.g., a Type V CRISPR/Cas protein as disclosed herein) may be inhibited. If total nucleic acids are present in large amounts, they may outcompete reporters for the programmable nucleases. In some instances, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some instances, the sample comprises amplified target nucleic acid. In some instances, the sample comprises an unamplified target nucleic acid. In some instances, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some instances, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Immobilized Reporter Systems

Disclosed herein are immobilized reporter systems, compositions, and methods of use thereof, e.g., for detection of a target nucleic acid or a plurality of target nucleic acids.

In some instances, systems comprise a Type V CRISPR/Cas protein and a reporter configured to undergo transcollateral cleavage by the Type V CRISPR/Cas protein. In some instances, systems comprise a Type VI CRISPR/Cas protein and a reporter configured to undergo transcollateral cleavage by the Type VI CRISPR/Cas protein. Transcollateral cleavage of the reporter may generate a signal from the reporter, alter a signal from the reporter, or trigger a downstream reaction capable of generating or changing a signal in response to cleavage of the reporter and release of a detection moiety therefrom. In some cases, the signal is an optical signal, such as a fluorescence signal or absorbance signal. Transcollateral cleavage of the reporter may alter the wavelength, intensity, and/or polarization of the optical signal. For example, the reporter may comprise a fluorophore and a quencher, such that transcollateral cleavage of the reporter separates the fluorophore and the quencher thereby increasing a fluorescence signal from the fluorophore. In some embodiments described herein is a method of assaying for a target nucleic acid in a sample comprising contacting the target nucleic acid with a programmable nuclease, a non-naturally occurring guide nucleic acid that hybridizes to a segment of the target nucleic acid, and a reporter, and assaying for a change in a signal, wherein the change in the signal is produced by cleavage of the reporter.

Reporter systems disclosed herein may comprise one or more reporters. Described herein are compositions and methods of use thereof comprising one or more reporter molecules. In some examples, the one or more reporter molecules comprise one or more different reporter molecules. In an example, the one or more reporter molecules comprise a first reporter molecule, a second reporter molecule, a third reporter molecule, and/or more reporter molecules or a plurality of each reporter molecule wherein each reporter molecule can be present in multiple copies (e.g., at a predefined concentration) in the composition. In some examples, the compositions and methods comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more reporter molecules or sequences.

By way of non-limiting and illustrative example, a reporter may comprise a single stranded nucleic acid and a detection moiety (e.g., a labeled single stranded RNA reporter), wherein the nucleic acid is capable of being cleaved by a programmable nuclease (e.g., a Type V or Type VI CRISPR/Cas protein as disclosed herein) or a multimeric complex thereof, releasing the detection moiety, and, generating a detectable signal. In some instances, the reporter additionally comprises a double stranded nucleic acid. As used herein, “reporter” is used interchangeably with “reporter molecule”. The programmable nucleases disclosed herein, activated upon hybridization of a guide RNA to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some instances, a reporter may be immobilized on a substrate. In some cases, a reporter may be immobilized to a surface of the substrate. In some cases, the reporter may be immobilized to a detection location of a substrate. In some instances, the reporter may be immobilized on the substrate. The reporter can be attached to a solid support. The solid support, for example, is a surface. A surface can be an electrode. Sometimes the solid support is a bead. In some cases, the bead is a magnetic bead. The surface can also be an array or a slide.

The reporter comprising a nucleic acid, in some cases, may be immobilized at the 5′end of the nucleic acid. In some cases, the reporter may be immobilized at the 3′ end of the nucleic acid. In some cases, the reporter may be immobilized at the 5′ and 3′ end of the nucleic acid.

In some instances, a reporter may be immobilized to a substrate via covalent bonding. In some cases, the reporter may comprise a thiol or an amine group for immobilization.

In some embodiments, the one or more detection reagents can be immobilized in discrete detection locations using NHS-amine chemistry as described herein. For example, a primary amine-modified guide nucleic acid and a primary amine-modified reporter may be conjugated to an NHS-coated surface of the detection region. In some cases, the amine may form an amide bond with the substrate. For example, the substrate may comprise graphene oxide. NHS-amine, in some cases, may have a structure of

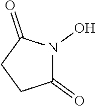

In some cases, the immobilization moiety of a reporter may comprise a thiol group. The thiol group may form an Au—S bond with the substrate. The substrate may comprise gold. In some embodiments, the one or more detection reagents may be immobilized using maleimide-thiol chemistry as described herein. For example, a thiol-modified guide nucleic acid and a thiol-modified reporter may be conjugated to a maleimide-coated surface of the detection region. Thiol group, in some cases, may have a structure of S—H, wherein S is sulfur.

In some cases, a reporter may be immobilized to a substrate via non-covalent bonding. In some embodiments, the one or more detection reagents may be immobilized using avidin/streptavidin-biotin chemistry as described herein. For example, a biotinylated reporter and a biotinylated guide nucleic acid may be immobilized to a streptavidin-coated surface of the detection region.

In some instances, the immobilization of the reporter on a substrate may comprise an immobilization moiety. In some cases, the reporter may comprise an amino group moiety, a peptide moiety, a polypeptide moiety, or a protein moiety. The amino group moiety, peptide moiety, polypeptide moiety, or protein moiety may be the immobilization moiety. The immobilization moiety may comprise an amino modifier. The immobilization moiety may be 5′ or 3′ of the nucleic acid of the reporter.

In some instances, a reporter may be immobilized by surface adsorption. In some cases, the reporter may be immobilized on the surface via electrostatic interaction between the reporter and the surface. For example, a reporter may comprise a negative charge and a surface may comprise a positive charge. In some cases, the surface of a substrate may be coated with a material. The coated material may comprise polyamine, poly-L-lysine, polypyrrole, polyaniline, polyethyleneimine, or a combination thereof.

In some instances, the immobilization moiety may also be used to immobilize a guide nucleic acid. In some cases, the immobilization moiety can be located at an end of a guide nucleic acid. In some cases, the immobilization moiety can be located at the 5′ end of a guide nucleic acid. In some cases, the immobilization moiety can be located at the 3′ end of a guide nucleic acid. In some cases, the immobilization moiety can be located at the 5′ and 3′ end of a guide nucleic acid.

### Reporter Design

In some instances, a reporter may comprise a nucleic acid. In some cases, the nucleic acid may have a polynucleotide sequence. In some cases, the polynucleotide sequence may comprise about 10 nucleotides. In some cases, the polynucleotide sequence may comprise about 11 nucleotides. In some cases, the polynucleotide sequence may comprise about 12 nucleotides. In some cases, the polynucleotide sequence may comprise about 13 nucleotides. In some cases, the polynucleotide sequence may comprise about 14 nucleotides. In some cases, the polynucleotide sequence may comprise about 15 nucleotides. In some cases, the polynucleotide sequence may comprise about 16 nucleotides. In some cases, the polynucleotide sequence may comprise about 17 nucleotides. In some cases, the polynucleotide sequence may comprise about 18 nucleotides. In some cases, the polynucleotide sequence may comprise about 19 nucleotides. In some cases, the polynucleotide sequence may comprise about 20 nucleotides. In some cases, the polynucleotide sequence may comprise about 21 nucleotides. In some cases, the polynucleotide sequence may comprise about 22 nucleotides. In some cases, the polynucleotide sequence may comprise about 23 nucleotides. In some cases, the polynucleotide sequence may comprise about 24 nucleotides. In some cases, the polynucleotide sequence may comprise about 25 nucleotides. In some cases, the polynucleotide sequence may comprise about 26 nucleotides. In some cases, the polynucleotide sequence may comprise about 27 nucleotides. In some cases, the polynucleotide sequence may comprise about 28 nucleotides. In some cases, the polynucleotide sequence may comprise about 29 nucleotides. In some cases, the polynucleotide sequence may comprise about 30 nucleotides. In some cases, the polynucleotide sequence may comprise about 31 nucleotides. In some cases, the polynucleotide sequence may comprise about 32 nucleotides. In some cases, the polynucleotide sequence may comprise about 33 nucleotides. In some cases, the polynucleotide sequence may comprise about 34 nucleotides. In some cases, the polynucleotide sequence may comprise about 35 nucleotides. In some cases, the polynucleotide sequence may comprise about 36 nucleotides. In some cases, the polynucleotide sequence may comprise about 37 nucleotides. In some cases, the polynucleotide sequence may comprise about 38 nucleotides. In some cases, the polynucleotide sequence may comprise about 39 nucleotides. In some cases, the polynucleotide sequence may comprise about 40 nucleotides. In some cases, the polynucleotide sequence may comprise about 41 nucleotides. In some cases, the polynucleotide sequence may comprise about 42 nucleotides. In some cases, the polynucleotide sequence may comprise about 43 nucleotides. In some cases, the polynucleotide sequence may comprise about 44 nucleotides. In some cases, the polynucleotide sequence may comprise about 45 nucleotides. In some cases, the polynucleotide sequence may comprise about 46 nucleotides. In some cases, the polynucleotide sequence may comprise about 47 nucleotides. In some cases, the polynucleotide sequence may comprise about 48 nucleotides. In some cases, the polynucleotide sequence may comprise about 49 nucleotides. In some cases, the polynucleotide sequence may comprise about 50 nucleotides. In some cases, the polynucleotide sequence may comprise about 51 nucleotides. In some cases, the polynucleotide sequence may comprise about 52 nucleotides. In some cases, the polynucleotide sequence may comprise about 53 nucleotides. In some cases, the polynucleotide sequence may comprise about 54 nucleotides. In some cases, the polynucleotide sequence may comprise about 55 nucleotides. In some cases, the polynucleotide sequence may comprise about 56 nucleotides. In some cases, the polynucleotide sequence may comprise about 57 nucleotides. In some cases, the polynucleotide sequence may comprise about 58 nucleotides. In some cases, the polynucleotide sequence may comprise about 59 nucleotides. In some cases, the polynucleotide sequence may comprise about 60 nucleotides. In some cases, the polynucleotide sequence may comprise about 61 nucleotides. In some cases, the polynucleotide sequence may comprise about 62 nucleotides. In some cases, the polynucleotide sequence may comprise about 63 nucleotides. In some cases, the polynucleotide sequence may comprise about 64 nucleotides. In some cases, the polynucleotide sequence may comprise about 65 nucleotides. In some cases, the polynucleotide sequence may comprise about 66 nucleotides. In some cases, the polynucleotide sequence may comprise about 67 nucleotides. In some cases, the polynucleotide sequence may comprise about 68 nucleotides. In some cases, the polynucleotide sequence may comprise about 69 nucleotides. In some cases, the polynucleotide sequence may comprise about 70 nucleotides. In some cases, the polynucleotide sequence may comprise about 71 nucleotides. In some cases, the polynucleotide sequence may comprise about 72 nucleotides. In some cases, the polynucleotide sequence may comprise about 73 nucleotides. In some cases, the polynucleotide sequence may comprise about 74 nucleotides. In some cases, the polynucleotide sequence may comprise about 75 nucleotides. In some cases, the polynucleotide sequence may comprise about 76 nucleotides. In some cases, the polynucleotide sequence may comprise about 77 nucleotides. In some cases, the polynucleotide sequence may comprise about 78 nucleotides. In some cases, the polynucleotide sequence may comprise about 79 nucleotides. In some cases, the polynucleotide sequence may comprise about 80 nucleotides. In some cases, the polynucleotide sequence may comprise about 81 nucleotides. In some cases, the polynucleotide sequence may comprise about 82 nucleotides. In some cases, the polynucleotide sequence may comprise about 83 nucleotides. In some cases, the polynucleotide sequence may comprise about 84 nucleotides. In some cases, the polynucleotide sequence may comprise about 85 nucleotides. In some cases, the polynucleotide sequence may comprise about 86 nucleotides. In some cases, the polynucleotide sequence may comprise about 87 nucleotides. In some cases, the polynucleotide sequence may comprise about 88 nucleotides. In some cases, the polynucleotide sequence may comprise about 89 nucleotides. In some cases, the polynucleotide sequence may comprise about 90 nucleotides. In some cases, the polynucleotide sequence may comprise about 91 nucleotides. In some cases, the polynucleotide sequence may comprise about 92 nucleotides. In some cases, the polynucleotide sequence may comprise about 93 nucleotides. In some cases, the polynucleotide sequence may comprise about 94 nucleotides. In some cases, the polynucleotide sequence may comprise about 95 nucleotides. In some cases, the polynucleotide sequence may comprise about 96 nucleotides. In some cases, the polynucleotide sequence may comprise about 97 nucleotides. In some cases, the polynucleotide sequence may comprise about 98 nucleotides. In some cases, the polynucleotide sequence may comprise about 99 nucleotides. In some cases, the polynucleotide sequence may comprise about 100 nucleotides. In some cases, the polynucleotide sequence may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, 500 or more nucleotides. In some cases, the polynucleotide sequence may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, or 500 nucleotides. For cleavage by a programmable nuclease comprising Cas13, a reporter can comprise any numbers of nucleotides described thereof. In some cases, a reporter can be 5, 8, or 10 nucleotides in length. For cleavage by a programmable nuclease comprising Cas12, a reporter can comprise any numbers of nucleotides described thereof. In some cases, a reporter can be about 10 nucleotides in length.

Reporters may comprise RNA. Reporters may comprise DNA. Reporters may also comprise both DNA and RNA. Reporters may be double-stranded. Reporters may be single-stranded. A reporter may comprise a single-stranded region. A reporter may comprise a double-stranded region. In some cases, reporters may comprise both single-stranded and doubles-stranded regions. In some instances, cleavage of the reporter produces, changes, or reduces a signal and thereby indicate the presence of the target nucleic acid in the sample. The systems and devices disclosed herein can be used to detect these signals, which can indicate whether a target nucleic acid is present in the sample.

The reporter can comprise a single-stranded nucleic acid sequence comprising at least one deoxyribonucleotide and at least one ribonucleotide. The reporter can comprise a double-stranded nucleic acid sequence comprising at least one deoxyribonucleotide and at least one ribonucleotide. The reporter can comprise a single-stranded nucleic acid sequence and a double-stranded nucleic acid region, each comprising at least one deoxyribonucleotide and at least one ribonucleotide.

In some instances, the single-stranded region of a reporter may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, 500 or more nucleotides. In some cases, the single-stranded region of the reporter may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, or 500 nucleotides. In some cases, the single-stranded region may comprise about 5 to about 15 nucleotides. In some cases, the single-stranded region may comprise about 5 to about 20 nucleotides. In some cases, the single-stranded region may comprise about 5 to about 25 nucleotides. In some cases, the single-stranded region may comprise about 5 to about 50 nucleotides. In some cases, the single-stranded region may comprise about 5 to about 100 nucleotides. In some cases, the single-stranded region may comprise about 5 to about 200 nucleotides. In some cases, the single-stranded region may comprise about 5 to about 500 nucleotides. In some cases, the single-stranded region may comprise about 4 to about 15 nucleotides. In some cases, the single-stranded region may comprise about 3 to about 15 nucleotides. In some cases, the single-stranded region may comprise about 2 to about 15 nucleotides. In some cases, the single-stranded region may comprise about 1 to about 15 nucleotides. In some cases, the single-stranded region may comprise about 1 nucleotides. In some cases, the single-stranded region may comprise about 2 nucleotides. In some cases, the single-stranded region may comprise about 3 nucleotides. In some cases, the single-stranded region may comprise about 4 nucleotides. In some cases, the single-stranded region may comprise about 5 nucleotides. In some cases, the single-stranded region may comprise about 6 nucleotides. In some cases, the single-stranded region may comprise about 7 nucleotides. In some cases, the single-stranded region may comprise about 8 nucleotides. In some cases, the single-stranded region may comprise about 9 nucleotides. In some cases, the single-stranded region may comprise about 10 nucleotides. In some cases, the single-stranded region may comprise about 11 nucleotides. In some cases, the single-stranded region may comprise about 12 nucleotides. In some cases, the single-stranded region may comprise about 13 nucleotides. In some cases, the single-stranded region may comprise about 14 nucleotides. In some cases, the single-stranded region may comprise about 15 nucleotides. In some cases, the single-stranded region may comprise about 20 nucleotides. In some cases, the single-stranded region may comprise about 30 nucleotides. In some cases, the single-stranded region may comprise about 40 nucleotides. In some cases, the single-stranded region may comprise about 50 nucleotides. In some cases, the single-stranded region may comprise about 100 nucleotides. In some cases, the single-stranded region may comprise about 150 nucleotides. In some cases, the single-stranded region may comprise about 200 nucleotides. In some cases, the single-stranded region may comprise about 500 nucleotides.

In some instances, the double-stranded region of a reporter may comprise at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 90, 100, 150, 200, 250, 300, 350, 400, 450, 500 or more nucleotide pairs. In some instances, the double-stranded region of a reporter may comprise at most about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 90, 100, 150, 200, 250, 300, 350, 400, 450, 500 or more nucleotide pairs.

In some cases, the double-stranded region may comprise a length of about 10 nucleotides. In some cases, the double-stranded region may comprise a length of about 15 nucleotides. In some cases, the double-stranded region may comprise a length of about 20 nucleotides. In some cases, the double-stranded region may comprise a length of about 25 nucleotides. In some cases, the double-stranded region may comprise a length of about 30 nucleotides. In some cases, the double-stranded region may comprise a length of about 35 nucleotides. In some cases, the double-stranded region may comprise a length of about 40 nucleotides. In some cases, the double-stranded region may comprise a length of about 45 nucleotides. In some cases, the double-stranded region may comprise a length of about 50 nucleotides. In some cases, the double-stranded region may comprise a length of about 55 nucleotides. In some cases, the double-stranded region may comprise a length of about 60 nucleotides. In some cases, the double-stranded region may comprise a length of about 65 nucleotides. In some cases, the double-stranded region may comprise a length of about 70 nucleotides. In some cases, the double-stranded region may comprise a length of about 75 nucleotides. In some cases, the double-stranded region may comprise a length of about 80 nucleotides. In some cases, the double-stranded region may comprise a length of about 85 nucleotides. In some cases, the double-stranded region may comprise a length of about 90 nucleotides. In some cases, the double-stranded region may comprise a length of about 95 nucleotides. In some cases, the double-stranded region may comprise a length of about 100 nucleotides. In some cases, the double-stranded region may comprise a length of about 150 nucleotides. In some cases, the double-stranded region may comprise a length of about 200 nucleotides. In some cases, the double-stranded region may comprise a length of about 300 nucleotides. In some cases, the double-stranded region may comprise a length of about 400 nucleotides. In some cases, the double-stranded region may comprise a length of about 500 nucleotides.

In some instances, the double-stranded region of a reporter may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, 500 or more nucleotide pairs. In some cases, the double-stranded region of the reporter may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, or 500 nucleotide pairs. In some cases, the double-stranded region may comprise a length of about 45 to about 55 nucleotides. In some cases, the double-stranded region may comprise a length of about 40 to about 55 nucleotides. In some cases, the double-stranded region may comprise a length of about 35 to about 55 nucleotides. In some cases, the double-stranded region may comprise a length of about 30 to about 55 nucleotides. In some cases, the double-stranded region may comprise a length of about 25 to about 55 nucleotides. In some cases, the double-stranded region may comprise a length of about 20 to about 55 nucleotides. In some cases, the double-stranded region may comprise a length of about 15 to about 55 nucleotides. In some cases, the double-stranded region may comprise a length of about 10 to about 55 nucleotides. In some cases, the double-stranded region may comprise a length of about 45 to about 60 nucleotides. In some cases, the double-stranded region may comprise a length of about 45 to about 70 nucleotides. In some cases, the double-stranded region may comprise a length of about 45 to about 80 nucleotides. In some cases, the double-stranded region may comprise a length of about 45 to about 90 nucleotides. In some cases, the double-stranded region may comprise a length of about 45 to about 100 nucleotides. In some cases, the double-stranded region may comprise a length of about 45 to about 200 nucleotides. In some cases, the double-stranded region may comprise a length of about 45 to about 500 nucleotides. In some cases, the double-stranded region may comprise a length of about 5 to about 15 nucleotides. In some cases, the double-stranded region may comprise a length of about 5 to about 20 nucleotides. In some cases, the double-stranded region may comprise a length of about 5 to about 25 nucleotides. In some cases, the double-stranded region may comprise a length of about 5 to about 50 nucleotides. In some cases, the double-stranded region may comprise a length of about 5 to about 100 nucleotides. In some cases, the double-stranded region may comprise a length of about 5 to about 200 nucleotides. In some cases, the double-stranded region may comprise a length of about 5 to about 500 nucleotides. In some cases, the double-stranded region may comprise a length of about 4 to about 15 nucleotides. In some cases, the double-stranded region may comprise a length of about 3 to about 15 nucleotides. In some cases, the double-stranded region may comprise a length of about 2 to about 15 nucleotides. In some cases, the double-stranded region may comprise a length of about 1 to about 15 nucleotides.

In some cases, the single-stranded region may comprise about 1 nucleotide, and the double-stranded region may comprise about 35 nucleotides. In some cases, the single-stranded region may comprise about 1 nucleotide, and the double-stranded region may comprise about 40 nucleotides. In some cases, the single-stranded region may comprise about 1 nucleotide, and the double-stranded region may comprise about 45 nucleotides. In some cases, the single-stranded region may comprise about 1 nucleotide, and the double-stranded region may comprise about 50 nucleotides. In some cases, the single-stranded region may comprise about 1 nucleotide, and the double-stranded region may comprise about 55 nucleotides. In some cases, the single-stranded region may comprise about 1 nucleotide, and the double-stranded region may comprise about 60 nucleotides. In some cases, the single-stranded region may comprise about 1 nucleotide, and the double-stranded region may comprise about 65 nucleotides. In some cases, the single-stranded region may comprise about 1 nucleotide, and the double-stranded region may comprise about 35 nucleotides. In some cases, the single-stranded region may comprise about 1 nucleotide, and the double-stranded region may comprise about 40 nucleotides. In some cases, the single-stranded region may comprise about 5 nucleotides, and the double-stranded region may comprise about 45 nucleotides. In some cases, the single-stranded region may comprise about 5 nucleotides, and the double-stranded region may comprise about 50 nucleotides. In some cases, the single-stranded region may comprise about 5 nucleotides, and the double-stranded region may comprise about 55 nucleotides. In some cases, the single-stranded region may comprise about 5 nucleotides, and the double-stranded region may comprise about 60 nucleotides. In some cases, the single-stranded region may comprise about 5 nucleotides, and the double-stranded region may comprise about 65 nucleotides. In some cases, the single-stranded region may comprise about 9 nucleotides, and the double-stranded region may comprise about 35 nucleotides. In some cases, the single-stranded region may comprise about 9 nucleotides, and the double-stranded region may comprise about 40 nucleotides. In some cases, the single-stranded region may comprise about 9 nucleotides, and the double-stranded region may comprise about 45 nucleotides. In some cases, the single-stranded region may comprise about 9 nucleotides, and the double-stranded region may comprise about 50 nucleotides. In some cases, the single-stranded region may comprise about 9 nucleotides, and the double-stranded region may comprise about 55 nucleotides. In some cases, the single-stranded region may comprise about 9 nucleotides, and the double-stranded region may comprise about 60 nucleotides. In some cases, the single-stranded region may comprise about 9 nucleotides, and the double-stranded region may comprise about 65 nucleotides. In some cases, the single-stranded region may comprise about 15 nucleotides, and the double-stranded region may comprise about 35 nucleotides. In some cases, the single-stranded region may comprise about 15 nucleotides, and the double-stranded region may comprise about 40 nucleotides. In some cases, the single-stranded region may comprise about 15 nucleotides, and the double-stranded region may comprise about 45 nucleotides. In some cases, the single-stranded region may comprise about 15 nucleotides, and the double-stranded region may comprise about 50 nucleotides. In some cases, the single-stranded region may comprise about 15 nucleotides, and the double-stranded region may comprise about 55 nucleotides. In some cases, the single-stranded region may comprise about 15 nucleotides, and the double-stranded region may comprise about 60 nucleotides. In some cases, the single-stranded region may comprise about 15 nucleotides, and the double-stranded region may comprise about 65 nucleotides. In some cases, the single-stranded region may comprise about 20 nucleotides, and the double-stranded region may comprise about 35 nucleotides. In some cases, the single-stranded region may comprise about 20 nucleotides, and the double-stranded region may comprise about 40 nucleotides. In some cases, the single-stranded region may comprise about 20 nucleotides, and the double-stranded region may comprise about 45 nucleotides. In some cases, the single-stranded region may comprise about 20 nucleotides, and the double-stranded region may comprise about 50 nucleotides. In some cases, the single-stranded region may comprise about 20 nucleotides, and the double-stranded region may comprise about 55 nucleotides. In some cases, the single-stranded region may comprise about 20 nucleotides, and the double-stranded region may comprise about 60 nucleotides. In some cases, the single-stranded region may comprise about 20 nucleotides, and the double-stranded region may comprise about 65 nucleotides.

In some cases, the reporter comprises a nucleic acid comprising at least one ribonucleotide residue at an internal position that functions as a cleavage site. In some cases, the reporter comprises at least 2, 3, 4, 5, 6, 7, 8, 9, or 10 ribonucleotide residues at an internal position. In some cases, the reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter comprises a nucleic acid comprising at least one deoxyribonucleotide residue at an internal position that functions as a cleavage site. In some cases, the reporter comprises at least 2, 3, 4, 5, 6, 7, 8, 9, or 10 deoxyribonucleotide residues at an internal position. In some cases, the reporter comprises from 2 to 10, from 3 to 9, from 4 to 8, or from 5 to 7 deoxyribonucleotide residues at an internal position. Sometimes the deoxyribonucleotide residues are continuous. Alternatively, the deoxyribonucleotide residues may be interspersed in between non-deoxyribonucleotide residues. In some cases, the reporter has only deoxyribonucleotide residues.

In some cases, the reporter has only deoxyribonucleotide residues. In some cases, the reporter comprises nucleotides resistant to cleavage by the programmable nuclease described herein. In some cases, the reporter comprises synthetic nucleotides. In some cases, the reporter comprises at least one ribonucleotide residue and at least one non-ribonucleotide residue. In some cases, the reporter comprises at least one uracil ribonucleotide. In some cases, the reporter comprises at least two uracil ribonucleotides. Sometimes the reporter has only uracil ribonucleotides. In some cases, the reporter comprises at least one adenine ribonucleotide. In some cases, the reporter comprises at least two adenine ribonucleotides. In some cases, the reporter has only adenine ribonucleotides. In some cases, the reporter comprises at least one cytosine ribonucleotide. In some cases, the reporter comprises at least two cytosine ribonucleotides. In some cases, the reporter comprises at least one guanine ribonucleotide. In some cases, the reporter comprises at least two guanine ribonucleotides. A reporter can comprise only unmodified ribonucleotides, only unmodified deoxyribonucleotides, or a combination thereof. A reporter can comprise a combination of modified and unmodified ribonucleotides and/or deoxyribonucleotides.

In some examples, a reporter molecule comprises a single stranded nucleic acid comprising a detection moiety, wherein the nucleic acid of the reporter molecule is capable of being cleaved by the activated programmable nuclease, thereby generating a first detectable signal. In some cases, the reporter molecule comprises a single-stranded nucleic acid sequence comprising ribonucleotides. In some cases, the reporter molecule comprises a single-stranded nucleic acid sequence comprising deoxyribonucleotides. In some cases, the reporter molecule comprises a single-stranded nucleic acid sequence comprising deoxyribonucleotides and ribonucleotides. As described herein, nucleic acid sequences can be detected using a programmable RNA nuclease, a programmable DNA nuclease, or a combination thereof, as disclosed herein. The programmable nuclease can be activated and cleave the reporter molecule upon binding of a guide nucleic acid to a target nucleic acid. Additionally, different compositions of reporter molecules can allow for multiplexing using different programmable nucleases (e.g., a programmable RNA nuclease and a programmable DNA nuclease). In some instances, the reporter may comprise any design detailed in Table 2 below or described herein.

TABLE 2
Exemplary Reporter Designs

| | | | | Complementary | | | |
|---|---|---|---|---|---|---|---|
| | | | | sequence to | | | |
| | | | | the that | | | |
| | | Sequence | Sequence | underlined | Immobil- | | |
| | | #1 (SEQ | #2 (SEQ | of Sequence | ization | Fluoro- | |
| Reporter | Design (5′-3′) | ID NO) | ID NO) | #1 (SEQ ID NO) | moiety* | phore* | Quencher* |
| Reporter | /5AmMC6/CT | CTTACC | TTTTTTT | GGGTTGTG | /5AmMC6/ | /i6- | /3IABKFQ/ |
| 203 | TACCCACCT | CACCTC | TT (SEQ | ATGTGAGT | | FAMK/ | |
| | CTCCCCCAA | TCCCCC | ID NO: 67) | TAGTGGTG | | | |
| | AACAAACA | AAAACA | | TTTGTTTT | | | |
| | CCACTAACT | AACACC | | GGGGGAG | | | |
| | CACATCAC | ACTAAC | | AGGTGGGT | | | |
| | AACCC/16- | TCACAT | | AAG (SEQ | | | |
| | FAMK/TTTT | CACAAC | | ID NO: 68) | | | |
| | TTTTT/3IABk | CC (SEQ | | | | | |
| | FQ/ (SEQ ID | ID NO: | | | | | |
| | NO: 93) | 62) | | | | | |
| Reporter | /5AmMC6/CT | CTTACC | n/a | GGGTTGTG | /5AmMC6/ | /36-FAM/ | n/a |
| 204 | TACCCACCT | CACCTC | | ATGTGAGT | | | |
| | CTCCCCCAA | TCCCCC | | TAGTGGTG | | | |
| | AACAAACA | AAAACA | | TTTGTTTT | | | |
| | CCACTAACT | AACACC | | GGGGGAG | | | |
| | CACATCAC | ACTAAC | | AGGTGGGT | | | |
| | AACCCTTTT | TCACAT | | AAG (SEQ | | | |
| | TTTTT/36- | CACAAC | | ID NO: 69) | | | |
| | FAM/ (SEQ | CCTTTT | | | | | |
| | ID NO: 94) | TTTTT | | | | | |
| | | (SEQ ID | | | | | |
| | | NO: 63) | | | | | |
| Reporter | /5AmMC6/TT | TTTTTTT | n/a | n/a | /5AmMC6/ | /36-FAM/ | n/a |
| 112 | TTTTTTTTTT | TTTTT | | | | | |
| | /36-FAM/ | (SEQ ID | | | | | |
| | (SEQ ID NO: | NO: 64) | | | | | |
| | 95) | | | | | | |
| Reporter | /5AmMC6//i6- | TTTTTTT | n/a | n/a | /5AmMC6/ | /i6- | /3IABKFQ/ |
| 136 | FAMK/TTTT | TTTTTTT | | | | FAMK/ | |
| | TTTTTTTTTT | TTTTT | | | | | |
| | TTTTT/3IABk | (SEQ ID | | | | | |
| | FQ/ (SEQ ID | NO: 65) | | | | | |
| | NO: 96) | | | | | | |
| Reporter | /5AmMC12/T | TTTTTTT | n/a | n/a | /5AmMC1 | /36-FAM/ | n/a |
| 200 | TTTTTTTTTT | TTTTTTT | | | 2/ | | |
| | TTTTTTTTTT | TTTTTTT | | | | | |
| | TTTTTTTTTT | TTTTTTT | | | | | |
| | TTTTTTTTTT | TTTTTTT | | | | | |
| | TTTTTTTTTT | TTTTTTT | | | | | |
| | TTTTTTTTT/ | TTTTTTT | | | | | |
| | 36-FAM/ | TTTTTTT | | | | | |
| | (SEQ ID NO: | TTTT | | | | | |
| | 97) | (SEQ ID | | | | | |
| | | NO: 66) | | | | | |

/36-FAM/: 3′ 6-Fluorescein (Integrated DNA Technologies)
/3IABKFQ/: 3′ Iowa Black FQ (Integrated DNA Technologies)
/16-FAMK/: internal 6-Fluorescein (Azide) with dT base (Integrated DNA Technologies)
/5AmMC6/: 5′ amino modifier with 6 carbons (Integrated DNA Technologies)
/5AmMC12/: 5′ amino modifier with 12 carbons (Integrated DNA Technologies)
*This Table refers to the fluorophore, quencher moiety, and immobilization moiety as their
tradenames and their source is identified. However, alternatives, generics, or non-tradename
moieties with similar function from other sources can also be used.

In some instances, the reporter may comprise a nucleic acid sequence at least about 50%, 55%, 60%, 65%, 70%, 75%, 80%, 85%, 90%, 95%, 96%, 97%, 98%, 99%, or 100% identical to any one of the sequences described in Table 2. The reporter may comprise a nucleic acid sequence at least about 50% identical to any one of SEQ IDs NO: 62-67. The reporter may comprise a nucleic acid sequence at least about 55% identical to any one of SEQ IDs NO: 62-67. The reporter may comprise a nucleic acid sequence at least about 60% identical to any one of SEQ IDs NO: 62-67. The reporter may comprise a nucleic acid sequence at least about 65% identical to any one of SEQ IDs NO: 62-67. The reporter may comprise a nucleic acid sequence at least about 70% identical to any one of SEQ IDs NO: 62-67. The reporter may comprise a nucleic acid sequence at least about 75% identical to any one of SEQ IDs NO: 62-67. The reporter may comprise a nucleic acid sequence at least about 80% identical to any one of SEQ IDs NO: 62-67. The reporter may comprise a nucleic acid sequence at least about 85% identical to any one of SEQ IDs NO: 62-67. The reporter may comprise a nucleic acid sequence at least about 90% identical to any one of SEQ IDs NO: 62-67. The reporter may comprise a nucleic acid sequence at least about 95% identical to any one of SEQ IDs NO: 62-67. The reporter may comprise a nucleic acid sequence at least about 96% identical to any one of SEQ IDs NO: 62-67. The reporter may comprise a nucleic acid sequence at least about 97% identical to any one of SEQ IDs NO: 62-67. The reporter may comprise a nucleic acid sequence at least about 98% identical to any one of SEQ IDs NO: 62-67. The reporter may comprise a nucleic acid sequence at least about 99% identical to any one of SEQ IDs NO: 62-67. The reporter may comprise a nucleic acid sequence of any one of SEQ IDs NO: 62-67.

In some instances, the reporter may comprise at least two nucleic acid sequences at least about 50%, 55%, 60%, 65%, 70%, 75%, 80%, 85%, 90%, 95%, 96%, 97%, 98%, 99%, or 100% identical to any two of SEQ IDs NO: 62-67. The reporter may comprise at least two nucleic acid sequences at least about 50% identical to any two of SEQ IDs NO: 62-67. The reporter may comprise at least two nucleic acid sequences at least about 55% identical to any two of SEQ IDs NO: 62-67. The reporter may comprise at least two nucleic acid sequences at least about 60% identical to any two of SEQ IDs NO: 62-67. The reporter may comprise at least two nucleic acid sequences at least about 65% identical to any two of SEQ IDs NO: 62-67. The reporter may comprise at least two nucleic acid sequences at least about 70% identical to any two of SEQ IDs NO: 62-67. The reporter may comprise at least two nucleic acid sequences at least about 75% identical to any two of SEQ IDs NO: 62-67. The reporter may comprise at least two nucleic acid sequences at least about 80% identical to any two of SEQ IDs NO: 62-67. The reporter may comprise at least two nucleic acid sequences at least about 85% identical to any two of SEQ IDs NO: 62-67. The reporter may comprise at least two nucleic acid sequences at least about 90% identical to any two of SEQ IDs NO: 62-67. The reporter may comprise at least two nucleic acid sequences at least about 95% identical to any two of SEQ IDs NO: 62-67. The reporter may comprise at least two nucleic acid sequences at least about 96% identical to any two of SEQ IDs NO: 62-67. The reporter may comprise at least two nucleic acid sequences at least about 97% identical to any two of SEQ IDs NO: 62-67. The reporter may comprise at least two nucleic acid sequences at least about 98% identical to any two of SEQ IDs NO: 62-67. The reporter may comprise at least two nucleic acid sequences at least about 99% identical to any two of SEQ IDs NO: 62-67. The reporter may comprise at least two nucleic acid sequences of any two of SEQ IDs NO: 62-67.

In some instances, the reporter may comprise at least two nucleic acid sequences at least about 50%, 55%, 60%, 65%, 70%, 75%, 80%, 85%, 90%, 95%, 96%, 97%, 98%, 99%, or 100% identical to SEQ ID NOs: 62, 63, 68, and 69. The reporter may comprise at least two nucleic acid sequences at least about 50% identical to SEQ ID NOs: 62, 63, 68, and 69. The reporter may comprise at least two nucleic acid sequences at least about 55% identical to SEQ ID NOs: 62, 63, 68, and 69. The reporter may comprise at least two nucleic acid sequences at least about 60% identical to SEQ ID NOs: 62, 63, 68, and 69. The reporter may comprise at least two nucleic acid sequences at least about 65% identical to SEQ ID NOs: 62, 63, 68, and 69. The reporter may comprise at least two nucleic acid sequences at least about 70% identical to SEQ ID NOs: 62, 63, 68, and 69. The reporter may comprise at least two nucleic acid sequences at least about 75% identical to SEQ ID NOs: 62, 63, 68, and 69. The reporter may comprise at least two nucleic acid sequences at least about 80% identical to SEQ ID NOs: 62, 63, 68, and 69. The reporter may comprise at least two nucleic acid sequences at least about 85% identical to SEQ ID NOs: 62, 63, 68, and 69. The reporter may comprise at least two nucleic acid sequences at least about 90% identical to SEQ ID NOs: 62, 63, 68, and 69. The reporter may comprise at least two nucleic acid sequences at least about 95% identical to SEQ ID NOs: 62, 63, 68, and 69. The reporter may comprise at least two nucleic acid sequences at least about 96% identical to SEQ ID NOs: 62, 63, 68, and 69. The reporter may comprise at least two nucleic acid sequences at least about 97% identical to SEQ ID NOs: 62, 63, 68, and 69. The reporter may comprise at least two nucleic acid sequences at least about 98% identical to SEQ ID NOs: 62, 63, 68, and 69. The reporter may comprise at least two nucleic acid sequences at least about 99% identical to SEQ ID NOs: 62, 63, 68, and 69. The reporter may comprise at least two nucleic acid sequences of SEQ ID NOs: 62, 63, 68, and 69.

In some embodiments, the reporter molecule comprises a detection moiety capable of generating a detectable signal. A signal can be a calorimetric, potentiometric, amperometric, optical (e.g., fluorescent, colorimetric, etc.), or piezo-electric. Suitable detectable labels and/or moieties that may provide a signal include, but are not limited to, an enzyme, a radioisotope, a member of a specific binding pair, a fluorophore, a fluorescent protein, a quantum dot, and the like.

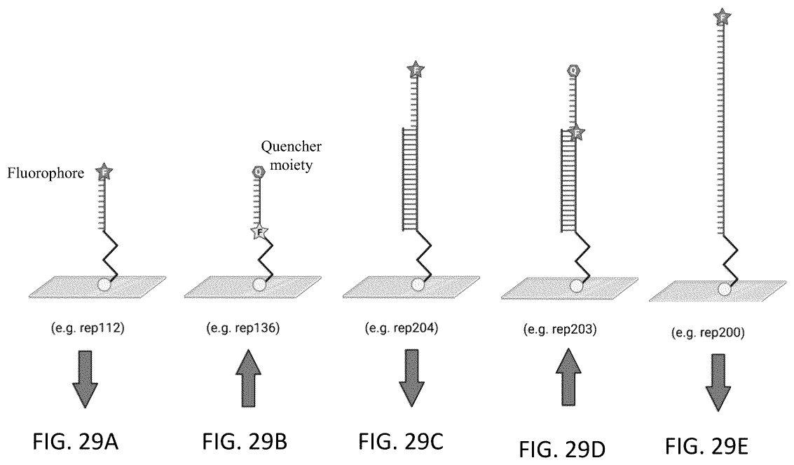

In some instances, a detection moiety can be located at an end of a reporter. In some cases, a detection moiety can be located at an end of a nucleic acid of a reporter. Cleavage of the nucleic acid can release the detection moiety, thereby decreasing the signal a reporter. In some cases, a detection moiety can be located at the 3′ end of a nucleic acid of a reporter. For example, a fluorophore can be located at the 3′ end of a reporter, as shown in FIG. 29A, 29C, or 29E. In other cases, the fluorophore can be at the 5′ end of a reporter. Optionally, a quenching moiety is on the other side of the cleavage site. Sometimes the quenching moiety is a fluorescence quenching moiety. For example, from 5′ to 3′, the reporter may comprise an immobilization moiety, a first nucleic acid, a fluorophore, a second nucleic acid, and a quencher moiety, as shown in FIG. 29B or 29D. The quencher moiety may quench the signal of the fluorophore. Cleavage of the second nucleic acid may release the quencher moiety, thereby increasing the signal of the reporter.

In some cases, the quenching moiety is 5′ to the cleavage site and the fluorophore is 3′ to the cleavage site. In some cases, the fluorophore is 5′ to the cleavage site and the quenching moiety is 3′ to the cleavage site. Sometimes the quenching moiety is at the 5′ terminus of the reporter molecule. Sometimes the fluorophore is at the 3′ terminus of the reporter molecule. In some cases, the fluorophore is at the 5′ terminus of the reporter molecule. In some cases, the quenching moiety is at the 3′ terminus of the reporter molecule.

### Multiplex Reporter Systems

In some instances, systems and methods may be used for multiple detection of target nucleic acids. In some cases, a substrate and/or detection region may comprise multiple reporters, multiple guide nucleic acids, or a combination thereof that are immobilized, dried, or otherwise deposited thereto. Localizing the guide nucleic acids and reporter may localize the detectable signal for each target nucleic acid to the detection spot, thus enabling the spatial multiplexing. For example, in some embodiments, the detection region may comprise an array of detection spots at discrete locations. Each detection spot of the array may comprise an immobilized reporter and a different immobilized guide nucleic acid which is complementary to a different target nucleic acid of a plurality of target nucleic acids. In some embodiments, at each detection spot of the array, upon addition of a programmable nuclease, the immobilized reporter is cleaved by a complex comprising the programmable nuclease and the different immobilized guide nucleic acid to generate a different signal of a plurality of signals. Each different signal may therefore be indicative of the presence or absence of a different target nucleic acid. The target nucleic acids may be freely available within the fluid volume of the detection region. In some embodiments, the array may comprise a number of spots within a range of about 1 to about 200, within a range of about 3 to about 200, or within a range of about 10 to about 200. In some embodiments, the array may comprise at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more spots. In some embodiments, multiple guide nucleic acids for a single target nucleic acid may be combined within a single detection spot in order to increase a rate of reaction.

In some instances, a substrate may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 same or different reporters and/or guide nucleic acids immobilized thereto. In some cases, a detection region may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 same or different reporters and/or guide nucleic acids immobilized thereto. In some cases, each detection location may comprise 1, 2, 3, 4, 5, 6, 7, 8, 9, 10 or more reporters and/or guide nucleic acids immobilized thereto.

### Buffers

The reagents described herein can also include buffers, which are compatible with the devices, systems, fluidic devices, kits, and methods disclosed herein. The buffers described herein are compatible for use in the devices described herein and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether the target nucleic acid is in the sample (e.g., DETECTR reactions). These buffers are compatible with the other reagents, samples, and support mediums as described herein for detection of an ailment, such as a disease, cancer, or genetic disorder, or genetic information, such as for phenotyping, genotyping, or determining ancestry. The methods described herein can also include the use of buffers, which are compatible with the methods disclosed herein. For example, a buffer may comprise HEPES, MES, TCEP, EGTA, Tween 20, KCl, MgCl², glycerol, TIPP, or any combination thereof. In some instances, a buffer may comprise Tris-HCl pH 8.8, VLB, EGTA, CH3COOH, TCEP, IsoAmp, (NH4)2SO4, KCl, MgSO4, Tween20, KOAc, MgOAc, BSA, or any combination thereof. In some instances the buffer may comprise from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer may comprise 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl². The buffer can comprise 0 to 25, 0 to 20, 0 to 10, 0 to 5, 5 to 10, 5 to 15, 5 to 20, 5 to 25, 5 to 30% glycerol. The buffer can comprise from 0% to 30%, from 5% to 30%, from 10% to 30%, from 15% to 30%, from 20% to 30%, from 25% to 30%, from 0% to 25%, from 2% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% glycerol. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Tris-HCl pH 8.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KOAc. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM MgOAc. In some instances the buffer may comprise from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM EGTA. The buffer can comprise from 0% to 30%, from 5% to 30%, from 10% to 30%, from 15% to 30%, from 20% to 30%, from 25% to 30%, from 0% to 25%, from 2% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% Tween 20.

### B. Programmable Nuclease-Based Assay

Described herein are various embodiments of a programmable nuclease-based assay. In some embodiments, a programmable nuclease-based assay is referred to as a DETECTR assay. In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter. Herein, detection (directly or indirectly) of reporter cleavage by a programmable nuclease to determine the presence of a target nucleic acid sequence may be referred to as ‘DETECTR’. The programmable nuclease can, in some cases, be referred to as an RNA-activated programmable RNA nuclease. In some instances, the programmable nuclease as disclosed herein can bind to a target DNA to initiate trans cleavage of an RNA reporter. Such a programmable nuclease can be referred to herein as a DNA-activated programmable RNA nuclease. In some cases, a programmable nuclease as described herein can be activated by a target RNA or a target DNA. For example, a programmable nuclease, e.g., a Cas enzyme, can be activated by a target RNA nucleic acid or a target DNA nucleic acid to cleave RNA reporters. In some embodiments, the Cas enzyme can bind to a target ssDNA which initiates trans cleavage of RNA reporters. In some instances, a programmable nuclease as disclosed herein can bind to a target DNA to initiate trans cleavage of a DNA reporter, and this programmable nuclease can be referred to as a DNA-activated programmable DNA nuclease.

The programmable nuclease can become activated after binding of a guide nucleic acid that is complexed with the programmable nuclease with a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of detector nucleic acids with a detection moiety. Once the target nucleic acid is cleaved by the activated programmable nuclease, the detection moiety can be released or separated from the reporter and can directly or indirectly generate a detectable signal. The reporter and/or the detection moiety can be immobilized, dried, or otherwise deposited on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

The devices, systems, fluidic devices, kits, and methods for detecting the presence of a target nucleic acid in a sample described herein may comprise a generation of a signal indicative of the presence or absence of the target nucleic acid in the sample. The generation of a signal indicative of the presence or absence of the target nucleic acid in the sample as described herein is compatible with the methods and devices described herein and may result from the use of compositions disclosed herein (e.g. programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). As disclosed herein, in some embodiments, detecting the presence or absence of a target nucleic acid of interest involves measuring a signal emitted from a detection moiety present in a reporter, after cleavage of the reporter by an activated programmable nuclease. Alternatively, or in combination, in some embodiments, detecting the presence or absence of a target nucleic acid of interest involves measuring a signal emitted from a conjugate bound to a detection moiety present in a reporter, after cleavage of the reporter by an activated programmable nuclease. The conjugate may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety or another affinity molecule of the cleaved detector molecule as described herein. Thus, the detecting steps disclosed herein may involve indirectly (e.g., via a reporter) measuring the presence of a target nucleic acid, quantifying how much of the target nucleic acid is present, and/or measuring a signal indicating that the target nucleic acid is absent in a sample. In some embodiments, a signal is generated upon cleavage of the reporter by the programmable nuclease. In other embodiments, the signal changes upon cleavage of the reporter by the programmable nuclease. In other embodiments, a signal may be present in the absence of reporter cleavage and disappear or reduce upon cleavage of the target nucleic acid by the programmable nuclease. For example, a signal may be produced in a microfluidic device or lateral flow device after contacting a sample with a composition comprising a programmable nuclease.

In some embodiments, the workflow method may comprise: (1) sample collection from the patient and delivery to the device, (2) optional lysis, (3) optional amplification of the target nucleic acids, and (4) detection/readout. In some embodiments, amplification and detection are carried out in one reaction volume and is referred to as a one-pot reaction. In some embodiments, sample amplification is carried in a first reaction volume and detection is carried in a second reaction volume. Such embodiments, wherein sample amplification is carried in a first reaction volume and detection is carried in a second reaction volume are referred to as two-pot reactions. In some embodiments, carrying out multiple reactions in multiple reaction volumes is referred to as a multi-pot reaction. Exemplary embodiments for one-pot, two-pot, and multi-pot reaction can be found in: PCT/US2021/033271 and PCT/US2021/035031, all of which are herein incorporated by reference in their entirety.

In some embodiments, the running of a programmable nuclease-based assay is referred to as a detection step. In some embodiments, the detection step may be preceded or coincide with nucleic acid amplification In some embodiments, nucleic acid amplification may comprise PCR. In some embodiments, nucleic acid amplification may comprise Loop-Mediated Isothermal Amplification (LAMP).

Described herein are various methods of nucleic acid amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In certain instances, methods include simultaneous amplification and detection in the same volume. In some embodiments, nucleic acid amplification may comprise LAMP amplification or a variation thereof. In some embodiments, nucleic acid amplification may comprise PCR amplification or a variation thereof.

In some embodiments, the limit of detection for a programmable nuclease-based assay is 1 copy of a target nucleic acid per reaction. In some embodiments, the limit of detection for a programmable nuclease-based assay is 10 copies of a target nucleic acid per reaction. In some embodiments, the limit of detection for a programmable nuclease-based assay is from 1 to 10 copies of a target nucleic acid per reaction. In some embodiments, the limit of detection for a programmable nuclease-based assay is from 10 to 20 copies of a target nucleic acid per reaction. In some embodiments, the limit of detection for a programmable nuclease-based assay is from 20 to 30 copies of a target nucleic acid per reaction. In some embodiments, the limit of detection for a programmable nuclease-based assay is from 30 to 40 copies of a target nucleic acid per reaction. In some embodiments, the limit of detection for a programmable nuclease-based assay is from 40 to 50 copies of a target nucleic acid per reaction. In some embodiments, the limit of detection for a programmable nuclease-based assay is from 50 to 100 copies of a target nucleic acid per reaction. In some embodiments, the limit of detection for a programmable nuclease-based assay is from 100 to 1000 copies of a target nucleic acid per reaction. In some embodiments, the limit of detection for a programmable nuclease-based assay is greater than 1000 copies of a target nucleic acid per reaction.

### Reagent Immobilization Strategies

Programmable nuclease-based diagnostic reactions are generally performed in solution where the programmable nuclease-guide nucleic acid complexes can freely bind target molecules and reporters. However, reactions where all components are in solution can limit the designs of nucleic acid diagnostic assays, especially in microfluidic devices. A system where various components of the programmable nuclease-based diagnostic reaction are immobilized on a surface may enable designs where multiple readouts can be accomplished within a single reaction chamber and/or in a single cartridge.

One or more components or reagents of a programmable nuclease-based detection reaction may be suspended in solution or immobilized on a surface. Programmable nucleases, guide nucleic acids, and/or reporters may be suspended in solution or immobilized on a surface. For example, the reporter, programmable nuclease, and/or guide nucleic acid can be immobilized on the surface of a chamber in a device as disclosed herein. In some cases, the reporter, programmable nuclease, and/or guide nucleic acid can be immobilized on beads, such as magnetic beads, in a chamber of a device as disclosed herein where they are held in position by a magnet placed below the chamber. An immobilized programmable nuclease can be capable of being activated and cleaving a free-floating or immobilized reporter. An immobilized guide nucleic acid can be capable of binding a target nucleic acid and activating a programmable nuclease complexed thereto. An immobilized reporter can be capable of being cleaved by the activated programmable nuclease, thereby generating a detectable signal.

Described herein are various methods to immobilize programmable nuclease-based diagnostic reaction components to the surface of a reaction chamber or other surface (e.g., a surface of a bead). Any of the devices described herein may comprise one or more immobilized detection reagent components (e.g., programmable nuclease, guide nucleic acid, and/or reporter). In certain instances, methods include immobilization of programmable nucleases (e.g., Cas proteins or Cas enzymes), reporters, and/or guide nucleic acids (e.g., gRNAs). In some embodiments, various programmable nuclease-based diagnostic reaction components are modified with biotin. In some embodiments, these biotinylated programmable nuclease-based diagnostic reaction components are tested for immobilization on surfaces coated with streptavidin. In some embodiments, the biotin-streptavidin interaction is used as a model system for other immobilization chemistries. In some embodiments, NHS-Amine chemistry is used for immobilization of programmable nuclease-based reaction components. In some embodiments, amino modifications are used for immobilization of programmable nuclease-based reaction components. In some embodiments, maleimide-thiol chemistry is used for immobilization of programmable nuclease-based reaction components. In some embodiments, epoxy-amine chemistry is used for immobilization of programmable nuclease-based reaction components. In some embodiments, hydrogels are used for immobilization of programmable nuclease-based reaction components.

In some embodiments, chemical modifications of amino acid residues in the Cas protein enable attachment to a surface. In some embodiments, guide nucleic acids are immobilized by adding various chemical modifications at the 5′ or 3′ end of the guide nucleic acids that are compatible with a selected surface chemistry. In some embodiments, fluorescence-quenching (FQ), or other reporter detection moiety chemistries, are attached to surfaces using similar chemical modifications as gRNAs. In some embodiments, these attached reporters are cleaved by a programmable nuclease, which leads to either activated molecules (e.g., detection moieties) that remain attached to the surface or activated molecules that are released into solution.

For some embodiments, described herein the programmable nuclease complex may be immobilized by a guide nucleic acid and cleave surrounding fluorophore-quencher reporters that are also immobilized to a surface. Here, the quencher may be released into solution, leaving a localized fluorescent signal. In other embodiments, the fluorophore may be released into solution, thereby generating a fluorescent signal in solution.

In some embodiments, the programmable nuclease, guide nucleic acid, reporter, or a combination thereof can be immobilized to a device surface (e.g., by a linkage). In some embodiments, the linkage comprises a covalent bond, a non-covalent bond, an electrostatic bond, an interaction (e.g., a covalent or noncovalent bond) between members of a binding pair (e.g., streptavidin and biotin), an amide bond, or any combination thereof. In embodiments where more than one element is immobilized to the surface (e.g., reporters and guide nucleic acid, programmable nuclease and reporters, or all three), the linkage may be the same or different for each species. For example, the guide nucleic acid may be immobilized to the surface by a single-stranded linker polynucleotide, and the reporters may be immobilized by the interaction between a first member of a binding pair on the reporters and a second member of a binding pair on the surface. In general, the term “binding pair” refers to a first and a second moiety that have a specific binding affinity for each other. In some embodiments, a binding pair has a dissociation constant Kd of less than or equal to about: 10⁻⁸ mol/L, 10⁻⁹ mol/L, 10⁻¹⁰ mol/L, 10⁻¹¹ mol/L, 10⁻¹² mol/L, 10⁻¹³ mol/L, 10⁻¹⁴ mol/L, 10⁻¹⁵ mol/L, or ranges including two of these values as endpoints. Non limiting examples of binding pairs include an antibody or an antigen-binding portion thereof and an antigen (e.g., fluorescein, digoxin, digoxigenin); a biotin (bio) moiety and an avidin (or streptavidin) moiety; a dinitrophenol (DNP) and an anti-DNP antibody; a hapten and an anti hapten; folate and a folate binding protein; vitamin B12 and an intrinsic factor; a carbohydrate and a lectin or carbohydrate receptor; a polysaccharide and a polysaccharide binding moiety; a lectin and a receptor; a ligand and a receptor; a drug and a drug receptor; complementary chemical reactive groups (e.g., sulfhydryl/maleimide, thiol/maleimide, sulfhydryl/haloacetyl derivative, amine/epoxy, amine/isotriocyanate, amine/succinimidyl ester, and amine/sulfonyl halides); an antibody (e.g., IgG) and protein A or protein G; a toxin and a toxin receptor; and an enzyme substrate and an enzyme. In some embodiments, the binding pair comprises biotin and either of avidin or streptavidin.

In some embodiments, the linkage utilizes non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface. In some embodiments, the programmable nuclease, guide nucleic acid, and/or the reporter are immobilized to or within a polymer matrix. The polymer matrix may comprise a hydrogel. Co-polymerization of the programmable nuclease, guide nucleic acid, or the reporter into the polymer matrix may result in a higher density of reporter/unit volume or reporter/unit area than other immobilization methods utilizing surface immobilization (e.g., onto beads, after matrix polymerization, etc.). Co-polymerization of the programmable nuclease, guide nucleic acid, or the reporter into the polymer matrix may result in less undesired release of the reporter (e.g., during an assay, a measurement, or on the shelf), and thus may cause less background signal, than other immobilization strategies (e.g., conjugation to a pre-formed hydrogel, bead, etc.). In at least some instances this may be due to better incorporation of reporters into the polymer matrix as a co-polymer and fewer “free” reporter molecules retained on the hydrogel via non-covalent interactions or non-specific binding interactions.

In some embodiments, a plurality of oligomers and a plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for free-floating programmable nucleases to diffuse into the hydrogel and access immobilized internal reporter molecules. The relative percentages and/or molecular weights of the oligomers may be varied to vary the pore size of the hydrogel. For example, pore size may be tailored to increase the diffusion coefficient of the programmable nucleases.

In some embodiments, the functional groups attached to the reporters and/or guide nucleic acids may be selected to preferentially incorporate the reporters and/or guide nucleic acids into the polymer matrix via covalent binding at the functional group versus other locations along the nucleic acid backbone of the reporter and/or guide nucleic acid. In some embodiments, the functional groups attached to the reporters and/or guide nucleic acids may be selected to favorably transfer free radicals from the functionalized ends of polymerizable oligomers to the functional group on the end of the reporter and/or guide nucleic acid (e.g., 5′ end), thereby forming a covalent bond and immobilizing the reporter and/or guide nucleic acid rather than destroying other parts of the reporter and/or guide nucleic acid molecules, respectively. In some embodiments, the functional group may comprise a single stranded nucleic acid, a double stranded nucleic acid, an acrydite group, a 5′ thiol modifier, a 3′ thiol modifier, an amine group, a I-Linker™ group, methacryl group, or any combination thereof. One of ordinary skill in the art will recognize that a variety of functional groups may be used depending on the desired properties of the immobilized components.

### Programmable Nuclease-Based Assay Multiplexing

Methods consistent with the present disclosure include a multiplexing method of assaying for a plurality of target nucleic acids in a sample. A multiplexing method may comprise a) contacting the sample to a programmable nuclease complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and b) assaying for a signal indicating cleavage of at least some reporters (e.g., protein-nucleic acids) of a population of reporter molecules (e.g., protein-nucleic acids), wherein the signal indicates a presence of the target nucleic acid in the sample and wherein absence of the signal indicates an absence of the target nucleic acid in the sample.

Multiplexing can comprise spatial multiplexing wherein multiple different target nucleic acids are detected at the same time, but the reactions are spatially separated. In some cases, the multiple target nucleic acids are detected using the same programmable nuclease, but different guide nucleic acids. The multiple target nucleic acids sometimes are detected using different programmable nucleases. In the case wherein multiple target nucleic acids are detected using different programmable nucleases, the method may involve using a first programmable nuclease, which upon activation (e.g., after binding of a first guide nucleic acid to a first target), cleaves a nucleic acid of a first reporter and using a second programmable nuclease, which upon activation (e.g., after binding of a second guide nucleic acid to a second target) cleaves a nucleic acid of a second reporter. Spatially separated reactions may, for example, occur within an array comprising a plurality of different detection locations each comprising one or more immobilized detection reaction component(s) thereon. In some embodiments, spatially separated reactions may occur in different wells of a detection region, e.g., within different microwells, each microwell containing different detection reaction components configured to detect different target nucleic acids.

Sometimes, multiplexing can be single reaction multiplexing, wherein multiple different target acids are detected in a single reaction volume. Often, at least two different programmable nucleases are used in single reaction multiplexing. For example, multiplexing can be enabled by immobilization of multiple categories of detector nucleic acids within a fluidic system, to enable detection of multiple target nucleic acids within a single fluidic system. Multiplexing allows for detection of multiple target nucleic acids in one kit or system. In some cases, the multiple target nucleic acids comprise different target nucleic acids from a virus, a bacterium, or a pathogen responsible for one disease. In some cases, the multiple target nucleic acids comprise different target nucleic acids associated with a cancer or genetic disorder. Multiplexing for one disease, cancer, or genetic disorder increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. Multiplexing, thus, allows for multiplexed detection of multiple genomic alleles. For example, multiplexing may comprise method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different pathogen strains, for example, HPV16 and HPV18. In some instances, multiplexing allows for discrimination between multiple target nucleic acids of different variants of a pathogen, for example, alpha and delta SARS-COV-2 variants. In some cases, the multiple target nucleic acids comprise target nucleic acids directed to different cancers or genetic disorders. Often, multiplexing allows for discrimination between multiple target nucleic acids, such as target nucleic acids that comprise different genotypes, for example, for a wild-type genotype and for SNP genotype. Multiplexing for multiple diseases, cancers, or genetic disorders provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel may comprise assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. In this context, a unique target nucleic acid refers to the sequence of a nucleic acid that has an at least one nucleotide difference from the sequences of the other nucleic acids in the plurality. Multiple copies of each target nucleic acid can be present. For example, a unique target nucleic population can comprise multiple copies of the unique target nucleic acid. Often the plurality of unique target nucleic acids is from a plurality of bacterial pathogens in the sample.

In some instances, the multiplexed devices, systems, fluidic devices, kits, and methods detect at least 2 different target nucleic acids in a single reaction. In some instances, the multiplexed devices, systems, fluidic devices, kits, and methods detect at least 3 different target nucleic acids in a single reaction. In some instances, the multiplexed devices, systems, fluidic devices, kits, and methods detect at least 4 different target nucleic acids in a single reaction. In some instances, the multiplexed devices, systems, fluidic devices, kits, and methods detect at least 5 different target nucleic acids in a single reaction. In some cases, the multiplexed devices, systems, fluidic devices, kits, and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit. In some instances, the multiplexed kits detect from 2 to 10, from 3 to 10, from 4 to 10, from 5 to 10, from 6 to 10, from 7 to 10, from 8 to 10, from 9 to 10, from 2 to 9, from 3 to 9, from 4 to 9, from 5 to 9, from 6 to 9, from 7 to 9, from 8 to 9, from 2 to 8, from 3 to 8, from 4 to 8, from 5 to 8, from 6 to 8, from 7 to 8, from 2 to 7, from 3 to 7, from 4 to 7, from 5 to 7, from 6 to 7, from 2 to 6, from 3 to 6, from 4 to 6, from 5 to 6, from 2 to 5, from 3 to 5, from 4 to 5, from 2 to 4, from 3 to 4, or from 2 to 3 different target nucleic acids in a single kit. Multiplexing can be carried out in a single-pot or “one-pot” reaction, where reverse transcription, amplification, in vitro transcription, or any combination thereof, and detection are carried out in a single volume. Multiplexing can be carried out in a “two-pot reaction”, where reverse transcription, amplification, in vitro transcription, or any combination thereof, are carried out in a first volume and detection is carried out in a second volume.

In some cases, multiplexing can comprise detecting multiple targets with a single probe. Alternatively, multiplexing can comprise detecting multiple targets with multiple probes. The multiple probes can be configured to detect a presence of a particular sequence, target nucleic acid, or a plurality of different target sequences or nucleic acids.

### Sample Preparation and Lyophilization Strategies

Described herein are various methods of sample preparation and reagent storage. Any of the devices described herein may comprise one or more sample preparation reagents. Any of the devices described herein may comprise sample preparation reagents as dried reagents. Dried reagents may comprise solids and/or semi-solids. In certain instances, dried reagents may comprise lyophilized reagents. Any of the devices described herein may comprise one or more lyophilized reagents (e.g., amplification reagents, programmable nucleases, buffers, excipients, etc.). In certain instances, methods include sample lysis, concentration, and/or filtration. In certain instances, methods include reconstitution of one or more lyophilized reagents. In some embodiments, lyophilized reagents may be in the form of lyophilized beads, spheres, and/or particulates. In some embodiments, the lyophilized bead, sphere, and/or particulate may comprise either single or multiple compounds. In some embodiments, the lyophilized bead, sphere, and/or particulate may be adjusted to various moisture levels or hygroscopy. In some embodiments, the lyophilized bead, sphere, and/or particulate may comprise assay internal standards. In some embodiments, the lyophilized bead, sphere, and/or particulate may have diameters between about 0.5 millimeters to about 5 millimeters in diameter.

Described herein are various embodiments of a programmable nuclease-based detection (e.g., DETECTR) reaction involving optimization of sample preparation and lyophilization. Such embodiments allow for adapting the buffer for binding a substrate to perform a concentration step. In some embodiments, experiments may be performed to evaluate the lysis (sample is evaluated directly in the assay) and binding (the sample is eluted from magnetic beads) characteristics of buffers with different components. In such embodiments, the input sample is the same concentration as the eluted sample.

In some embodiments, crude lysis buffer is used in a one-pot assay with Cas14a.1 (SEQ ID NO: 3).

In some embodiments, the enzyme having SEQ ID NO: 17 is used with the programmable nuclease-based detection assay.

In some embodiments, a control study involving sample preparation optimization of the LANCR (multiplexed isothermal amplification) assay. In such embodiments, crude lysis involves: 25 μL sample+25 μL lysis buffer and incubation at 25 C for 1 minute. In some embodiments, the LANCR reaction is run as follows: 5 μL sample in a 25 μL reaction volume (standard conditions). In some embodiments the DETECTR reaction is run as follows: 2 μL LANCR product in 20 μL reaction volume (standard conditions). Sample: 250 copies/rxn SeraCare SARS-COV-2 reference RNA.

In some embodiments, Trehalose, Raffinose, PVP 40, sorbitol, Mannitol, Mannose, or a combination thereof are used for lyophilization. In some embodiments, Trehalose may be used to control the rate of the reaction.

Described herein are various methods and devices for carrying out programmable nuclease-based assays. In some embodiments, programmable nuclease-based assays utilize a Cas12 protein, a Cas13 protein, a Cas14 protein, or a CasPhi protein. In some embodiments, amplification (e.g., RT-LAMP) and programmable nuclease-based detection (e.g., DETECTR) master mixes of reagents are lyophilized in the same combined master mix. In some embodiments, amplification and programmable nuclease-based detection master mixes of reagents and target may be lyophilized in the same combined master mix. In some embodiments, one-pot refers to the combination of both amplification (e.g., RT-LAMP) and detection (e.g., DETECTR) reaction reagents in one volume. In some embodiments, an excipient is used to confirm reagent stability throughout the lyophilization process, comprising freezing and drying steps. In some embodiments, excipients are sugars.

In some embodiments, a master mix of assay reagents may be reconstituted after lyophilization. In some embodiments, a master mix of DETECTR assay reagents may be reconstituted after lyophilization. In some embodiments, a master mix of DETECTR assay reagents, including a Cas12 protein for example, may be reconstituted after lyophilization. In some embodiments, a master mix of amplification and programmable nuclease-based detection (e.g., DETECTR) assay reagents, including a Cas12 for example, may be reconstituted after lyophilization. In some embodiments, a master mix of amplification (e.g., RT-LAMP) and programmable nuclease-based detection (e.g., DETECTR) assay reagents, including a Cas12 protein, may be reconstituted after lyophilization.

In some embodiments, the master mix of reagents and target for one assay is lyophilized. In some embodiments, the master mixes from more than one assay may be pooled and lyophilized.

In some embodiments, lyophilized master mixes of reagents from more than one assay may be prepared in volumes of less than 1 mL. In some embodiments, lyophilized master mixes of reagents from more than one assay may be prepared in volumes of less than 250 μL. In some embodiments, lyophilized master mixes of reagents from more than one assay may be prepared in volumes of less than 25 μL. In some embodiments, lyophilized master mixes of reagents from more than one assay may be prepared in volumes of less than 10 μL.

In some embodiments, an excipient is used to stabilize the sample throughout the lyophilization process that may comprise freezing and drying steps.

In some embodiments, a viral, bacterial, and/or high temperature inactivator may be used.

In some embodiments, programmable nuclease-based assays may comprise one or more controls. In some embodiments, the assay controls may comprise non-specific binding controls. In some embodiments, RNase may be used as a control against the programmable nuclease for cleavage activity. In some embodiments, DNase may be used as a control against the programmable nuclease for cleavage activity. In some embodiments, an assay control may comprise a no-target-control (NTC), wherein a particular control sample does not contain at least one target. In some embodiments, a control sample may comprise target nucleic acids added to the control sample at known concentrations.

Described herein are exemplary methods for programmable nuclease-based detection. The method can comprise collecting a sample. The sample can comprise any type of sample as described herein. The method can comprise preparing the sample. Sample preparation can comprise one or more sample preparation steps. The one or more sample preparation steps can be performed in any suitable order. The one or more sample preparation steps can comprise physical filtration of non-target materials using a macro filter. The one or more sample preparation steps can comprise nucleic acid purification. The one or more sample preparation steps can comprise nucleic acid concentration. The one or more sample preparation steps can comprise lysis. The one or more sample preparation steps can comprise heat inactivation. The one or more sample preparation steps can comprise chemical inactivation. The one or more sample preparation steps can comprise neutralization. The one or more sample preparation steps can comprise adding one or more enzymes or reagents to prepare the sample for target detection.

The method can comprise generating one or more droplets, aliquots, volumes, or subsamples from the sample. The one or more droplets, aliquots, volumes, or subsamples can correspond to a volumetric portion of the sample. The sample can be divided into 1, 2, 3, 4, 5, 6, 7, 8, 9, 10, 20, 30, 40, 50, 60, 70, 80, 90, 100, 200, 300, 400, 500, 600, 700, 800, 900, 1000, or more droplets, aliquots, volumes, or subsamples. In some embodiments, the sample is not divided into subsamples.

The method can comprise amplifying one or more targets within the sample. The method can comprise amplifying one or more targets within each droplet, aliquot, volumes, or subsample. Amplification of the one or more targets within each droplet or volume can be performed in parallel and/or simultaneously for each droplet or volume. Dividing the sample into a plurality of droplets or volumes can enhance a speed and/or an efficiency of the amplification process (e.g., a thermocycling process) since the droplets comprise a smaller volume of material than the bulk sample introduced. Amplifying the one or more targets within each individual droplet or volume can also permit effective amplification of various target nucleic acids that cannot be amplified as efficiently in a bulk sample containing the various target nucleic acids if the bulk sample were to undergo a singular amplification process. In some embodiments, amplification is performed on the bulk sample without first dividing the sample into subsamples.

The method can further comprise using a CRISPR-based or programmable nuclease-based detection module to detect one or more targets (e.g., target sequences or target nucleic acids) in the sample. In some cases, the sample can be divided into a plurality of droplets, aliquots, volumes, or subsamples to facilitate sample preparation and to enhance the detection capabilities of the devices of the present disclosure. In some cases, the sample is not divided into subsamples. In some cases, the sample is divided into subsamples and recombined prior to detection.

In some embodiments, the sample can be provided manually to the detection device of the present disclosure. For example, a swab sample can be dipped into a solution and the sample/solution can be pipetted into the device. In other embodiments, the sample can be provided via an automated syringe. The automated syringe can be configured to control a flow rate at which the sample is provided to the detection device. The automated syringe can be configured to control a volume of the sample that is provided to the detection device over a predetermined period.

In some embodiments, the sample can be provided directly to the detection device of the present disclosure. For example, a swab sample can be inserted into a sample receiver and/or a sample chamber on the detection device.

The sample can be prepared before one or more targets are detected within the sample. The sample preparation steps described herein can process a crude sample to generate a pure or purer sample. Sample preparation can comprise one or more physical or chemical processes, including, for example, nucleic acid purification, lysis, binding, washing, and/or elution. In certain instances, sample preparation can comprise the following steps, in any order, including sample collection, nucleic acid purification, heat inactivation, neutralization, and/or base/acid lysis.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (e.g., RNase and/or DNase), and/or removing contaminants (e.g., proteins, carbohydrates, lipids, biological or environmental elements, unwanted nucleic acids, and/or other cellular debris). In some embodiments, nucleic acid purification may involve liquid-liquid extraction, solid-liquid extraction, and/or solid-phase extraction techniques. In at least some instances, solid-phase extraction may be preferred for smaller sample volumes and/or improved cartridge-based performance (e.g., by reducing solvent volumes and/or simpler workflow automation). Solid-phase nucleic acid purification may utilize a silica-based method such as the Boom method. In some embodiments, silica particles (e.g., silica beads or silica magnetic beads), glass fiber filters, silica-coated membranes/meshes/filters, etc. may be used to capture nucleic acids from the sample prior to elution therefrom. Alternatively, or in combination, solid-phase nucleic acid purification may utilize charge switching for nucleic acid concentration. In some embodiments, particles (e.g., beads or magnetic beads) or surfaces (e.g., membranes, meshes, filters, etc.) may be coated with an ionizable coating such as chitosan. In some embodiments, the sample may be agitated (e.g., mixed, shaken, flown, sonicated, etc.) during purification and/or elution.

In some embodiments, lysis of a collected sample can be performed. Lysis can be performed using a protease (e.g., a Proteinase K or PK enzyme, and/or a savinase). In some cases, a solution of reagents can be used to lyse the cells in the sample and release the nucleic acids so that they are accessible to the programmable nuclease. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate·2H₂O, 0.5% (w/v) sodium lauryl sarcosinate, and 0.1 M β-mercaptoethanol), but numerous commercial buffers for different cellular targets can also be used. Alkaline buffers (i.e., buffers with a pH above 7) can also be used, and may be particularly effective for cells with hard shells, such as may be present in environmental samples. In some embodiments, the alkaline buffer has a pH of about or more than about 8, 8.5, 9, 9.5, 10, or higher. In some embodiments, acidic buffers (i.e., buffers with a pH below 7) are used for lysis. In some embodiments, the alkaline buffer has a pH of about 10. Any suitable base may be used for the alkaline buffer, including organic or inorganic bases. In some embodiments, the base is sodium hydroxide. In some embodiments, the acidic buffer has a pH of about or less than about 6, 5.5, 5, 4.5, 4, or lower. In some embodiments, the acidic buffer has a pH of about 5. Any suitable acid may be used for the acidic buffer, including organic or inorganic acids. In some embodiments, the acid is acetic acid. Detergents such as sodium dodecyl sulfate (SDS) and cetyl trimethylammonium bromide (CTAB) can also be implemented to chemical lysis buffers. Cell lysis can also be performed by physical, mechanical, thermal, or enzymatic means, in addition to or instead of chemically-induced cell lysis mentioned previously. In some cases, depending on the type of sample, nanoscale barbs, nanowires, acoustic generators (e.g., ultrasonic horns), integrated lasers, integrated heaters, and/or microcapillary probes can be used to perform lysis. In some embodiments, the sample may be agitated (e.g., mixed, shaken, flown, sonicated, etc.) during lysis. In some embodiments, lysis comprises exposure to both an acidic and an elevated temperature (e.g., 65° C. to 95° C., 70° C. to 90° C., or 75° C. to 85° C.).

In certain instances, inactivation can be performed on the sample to inactivate or neutralize conditions employed in a lysis step. In some embodiments, a processed/lysed sample can undergo heat inactivation to inactivate, in the lysed sample, the proteins used during lysing (e.g., a PK enzyme or a lysing reagent). In some embodiments, the sample may be agitated (e.g., mixed, shaken, flown, sonicated, bubbled, etc.) during heat-inactivation or neutralization. In some cases, a heating element integrated into the detection device or disposed within the instrument can be used for heat-inactivation. The heating element can be powered by a battery or another source of thermal or electric energy that is integrated with the detection device or instrument. In some embodiments, the heating element for heat-inactivation is the same as the heating element for lysis, amplification, and/or detection. In some embodiments, where a protease is used in the lysis step, inactivation may comprise introducing a protease inhibitor. In some embodiments, where an alkaline buffer is used for the lysis step, inactivation may comprise a neutralization step that lowers the pH of the solution, such as to a pH of about 7, 7.5, 8, or 8.5. In some embodiments, where an alkaline buffer is used for the lysis step, inactivation may comprise a neutralization step that lowers the pH of the solution, such as to a pH of about 8.8. In some embodiments, where an acidic buffer is used for the lysis step, inactivation may comprise a neutralization step that raises the pH of the solution, such as to a pH of about 7, 7.5, 8, or 8.5. In some embodiments, where an alkaline buffer is used for the lysis step, inactivation may comprise a neutralization step that raises the pH of the solution, such as to a pH of about 8.8. Neutralization may utilize any suitable base (e.g., potassium hydroxide or Tris) or acid (e.g., potassium acetate), depending on the pH of the lysis buffer, which may be provided in any suitable form (e.g., pellet or concentrated solution). A filtering step to remove debris may be included between lysis and inactivation, after inactivation, or both.

In some cases, a target nucleic acid within the sample can undergo amplification before binding to a guide nucleic acid, for example a crRNA of a CRISPR enzyme. The target nucleic acid within a purified sample can be amplified to generate a target amplicon. In some instances, amplification can be accomplished using loop mediated amplification (LAMP), isothermal recombinase polymerase amplification (RPA), and/or polymerase chain reaction (PCR). In some instances, digital droplet amplification can used. Such nucleic acid amplification of the sample can improve at least one of a sensitivity, specificity, or accuracy of the detection of the target nucleic acid. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes.

In some embodiments, amplification can comprise thermocycling of the sample. Thermocycling can be carried out for one or more droplets or volumes of the sample in parallel and/or independently in separate locations. In some embodiments, this can be accomplished by methods such as (1) by holding droplets or volumes stationary in locations where a heating element is in close proximity to the droplet or volume on one of the droplet or volume sides and a heat sink element is in close proximity to the other side of the droplet or volume, or (2) flowing the droplet of volume through zones in a fluid channel where heat flows across it from a heating source to a heat sink. In some cases, one or more resistive heating elements can be used to perform thermocycling. In some cases, thermocycling may comprise one or more reactions at different temperatures. In some cases, the reactions can include an annealing reaction, a denaturation reaction, and/or an extension reaction.

In some cases, an annealing temperature of the thermocycling reaction may be performed at a temperature around 45° C. to 75° C. In some embodiments, the annealing temperature may be at a temperature of about 45° C., about 47° C., about 48° C., about 49° C., about 50° C., about 52° C., about 54° C., about 56° C., about 58° C., about 60° C., about 62° C., about 64° C., about 66° C., about 68° C., about 70° C., about 72° C., about 74° C., or about 76° C.

In some cases, a denaturation temperature of the thermocycling reaction may be performed at a temperature around 90° C. to about 110° C. In some embodiments, the denaturation temperature may be at a temperature of about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., or about 110° C.

In some cases, an extension temperature of the thermocycling reaction may be performed at a temperature from around 55° C. to about 85° C. In some embodiments, the extension temperature may be at a temperature of about 55° C., about 57° C., about 59° C., about 60° C., about 61° C., about 62° C., about 63° C., about 64° C., about 65° C., about 66° C., about 68° C., about 70° C., about 71° C., about 72° C., about 73° C., about 75° C., about 76° C., about 78° C., about 80° C., about 81° C., about 82° C., about 83° C., about 84° C., or about 85° C.

In some embodiments, amplification can comprise isothermal amplification of the sample. Isothermal amplification ca be carried out for one or more droplets or volumes of the sample in parallel and/or independently in separate locations. In some embodiments, this can be accomplished by holding droplets or volumes stationary in locations where a heating element is in close proximity to the droplet or volume on one of the droplet or volume sides so as to maintain a constant temperature within the droplet or volume. In some cases, one or more resistive heating elements can be used to perform isothermal amplification. Sometimes, the nucleic acid amplification reaction is performed at a temperature of around 20-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 65° C. In some cases, the nucleic acid amplification reaction can be performed at a temperature that ranges from about 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C., from 45° C. to 65° C., from 50° C. to 65° C., from 55° C. to 65° C., from 60° C. to 65° C., from 40° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C.

Additionally, target nucleic acid can optionally be amplified before binding to the guide nucleic acid (e.g., crRNA) of the programmable nuclease complex (e.g., CRISPR enzyme). This amplification can, for example, be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

In some embodiments, a target nucleic acid within the sample can undergo reverse transcription before binding to a guide nucleic acid, for example a crRNA of a CRISPR enzyme. The target nucleic acid within a purified sample can be reverse transcribed. In some instances, reverse transcription can be accomplished using a reverse transcriptase. Reverse transcription can be combined with any of the amplification techniques described herein. In some cases, a reverse transcription step may be performed at a temperature of around 45° C. to about 75° C. In some embodiments, the reverse transcription may be at a temperature of about 45° C., about 47° C., about 48° C., about 49° C., about 50° C., about 52° C., about 54° C., about 55° C., about 57° C., about 59° C., about 60° C., about 61° C., about 63° C., about 65° C., about 66° C., about 68° C., about 70° C., about 72° C., about 73° C., or about 75° C.

### III. INSTRUMENT

### A. System Integration and User Interface

Described herein are various systems and devices for performing one or more nucleic acid-based assays. In some embodiments, the nucleic acid-based assay may be a programmable nuclease-based assay. Systems may comprise any of the instruments and/or cartridges described herein. In some embodiments, the instrument and the cartridge may be configured to mate with one another to enable mechanical manipulation, activation, control, and/or communication between electronic circuits (e.g., in the cartridge and the instrument), thermal interfaces, ultrasonic interfaces, and/or optical interfaces. In some embodiments, the instrument may comprise a receptacle (e.g., an open slot or dock) configured (e.g., sized and shaped) to receive the cartridge. In some embodiments, the cartridge may be self-aligning with and/or locked within the instrument after insertion into its receptacle. In some embodiments, there are mechanical features provided within the instrument or on the cartridge to facilitate correct relative alignment of the instrument and cartridge. In some embodiments, the self-alignment process may optionally connect one or more pressure ports of the cartridge and/or instrument. Alternatively, or in combination, the self-alignment process may engage one or more heat sources (e.g., thermal cycling heaters) of the instrument onto the cartridge.

FIG. 3 depicts a schematic diagram illustrating exemplary functions which may be performed by any of the systems 100 for nucleic acid analysis described herein.

In some embodiments, the instrument may receive information from a user and/or deliver information to the user via a graphical user interface (GUI). Alternatively, or in combination, the instrument may send and/or receive information via a network connection (e.g., via WiFi and/or Bluetooth connection(s)).

In some embodiments, the cartridge and instrument perform functions together to process a nucleic acid test on a sample inserted into the cartridge. In some embodiments, the instrument will also receive information such as patient name and other demographic information such as age, gender, etc. from a user. This information can be entered via the touch screen display, a keypad, a separate mobile device, a laboratory information management (LIM) system connected to the instrument by a local area network connection (e.g., WiFi or Bluetooth), or the like, or any combination thereof. In some embodiments, the instrument will output information to the user. In some embodiments, the information may include test results and may be provided to the patient being tested and/or to a health care provider (HCP) if the HCP is involved in the testing. In some embodiments, the instrument will transmit information via the touch screen display and/or via a local area network connection such as WiFi or Bluetooth. In some embodiments, the instrument may be used in a clinic, lab, or office with HCP physically present during the assay. Alternatively, or in combination, the instrument may interface remotely with one or more HCPs via the cloud (e.g., using a telehealth or remote health environment) and test results and patient information may be transmitted via a secure encrypted internet protocol.

In some embodiments, there are electronic contacts provisioned as part of the interface between the instrument and the cartridge. The contacts may facilitate information transfer between the cartridge and the instrument. For example, the electronic contacts may facilitate transfer of information about the cartridge itself, including, but not limited to, date of manufacture, expected lifetime (or “use by” date), lot number, assay identification, and/or any relevant assay parameters (e.g., information about assay temperatures, sequence timings, etc.), or the like. Alternatively, or in combination, the contacts may carry digital, power, and/or analog signals between the instrument and the cartridge. Alternatively, or in combination, transfer of electronic information and/or power may be accomplished through near-field communication (NFC) and/or radio-frequency identification (RFID) electronic components.

### B. Fluidic Drives

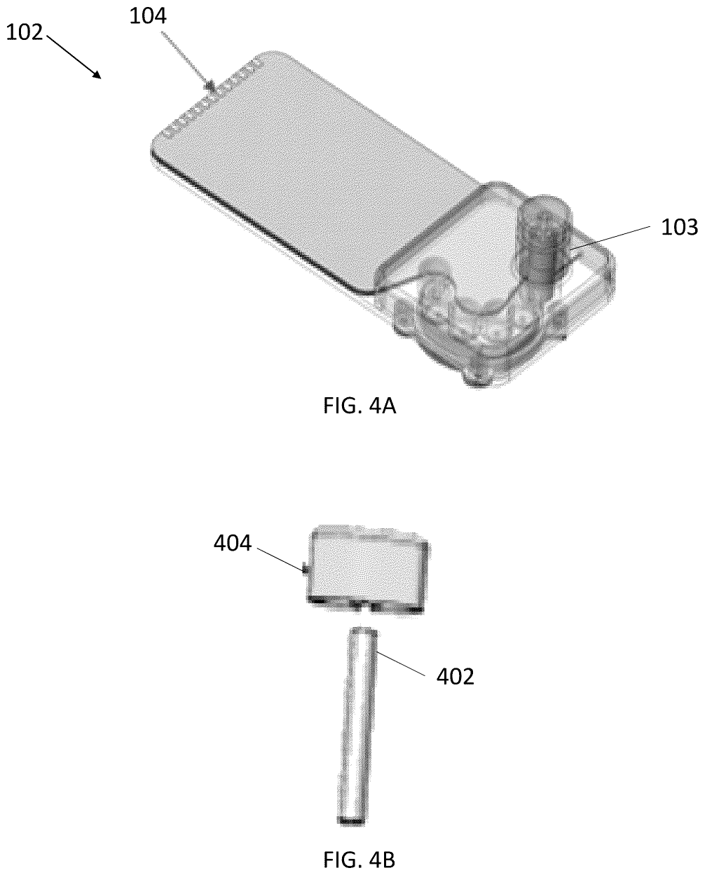

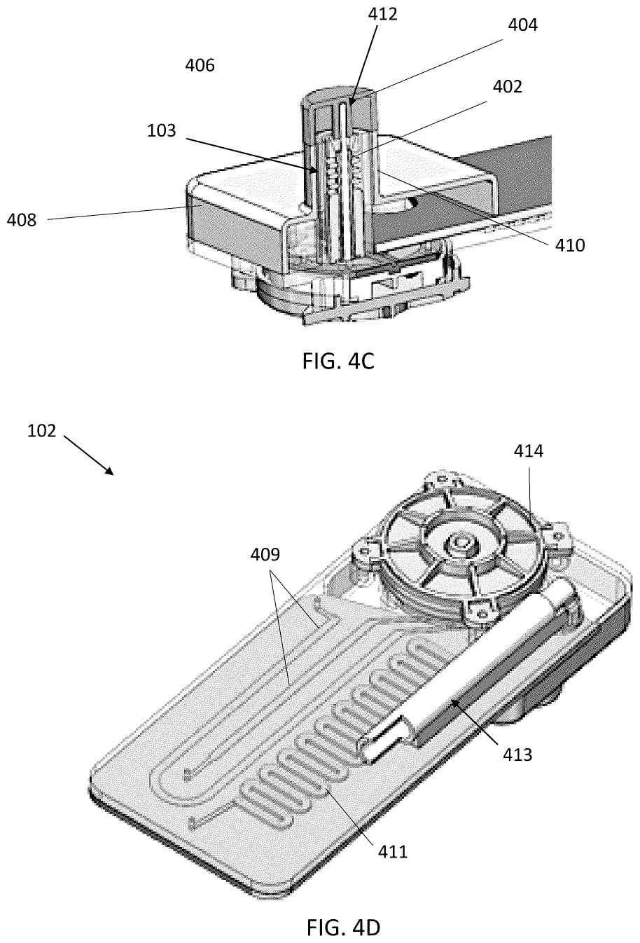

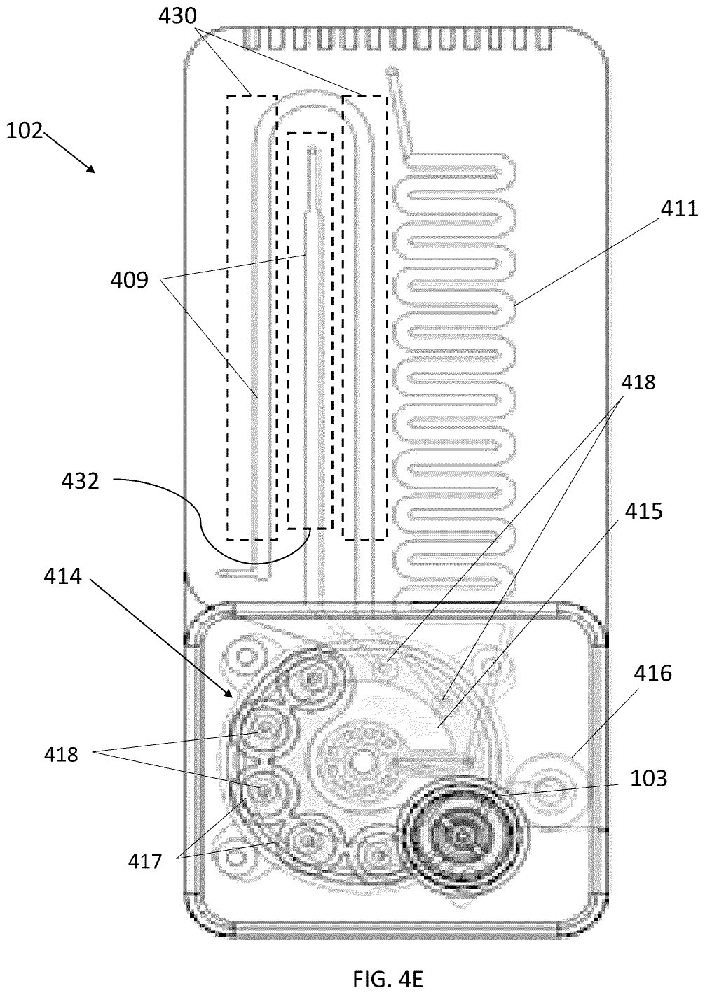

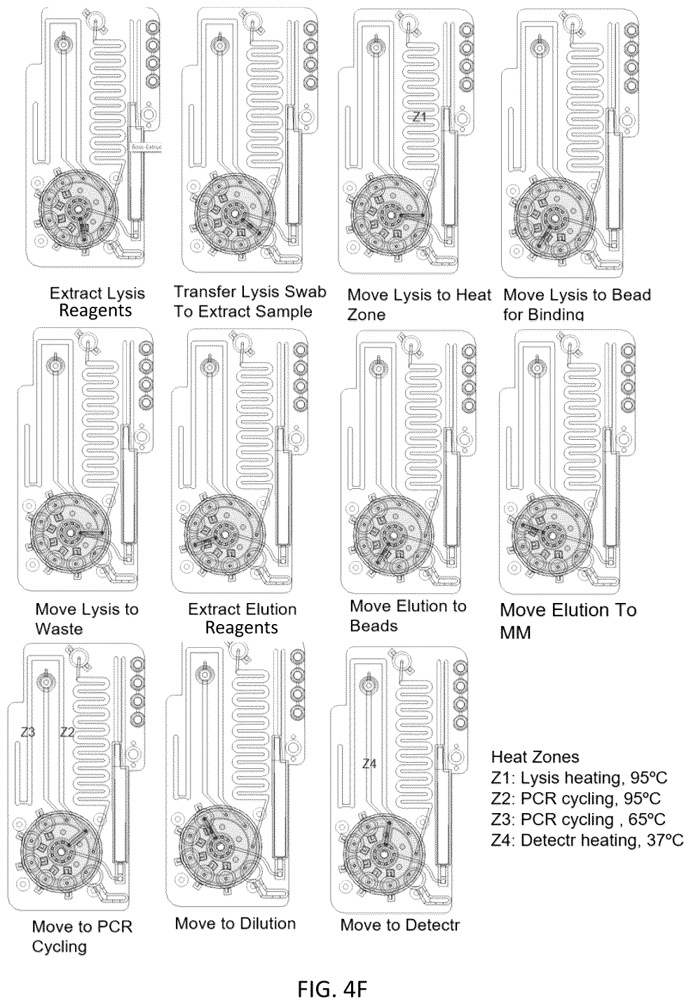

In some embodiments, the instrument comprises one or more fluidic drives. In some embodiments, a motorized XYZ gantry will be used to drive an actuator platform coupled to one or more moveable components of the cartridge (e.g., the sample reservoir, fluid jumpers, rotary valves, reagent reservoirs, syringes, etc.). In some embodiments, the X-direction motor will drive forward/backward movement, and the Y-direction motor will drive left/right movement. In some embodiments, the Z motor may function as an actuator to drive a plunger up/down (e.g., to dispense from the reagents capsules as described herein). In some embodiments, the actuator will be used to drive a syringe capsule (e.g., the syringe 413 on the cartridge as shown in FIG. 4D) that generates positive and/or negative pressure to move fluids within the cartridge. Alternatively, or in combination, a pump in the instrument may be used to generate positive and/or negative pressure. In some embodiments, positive pressure can also be used to generate bubbles within a reservoir (e.g., to mix samples with lysing reagents and/or silica beads). In some embodiments, the instrument may comprise one or more sensors for monitoring pressure within the cartridge, which may be used to provide feedback and/or control a “kill switch” configured to stop operation should the pressure within the cartridge be outside of operating limits. In some embodiments, multiple locations within the cartridge might utilize one or more magnets of the instrument (e.g., to capture magnetic silica beads within a fluid reservoir, chamber, or channel as described herein). In some embodiments, these magnets may engage and disengage the cartridge via a motor or a solenoid. In some embodiments, an ultrasonic horn, waveguide, or probe may be used to agitate the sample and/or move fluids within the cartridge as described herein. In some embodiments, a single ultrasound source may be provided within the instrument and a multi-location waveguide may direct the ultrasound energy to various locations within the cartridge (e.g., the sample receiver interface, a sample reservoir, etc.).

### C. Thermal Control

In some embodiments, the thermal environment of the cartridge may be controlled by the instrument. In some embodiments, the thermal system primarily has a controllable heating element and/or a controllable cooling element. In some embodiments, the thermal system may comprise a control loop configured for implementing direct and/or indirect temperature measurement of the fluid and/or heating or cooling the fluid to reach a pre-determined temperature. In some embodiments, the control loop can include one or more feedback element to facilitate temperature control. In some embodiments, the control loop can include one or more model-based control elements to facilitate temperatures control. In some embodiments, the thermal system may be configured to perform thermocycling and/or maintain a constant temperature within one or more regions or zones of the cartridge as described herein.

In some embodiments, the instrument uses Peltier/thermoelectric (TEC) coolers to heat and/or cool one or more regions or zones of the cartridge. The TEC coolers may engage with the cartridge upon insertion, thereby achieving intimate contact with the cartridge at the desired region(s) or zone(s). In some embodiments, the cartridge may be configured to provide improved thermal conductivity and/or time response for use with the various heating/cooling systems described herein. For example, a surface of the cartridge may comprise a thin film for heat exchange with the heater/cooler(s) of the instrument. In some embodiments, the cartridge may be configured to enable temperature measurement(s).

In some embodiments, the instrument may use one or more thermal reservoirs to heat and/or cool one or more regions or zones of the cartridge. The thermal reservoir(s) may be moved in and out of contact with a surface of the cartridge in order to conduct heat therebetween. In some embodiments, one or more thermal reservoirs may be moved in and out of contact with the surface in order to heat and cool the fluid in a cyclic manner (e.g., for thermocycling-based amplification). In some embodiments, one or more thermal reservoirs may be moved in and out of contact with the surface in order to maintain a desired temperature of the fluid for a pre-determined length of time (e.g., for isothermal amplification). In some embodiments, one or more thermal reservoirs may be moved into contact with a surface of the cartridge to generate one or more heat zones and the fluid may be moved into and out of the heat zones (and optionally between heat zones when multiple thermal reservoirs are provided) in order to heat and/or cool the fluid in a cyclic manner and/or for a pre-determined length of time. The thermal reservoir(s) may have a higher thermal capacity that the fluid(s) disposed within the one or more regions or zones of the cartridge, thereby enabling rapid heat transfer from the thermal reservoir(s) to the fluid(s). In at least some instances, moving the thermal reservoir(s) into and out of contact with the cartridge may provide a simpler alternative to moving the fluid (e.g., via pumping or by moving the cartridge itself) between different temperature zones to change the temperature of the fluid.

In some embodiments, the instrument uses optical or photonic heating. In some embodiments, the instrument directly heats the reaction mixture by irradiating the liquid with optical wavelengths that are strongly absorbed by water (e.g., infrared wavelengths targeting water absorption peaks below 1500 nm) or by transducing light to heat using particles (e.g., gold nanoparticles) dispersed throughout the heated region(s) or zone(s) of the cartridge (e.g., using light of additional wavelengths). In at least some instances, direct heating may offer advantages in thermal speed compared to some indirect heating methods by directly heating the reaction mixture instead of controlling the temperature of a surface in contact with the reaction mixture. In at least some instances, direct heating may reduce or avoid time delays associated with fluids inside a chamber equilibrating with a surface temperature.

In some embodiments, optical temperature measurement may be performed within the heated region(s) or zone(s), e.g., by a spot measurement or a camera. In some embodiments, the optical temperature measurement device measures the temperature by quantifying infrared radiation emitted by the fluid and the chamber enclosing the fluid. In at least some instances, optical temperature measurement may be advantageous in terms of speed, as measurement loops involving such devices may have the potential to be faster than some other temperature measurement devices. This may allow faster control loop response times and therefore faster transitions between temperatures within a region or zone.

In some embodiments, the cartridge may comprise one or more resistive heating elements and the instrument may be configured to apply an electric current to the one or more resistive heating elements to heat the fluid. In some embodiments, the resistive heating element(s) may be disposed adjacent a surface of one or more regions or zones of the cartridge to be heated. The electrical contacts of the instrument may engage with one or more contact pads of the resistive heating elements of the cartridge upon insertion, thereby achieving electrical communication with the cartridge in order to heat the desired region(s) or zone(s). In at least some instances, resistive heating may provide a simple and flexible alternative or addition to other heating mechanisms which may require more complex instrumentation, a larger instrument and/or cartridge footprint/form factor, and/or more complicated fluidic designs.

In some embodiments, the instrument may use one or more fans to facilitate cooling of the fluid within the cartridge (e.g., using forced convection). The fan(s) may be used in combination with any of the heating mechanisms described herein to control the temperature of the fluid within the cartridge.

In some embodiments, temperature control may be important for controlling one or more reactions taking place on the cartridge. For example, temperature control may be important for controlling a lysis reaction, an amplification reaction(s) in the amplification region, and/or a detection reaction in the detection region. In some embodiments, temperature control of +/−0.5° C. may be desired for some assays steps, such as during PCR annealing (which may be conducted at a temperature within a range from about 45.0° C. to about 70.0° C.). In some embodiments, other assay steps may have less stringent temperature control designs. For example, at higher temperatures used for denaturation (e.g., from about 80° C. to about 98° C.), the temperature control system may be sufficient with a temperature accuracy of +/−2.0° C. In some embodiments, the precision for temperature control within the system may be to a tenth of a degree Celsius.

### D. Detection

In some embodiments, the instrument may comprise a detector/sensor system to read out the spatial multiplexing implemented in the cartridge. In some embodiments, the detector system may comprise a source and a detector. In some embodiments, the source may comprise an illumination source. In some embodiments, the source may comprise an electrical signal source.

In some embodiments, the detector comprises an optical sensor or optical detector. In some embodiments, the detector is an image sensor (e.g., a camera, photomultiplier tube, charge-coupled device, active-pixel sensor, photodiode, complementary metal-oxide semiconductor (CMOS), or the like). In some embodiments, the detector may comprise an array of discrete optical detectors, one for each detection spot or chamber within the detection region of the cartridge. In some embodiments, the detector may comprise one or more optical channels. For example, a first optical channel may be used to detect cleavage of a reporter and a second optical channel may be used to detect background signal (e.g., autofluorescence). In some embodiments, one or more lenses and/or filters may be implemented to create an image with sufficient resolution and brightness for detection of reporter cleavage. In some embodiments, one or more digital masks may be used to process an optical signal detected by the detector (e.g., a digital mask may be used to remove bubbles from the analysis)

In some embodiments, the resolution of the detector may be about 50 micrometers. In some embodiments, the resolution of the detector may be less than about 50 micrometers. In some embodiments, the resolution of the detector may be less than about 25 micrometers. In some embodiments, the resolution of the detector may be less than about 5 micrometers.

In some embodiments, the signal is selected from the group consisting of an optical signal, a fluorescent signal, a colorimetric signal, a potentiometric signal, an amperometric signal, and a piezo-electric signal. In some embodiments, the signal is associated with a change in an index of refraction of a solid or gel volume in which said at least one programmable nuclease probe is disposed. Cleavage of a reporter (e.g., a protein-nucleic acid) can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

In some embodiments, the method for detection is fluorescence. In some embodiments, the detector (or plurality of detectors) may detect a change in wavelength (e.g., a change in color), intensity (e.g., brightness), or a degree of wavelength change (e.g., with appropriate dispersion elements to access wavelength space). In some embodiments, the system may be configured to detect one or more wavelengths (e.g., one for each fluorophore in a fluorescence-based system). In order to enable this fluorescence detection, optical filtering of illumination light and/or detection light may be implemented to block unwanted crosstalk (e.g., illumination light being detected in detection systems).

In some embodiments, one or more optical elements may be implemented in the cartridge and/or camera to aid in image acquisition and control. Such elements may include alignment features, fiduciary marks, and/or controls for detector level-setting (e.g., high, low, and zero response spots or chambers within the device). In some embodiments, the one or more optical elements may facilitate image registration, level calibration, and/or error detection on an automated basis.

In some embodiments, detection mechanisms may comprise interferometry, surface plasmon resonance, electrochemical detection such as potentiometry, or other detection mechanisms.

### IV. CARTRIDGE

Described herein are various embodiments of a cartridge for the analysis and detection of one or more nucleic acids in a sample. In some embodiments, the cartridge may be coupled to an instrument in order to analyze the one or more nucleic acids as described herein. In some embodiments, the cartridge may be self-sufficient and may not need to be interfaced with another instrument in order to analyze the one or more nucleic acids. The cartridge may be configured to receive a sample and perform one or more reactions on the sample in order to detect one or more target nucleic acids. In some embodiments, the cartridge may be configured to perform one or more of the following steps: sample collection, sample extraction, sample lysis, protein degradation, nucleic acid extraction, nucleic acid purification, nucleic acid concentration, waste removal, nucleic acid elution, nucleic acid amplification, a programmable nuclease-based detection reaction, target detection, and/or reporter detection, or any combination thereof.

In some embodiments, one or more elements or components of the cartridge may be modular (or have a modular design). Any of the cartridges described herein may comprise one or more modules. The one or more modules may be independently created, modified, replaced, and/or exchanged with other modules or between different systems. The one or more modules may be selected or replaced as desired in order for the cartridge to perform a desired function and/or assay. In some embodiments, the cartridge may comprise a plurality of different modules. In some embodiments, the cartridge may comprise one or more of a sample receiver module, a reagent module, a sample preparation/concentration module, an amplification module, a mixing module, a detection module, or any combination thereof. For example, the cartridge may comprise a sample receiver module, a reagent module, a sample preparation module, an amplification module, and a detection module. In some embodiments, the cartridge may comprise more than one of the same type of module (e.g., two or more reagent modules, etc.). In some embodiments, two or more of the modules may be in fluid communication with each other. Alternatively, or in combination, two or more of the modules may be capable of being put in fluid communication with each other (e.g., via a valve such as a rotary valve, jumper valve, or the like). In some embodiments, the one or more modules may be physically distinct from one another. In some embodiments, the one or more modules may be functionally distinct from one another. In some embodiments, the one or more modules may be manufactured individually or as a unitary construction (in which the modules are functionally distinct but physically part of a same physical construct).

In some embodiments, the cartridge may comprise one or more pieces or segments configured to be coupled to one another to form a complete cartridge. In at least some instances, providing the cartridge in more than one piece may facilitate proper reagent storage. For example, some of the reagents needed for the assay may be in liquid form while other reagents may be in dried, vitrified, or lyophilized form. Preferred storage conditions for liquid form reagents may be sufficiently different from preferred storage conditions for dried, vitrified, or lyophilized reagents so as to make it difficult or impossible to store each reagent in its preferred condition. Separating the liquid reagents from the dried, vitrified, and/or lyophilized reagents onto different cartridge segments may enable proper reagent storage for each segment. In exemplary embodiment shown in FIG. 3, a first cartridge segment may store liquid reagents including reagents for extraction, reagents for concentration, and/or reagents for amplification. The first cartridge segment may further comprise a sample interface, a sample receiver, and/or a sample preparation module. The first cartridge segment may be individually packaged in a vapor impermeable bag to control or prevent liquid evaporation from the liquid storage reservoirs (e.g., capsules). A second, separate cartridge segment may house dry, vitrified, or lyophilized reagents for amplification and/or detection. The second cartridge segment may further comprise an amplification region and/or a detection region. The second cartridge segment may be individually packaged so as to maintain the components and modules for amplification and/or detection in a dry environment. In some embodiments, the second cartridge segment may be packaged using desiccant materials inside a vapor impermeable bag to keep water molecules away from the second cartridge segment. In some embodiments, the lyophilized and/or dried reagents may comprise biological enzymes and/or synthetic oligonucleotides of nucleic acids as described herein. In some embodiments, the sample preparation region and waste collection functions of the cartridge may be satisfied in either or both the first and second cartridge segments.

In some embodiments, the cartridge's functions can be schematically represented as in FIG. 3. The arrow denotes the order of actions. Left to right in the direction of the arrow is verbally communicated as functionally downstream in the following descriptions. Liquid waste collection may occur in any of the functional blocks. It will be apparent to one of ordinary skill in the art that different module configurations may be utilized, depending on the assay to be performed, while still being within the scope of the teachings described herein. For example, reagents for amplification may not be provided on the cartridge when nucleic acid amplification is not performed.

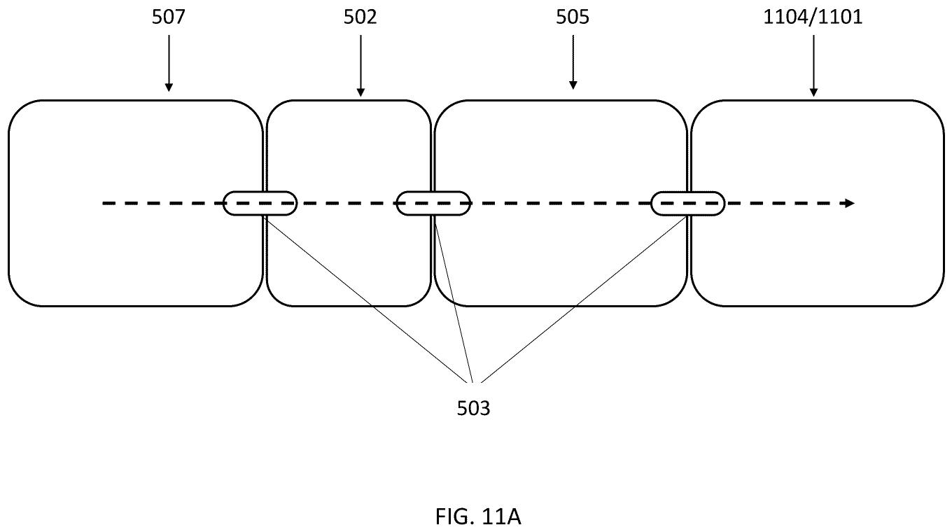

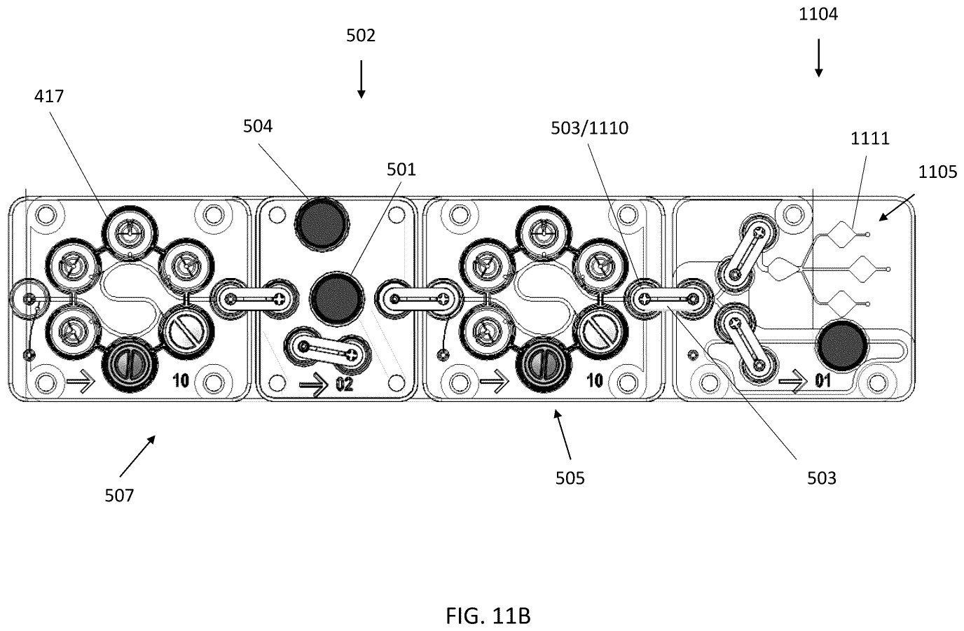

In general terms, the cartridge in accordance with some embodiments provides one or more of: a reagent reservoir, a sample interface in fluid communication with the reagent reservoir, the sample interface configured to receive a sample, an amplification region in fluid communication with one or more of the sample interface or the reagent reservoir and configured to amplify one or more nucleic acids in the sample, and a detection region in fluid communication with the amplification region. Each of the reagent reservoir, the sample interface, the amplification region and the detection region may be provided as one or more separate modules, as described herein. For example, FIG. 11A shows an embodiment in which the sample interface is provided as a sample receiver module 502, the reagent reservoir is provided in one or both of a first reagent module 507 and a second reagent module 505. The amplification region is provided in an amplification module 1104. The detection region might be provided as part of or instead of the amplification module 1104 or might be provided as a further detection module 1101. The dashed arrow in FIG. 11A illustrates the general direction of fluid flow through the cartridge. Fluid communication between the modules may be provided by jumpers 503 or the like. Further detail of such an embodiment is provided below in connection with FIG. 11B, and particular embodiments of each of the modules are discussed in further detail below. It will be appreciated that since each of the modules may be independently created, modified, replaced, and/or exchanged with other modules or between different systems, then the design of each of the modules may vary, or certain modules may be omitted entirely. It will further be appreciated that in some embodiments the modules are separable (i.e., the cartridge comprises separate, detachably coupled modules). In other embodiments the cartridge may be modular by design, but constructed such that the modules are unitary with each other or are not detachable from each other.

FIGS. 4A-4F depict various components of an exemplary cartridge device 102.

FIG. 4A shows a perspective view of the cartridge 102 including a sample interface 103 and electrical contacts 104 configured to interface with an instrument (e.g., any of the instruments described herein). The sample interface 103 may be configured to receive a sample as described herein. The sample interface 103 may also be referred to as a sample receiver. In some embodiments, the sample interface 103 may be configured to receive a sample collector 105, such as a swab, vial, tube, or syringe, containing the sample. In some embodiments, the sample interface 103 may be configured to directly receive a sample, such as a blood sample, a urine sample, or a saliva sample via a syringe or pipette, at the sample interface 103. The electrical contacts 104 may be configured to interface with corresponding electrical contacts on the instrument when the cartridge 102 is coupled thereto (e.g., via insertion) as described herein. The electrical contacts 104 may, for example, allow for communication between the instrument and the cartridge 102. Alternatively, or in combination, the electrical contacts 104 may supply power to the cartridge 102 (e.g., when resistive heating is used within the cartridge).

FIG. 4B shows a perspective view of a custom swab 402 with a cap 404 which may be configured for insertion into the sample interface 103 of the cartridge 102. A sample collector, e.g., a swab, can be used to collect a sample from a user, e.g., a patient, and inserted into a chamber of the sample receiver for collection.

FIG. 4C shows a perspective cross-sectional view of the sample interface 103 with swab 402 inserted therein. The swab 402 may be disposed within a swab channel 412 extending from the swab cap 402 to the bottom of the sample receiver silo 410. The swab cap 404 may be configured to mate with the portion of the housing 408 surrounding the sample interface 103, e.g., the sample receiver silo 410. In some embodiments, the cartridge 102 may include a sample interface 103 configured to receive a swab 402 as described herein. The swab 402 can be connected to the receiver cap 404 prior to use and, after collection, the swab 402 can be inserted into the sample receiver 103 and the cap 404 can be secured (e.g., screwed onto, press-fit, etc.) to the silo 410 to seal the chamber therein. The swab 402 is preferably retained in the sample receiver 103 throughout the remainder of the device's use. One or more locking features may be provided on the sample receiver 103 and/or sample collector to prevent removal of the sample collector and/or exposure of the contents of the sample receiver 103 chamber to the system or a user. The sample interface 103 may be configured to facilitate collection of the sample from the swab 402. In some embodiments, the sample interface 103 for swab 402 may contain one or more features configured to facilitate elution of virions or cells from the swab 402.

In some embodiments, the sample interface 103 may comprise a scraper 406. The scraper 406 may agitate the swab 402 when the swab 402 is inserted into the sample interface 103 in order to physically displace the sample from the swab 402 and transfer the sample into the cartridge 102. The scraper 406 may be disposed within the chamber of the sample receiver silo 410 and may define a channel 412 into which a sample collector (e.g., swab 402) is disposed when the sample receiver 103 is in use. The scraper 406 may include geometric interfaces (e.g., annular rings along the long axis of the scraper, spiral rings along the long axis of the scraper, vertical rungs running parallel to the long axis of the scraper, protrusions, etc.) disposed within the swab channel 412 and configured to extract the sample from the swab 402. The geometric features may scrape the swab 402 as it passes into the channel 412 and agitate the swab 402 in order to physically displace the sample from the swab 402 and transfer the sample into the chamber of the sample receiver 103. It will be apparent to one of ordinary skill in the art that the scraper 406 may comprise a variety of forms, shapes, and sizes while retaining its function of agitating the input sample. In some embodiments, the sample interface 103 may comprise a sample reservoir into which the sample may be transferred from the sample collector. The sample receiver chamber or reservoir can include a liquid (e.g., lysis buffer, nucleic acid extraction buffer, etc.) prior to insertion of the swab into the channel or a liquid can be added after the swab has been inserted and the chamber has been sealed. In some instances, flushing a liquid over the swab can facilitate sample collection. In some cases, the sample receiver can comprise, or be coupled to, an ultrasonic horn configured to further agitate and/or lyse the sample. Alternatively, or in combination, the sample receiver can comprise a vent and be coupled to a gas source in order to enable bubbling within the chamber to further agitate and/or mix the sample liquid therein. In some embodiments, the sample interface 103 may be connected by a liquid channel (e.g., via a rotary valve, jumper valve, etc.) to a reservoir containing extraction and/or lysis liquids. In some embodiments, the sample interface may be thermally coupled to a heater of the instrument in order facilitate sample transfer from the sample collector into the cartridge.

In some embodiments, a liquid specimen may be input into the sample interface 103 instead of a swab 402. In such embodiments, the sample interface 103 may be configured to receive liquids via a transfer pipette or other fluid transfer mechanism as will be understood by one or ordinary skill in the art.

FIG. 4D shows a perspective view of the cartridge 102 with housing 408 and sample interface 103 removed for clarity. The cartridge 102 may comprise a sample preparation region comprising one or more reservoirs (e.g., reagent reservoirs 417, sample reservoirs 501, etc.), a lysis region, an amplification region, a mixing region, and/or a detection region as described herein. In some embodiments, one or more elements or regions of the cartridge 102 may be modular as described herein. In some embodiments, one or more fluid channels 409 may provide the lysis, amplification, and detection regions as shown in FIG. 4D. For example, a serpentine channel 411 may be configured to heat a sample to a pre-determined temperature (e.g., for isothermal amplification or lysis) or to cycle between temperatures (e.g., for thermocycling-based amplification). Use of a serpentine channel 411 may, for example, provide increased surface area contact between the channel and a heater compared to an elongate channel in at least come instances. Alternatively, or in combination, one or more elongate fluid channels 409 may be used for PCR cycling (e.g., by flowing a fluid within the channel 409 between different temperature zones) or detection as described herein. Fluid flow within the device may be provided via positive or negative pressure applied by a syringe 413. The syringe may be actuated by the instrument (e.g., via an XYZ gantry). The rotary valve 414 may selectively open and close fluid flow pathways at predetermined times via a linker channel as described herein.

FIG. 4E shows a plan view of the cartridge 102 highlighting the internal components of the rotary valve 414 in the cartridge 102. The rotary valve 414 may be configured to regulate fluid communication between the various components of the cartridge 102. For example, the valve may be configured to divert sample to and from one chamber or channel 409 to one or more other chambers or channels 409. Rotation of the rotary valve 414 may bring a rotating channel linker 415 in and out of alignment with ports 418 within the various reagent reservoirs 417, chambers or channels 409, and the syringe interface 416. The rotary valve 414 may couple a desired port 418 to the syringe interface 416 for direction of fluid flow thereby. In some embodiments, fluid may move in and out of the syringe 413 via the fluid pathway defined by the rotary valve channel linker 415 position. Fluid may be moved sequentially through the cartridge 102.

It will be understood by one of ordinary skill in the art that other valves may be utilized to regulate fluid flow within the cartridge 102. For example, the rotary valve shown in FIG. 4E may be replaced or used in conjunction with one or more fluid jumpers, or other valves.

In general terms, the cartridge 102 in accordance with some embodiments provides one or more of a reagent reservoir 417, a sample interface 103, an optional amplification region, and a detection region. The sample interface 103 and reagent reservoir(s) 417 are indicated in FIG. 4E. As explained above in connection with FIG. 4D, the amplification region and/or detection region may be provided by one or more fluid channels 409. By way of example only, as illustrated in FIG. 4E and explained above in relation to FIG. 4D, the amplification region might be provided by one or more of the serpentine channel 411 and the outer fluid channels 430. The detection region may be provided by the inner fluid channel 432. Fluid communication between the reagent reservoir(s) 417, the sample interface 103, the amplification region(s) 430, and the detection region 432 is provided by the rotary valve 414.

FIG. 4F shows exemplary valve 414 positioning of the cartridge 102 at various stages during nucleic acid analysis. From top left to bottom right: lysis buffer may be extracted from a reagent reservoir 417; the lysis buffer may then be transferred to the sample interface 103 to contact the swab 402 and extract the sample; the lysis buffer comprising the sample may then be transferred to the serpentine channel 411 for heating in heat zone Z1 comprising the serpentine channel 411, which may facilitate sample lysis; the lysate may be moved into a reservoir 417 comprising one or more beads configured to bind nucleic acids as described herein; the unbound lysate may be transferred to a waste chamber (which may, for example, comprise the serpentine channel 411 or another waste chamber as described herein); elution buffer may be extracted from a reagent reservoir 417; the elution buffer may then be transferred to the bead reservoir 417 and the nucleic acids may be eluted from the beads; the eluate may be moved to another reservoir MM/417 to separate the eluate from the beads and/or to contact the eluate to one or more additional reagents (e.g., concentrator reagents, amplification reagents, inactivation reagents, etc.); the eluate may be moved into a fluid channel 430 for thermocycling-based amplification between heat zones Z2 and Z3; the solution may then be transferred into a dilution reservoir 417 comprising one or more reagents (e.g., diluents, detection reagents, etc.); finally, the mixture may be transferred into a fluid channel 432 comprising one or more detection reagents and a programmable nuclease-based detection reaction may be performed on the sample as described herein. Fluid flow within the device may be provided via positive or negative pressure applied by the syringe 413 as described herein. The rotary valve 414 may selectively open and close fluid flow pathways at predetermined times via a linker channel 415 as described herein.

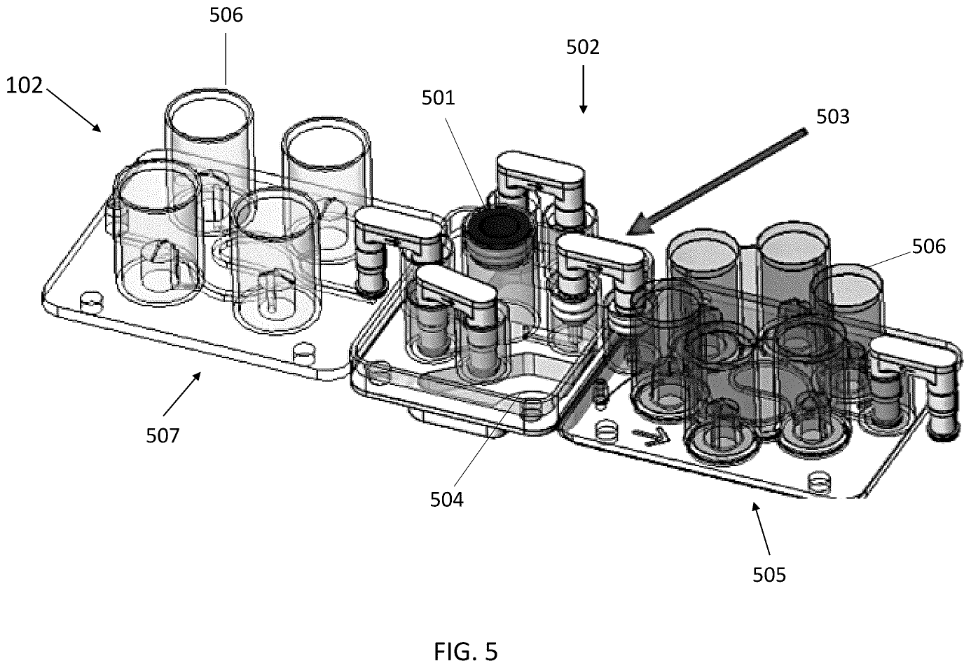

FIG. 5 depicts an exemplary arrangement of reagent reservoirs and a sample reservoir 501 of an exemplary cartridge device 102. Any of the cartridges 102 described herein may comprise one or more modules as described herein. In an exemplary embodiment, a cartridge 102 may comprise a sample preparation region comprising a sample receiver module 502 and one or more reagent modules (505, 507). The modules may be coupled to one another so as to provide fluid communication therebetween. In some embodiments, fluid communication may be regulated between the modules by one or more valves. For example, one or more fluid jumpers 503 may be used to valve fluid flow between and/or within the modules. The fluid jumpers 503 may be configured to provide one-way or bi-directional fluid flow between the modules. For example, the fluid jumpers 503 may be configured and arranged to regulate fluid flow from a reagent reservoir to the sample reservoir 501 and prevent backflow from the sample reservoir 501 to the reagent reservoir. Alternatively, or in combination, one or more fluid jumpers 503 may be configured to regulate fluid flow from the sample reservoir 501 to a waste reservoir 504 on the sample receiver module 502 and prevent backflow from the waste reservoir 504 to the sample reservoir 501. In some embodiments, one or more fluid jumpers 503 may be configured and arranged to regulate fluid flow from an amplification module to a detection module, as described herein, and prevent backflow from the detection module to the amplification module.

In some embodiments, one or more reagent reservoir may comprise a reagent capsule as described herein. The reagent capsule may be disposed within a reagent silo 506 as described herein. In some embodiments, each liquid reagent may be individually filled and sealed in a reagent capsule. In some embodiments, the reagent capsule may be filled with a dry reagent.

The number of reagent reservoirs (e.g., reagent capsules) disposed on a reagent module may be tailored to the specific reagents required by the assay being performed by the system. For example, the number of reagent reservoirs in some embodiments may vary from 4 to 6 in number. In some embodiments, there may be at least 1, 2, 3, 4, 5, 6, 7, 8, 9, or 10 reagent reservoirs per module.

In some embodiments, the cartridge 102 may comprise a sample receiver module 502 configured for sample preparation. In some embodiments, the cartridge 102 may comprise a first reagent module 507 comprising one or more lysis reagents. In some embodiments, the lysis reagent module 507 may comprise one or more lysis reagents located in one or more reagent capsules. In some embodiments, the cartridge 102 may comprise one or more waste reservoirs 504 in fluid communication with the sample receiver module 502. In some embodiments, the cartridge 102 may comprise a second reagent module comprising one or more wash buffers and/or elution buffers (505). In some embodiments, the second reagent module 505 may comprise wash buffers and elution buffers located in one or more reagent capsules. In some embodiments, the first (lysis) reagent module 507, the waste reservoir 504, and the second (wash/elution) reagent module 505 may be connected to the sample receiver module 502 by jumpers 503.

In some embodiments, fluid flow within the cartridge 102 may be unidirectional. For example, a sample collector may be inserted into the sample interface as described herein. The sample interface 103 may comprise a sample reservoir 501. The sample interface may optionally comprise a scraper as described herein. Lysis reagents may be transferred from one or more reagent reservoirs of a first reagent module to the sample reservoir 501 for lysis of the sample as described herein. In some embodiments, lysis of the sample may completely kill any live pathogens within the sample. In some embodiments, additional chemical inactivation reagents may be transferred from the first reagent module into the sample reservoir 501 in order to ensure complete inactivation (e.g., killing) of the sample pathogen. Alternatively, or in combination, the sample reservoir 501 or a downstream location therefrom may be heated to a predetermined temperature for a predetermined amount of time in order to ensure complete sample inactivation. In some embodiments, one or more additional reagents (e.g., PK for protein lysis, alcohol for nucleic acid aggregation, etc.) may be transferred from the first reagent module to the sample reservoir 501 before the sample is transferred downstream. In some embodiments, nucleic acids of the sample may be captured by a concentrator (e.g., a filter, column, beads, mesh, etc.) on a second reagent module (e.g., within a jumper 503 connecting the second reagent module to an amplification module). In some embodiments, the sample may be transferred to a separate sample concentration module comprising a concentrator downstream of the second reagent module. Wash and/or elution reagents may be transferred from one or more reagent reservoir of the second reagent module to the concentrator for washing and elution of the sample as described herein. Elution of the nucleic acids from the concentrator may move the purified nucleic acids downstream into an optional amplification module or detection module. The amplification module may comprise one or more amplification channels or chambers as described herein. The nucleic acids may be amplified as described herein and then transferred downstream to a detection module. In some embodiments, the nucleic acids may be amplified in the detection module prior to or during detection. In some embodiments, the nucleic acids may not be amplified. One or more target nucleic acids may then be detected in a multiplexed fashion as described herein.

### A. Reagent Reservoir

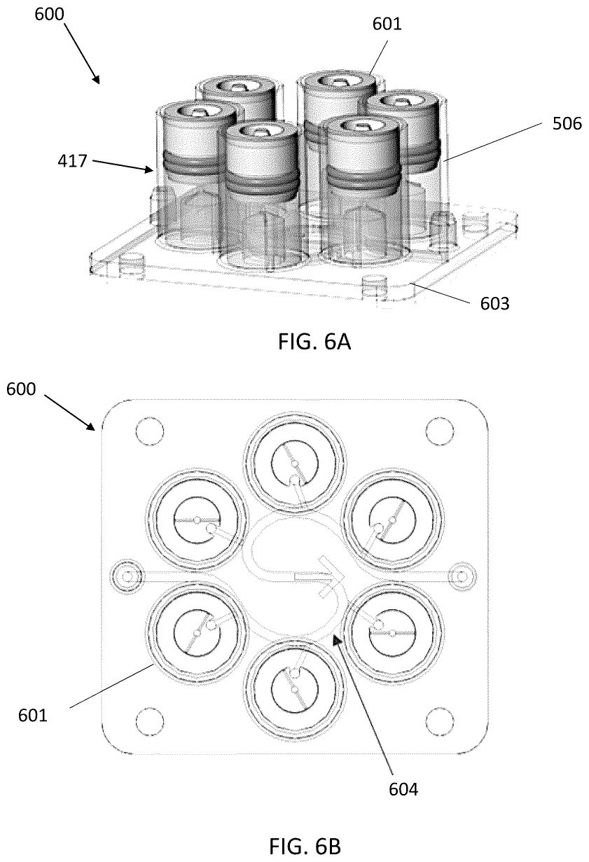

FIGS. 6A-6B show a perspective view and a top view, respectively, of a plurality of reservoirs 417 optionally comprising reagent capsules 601. The plurality of reservoirs 417 may be arranged on a reagent module 600, which may be coupled to a sample receiver module via a jumper as shown in FIG. 5 (e.g., reagent module 505 or 507). In the exemplary embodiments, six reagent capsules 601 are radially arranged around a central s-shaped fluid channel 604. The capsules 601 may be disposed within silos 506 arranged on a support 603 (e.g., in a radial pattern as shown). Each reagent capsule 601 may be in fluid communication with the s-shaped fluid channel 604 via a dedicated side channel. The s-shaped fluid channel 604 may be configured to connect to one or more other modules via a jumper as described herein. It will be understood by one of ordinary skill in the art, however, that the number and arrangement of the reagent capsules 601, as well as their connection to one another and/or other modules, may be varied based on the number of reagents, the desired module footprint, the desired fluid flow patterns and pathways, and/or any of a variety of other parameters which may make other permutations for favorable for a desired application.

In this embodiment there are six different liquid capsules shown. In some embodiments, there may be fewer capsules per reagent module 600 and/or some of the capsule silos 506 may be used to collect waste material from the nucleic acid extraction steps.

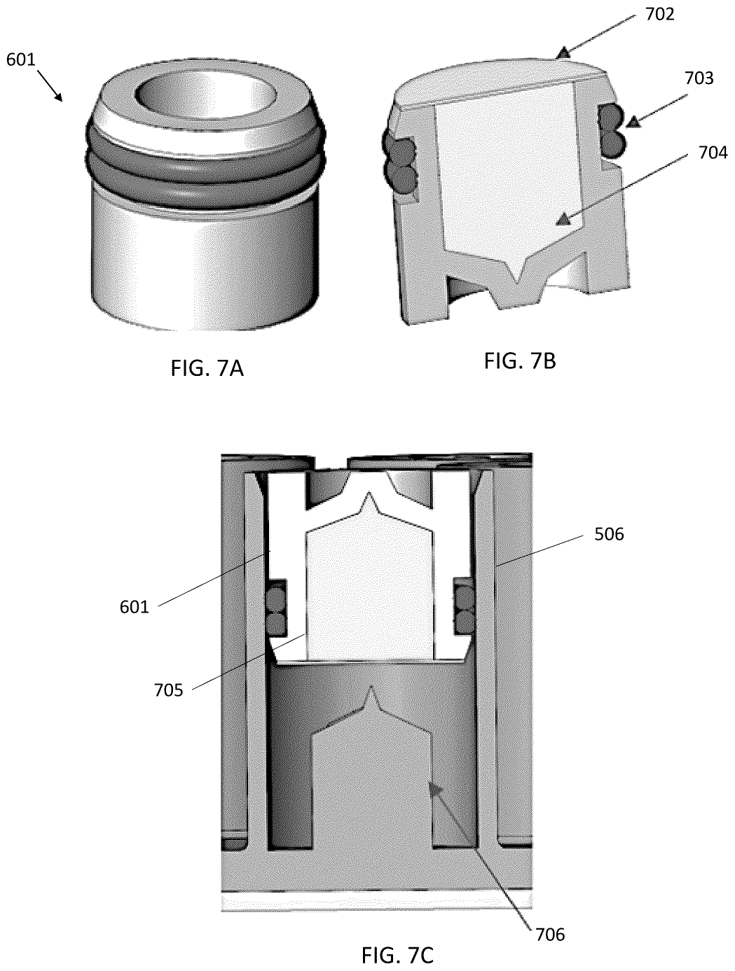

FIGS. 7A-7C depict an exemplary reagent capsule 601, which may be used with any of the embodiments of the cartridge disclosed herein. FIG. 7A shows a perspective view of an unfilled reagent capsule 601. FIG. 7B shows a perspective cross-sectional view of a filled and sealed reagent capsule 601. FIG. 7C shows a cross-sectional view of a reagent capsule 601 disposed within a silo 506 above a piercer core 706. Any of the devices described herein may comprise one or more reagent capsules 601. Each capsule 601 may be slidably disposed within a capsule silo 506 comprising a piercer core 706. In some embodiments, the capsule 601 may comprise a cylindrical capsule with one end open for reagent filling prior to disposal within a capsule silo 506. In some embodiments, the capsule 601 may be oval-shaped. In at least some cases, it may be preferable to avoid sharp internal angles within the capsule 601 in order to avoid issues when sealing the capsule 601. Each silo 506 may comprise a piercing mechanism 706 configured to pierce through the pierceable cover 702 of the corresponding reagent module capsule 601 when the capsule is translated from a closed configuration to an open configuration, so as to release a reagent therefrom. In some embodiments, the capsule 601 may comprise an elastomer sealing ring 703 or other sealing mechanism (e.g., press-fit or interference fit seals 2101 as shown in FIG. 21, etc.) configured to seal against the capsule silo 506. Sealing the capsule 601 to the silo 506 may reduce or prevent fluid loss when the capsule 601 is moved from the closed configuration to the open configuration. In some embodiments, the open end may be sealed with a pierceable cover 702 (e.g., a foil) after reagent filling thereof. The pierceable cover 702 may comprise a laminate. For example, the pierceable cover 702 may comprise an HDPE/foil laminate. In at least some instances, the pierecable cover 702 (e.g., a foil layer of the HDPE/foil laminate) may be configured to act as a vapor barrier to reduce or prevent reagent loss from the reagent capsule 601. In some embodiments, the capsule 601 may be filled with a fluid 704. In some embodiments, the capsule 601 may be filled with a dried powder. In some embodiments, the capsule 601 may be filled with a gas. In some embodiments, the capsule 601 may be filled with a gas, a fluid, a liquid, a dried powder 704, or any other format of reagent. In some embodiments, the capsule 601 may be filled and stored until needed for assembly on the cartridge. The capsule 601 may be filled and assembled into the capsule silo 506 of a cartridge or other solid support. In some embodiments, the capsules 601 may be assembled into a cartridge with silos 506 disposed in a radial pattern (e.g., a reagent wheel) on a surface thereof as shown in FIGS. 6A-6B. In some embodiments, the assembled capsules 601 (as shown in FIG. 7C) may be pierced by translating them down the silo 506 wall so as to contact the piercable cover 702 against a piercing mechanism 706 disposed within the silo 506. In some embodiments, the piercing mechanism 706 may comprise a sharp piercer core. The piercer core 706 may be correspondingly sized and shaped to the fluid chamber 705 of the reagent capsule 601. Continued downward translation of the capsule 601 onto the piercer core 706 following initial disruption of the pierceable cover 702 may seat the piercer core 706 fully inside the capsule chamber 705, thereby ensuring dispensation of the full fluid volume 704 from the capsule 601 into the cartridge.

In some embodiments, translation of the reagent capsule(s) 601 may be actuated by an actuator on the instrument (e.g., by an XYZ gantry of the instrument). In some embodiments, one or more reagent capsule 601 may be positioned at a defined location such that the instrument's actuator may be shared by stepper motors or solenoids for the movement.

In some embodiments, the capsules 601 may be arranged above a piercer core 706 as depicted as in FIG. 7C. In some embodiments, pushing the capsule 601 to the bottom of the capsule silo 506 may cause the piercer core 706 to displace the fluid 704 inside the capsule 601 and squeeze it out into a channel connected to the silo 506. In some embodiments, a secondary pressure from the instrument may be utilized move the fluid 704 further downstream.

Any of the cartridges described herein may comprise one or more reagents for extraction of a target nucleic acid from a sample. In some embodiments, the liquid reagents needed for the nucleic acid test may include one or more chemical agents to digest proteins, one or more chemical agents to lyse cellular membranes, and/or one or more alcohols to solvate and aggregate the nucleic acids. In some embodiments, the one or more reagents may comprise a plurality of silica-coated beads configured to bind nucleic acids. In some embodiments, the silica-coated beads may be loaded into a reagent capsule 601. In some embodiments, the silica-coated beads may be loaded in the reagent silo 506 or a channel connecting thereto in a dry powder format. A corresponding reagent capsule 601 may comprise a reconstitution liquid in which the silicon-coated beads may be reconstituted upon piercing of pierceable foil 702 by the piercer core 706 and release of the reconstitution liquid into the reagent silo 506. In some embodiments, the silica-coated beads may be loaded as a liquid suspension into a reagent capsule 601. In some embodiments, all of the reagents for extraction may be stored functionally upstream of the sample reservoir.

In some embodiments, the number of capsules within the cartridge may vary depending on the number and volume of reagents. In some embodiments, a module may comprise a plurality of capsules varying from 4 to 8 in number. In some embodiments, there may be at least 1, 2, 3, 4, 5, 6, 7, 8, 9, or 10 capsules per module.

In some embodiments, the fluid volume in each capsule may vary from about 50 μL to 1 mL per capsule. In some embodiments, the fluid volume in each capsule may be at least about 100 μL, about 200 μL, about 300 μL, about 400 μL, about 500 μL, about 600 μL, about 700 μL, about 800 μL, about 900 μL, or about 1000 μL.

In some embodiments, the fluid volume filled in a capsule may vary from about 50 μL to 500 μL. In some embodiments, the fluid volume in a capsule may be at least about 50 μL, about 100 μL, about 150 μL, about 200 μL, about 250 μL, about 300 μL, about 350 μL, about 400 μL, about 450 μL, or about or 500 μL.

In some embodiments, one or more reagent capsules may be filled with more liquid than is needed for its intended purpose. In some embodiments, only a portion of this volume may be used for an assay and excess residual volume may be left in the reagent capsule (i.e., the reagent capsule may be configured to dispense a fraction of the liquid disposed therein) and/or routed to a waste reservoir on the cartridge or instrument.

In some embodiments, the capsule(s) may be fully assembled onto the cartridge during manufacturing. In some embodiments, the capsule(s) may be provided with the cartridge in a modular fashion so as to allow a user to mix and match reagents as desired.

In some embodiments, during a nucleic acid assay test, the instrument may be configured to open each capsule inside the cartridge at a predetermined time at which the liquid inside the capsule is needed. In some embodiments, the instrument may provide fluid movement from the capsule by translating the capsule within the silo from a closed configuration to an open configured, thereby displacing the liquid therefrom into the space within the silo as described herein. In some embodiments, moving the capsule within the silo to pierce the pierceable cover may open a vent and improve subsequent fluid movement from the capsule.

FIG. 21 shows an exemplary reagent reservoir/capsule. The reagent reservoir/capsule may be substantially similar to the reagent reservoir/capsule described in FIGS. 7A-7C, with the addition of press-fit or interference-fit sealing mechanism(s) 2101 configured to seal the capsule 601 to its capsule silo when disposed therein. Each capsule 601 may be slidably disposed within a capsule silo comprising a piercer core as described herein. In some embodiments, the capsule 601 may comprise one or more sealing mechanisms 2101 (e.g., two press-fit or interference fit seals as shown) configured to seal against the capsule silo as described herein. In some embodiments, the capsule 601 may comprise a cylindrical capsule with one end open for reagent filling prior to disposal within a capsule silo as described herein. In some embodiments, the open end may be sealed with a piercable cover 702 (e.g., a foil) after reagent filling thereof as described herein. When the capsule 601 is translated from a closed configuration to an open configuration the piercing mechanism of the silo may pierce through the pierceable cover 702 to release a reagent from the reagent reservoir as described herein.

### B. Sample Reservoir

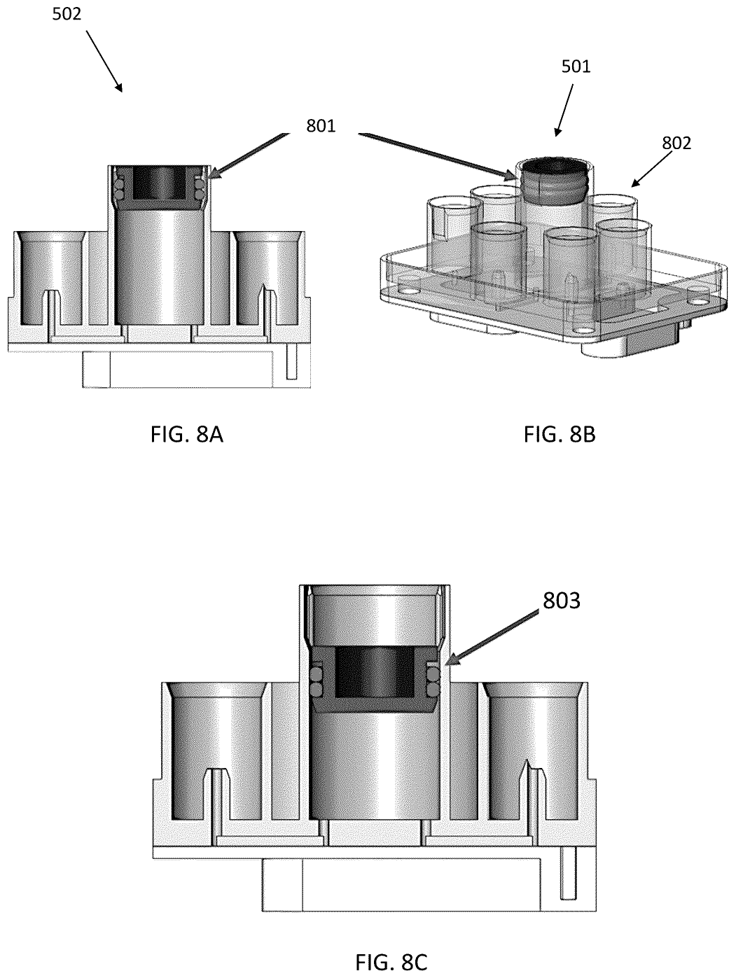

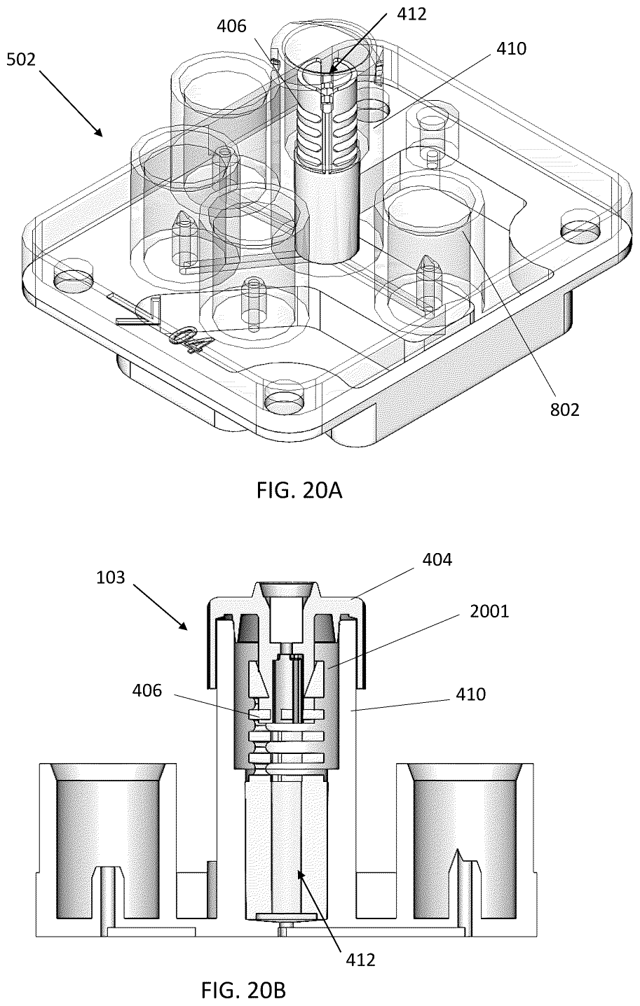

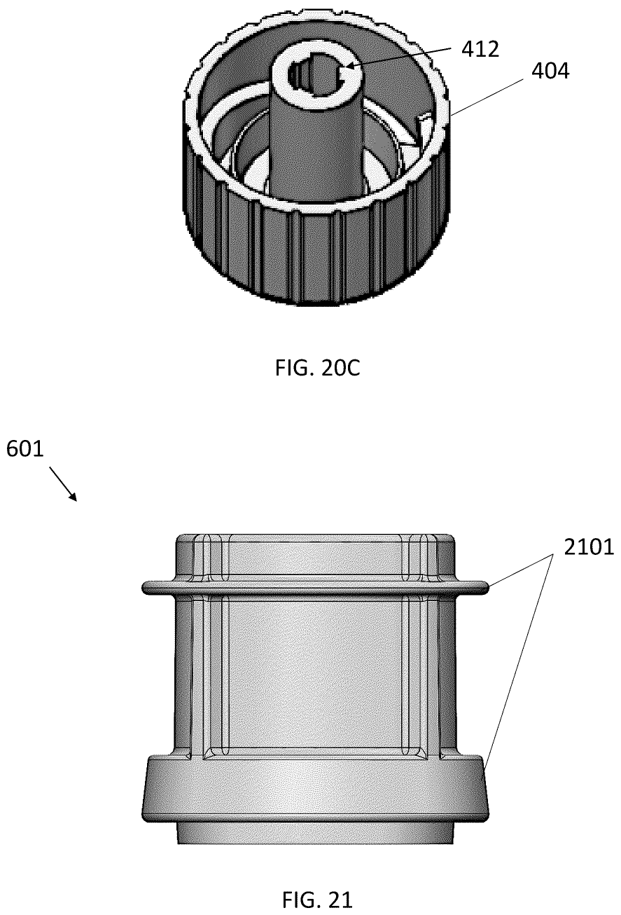

FIGS. 8A-8C show an exemplary sample reservoir 501 and a plurality of jumper silos 802 disposed therearound. FIG. 8A shows a cross-sectional view of a pair of jumper silos 802 and a sample reservoir 501 with vent 801 open. FIG. 8B shows a perspective view of the sample reservoir 501 with vent 801 open and jumper silos 802 therearound. FIG. 8C shows a cross-sectional view of the jumper silos 802 and the sample reservoir 501 with vent 801 closed 803. Any of the sample interfaces described herein may comprise a sample reservoir 501. In some embodiments, a scraper of the sample interface may be disposed within the sample reservoir 501 (e.g., as shown in FIGS. 4C and 20A-20B). In some embodiments, the sample reservoir 501 may be part of a sample receiver module 502 as described herein. The sample reservoir 501 may comprise an open configuration (shown in FIGS. 8A-8B) and a closed configuration (shown in FIG. 8C). In the open configuration, the sample reservoir 501 may be in fluid communication with its surroundings (e.g., atmosphere) via a vent 801 at a proximal end thereof. In the closed configuration, when the vent 801 is translated distally, the vent 801 may be configured to form an interference fit 803 with (or otherwise be mated to) an interior wall of the sample reservoir 501 and thereby be closed to fluid communication with the cartridge's surroundings. Fluid flow from the sample reservoir 501 into downstream fluid channels may be driven by the application of positive or negative pressure to the sample reservoir 501 when the vent 801 is in the closed position.

In some embodiments, mixing in the sample interface reservoir 501 may be facilitated by the generation of bubbles within the liquid volume while the vent 801 is in the open configuration. Gas may be released from the sample reservoir 501 via a vent 801 during mixing. In some embodiments, the vent 801 may then be sealed 803 (e.g., by pressing down on the vent plug) and the bubbles may be used to generate positive pressure inside the sample reservoir 501 (when the related jumper is in a closed configuration), which may drive fluid flow out of the sample reservoir 501 and into the downstream fluidics (when the related jumper is in an open configuration).

In some embodiments, mixing and/or sample lysis within the sample interface reservoir 501 may be facilitated by ultrasound (e.g., the sample reservoir 501 may interface with an ultrasound horn/probe/waveguide disposed in the instrument). Application of ultrasonic energy to the sample interface reservoir 501 may agitate the sample to provide mixing and/or lysis.

In some embodiments, the sample reservoir 501 may interface with a heater (e.g., a heating element disposed on the cartridge or in the instrument) which may heat the fluid within the sample. In some embodiments, heating the sample fluid may facilitate lysing. In some embodiments, mixing within the sample reservoir 501 may be driven by convective mixing. For example, the instrument may change the temperature of a heater via heater on/off profiles, thereby leading to thermal convection within the liquid.

In some embodiments, the sample reservoir 501 may interface with a magnet on the instrument in order to immobilize one or more magnetic elements within the system. For example, the magnet may be used to immobilize silica-coated magnetic beads disposed within the sample reservoir 501 for nucleic acid capture, purification, and/or concentration as described herein. In some embodiments, the magnet may be moved away from the cartridge by the instrument when the beads are to be released downstream. In some embodiments, the cartridge may contain a filter mesh configured to capture the beads. In some embodiments, a liquid back flow may move the beads functionally downstream to a sample concentration module. In some embodiments, the magnetic beads may remain immobilized throughout the sample preparation process and the sample nucleic acids may be eluted therefrom and flown downstream while the magnetic beads remain immobilized in the sample reservoir.

FIGS. 20A-20C show an exemplary sample reservoir 501 and a plurality of jumper silos 802. FIG. 20A shows a perspective view of a sample receiver comprising a sample receiver silo 410 with a scraper 406 disposed therein. FIG. 20B shows a cross-sectional view of the assembled sample receiver including swab cap 404 disposed on the sample receiver silo 410 to seal the chamber 2001 therewithin. FIG. 20C shows a perspective underside view of a swab cap 404 that is configured to hold a sample collecting swab (e.g., a nasopharyngeal swab) and couple to the sample receiver silo 410 after the sample has been collected by the swab. The swab may be disposed within a swab channel 412 extending from the swab cap 402 to the bottom of the sample receiver silo 410. The swab cap 404 may be configured to mate with the portion of the housing 408 surrounding the sample interface 103, e.g., the sample receiver silo 410. The swab can be connected to the receiver cap 404 prior to use and, after collection, the swab can be inserted into the sample receiver 103 and the cap 404 can be secured (e.g., screwed onto, press-fit, etc.) to the silo 410 to seal the chamber 2001 (e.g., sample reservoir 501) therein. The swab is preferably retained in the sample receiver 103 throughout the remainder of the device's use. One or more locking features may be provided on the sample receiver 103 and/or sample collector to prevent removal of the sample collector and/or exposure of the contents of the sample receiver chamber 2001 to the system or a user. The sample interface 103 may be configured to facilitate collection of the sample from the swab. In some embodiments, the sample interface 103 for swab may contain one or more features configured to facilitate elution of the sample (e.g., virions or cells) from the swab.

In some embodiments, the sample interface 103 may comprise a scraper 406 as described herein. The scraper 406 may agitate the swab when the swab is inserted into the sample interface 103 in order to physically displace the sample from the swab and transfer the sample into the cartridge 102. The scraper 406 may be disposed within the chamber of the sample receiver silo 410 and may define a channel 412 into which a sample collector (e.g., swab 402) is disposed when the sample receiver 103 is in use. The scraper 406 may include geometric interfaces (e.g., annular rings along the long axis of the scraper, spiral rings along the long axis of the scraper, vertical rungs running parallel to the long axis of the scraper, protrusions, etc.) disposed within the swab channel 412 and configured to extract the sample from the swab. The geometric features may scrape the swab as it passes into the channel 412 and agitate the swab in order to physically displace the sample from the swab and transfer the sample into the chamber 2001 of the sample receiver 103. It will be apparent to one of ordinary skill in the art that the scraper 406 may comprise a variety of forms, shapes, and sizes while retaining its function of agitating the input sample. In some embodiments, the sample interface 103 may comprise a sample reservoir 501 into which the sample may be transferred from the sample collector. The sample receiver chamber or reservoir 2001 (e.g., sample reservoir 501) can include a liquid (e.g., lysis buffer, nucleic acid extraction buffer, etc.) prior to insertion of the swab into the channel or a liquid can be added after the swab has been inserted and the chamber 2001 has been sealed. In at least some instances, it may be preferred to insert the sample collector into a dry sample receiver and then add liquid later so as to improve usability and/or reduce the change of contact between the reagents and the environment outside the cartridge. In some embodiments, flushing a liquid over the swab can facilitate sample collection. In some cases, the sample receiver can comprise, or be coupled to, an ultrasonic horn configured to further agitate and/or lyse the sample. Alternatively, or in combination, the sample receiver can comprise a vent and be coupled to a gas source in order to enable bubbling within the chamber to further agitate and/or mix the sample liquid therein. In some embodiments, the sample interface 103 may be connected by a liquid channel (e.g., via a rotary valve, jumper valve, etc.) to a reservoir containing extraction and/or lysis liquids. In some embodiments, the sample interface may be thermally coupled to a heater of the instrument in order facilitate sample transfer from the sample collector into the cartridge.

In some embodiments, a liquid specimen may be input into the sample interface 103 instead of a swab. In such embodiments, the sample interface 103 may be configured to receive liquids via a transfer pipette or other fluid transfer mechanism as will be understood by one or ordinary skill in the art.

In some embodiments, the fluid volume in the sample reservoir may vary from about 100 μL to 2 mL. In some embodiments, the fluid volume in the sample reservoir may be at least about 100 μL, about 200 μL, about 300 μL, about 400 μL, about 500 μL, about 600 μL, about 700 μL, about 800 μL, about 900 μL, about 1000 μL, about 1.1 mL, about 1.2 mL, about 1.3 mL, about 1.4 mL, about 1.5 mL, about 1.6 mL, about 1.7 mL, about 1.8 mL, about 1.9 mL, or about 2 mL.

In some embodiments, the sample receiver interface may comprise a cylindrical silo 410 with one end open for sample collection. In some embodiments, the silo 410 may be oval-shaped. In at least some cases, it may be preferable to avoid sharp internal angles within the silo in order to avoid issues when sealing the interface and/or collecting the sample from the sample reservoir/chamber defined thereby.

### C. Sample Concentrator

The cartridge may comprise a sample concentrator. Any of the cartridges described herein may comprise any of the sample concentrators described herein. In some embodiments, the sample concentrator may be downstream of the sample interface and may comprise one or more structures configured to capture, purify, and/or concentrate nucleic acids of the sample. In some embodiments, the sample concentrator may be upstream of the amplification region. In some embodiments, the sample concentrator may be in a sample concentration module. In some embodiments, the sample concentrator may be in a reagent module. In some embodiments, the sample concentration may be in the sample reservoir, a jumper, or a concentrator reservoir. In some embodiments, the sample concentrator may be in fluid communication with one or more reagent reservoirs comprising one or more nucleic acid concentration reagents. In some embodiments, the concentrator may comprise one or more of a filter, a membrane, a column, a mesh, a surface, or one or more beads configured to capture nucleic acids dispose within or downstream of the sample reservoir and configured to capture, purify, and/or concentrate nucleic acids from the sample.

In some embodiments, the sample concentrator may be configured to facilitate liquid-liquid extraction, solid-liquid extraction, and/or solid-phase extraction techniques. In at least some instances, solid-phase extraction may be preferred for smaller sample volumes and/or improved cartridge-based performance (e.g., by reducing solvent volumes and/or simpler workflow automation).

Solid-phase nucleic acid purification may utilize a silica-based method such as the Boom method. In some embodiments, the concentrator may comprise one or more silica particles (e.g., silica beads or silica magnetic beads), glass fiber filters, silica-coated membranes/meshes/filters, or the like. Application of chaotropic salts such as guanidinium thiocyanate or guanidinium hydrochloride may facilitate adsorption of the nucleic acids onto the silica-based concentrator. Alcohol may be used to wash away salts and cellular debris that may contaminate or inhibit downstream reactions such as amplification. The purified nucleic acids may then be eluted off the concentrator (e.g., using a moderate salt buffer).

Alternatively, or in combination, solid-phase nucleic acid purification may utilize charge switching for nucleic acid concentration. In some embodiments, the concentrator may comprise one or more particles (e.g., beads or magnetic beads) or surfaces (e.g., membranes, meshes, filters, etc.) functionalized with an ionizable material such as chitosan. The ionizable material may, for example, be provided as a coating on the one or more particles or surfaces. The ionizable material may be pH-responsive and allow nucleic acid capture in moderately low pH (in which the ionizable material is cationic) and nucleic acid release in moderately high pH. In some instances, the purified nucleic acids may be eluted off the concentrator with a buffer compatible with downstream reactions such as amplification. In at least some instances, charge switching may provide a less complicated workflow and/or reduce or eliminate the use of inhibiting reagents compared to some other purification methods.

In some embodiments, the cartridge as described herein may comprise a sample concentration module. In some embodiments, the sample concentration module provides some similar functions of the sample receiver module. In some embodiments, the concentration module may comprise a concentration reservoir comprising one or more of a filter, a column, a membrane, a mesh, a surface, or one or more beads configured to capture nucleic acids.

The concentrator may comprise one or more of a filter, a column, a membrane, a mesh, a surface, or one or more beads configured to capture nucleic acids. In some embodiments, the beads may be silica beads. In some embodiments, the beads may be silica-coated beads. In some embodiments, the beads may be silica-coated magnetic beads. In some embodiments, the beads may be chitosan-coated beads. In some embodiments, the beads may be chitosan-coated magnetic beads. In some embodiments, the filter may be a glass fiber filter. In some embodiments, the membrane may be a chitosan-functionalized membrane (e.g., a nylon membrane coated with chitosan). In some embodiments, the concentrator may comprise more than one type of material configured to capture nucleic acids. For example, the concentrator may comprise one or more silica bead and one or more chitosan-coated beads.

In some embodiments, the concentrator may comprise one or more magnetic beads. In some embodiments, the concentrator may comprise one or more silica-coated magnetic beads. In some embodiments, the sample concentration module includes a liquid reservoir configured to interface with a magnet on the instrument to immobilize the magnetic beads to a surface of the cartridge. In some embodiments, the magnet may be moved away from the cartridge by the instrument when the beads are to be released downstream to the amplification module. In some embodiments, the cartridge may contain a filter mesh downstream of the magnet to capture the magnetic beads. In some embodiments, a liquid back flow may move the magnetic beads functionally downstream to the amplification module. In embodiments, the magnetic beads may remain immobilized throughout the sample concentration process and the sample nucleic acids may be eluted therefrom and flown downstream while the magnetic beads remain immobilized in the concentration region.

In some embodiments, sample concentration may include one or more washing steps (e.g., to remove unwanted proteins and/or salts from the nucleic acids bound to the concentrator).

In some embodiments, concentrated nucleic acids with 260/280 and 260/230 ratios may be released by the concentrator by an elution buffer as described herein. In some embodiments, during washing, the sample concentrator may retain bound nucleic acids to beads in a filter mesh or with a magnet as described herein in order to allow for a thorough elimination of unwanted salts and proteins that might interfere with downstream amplification and/or detection reactions. In some embodiments, the beads may be magnetized. In some embodiments, the beads may not be magnetized. In some embodiments, the last step of the concentration may include a release of the nucleic acids from the magnetic beads or filter. The elution liquid may be a low to no salt reagent configured to release the nucleic acids from the concentrator surfaces.

In some embodiments, any of the cartridges described herein may comprise one or more reagents for concentration of nucleic acids. In some embodiments, one or more of the concentration reagents may be liquid reagents. In at least some instances, liquid reagents for concentration of nucleic acids may purify and/or concentrate the nucleic acids with a high degree of purity. In some embodiments, the liquid reagents may include a series of buffers having different pH. In some embodiments, the liquid reagents may include a series of wash reagents of different ionic strength with alcohols of different concentrations. In some embodiments, high purities of nucleic acids may be obtained by gradually changing the ionic strength and alcohol concentration of the reagents until the final reagent, known as the elution buffer, facilitates the release of the purified nucleic acids from the concentrator. In some embodiments, the purity of the released nucleic acids may be measured spectrophotometrically at 260 nanometers. In some embodiments, impurities may be measured at 230 nanometers and 280 nanometers. In some embodiments, ratios of signal at 260/280 should be 1.8 for DNA and 2.0 for RNA. Lower 260/280 ratios may indicate a low capture of nucleic acid or a high amount of protein in the eluate. In some embodiments, ratios of signal at 260/230 should be within a range of about 2.0 to 2.2. In some instances, a low signal at 260/230 could mean a high amount of salt such as guanidine in the elution. In some embodiments, unwanted salts or proteins can inhibit the downstream amplification and contamination reactions.

In some embodiments, the reagents for concentration may be housed in a different set of reagent capsules 601 (e.g., on a different reagent module) than the initial sample preparation reagents (e.g., lysis reagents, etc.) as described herein. As will be understood by one of ordinary skill in the art, the number and volume of reagent capsules 601 used for nucleic acid concentration may be different (e.g., fewer in number) and/or of a larger or smaller volume than those used for sample collection and lysis.

In some embodiments, the number of capsules for a concentration module may vary from 4 to 8 in number. In some embodiments, there may be at least 1, 2, 3, 4, 5, 6, 7, 8, 9 or 10 capsules.

In some embodiments, the fluid volume in each capsule may vary from 100 μL to 1 mL per capsule. In some embodiments, the fluid volume in each capsule may be at least about 100 μL, about 200 μL, about 300 μL, about 400 μL, about 500 μL, about 600 μL, about 700 μL, about 800 μL, about 900 μL, or about 1000 μL.

In some embodiments, the cartridge may comprise a filter, mesh, magnet, etc. configured to prevent downstream travel of the concentrator(s). In some embodiments, the concentrator may comprise the filter or mesh. In some embodiments, the concentrator may comprise a magnetic particle capable of being immobilized with a magnet of the instrument. In some embodiments, the concentrator may comprise a particle sized and shaped for capture by the filter or mesh.

### D. Jumpers

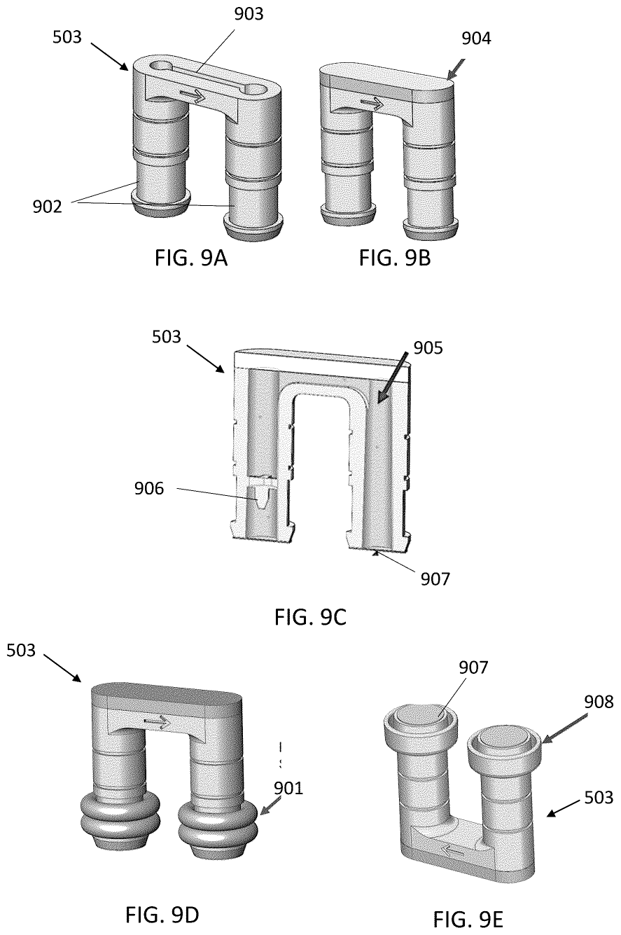

FIGS. 9A-9E show an exemplary fluid channel jumper valve 503 configured to regulate fluid flow within the cartridge. FIG. 9A shows a perspective view of a jumper 503. The jumper 503 may comprise two cylindrical channels 902 joined to one another at their respective proximal ends by a horizontal channel 903. In some embodiments, the jumper 503 may comprise two ovoid channels joined to one another at their respective proximal ends by a horizontal channel 903. FIG. 9B shows a perspective view of the jumper 503 further comprising a proximal surface coupled the horizontal surface to complete the jumper 503. In some embodiments, the top may be sealed 904 by welding a flat piece onto the horizontal channel 903 to complete the u-shaped channel 905. FIG. 9C shows a perspective cross-sectional view of the jumper 503. The two cylindrical channels 902 and horizontal channel 903 may form a u-shaped fluid pathway 905 through which fluid may flow. In some embodiments, the jumper 503 may be configured for unidirectional fluid flow. In some embodiments, the jumper 503 may be configured for bidirectional fluid flow. Fluid flow may be regulated by a seal pin 906 disposed within one of the cylindrical channels 902 as described herein. In some embodiments, the distal ends of the cylindrical channels 902 may be configured to interface with jumper silos on the solid support of the cartridge as described herein. FIG. 9D shows a perspective view of a jumper 901 comprising elastomer seals 901 around the distal ends of each cylinder 902 for interfacing with their respective jumper silos. FIG. 9E shows a perspective view of an alternative sealing embodiment utilizing flexure seal rings 908 around the distal ends of each cylinder 902 for interfacing with their respective jumper silos. In some embodiments, one or both of the distal ends of the cylindrical channels 902 may be sealed with a pierceable seal 907 (e.g., a foil seal) so as to prevent unintentional fluid movement therethrough or early release of jumper-stored reagents therefrom.

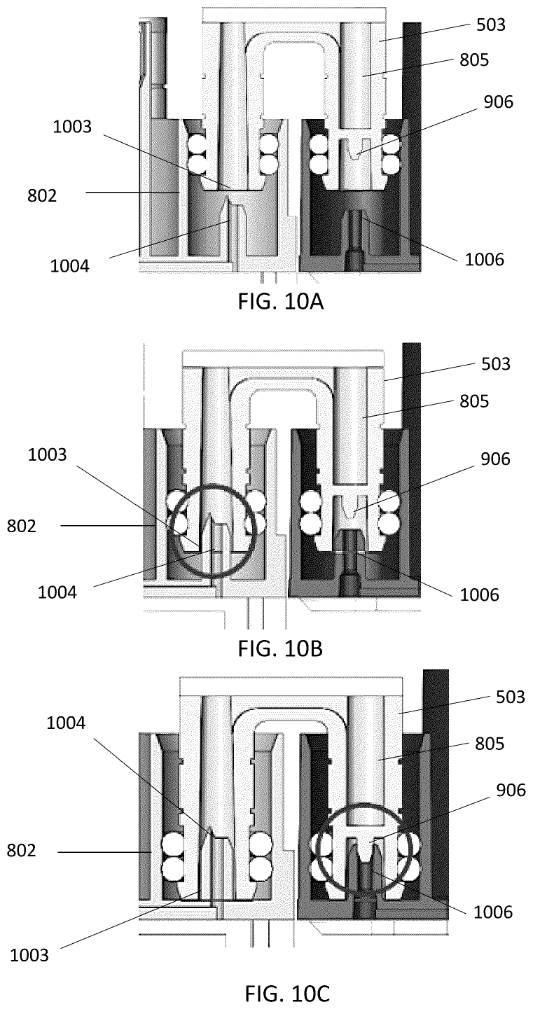

FIGS. 10A-10C shows cross-sectional views of a jumper 503 in use. FIG. 10A shows the jumper 503 in an initial closed configuration prior to actuation. A first end of the jumper 503 may be located within a first jumper silo 802 and a second end of the jumper 503 may be located within a second jumper silo 802. In the initial closed configuration, the jumper channel may not be in fluid communication with channels coupled to the jumper silos 802. The first end of the jumper 503 may be slidably disposed within the first jumper silo 802 and the second end of the jumper 503 may be slidable disposed within the second jumper silo 802. FIG. 10B shows the jumper 503 after actuation to an open configuration which links previously discontinuous fluid pathways together and enables fluid flow there between via the jumper channel 805. Translating the jumper 503 relative to the jumper silo 802 may move the jumper 503 from the initial closed configuration to the open configuration. In the open configuration, the jumper channel 805 may be in fluid communication with one or more channels or chambers coupled to the jumper silos 802 and fluid flow may be allowed therethrough. FIG. 10C shows the jumper 503 after further actuation to a final closed configuration which shuts off fluid flow therethrough. Further translating the jumper 503 relative to the jumper silo 802 may move the jumper 503 from the open configuration to the second closed configuration. In some embodiments, translation of the jumper 503 relative to the jumper silo 802 may be facilitated by an actuator on the cartridge and/or instrument (e.g., the XYZ gantry described herein).

In some embodiments, any of the modules described herein may be regulated by one or more jumpers 503 spanning adjacent modules (e.g., jumpers disposed in silos disposed on different modules). In some embodiments, fluidic channel jumpers 503 can be used to link discontinuous fluid channels together (acting as a valve), thereby enabling establishment of fluid pathways across different modules. In some embodiments, pathways can be opened and/or closed at pre-determined times/intervals in order to affect the desired fluid movement within the cartridge. In some embodiments, jumpers 503 may start in a closed position such that the modules are not prevented from flowing fluid therebetween. The jumpers 503 can then be depressed (e.g., with instrument's XYZ gantry) to first stage (e.g., the open configuration as shown in FIG. 10B), where a piercer comprising a piercer port 1004 may pierce the pierceable cover disposed over the jumper port 1003 at the distal end of the first end of the jumper 503. Engagement of the jumper port 1003 with the piercer port 1004 may open the jumper channel 805 and connect ports on different modules to allow one-directional or bi-directional fluid movement therebetween. In some embodiments, a pin plug comprising a pin plug port 1006 of the second silo 802 may also engage a second jumper port 1003 at the distal end of the second end of the jumper 503. In some embodiments, pressing down to the second stage (e.g., the closed configuration shown in FIG. 10C) may engage the seal pin 906 disposed adjacent the second end of the jumper 503 with a pin plug port 1006 of the second silo and close the jumper channel 805 to prevent further fluid movement therethrough.

In some embodiments, one or more reagents can be stored inside the jumper 503. The one or more reagents may have any formulation described herein (e.g., gas, liquid, pellet, dry powder, etc.). In at least some instances, it may be preferable to store dry or pelleted reagents within the jumper valve channel that may be reconstituted when a liquid is flown therethrough.

In some embodiments, a concentrator (e.g., one or more beads, columns, membranes, meshes, surfaces, and/or filters) can be added to the jumper 503 to capture nucleic acids flowing therethrough in order to facilitate nucleic acid purification as described herein.

In some embodiments, the two cylindrical channels may be sealed against two jumper silos 802, respectively, with a sealing mechanism (e.g., an elastomer seal, a flexure seal ring, an interference fit seal, etc. as depicted in FIGS. 9D-9E, 21, and 22A-22C). Sealing of the jumper channels 902 to the jumper silos 802 may facilitate translation of the jumper channels 802 relative to the jumper silos 802 while maintaining adequate contact therebetween so as to prevent unwanted disengagement thereof (e.g., via transport and handling).

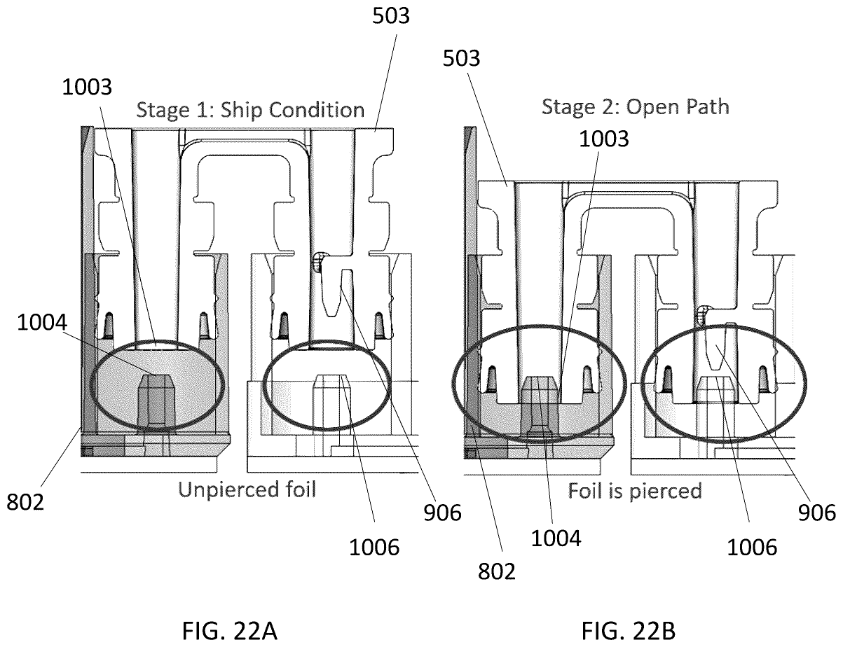

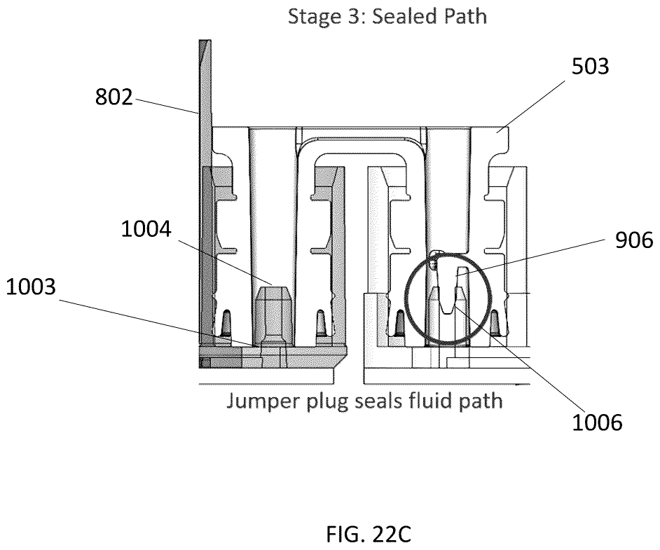

FIGS. 22A-22C show cross-sectional views of a jumper 503 in use. FIG. 22A shows the jumper 503 in an initial closed configuration prior to actuation. A first end of the jumper 503 may be located within a first jumper silo 802 and a second end of the jumper 503 may be located within a second jumper silo 802. FIG. 22B shows the jumper 503 after actuation to an open configuration which links previously discontinuous fluid pathways together and enables fluid flow there between via the jumper channel as described herein. FIG. 22C shows the jumper 503 after further actuation to a final closed configuration which shuts off fluid flow therethrough. The jumper 503 may be substantially similar to the jumper in FIGS. 9A-10C, with the addition of press-fit or interference-fit sealing mechanism(s) configured to seal the jumper 503 to its jumper silo 802 when disposed therein. The jumper silo 802 may be substantially similar to the jumper in FIGS. 9A-10C, with the exception that the piercer port 1004 and the pin plug port 1006 are correspondingly shaped so as to be interchangeable, which may enable greater flexibility of cartridge design and/or manufacturing but otherwise have little to no effect on the functions thereof. The jumper 503 may comprise two cylindrical or ovoid channels joined to one another at their respective proximal ends by a horizontal channel and sealed at the top to form a generally u-shaped channel as described herein. Translating the jumper 503 relative to the jumper silo 802 may move the jumper 503 from the initial closed configuration to the open configuration where a piercer comprising piercer port 1004 may pierce the pierceable cover disposed over the jumper port 1003 at the distal end of the first leg of the jumper 503 and a plug pin comprising a pin plug port 1006 may pierce a pierceable cover disposed over the jumper port 1003 at the distal end of the second leg of the jumper 503. In the open configuration, the jumper channel may be in fluid communication with one or more channels or chambers coupled to the jumper silos 802 and fluid flow may be allowed therethrough as described herein. Further translating the jumper 503 relative to the jumper silo 802 may move the jumper 503 from the open configuration to the second closed configuration in which the seal pin 906 disposed adjacent the second end of the jumper 503 engages with a pin plug port 1006 of the second silo and closes the jumper channel to prevent further fluid movement therethrough as described herein.

E. Amplification region

The cartridge may comprise an amplification region. Any of the cartridges described herein may comprise any amplification region described herein. In some embodiments, the amplification region may be downstream of the sample reservoir, reagent reservoir(s), and/or concentrator. In some embodiments, the amplification region may upstream of the detection region. In some embodiments, the amplification region may overlap with the detection region (e.g., for assays in which amplification and detection occur in the sample location). In some embodiments, the amplification region may be in an amplification module. In some embodiments, the amplification region may be in an amplification/detection module. The amplification region may comprise one or more amplification chambers or channels (e.g., as shown in FIGS. 4D-4E and FIGS. 23A-24C). In some embodiments, the amplification region may comprise one or more amplification reagents. Alternatively, or in combination, one or more amplification reagents may be stored in one or more reagent reservoirs and mixed with the sample prior to, during, or after transfer of the sample to the amplification region. In some embodiments, the system may be configured to amplify one or more target nucleic acids in the amplification region using the one or more amplification reagents. For example, one or more target nucleic acids may be specifically reproduced in the amplification via an isothermal reaction, such as LAMP, or a thermocycling reaction, such as polymerase chain reaction (PCR), as described herein. In some embodiments, the amplification reaction may include reverse transcription (RT), for example when amplifying an RNA target nucleic acid.

In some embodiments, the amplification region may comprise two or more independently controllable temperature zones corresponding to two or more separate channels or chambers therein. In some embodiments, the amplification region may include at least three separate channels or chambers with independent temperature control. In at least some instances, independent temperature control may facilitate the performance of different amplification reactions in each of the three zones.

In some embodiments, one or more amplification reagent (e.g., one or more of amplification enzyme and/or oligonucleotide primer) may be deposited in the amplification channel or chamber as a dried, vitrified, or lyophilized reagent. Introduction of the liquid sample (e.g., nucleic acid eluate from the sample reservoir or sample preparation module) to the reagents for amplification may hydrate the dried, vitrified, or lyophilized reagent(s). In some embodiments, one or more amplification reagents may be dried, vitrified, or lyophilized in situ (e.g., after deposition in the amplification channel or chamber or in a jumper upstream of the amplification channel or chamber).

In some embodiments, the amplification reagents may include one or more amplification enzymes, oligonucleotide primers, nucleotides, etc. as described herein. In some embodiments, the reagents may include a small amount of liquid configured to provide a specific amount of activator salts (e.g., metal ions) in liquid form to facilitate the amplification reaction. In some embodiments, the amount of activator salts may vary from 10 μL to 25 μL. In some embodiments, the liquid may be pre-aliquot into three separate volumes for each of the three amplification channels (in embodiments comprising three amplification channels, for example) in the amplification region. In some embodiments, the sample liquid and the amplification reagents may be mixed in the amplification region and/or in the microfluidic channel between the reagent reservoir(s) and the amplification channel or chamber.

FIG. 11B shows an exemplary cartridge arrangement comprising a sample receiver module 502, two reagent modules 505 and 507, and an amplification module 1104. In an exemplary embodiment, a cartridge may comprise a first reagent module 507, a sample receiver module 502 downstream of the first reagent module 507, a second reagent module 505 downstream of the sample receiver module 502, and an amplification module 1104 downstream of the second reagent module 505. The amplification module 1104 may be upstream of a detection module as described herein. The modules may be coupled to one another so as to provide fluid communication therebetween. In some embodiments, fluid communication may be regulated between the modules by one or more valves. For example, one or more fluid jumpers 503 may be used to valve fluid flow between and/or within the modules. The fluid jumpers 503 may be configured to provide one-way or bi-directional fluid flow between the modules. For example, the fluid jumpers 503 may be configured and arranged to regulate fluid flow from a reagent reservoir 417 to the sample reservoir 501 and prevent backflow from the sample reservoir 501 to the reagent reservoir 417. Alternatively, or in combination, one or more fluid jumpers 503 may be configured to regulate fluid flow from the sample reservoir 501 to a waste reservoir 504 on the sample receiver module 502 and prevent backflow from the waste reservoir 504 to the sample reservoir 501.

A sample may be applied to the sample reservoir 501 as described herein. The sample interface may optionally comprise a scraper as described herein. Lysis reagents may be transferred from one or more reagent reservoirs of the first reagent module to the sample reservoir 501 for lysis of the sample as described herein. In some embodiments, one or more additional reagents (e.g., PK for protein lysis, alcohol for nucleic acid aggregation, etc.) may be transferred from the first reagent module to the sample reservoir 501 before the sample is transferred downstream. In some embodiments, nucleic acids of the sample may be captured by a concentrator 1110 (e.g., a filter, column, beads) on the second reagent module 505 (e.g., within a jumper 503 connecting the second reagent module 505 to the amplification module 1104). In some embodiments, the sample may be transferred to a separate sample concentration module comprising a concentrator downstream of the second reagent module 505. Wash and/or elution reagents may be transferred from one or more reagent reservoir 417 of the second reagent module 505 to the concentrator 1110 for washing and elution of the sample as described herein. Elution of the nucleic acids from the concentrator 1110 may move the purified nucleic acids downstream into the amplification module 1104. In some embodiments, in addition to or instead of concentration of the nucleic acids, nucleic acids of the sample may be separated from debris following lysis and/or inactivation by one or more filters (e.g., filters located within a jumper as described herein with respect to concentrators) prior to being flown downstream. The amplification module 1104 may comprise one or more amplification regions 1105. In some embodiments, the one or more amplification regions 1105 may comprise one or more amplification channels or chambers 1111 as described herein (e.g., three chambers as shown). The nucleic acids may be amplified as described herein and then transferred downstream to a detection module. One or more target nucleic acids may then be detected in a multiplexed fashion as described herein.

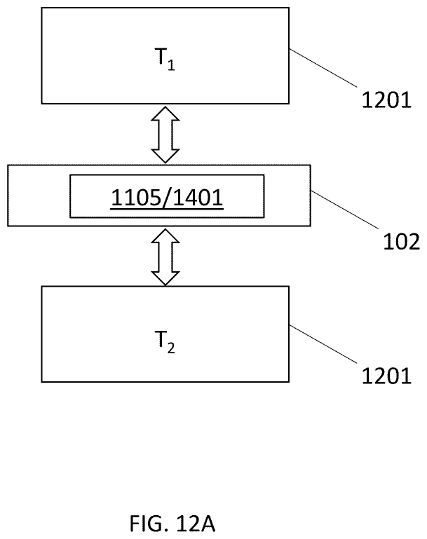

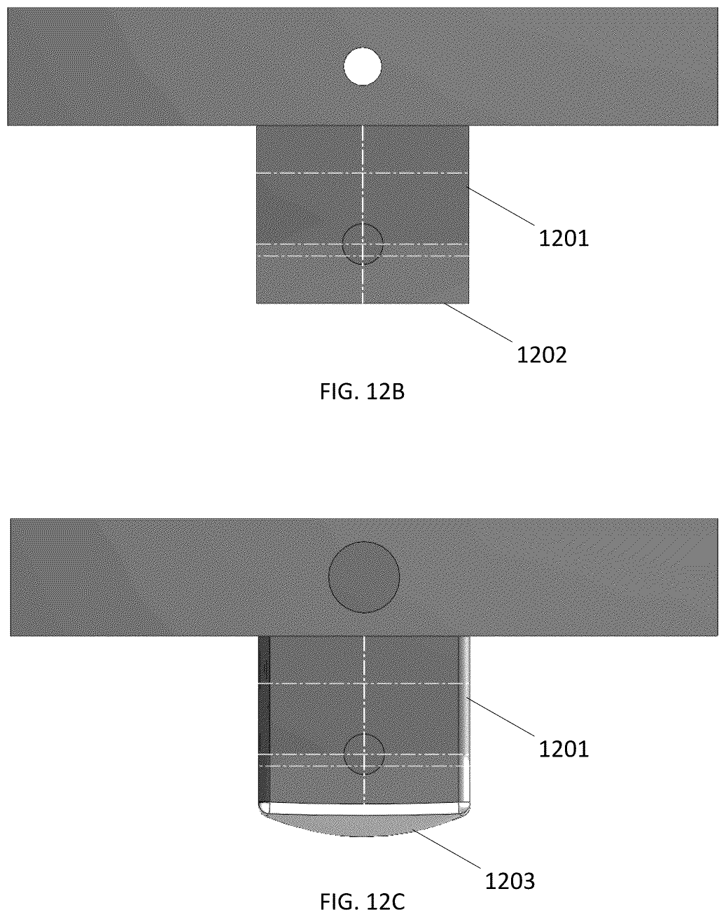

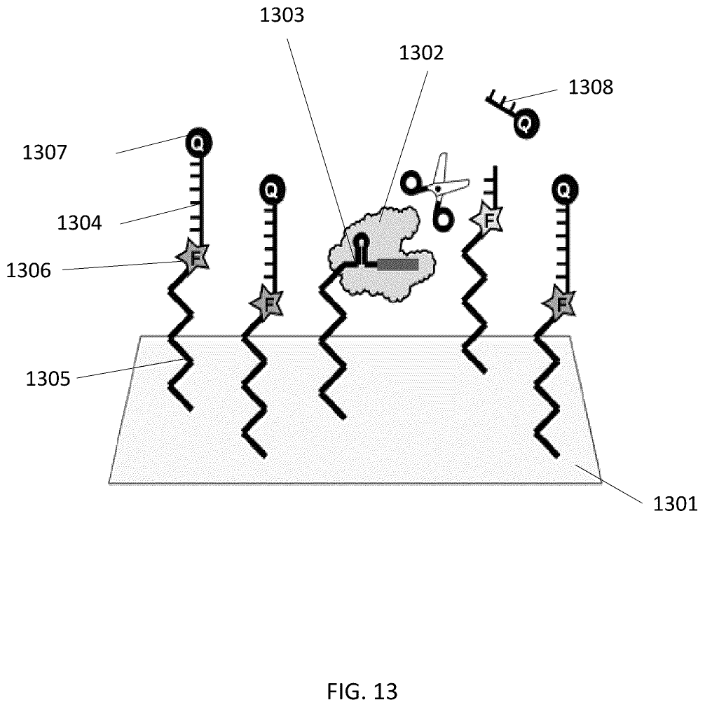

FIG. 12A shows an exemplary thermocycling system comprising a plurality of moveable thermal reservoirs 1201. In some embodiments, the instrument may use one or more thermal reservoirs 1201 to heat and/or cool one or more regions or zones of the cartridge 102 as described herein. In some embodiments, the one or more regions or zones of the cartridge 102 may include a sample interface and/or sample reservoir (e.g., for sample lysis), one or more fluid channels or chambers, an amplification region (e.g., amplification module, chamber, or channel), and/or a detection region (e.g., detection module, chamber, channel, or location). In some embodiments, the one or more thermal reservoirs 1201 may be configured to maintain a desired fluid temperature within a region for a predetermined amount of time. In some embodiments, the one or more thermal reservoirs 1201 may be configured to thermally cycle the fluid temperature within a region for a predetermined number of cycles. For example, a first thermal reservoir 1201 may be held at a first temperature T₁ and a second thermal reservoir 1201 may be held at a second temperature T2. In an alternating manner, the first thermal reservoir 1201 may be contacted to a first surface in thermal communication with a first region or zone of the cartridge and the second thermal reservoir 1201 may be contacted to a second surface in thermal communication with the first region or zone of the cartridge. When in contact with their respective surfaces, heat may flow between the thermal reservoirs 1201 and the fluid within the first region or zone of the cartridge. In this manner, the first and second thermal reservoirs 1201 may perform thermocycling on the fluid within the first region or zone of the cartridge.

FIG. 12B shows a cross-sectional view of a thermal reservoir 1201 comprising a flat contact surface 1202. Any of the thermal reservoirs 1201 described herein may comprise a flat contact surface 1202. The flat contact surface 1202 may be configured to provide good contact with a contact surface of the cartridge in order to conduct heat therethrough as described herein.

FIG. 12C shows a cross-sectional view of a thermal reservoir 1201 comprising a curved contact surface 1203. Any of the thermal reservoirs 1201 described herein may comprise a curved contact surface 1203. The curved contact surface 1203 may be configured to provide good contact with a contact surface of the cartridge in order to conduct heat therethrough as described herein. In at least some instances, it may be beneficial to provide a thermal reservoir 1201 having a curved contact surface 1203 so as to reduce or prevent bending of the cartridge during contact therewith, which may be more likely to occur when a flat contact surface 1202 of the thermal reservoir 1201 is pushed against a flat contact surface 1202 of the cartridge.

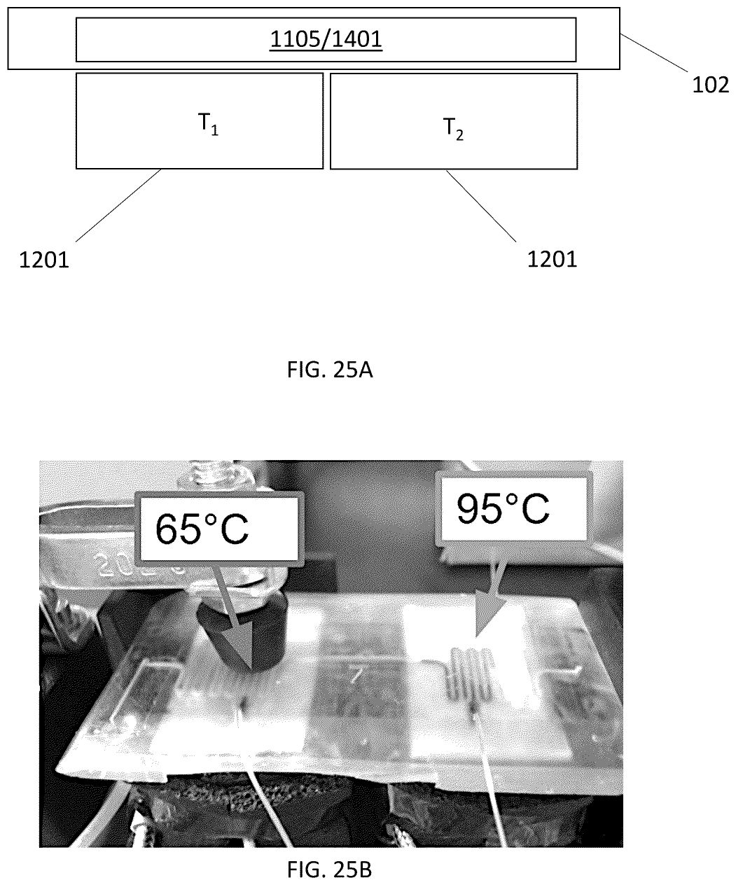

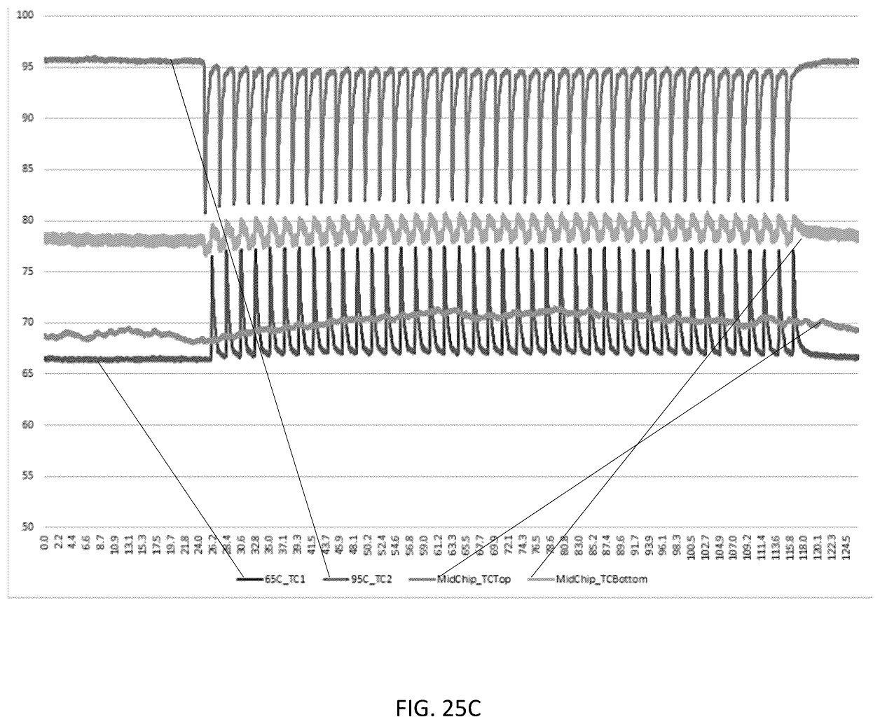

FIGS. 25A-25B shows an exemplary thermocycling system comprising a plurality of thermal reservoirs 1201 and serpentine channels for interzone cycling. In some embodiments, the instrument may use one or more thermal reservoirs 1201 to heat and/or cool one or more regions or zones of the cartridge 102 as described herein. In some embodiments, the one or more regions or zones of the cartridge may include a sample interface and/or sample reservoir (e.g., for sample lysis), one or more fluid channels or chambers, an amplification region 1105 (e.g., amplification module, chamber, or channel), and/or a detection region 1401 (e.g., detection module, chamber, channel, or location). In some embodiments, the one or more thermal reservoirs 1201 may be configured to maintain a desired fluid temperature within a region for a predetermined amount of time. In some embodiments, the one or more thermal reservoirs 1201 may be configured to thermally cycle the fluid temperature within a region for a predetermined number of cycles. For example, a first thermal reservoir 1201 may be held at a first temperature T₁ and a second thermal reservoir 1201 may be held at a second temperature T2. The amplification region may comprise a plurality of fluid channels or chambers configured to be maintained at different temperatures, with fluid being moved from one channel or chamber to another in order to thermally cycle the fluid. The first thermal reservoir 1201 may be in contact with a first temperature zone comprising a first channel and the second thermal reservoir 1201 may be in contact with a second temperature zone comprising a second channel. In some embodiments, the channels may be serpentine so as to increase the surface area of the channel within the amplification region in contact with the thermal reservoirs 1201. In some embodiments, the thermal reservoir(s) 1201 may be in thermal communication with the cartridge as soon as the cartridge is inserted into the instrument. In some embodiments, the thermal reservoir(s) 1201 may be moved into and/or out of thermal communication with the cartridge during the assay (e.g., prior to, during, or after amplification) as described herein. In some embodiments, the first thermal reservoir 1201 may be contacted to a first surface in thermal communication with a first temperature region or zone of the cartridge and the second thermal reservoir 1201 may be contacted to a second surface in thermal communication with a second temperature region or zone of the cartridge. In an alternating manner, the fluid may be pumped back and forth (e.g., using a pneumatic system of the instrument) between the first and second temperature zones in order to thermocycle the fluid.

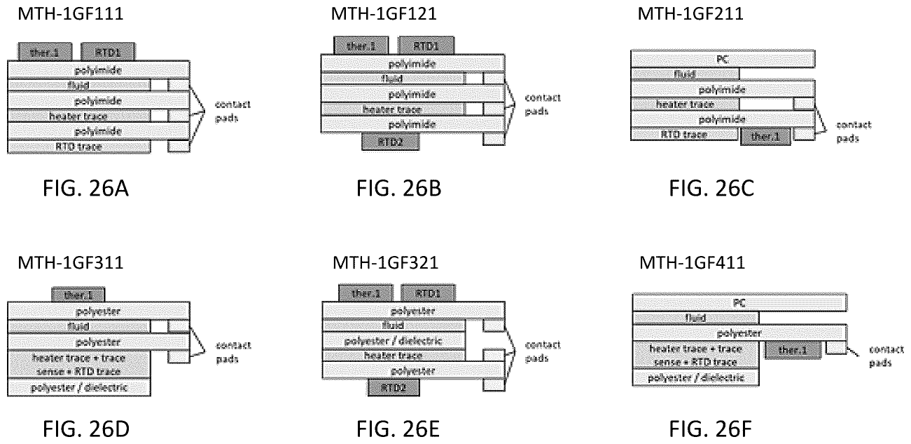

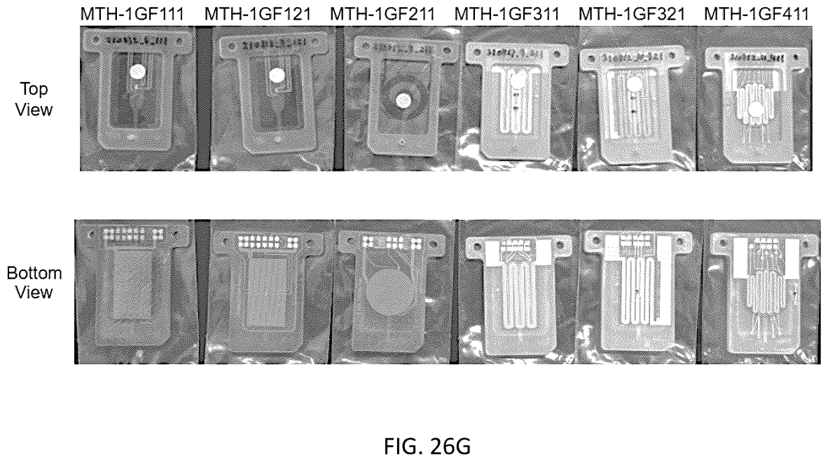

FIGS. 26A-26F show exemplary low mass thermocycling systems. Table 3 describes the various configurations of FIGS. 26A-26F. FIG. 26G shows top and bottom views of the six low mass thermocycling chip configurations of FIGS. 26A-26F. In some embodiments, the cartridge may comprise one or more resistive heating elements and the instrument may be configured to apply an electric current to the one or more resistive heating elements to heat the fluid. In some embodiments, the resistive heating element(s) may be disposed adjacent a surface of one or more regions or zones of the cartridge to be heated. The electrical contacts of the instrument may engage with one or more contact pads of the resistive heating elements of the cartridge upon insertion, thereby achieving electrical communication with the cartridge in order to heat the desired region(s) or zone(s). Rapid cooling may be aided by forced convection. In at least some instances, resistive heating may provide a simple and flexible alternative or addition to other heating mechanisms which may require more complex instrumentation, a larger instrument and/or cartridge footprint/form factor, and/or more complicated fluidic designs.

TABLE 3
Exemplary low mass thermocycling configurations

| | | | | Temperature Sense Type | | | |
|---|---|---|---|---|---|---|---|
| | | Circuit | Channel | Trace | Trace | RTD | Thermistor |
| Figure | Chip | Type | Type | Sense | RTD | Part | Part |
| FIG. 26A | MTH-1GF111 | Flex | Straight | | ✓ | ✓ (1x) | ✓ |
| FIG. 26B | MTH-1GF121 | | | | | ✓ (2x) | ✓ |
| FIG. 26C | MTH-1GF211 | | Circular | | ✓ | | ✓ |
| FIG. 26D | MTH-1GF311 | Screen- | Straight | ✓ | ✓ | | ✓ |
| FIG. 26E | MTH-1GF321 | Printed | | | | ✓ (2x) | ✓ |
| FIG. 26F | MTH-1GF411 | | Circular | ✓ | ✓ | | ✓ |

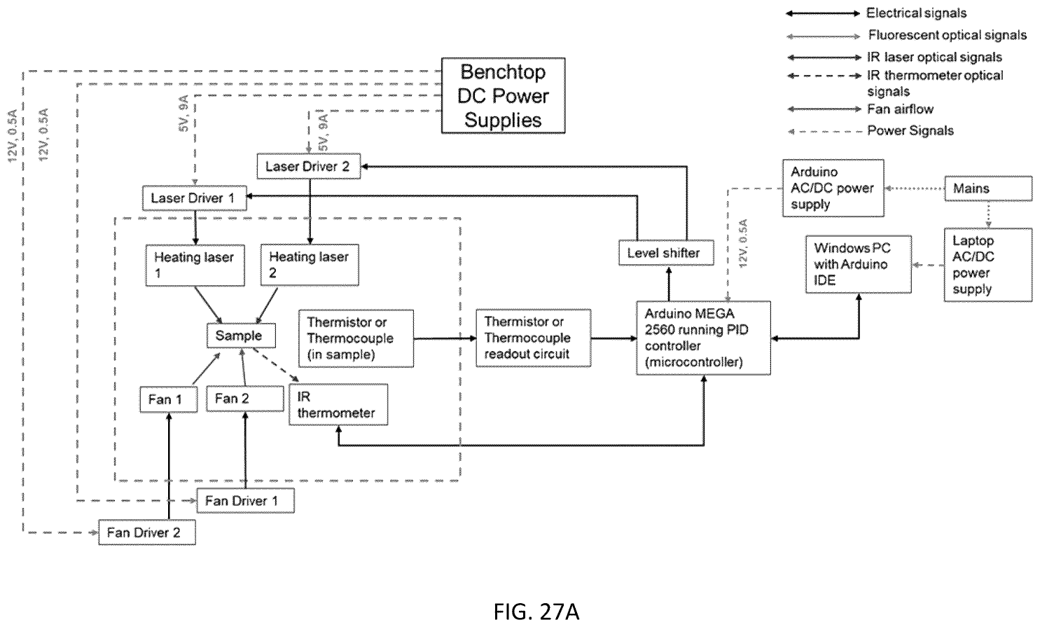

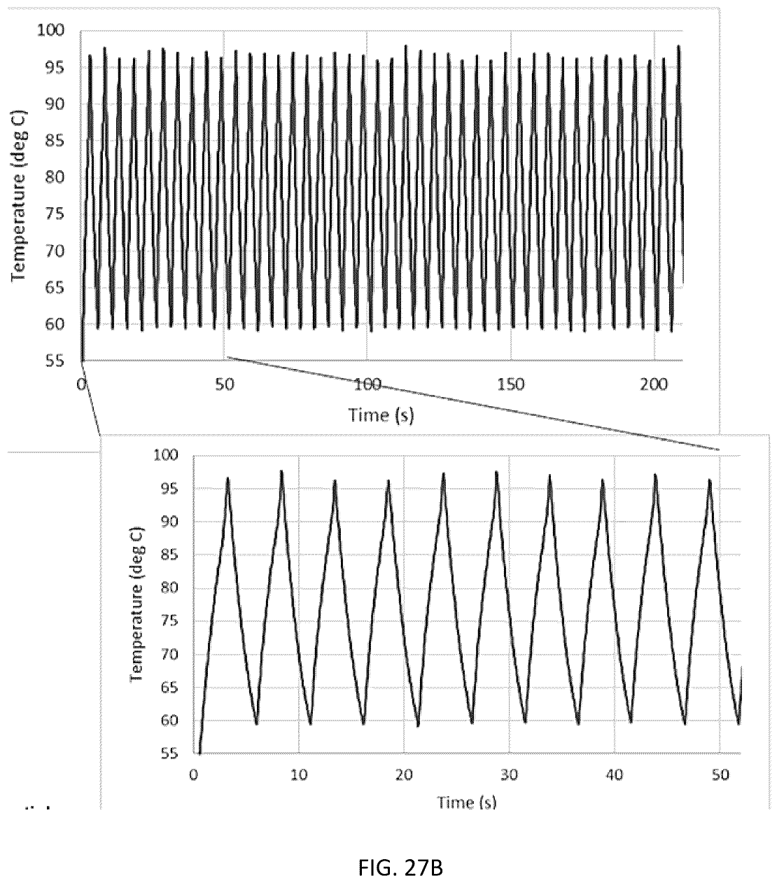

FIG. 27A shows an exemplary photonic thermocycling system. In some embodiments, the instrument uses optical or photonic heating. The instrument may directly heat the reaction mixture by irradiating the liquid with optical wavelengths that are strongly absorbed by water (e.g., infrared wavelengths targeting water absorption peaks below 1500 nm) or by transducing light to heat using particles (e.g., gold nanoparticles) dispersed throughout the heated region(s) or zone(s) of the cartridge (e.g., using light of additional wavelengths). For example, 1450 nm to 1470 nm short wave infrared laser light may be used to heat the sample. In some embodiments, one or more 1465 nm laser diodes may be used to heat the sample as the wavelength is around the peak of absorption for water (assumed primary component of the sample). Cooling may be performed by forced air, while sensing of the liquid temperature may be performed with an infrared sensing thermometer. Since the water absorbs much more strongly than the consumable it resides in, very rapid heating and cooling of the water can be performed. An electronics sub-system may be used to provide power, drive the lasers, and perform the PID temperature control. In at least some instances, direct heating may offer advantages in thermal speed compared to some indirect heating methods by directly heating the reaction mixture instead of controlling the temperature of a surface in contact with the reaction mixture. In at least some instances, direct heating may reduce or avoid time delays associated with fluids inside a chamber equilibrating with a surface temperature.

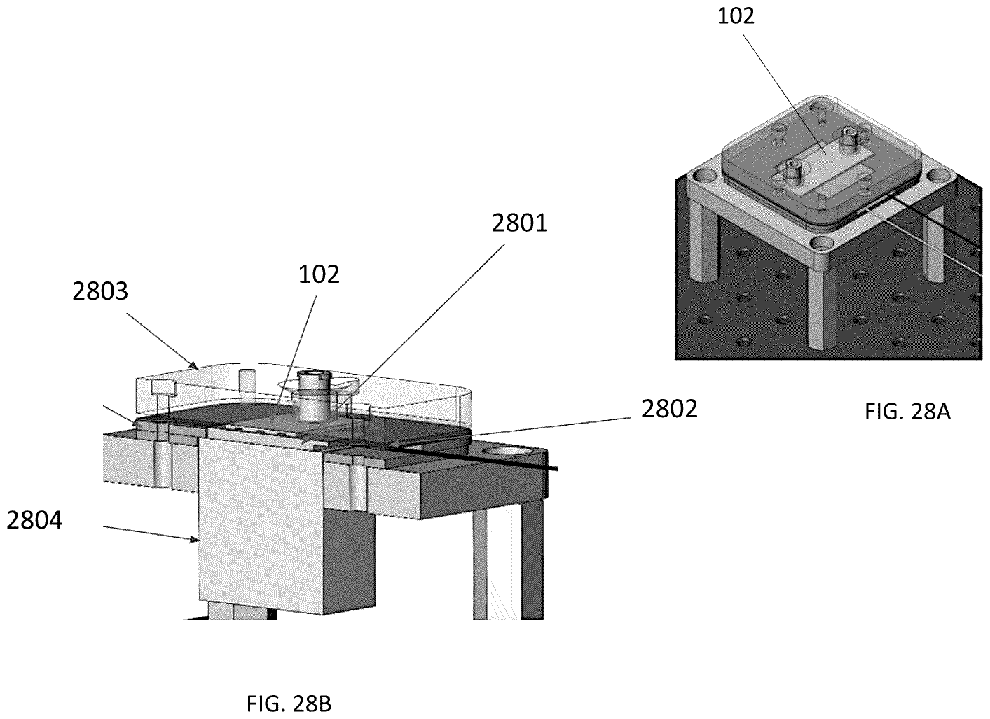

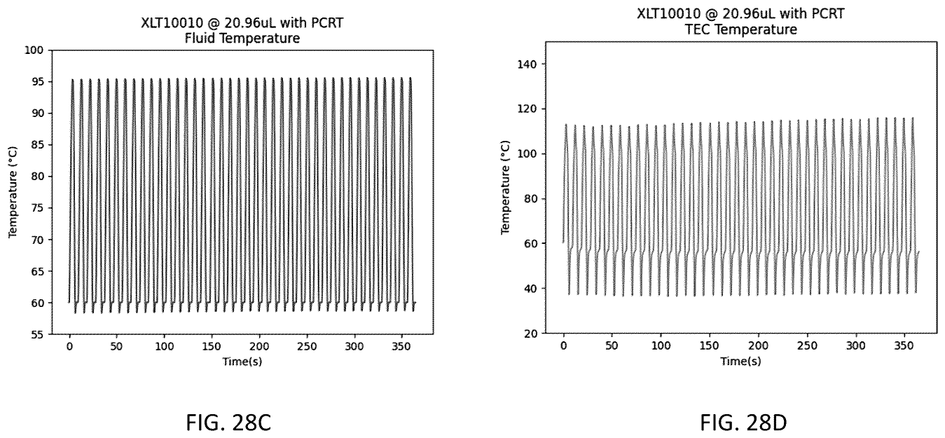

FIGS. 28A-28B show an exemplary thermoelectric thermocycling system. In some embodiments, the instrument uses Peltier/thermoelectric (TEC) coolers 2801 to heat and/or cool one or more regions or zones of the cartridge 102. The TEC coolers 2801 may engage with the cartridge 102 upon insertion, thereby achieving intimate contact with the cartridge 102 at the desired region(s) or zone(s). For example, each temperature zone of the cartridge 102 may be in thermal communication with a TEC cooler 2801 (e.g., three amplification chambers may utilize three individually controllable TEC coolers). In some embodiments, the cartridge 102 may be configured to provide improved thermal conductivity and/or time response. For example, a surface of the cartridge 102 may comprise a thin film for heat exchange with the heater/cooler(s) of the instrument. In some embodiments, the film may be an aluminized film in order to provide improved and/or more consistent thermal transfer. In some embodiments, the film may be a PCR tape or film. In some embodiments, the film may be an aluminized pressure-sensitive adhesive (PSA). In some embodiments, the TEC cooler 2801 may sit on a compressible silicone pad 2802 configured to compress when the cartridge 102 is in contact with the TEC cooler 2801. A compression platen 2803 may be provided by the instrument to apply pressure to the cartridge 102 in order to ensure good contact with the TEC cooler 2801 and consistent thermal communication therebetween. In some embodiments, a heat sink 2804 may be provided between the TEC cooler 2801 and the rest of the instrument to prevent overheating of the instrument during use of the TEC cooler 2801.

### F. Detection Region

The cartridge may comprise a detection region. Any of the cartridges described herein may comprise any detection region described herein. In some embodiments, the detection region may be downstream of an amplification region. In some embodiments, the detection region may be co-localized with the amplification region (e.g., when amplification and detection occur in the same location and/or at the same time). In some embodiments, the detection region may be in a detection module. In some embodiments, the detection region may be in an amplification/detection module. The detection region may comprise one or more detection locations. In some embodiments, a detection location may comprise one or more detection reagents. For example, the detection location may comprise a reporter, a guide nucleic acid, and/or a programmable nuclease. The reporter may be any of the reporters, or any combination of reporters, described herein. The guide nucleic acid may be any of the guide nucleic acids, or any combination of guide nucleic acids, described herein. The programmable nuclease may be any of the programmable nucleases, or any combination of programmable nucleases, described herein. Any one of several programmable nucleases (e.g., Cas proteins) may be used individually or in combination with other programmable nucleases. In some embodiments, the programmable nuclease may comprise a Cas12, Cas13, Cas14, or CasPhi family Cas protein. In some embodiments, the programmable nucleases may be different in the different detection locations.

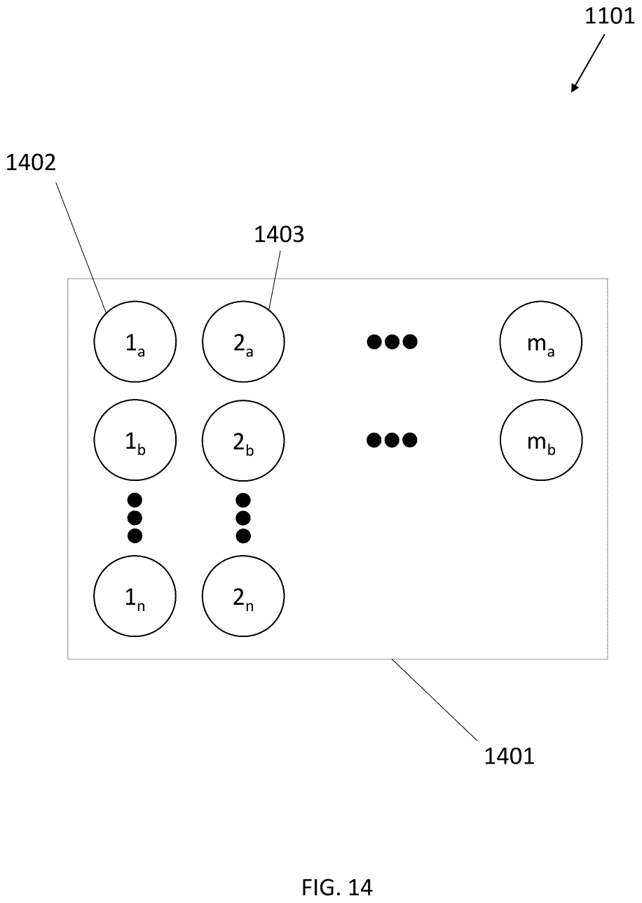

In some embodiments, the detection region may be configured to detect one or more signals from a liquid-based programmable nuclease-based detection reaction or an immobilized array programmable nuclease-based detection reaction as described herein. For example, the detection region may comprise one or more detection channels, chambers, microwells, nanowells, or the like comprising detection reagents suitable for a liquid-based assay (e.g., the fluid channel(s) shown in FIGS. 4D-4E, or the microwells shown in FIG. 14 or 39A-47D). In another example, the detection region may comprise one or more detection locations (e.g., the detection spots, or microwells of FIG. 14 or 39A-47D) comprising one or more immobilized detection reagents.

FIG. 13 depicts a surface 1301 comprising an immobilized programmable nuclease-guide nucleic acid complex (1302, 1303) and a plurality of reporters 1304, with one reporter having been cleaved by an activated programmable nuclease 1302. In some embodiments, one or more detection reagents may be immobilized on a surface 1301 of the detection region. For example, one or more programmable nuclease 1302, one or more guide nucleic acid 1303, and/or one or more reporter 1304 may be immobilized on a surface 1301 of the detection region. In some embodiments, one or more detection reagents may be immobilized on a surface 1301 of the detection region via a linker 1305. The programmable nuclease 1302 may be complexed with a guide nucleic acid 1303 complementary to a specific target nucleic acid sequence as described herein. When a target nucleic acid is present in the sample, the programmable nuclease 1302 may be activated by binding of the guide nucleic acid 1303 (which is complexed thereto) to the target nucleic acid. Activation of the programmable nuclease 1302 may enable trans-cleavage of the reporter 1304 as described herein. In some embodiments, the reporter 1304 may comprise a detection moiety 1306 as described herein. In some embodiments, the reporter 1304 may comprise a detection moiety 1306 (e.g., a fluorophore) and a quencher moiety 1307 (e.g., a quencher) configured to generate a detectable signal when separated from one another by release of a cleaved portion 1308. In some embodiments, trans-cleavage of the reporter 1304 by the activated programmable nuclease 1302 may release the detection moiety 1306 (or a quencher moiety 1307, depending on the signal), thereby generating a signal indicative of the presence or absence of the target nucleic acid in the sample as described herein. The reporter 1304 may comprise any of the reporters 1304 described herein, and may comprise any detection moiety 1306 described herein (and/or other moieties or molecules described herein which facilitate signal detection). As shown in FIG. 13, cleavage of the reporter 1304 may release the quencher 1307 into solution, thereby allowing the fluorophore 1306 which was quenched when the reporter 1304 was intact to fluoresce. The presence of fluorescence at the detection location may therefore indicate the presence of the target nucleic acid. In another example, the reporter 1304 may not comprise a quencher 1307 and cleavage of the reporter 1304 may instead release a fluorophore into solution, thereby reducing fluorescence at the detection location. The absence of fluorescence at the detection location may therefore indicate the presence of the target nucleic acid. In some embodiments, cleavage of the reporter 1304 may result in a non-fluorescent signal as described herein. For example, cleavage of the reporter 1304 can produce a calorimetric signal, a potentiometric signal, an amperometric signal, a colorimetric signal, or a piezo-electric signal.

In some embodiments, the one or more detection reagents can be immobilized in discrete detection locations using NHS-amine chemistry as described herein. For example, a primary amine-modified guide nucleic acid and a primary amine-modified reporter may be conjugated to an NHS-coated surface of the detection region.

In some embodiments, the one or more detection reagents may be immobilized using streptavidin-biotin chemistry as described herein. For example, a biotinylated reporter and a biotinylated guide nucleic acid may be immobilized to a streptavidin-coated surface of the detection region.

In some embodiments, the one or more detection reagents may be immobilized using maleimide-thiol chemistry as described herein. For example, a thiol-modified guide nucleic acid and a thiol-modified reporter may be conjugated to a maleimide-coated surface of the detection region.

In some embodiments, the one or more detection reagents may be immobilized using epoxy-amine chemistry as described herein. For example, an amine-modified guide nucleic acid and an amine-modified reporter may be conjugated to an epoxy-coated surface of the detection region.

In some embodiments, the one or more detection reagents may be immobilized using hydrogels as described herein. For example, an acrydite-modified guide nucleic acid and an acrydite-modified reporter may be co-polymerized with an acrylate-modified oligomer (e.g., PEG-diacrylate) prior to deposition on the surface 1301 of the detection region or in situ on the surface 1301 of the detection region.

FIG. 14 shows an exemplary detection region 1401 of a detection module 1101. In some embodiments, the detection region 1401 may comprise one or more discrete detection locations. The detection location(s) may comprise a detection spot, a detection chamber, a detection channel, a detection well (e.g., a microwell), or the like, or any combination thereof. In some embodiments, the detection region 1401 may comprise a plurality of detection spots or microwells, e.g., a first detection spot or microwell 1402, a second detection spot or microwell 1403, etc., that are spatially separated from one another at discrete detection locations. Spatial separation of the detection locations may enable multiplexed target nucleic acid analysis of the sample within the device.

In some embodiments, one or more detection reagents may be immobilized, dried, or otherwise deposited on a surface of the detection region 1401 at one or more detection locations. For example, one or more programmable nuclease, one or more guide nucleic acid, and/or one or more reporter may be immobilized, dried, in situ lyophilized, or otherwise deposited on a surface of the detection region 1401 at one or more detection locations. Alternatively, or in combination, one or more of the detection reagents at the detection location(s) may be in a dried or lyophilized form prior to mixing with the sample fluid. Each programmable nuclease may be complexed with a guide nucleic acid complementary to a specific target nucleic acid sequence. In some embodiments, each detection location may comprise a different combination of detection reagents so as to provide a plurality of spatially separated multiplex-capable detection locations. For example, one or more (e.g., each) detection location may comprise a different guide nucleic acid configured to bind to a different target nucleic acid. Alternatively, or in combination, one or more (e.g., each) detection location may comprise a different programmable nuclease. Alternatively, or in combination, one or more (e.g., each) detection location may comprise a different reporter. In some embodiments, each detection location may comprise a plurality of reporters. In some embodiments, each detection location may comprise detection reagents configured to detect a different target nucleic acid of a plurality of target nucleic acids.

In some embodiments, one or more detection reagent may be immobilized to a surface (e.g., surface 1301 shown in FIG. 13) of the detection location as described herein. Alternatively, or in combination, one or more detection reagents may be dried, vitrified, or lyophilized and deposited on a surface of the detection location. In some embodiments, deposition of the dried, vitrified, or lyophilized may be accomplished using photolithography, inkjet printing, or other patterning techniques. In some embodiments, one or more detection reagents may be dried, vitrified, or lyophilized in situ (e.g., after deposition on a surface of the detection location).

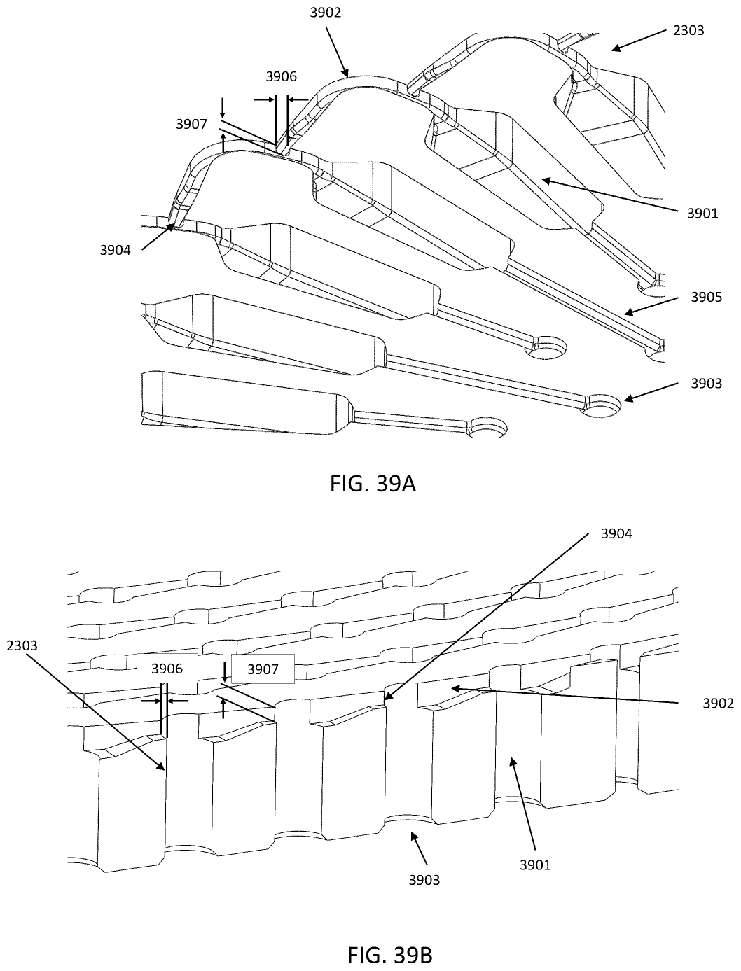

In some embodiments, the reagents may be spotted in a microwell array (e.g., as shown in FIGS. 39A-47D). Specific wells may contain reagents for specific target nucleic acids. In some embodiments, the one or more detection reagents (e.g., the guide nucleic acid and reporter) can be synthesized in arrayed patterns using photolithography.

In some embodiments, the detection locations may be patterned as an array. For example, an array of detection locations may comprise a two-dimensional array of detection spots, chambers, or microwells arranged in orthogonal directions. The array may be an m×n array having m columns of n detection spots, chambers, or microwells arranged in rows. In some embodiments, m and n may be different. In some embodiments, m and n may be the same. In some embodiments, an array of detection locations may be asymmetrical (e.g., detection locations may be patterned to minimize the usage of space in the detection region 1401 with regard for symmetry). It will be apparent to one of ordinary skill in the art that the detection region 1401 may comprise any suitable number of detection locations and the detection locations may be arranged in any suitable manner so as to enable multiplexed target nucleic acid analysis.

In some embodiments, the detection reagents may be provided on a surface of the detection region as an immobilized array. In some embodiments, the array may comprise a number of detection locations within a range of about 1 to about 200, within a range of about 3 to about 200, or within a range of about 10 to about 200. In some embodiments, the array may comprise at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more detection locations.

In some embodiments, one or more locations may comprise the same detection reagents (e.g., detection reagents specific to the same target nucleic acid). In some embodiments, 1 to 12 detection locations may comprise the same detection reagents. For example, an exemplary detection region may comprise anywhere from 1 to 12 replicate spots for each target nucleic acid to be detected.

In some embodiments, the detection region may comprise an array of detection spots. Each detection spot of the array may comprise a reporter and a different programmable nuclease of a plurality of programmable nucleases as described herein. In some embodiments, each of the different programmable nucleases of the plurality of programmable nucleases may comprise a different guide nucleic acid which is complementary to a different target nucleic acid of a plurality of target nucleic acids. In some embodiments, the reporter and each different programmable nuclease and/or guide nucleic acid of each detection spot of the array may be immobilized to a surface of the detection region. In some embodiments, at each detection spot of the array, each different programmable nuclease may be configured to cleave an adjacent reporter and generate a different signal of a plurality of signals. Each different signal may therefore be indicative of the presence or absence of a different target nucleic acid. In some embodiments, the reporter may comprise a fluorophore and a quencher as described herein. The guide nucleic acid and reporter may each be immobilized to a surface of a detection spot with a linker as described herein. The target nucleic acids may be freely available within the fluid volume of the detection region. In some embodiments, multiple guide nucleic acids for a single target nucleic acid may be combined within a single detection spot in order to increase a rate of reaction. Localizing the guide nucleic acids and reporter may localize the detectable signal for each target nucleic acid to the detection spot, thus enabling the spatial multiplexing.

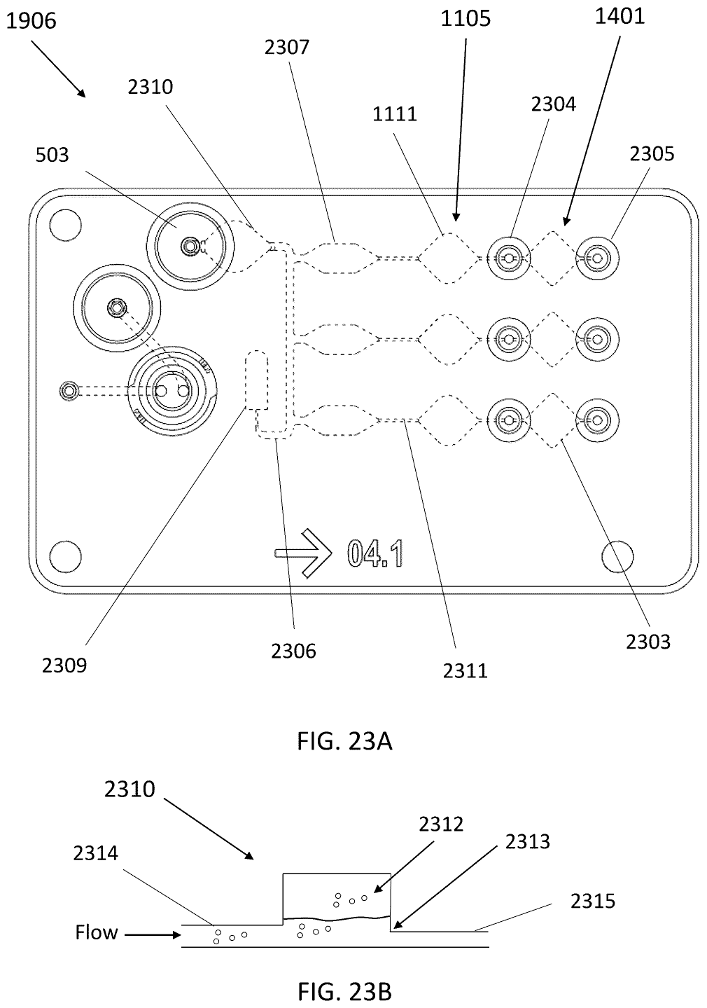

In some embodiments, the detection region may comprise a plurality of chambers (e.g., chambers 2303 shown in FIG. 23A or microwells 3901 shown in FIGS. 39A-47D). In some embodiments, each chamber may comprise a reporter and a different programmable nuclease of a plurality of programmable nucleases. In some embodiments, each of the different programmable nucleases of the plurality of programmable nucleases may comprise a different guide nucleic acid which is complementary to a different target nucleic acid of a plurality of target nucleic acids. In some embodiments, one or more programmable nuclease, one or more reporter, and/or one or more guide nucleic acid may be lyophilized, vitrified, or dried as described herein. In some embodiments, each different programmable nuclease may be configured to cleave an adjacent reporter and generate a different signal of a plurality of signals. Each different signal may therefore be indicative of the presence or absence of a different target nucleic acid as described herein.

In some embodiments, the detection region may comprise a plurality of chambers. Each of the plurality of chambers may have a volume within a range of about 0.5 μL to about 10 ml. For example, each of the plurality of chambers may have a volume of about 0.75 μL, about 1 μL, about 1.5 μL, about 3 μL, about 5 μL, or about 10 μL. The volume of each chamber may be within a range of about 0.5 μL to about 10 ml while maintaining an aspect ratio sufficient to enable filling of the chambers while still preventing cross-talk between chambers as described herein.

In some embodiments, the plurality of chambers may comprise a plurality detection channels, chambers, microwells, nanowells, or the like. For example, the plurality of chambers may comprise a plurality of microwells disposed downstream of an amplification chamber (or downstream of nucleic acid purification if amplification is not performed or is performed in the detection microwells themselves). The plurality of microwells may be arranged and filled in series or in parallel with one another following nucleic acid amplification. For example, the amplification channel may be connected to the plurality of chambers by one or more loading channels. In some embodiments, each loading channel may lead to a single detection chamber. In some embodiments, each loading channel may lead to a plurality of detection chambers (e.g., similar to the loading channel 2306 leading to a plurality of amplification chambers 1111 shown in FIG. 23A). In the illustrative example of FIG. 23A, there are three of each of a loading chamber 2307, an amplification chamber 1101, a lyophilized reagent region 2311, a 1^{st} stage vent 2304, a detection region 1401, and a 2^{nd} stage vent 2305 (though only one of each is labeled for simplicity). In some embodiments, the plurality of detection chambers may begin filling sequentially. In some embodiments, the plurality of detection chambers may be filled simultaneously.

One or more capillary valves (e.g., capillary valve 3904 shown in FIGS. 39A-39B) may be arranged at the inlet to each detection chamber (e.g., microwell 3901 shown in FIGS. 39A-39B). The capillary valves may facilitate controlled loading of detection chambers and/or minimize cross-talk between detection chambers. For example, a loading channel (e.g., loading channel 3902 shown in FIGS. 39A-39B) may be coupled to a plurality of detection chambers disposed in series along the length of the loading channel. A capillary valve may be positioned at an inlet to each of the plurality of detection chambers. The capillary valve inlet area may be much smaller than that of the main loading channel. The fluid typically at least partially (e.g., 25-50%) fills a first detection chamber before the fluid has enough pressure build up to start going into the capillary valve. The fluid may then continue down the path provided by the loading channel from one detection chamber/capillary valve to the next. The narrower the detection chamber gets (given a constant depth, so a higher aspect ratio) the better performing the detection chambers will be at loading evenly and consistently. When the fluid volume input to the detection region is such that the volume is equal to the amount of volume in each detection chamber, air clears the plenum behind each detection chamber. If pressure is held, the fluid in each detection chamber cannot come out of the detection chamber and interfere with the reactions occurring in a separate detection chamber.

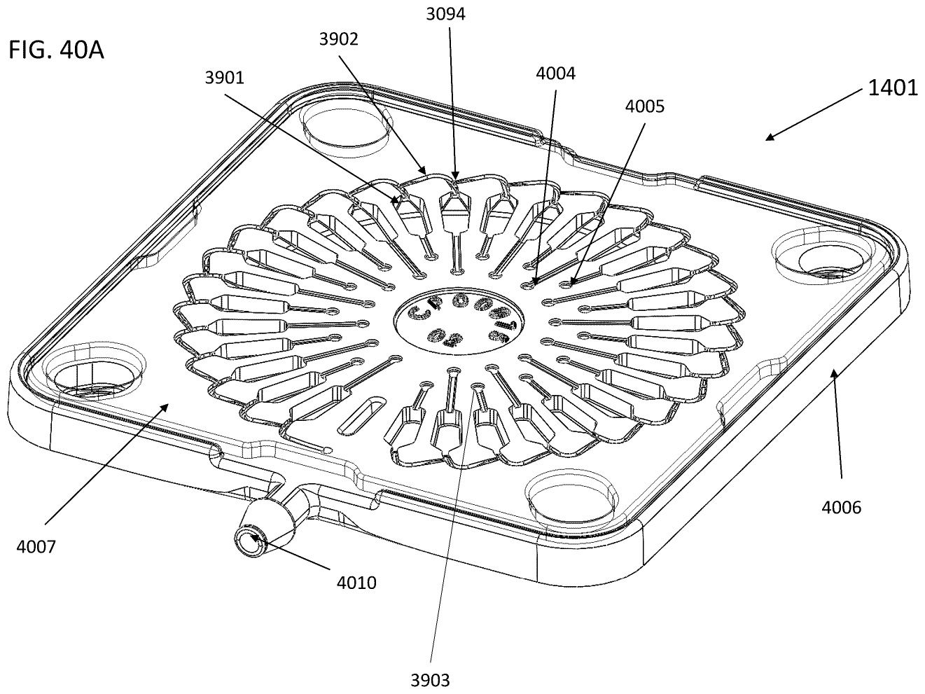

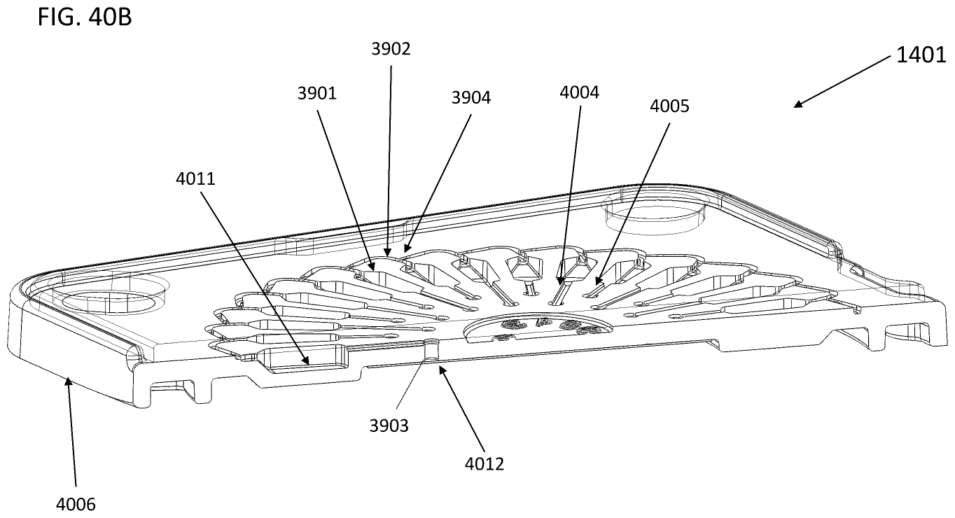

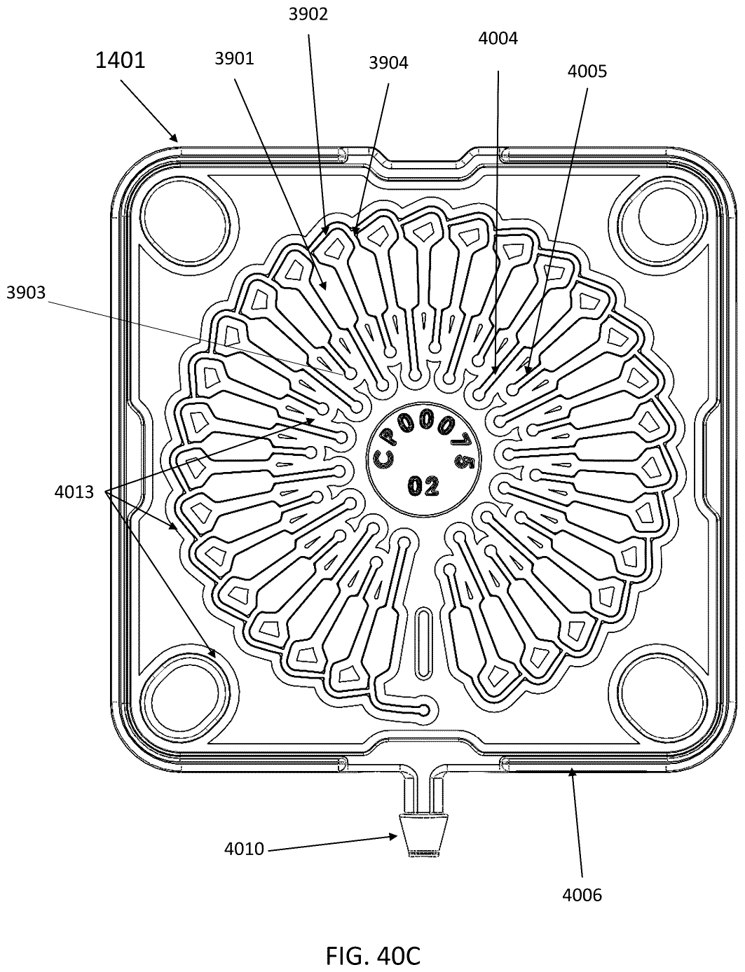

In some embodiments, a surface of the detection region may comprise an air-permeable/liquid-impermeable membrane such as a hydrophobic membrane or a plurality of hydrophobic membranes (e.g., membrane 4012 shown in FIG. 40B). The surface may be a top or bottom surface of the detection region. The surface may interface with a heater or be opposite a surface which interfaces with the heater. The air-permeable/liquid-impermeable membrane may be configured enable gas to pass therethrough for venting but reduce or prevent liquid from exiting the detection region. In some embodiments, each detection chamber (e.g., each microwell) may have a surface (e.g., a bottom surface) comprising air-permeable/liquid-impermeable membrane (e.g., a hydrophobic membrane). The air-permeable/liquid-impermeable membrane may comprise any material which enables gas but not liquid to vent therethrough. In some embodiments, a hydrophobic membrane may comprise a woven polypropylene or polyethylene membrane having an average pore size of about 0.20 microns. The burst pressure of the membrane may be selected based on the loading pressures used to fill the detection region in order to facilitate loading without substantial liquid loss through the membrane. In some embodiments, a sample liquid may include a surfactant or other additive that reduces surface tension (e.g., TWEEN). For liquids with lower surface tension, membranes that are more hydrophobic and/or oleophobic may be preferred in order to avoid wetting. In some embodiments, the membrane comprises polytetrafluoroethylene (PTFE).

In some embodiments, the air-permeable/liquid-impermeable membrane may comprise the surface of the detection region configured to interface with a heater. Without being bound by any particular theory, it is believed that after loading with the reaction liquid the wetted membrane may bring thermal transfer advantages as the liquid causing it to swell may improve thermal transfer between the heater and the reaction liquid during isothermal reactions by improving temperature accuracy, time to reach a desired temperature, and/or temperature stability.

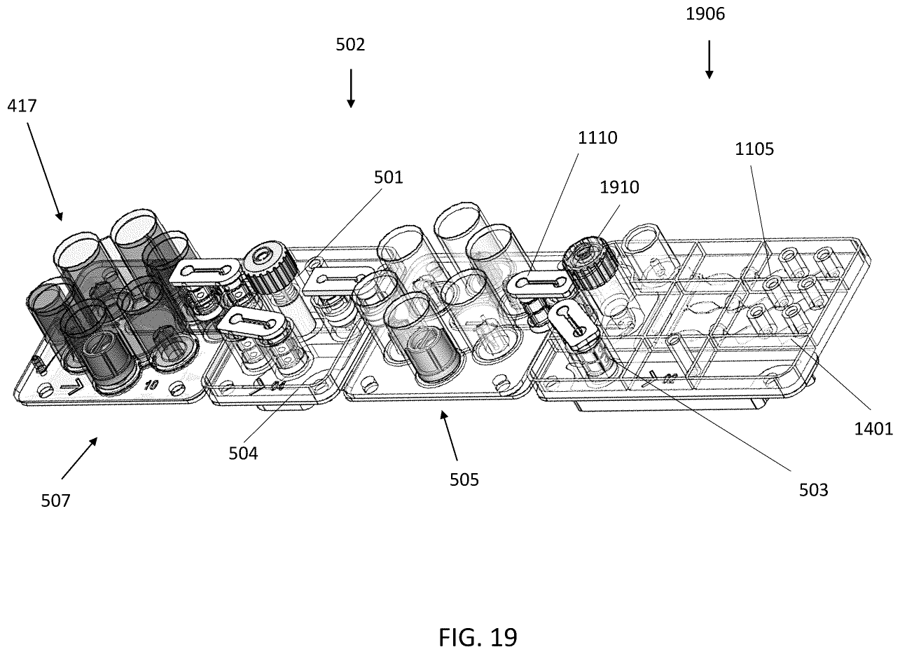

FIG. 19 shows an exemplary cartridge arrangement comprising a sample receiver module 502, two reagent modules 505 and 507, and an amplification/detection module 1906 (which may be substantially similar to amplification module 1104, detection module 1101, or a combination thereof, as described herein). In an exemplary embodiment, a cartridge 102 may comprise a first reagent module 507, a sample receiver module 502 downstream of the first reagent module 507, a second reagent module 505 downstream of the sample receiver module 502, and a combined amplification/detection module 1906 downstream of the second reagent module 505. The amplification/detection module 1906 may comprise one or more amplification regions 1105 upstream of one or more detection regions 1401 within a single module. In some embodiments, amplification is optional and only a detection region(s) 1401 is provided by the amplification/detection module 1906. The modules may be coupled to one another so as to provide fluid communication therebetween. In some embodiments, fluid communication may be regulated between the modules by one or more valves. For example, one or more fluid jumpers 503 may be used to valve fluid flow between and/or within the modules. The fluid jumpers 503 may be configured to provide one-way or bi-directional fluid flow between the modules. For example, the fluid jumpers 503 may be configured and arranged to regulate fluid flow from a reagent reservoir 417 to the sample reservoir 501 and prevent backflow from the sample reservoir 501 to the reagent reservoir 417. Alternatively, or in combination, one or more fluid jumpers 503 may be configured to regulate fluid flow from the sample reservoir 501 to a waste reservoir 504 on the sample receiver module 502 and prevent backflow from the waste reservoir 504 to the sample reservoir 501.

A sample may be applied to the sample reservoir 501 as described herein. The sample interface may optionally comprise a scraper as described herein. Lysis reagents may be transferred from one or more reagent reservoirs 417 of the first reagent module 507 to the sample reservoir 501 for lysis of the sample as described herein. In some embodiments, one or more additional reagents (e.g., PK for protein lysis, alcohol for nucleic acid aggregation, etc.) may be transferred from the first reagent module 507 to the sample reservoir 501 before the sample is transferred downstream. In some embodiments, nucleic acids of the sample may be captured by a concentrator 1110 (e.g., a filter, column, membrane, beads, etc.) on the second reagent module 505 (e.g., within a jumper 503 connecting the second reagent module 505 to the amplification/detection module 1906). In some embodiments, the sample may be transferred to a separate sample concentration module comprising a concentrator downstream of the second reagent module 505. Wash and/or elution reagents may be transferred from one or more reagent reservoir 417 of the second reagent module 505 to the concentrator 1110 for washing and elution of the sample as described herein. Elution of the nucleic acids from the concentrator 1110 may move the purified nucleic acids downstream into the amplification/detection module 1906. In some embodiments, concentration of the nucleic acids may not be performed and nucleic acids of the sample may be separated from debris following lysis and/or inactivation by one or more filters (e.g., filters located within a jumper as described herein with respect to concentrators) prior to being flown downstream. The amplification/detection module 1906 may comprise one or more amplification channels or chambers as described herein (e.g., three chambers as shown). The nucleic acids may be amplified as described herein and then transferred downstream to a detection region 1401 of the amplification/detection module 1906. One or more target nucleic acids may then be detected in a multiplexed fashion as described herein.

FIG. 23A shows a plan view of an exemplary amplification/detection module 1906 (which is also shown as part of the cartridge illustrated in FIG. 19). The amplification/detection module 1906 may comprise one or more amplification regions 1105 upstream of one or more detection regions 1401 within a single module. In some embodiments, amplification is optional and only a detection region(s) 2303 is provided by the amplification/detection module. The module may be coupled to another module as described herein so as to provide fluid communication therebetween. In some embodiments, fluid communication may be regulated between the modules by one or more valves. For example, one or more fluid jumpers 503 may be used to valve fluid flow between and/or within the modules. The fluid jumpers 503 may be configured to provide one-way or bi-directional fluid flow between the modules. For example, the fluid jumpers 503 may be configured and arranged to move a liquid containing purified nucleic acids from an elution module to the amplification/detection module. The jumper may be coupled to a loading channel/chamber configured to facilitate loading of the nucleic acids into the amplification chamber. In some embodiments, the loading channel/chamber may comprise a loading channel 2306 coupled to one or more loading chambers 2307. The nucleic acids may be loaded into a plurality of loading chambers 2307 via a loading channel 2306. In some embodiments, a bubble trap or bubble purge 2310 is included at one or more points along the fluid path within the device. In some embodiments, a bubble trap or purge 2310 may be provided before the loading channel/chamber in order to remove any bubbles from the liquid which may have been generated during sample processing. In some embodiments, the loading channel 2306 may end in a dead end 2309 configured to capture any gas within the system and/or overflow liquid that does not load into the loading chambers 2307. Gas trapped at the dead end 2309 may in at least some instances facilitate loading of the loading chambers 2307 by providing resistance to flow theretowards and encouraging the liquid being loaded to flow into the loading chambers 2307 where the fluidic resistance is lower. One or more vents may be provided downstream of the loading chambers 2307 to allow gas within the fluidic systems to escape as the liquid fills the loading channel(s) or chamber(s), amplification channel(s) or chamber(s), and detection chamber(s) or region(s). In some embodiments, a loading chamber 2307 may be upstream of an amplification chamber 1111, which may be upstream of a detection chamber 2303 as described herein. A first vent 2304 may be provided downstream of the amplification chamber 1111 in order to encourage fluid flow from the loading chamber 2307 to the amplification chamber 1111 when in an open configuration. Once the nucleic acids reach the amplification chamber 1111, the vent 2304 may be closed, and amplification may proceed if required by the assay being performed. Following amplification of the nucleic acids, a second vent 2305 downstream of a detection chamber 2303 in fluid communication with the amplification chamber 1111 may be opened and positive and/or negative pressure may be applied to move the amplified nucleic acids into the detection chamber 2303 where a programmable nuclease-based detection assay may be performed as described herein. In some embodiments, fluid may be agitated within the detection region 1401 during detection (e.g., via a spring-loaded valve piston coupled to the second vent 2305) in order to facilitate binding of the programmable nuclease to the guide nucleic acid, binding of the guide nucleic acid to the target nucleic acid or target amplicon, and/or access to and cleavage of the reporter by the activated programmable nuclease.

The loading channel 2306 may be configured (e.g., sized and shaped) to load the plurality of loading chambers 2307 with a pre-determined volume of liquid comprising nucleic acids. In some embodiments, each amplification or detection chamber 1111, 2303 may be fluidly independent of every other amplification or detection chamber, respectively, in order to facilitate target analysis multiplexing. In some embodiments, amplification of one or more target nucleic acids may be performed in each amplification chamber. In some embodiments, the amplification reagents of each reaction chamber may be different and may be designed to amplify different target nucleic acids of a plurality of target nucleic acids. The amplification chambers may be heated (e.g., isothermal or thermocycling) to amplify the one or more target nucleic acids as described herein. In some embodiments, heating may facilitate programmable nuclease-based detection reaction as described herein. In some embodiments, the amplification and detection reactions may be performed in the same reaction chamber (e.g., as a one-pot or hot pot reaction) or in separate amplification and detection chambers as described herein.

In some embodiments, the number of amplification regions 1105 and/or amplification channels or chambers 1111 within said amplification regions 1105 equal the number of detection regions 1401 and/or detection chambers or channels 2303 within said detection regions 1401. In some embodiments, the number of amplification regions 1105 and/or amplification channels or chambers 1111 within said amplification regions 1105 is more or less than the number of detection regions 1401 and/or detection chambers or channels 2303 within said detection regions 1401. For example, an amplification region 1105 may comprise three parallel amplification chambers and a detection region 1401 may comprise three parallel detection chambers, each amplification region 1105 being coupled to a single detection region 1401. In another example, an amplification region 1105 may comprise a single amplification chamber and a detection region 1401 may comprise a plurality of microwells or detection chambers, where the amplification chamber is in fluid communication with each of the plurality of microwells or detection chambers. In another example, an amplification region 1105 may comprise a plurality of amplification chambers and a detection region 1401 may comprise a single detection array. One of ordinary skill in the art will recognize that the relative numbers of and relationships between amplification region(s) 1105 and detection region(s) 1401 may be varied depending on the assay(s) being performed.

In some embodiments, reagents for a reaction in a given region are immobilized, dried, or otherwise deposited thereto. In some embodiments, one or more dried or lyophilized amplification and/or detection reagents (e.g., polymerases, programmable nucleases, guide nucleic acids, primers, etc.) may be disposed within the amplification region 1105 and/or detection region 1401 and may be reconstituted when the nucleic acids are flow therethrough. For example, a channel disposed between the loading chamber 2307 and the amplification chamber may comprise lyophilized pellets containing polymerases, reverse transcriptases, primers, dNTPs, and/or programmable nucleases. The detection region 1401 may comprise detection reagents that are immobilized, dried, or otherwise deposited thereto, including guide nucleic acids and/or reporters. In some embodiments, the detection region 1401 may comprise one or more dried and/or immobilized amplification reagents including primers, polymerases, reverse transcriptase, and/or dNTPs.

FIG. 23B shows a cross-sectional side view of a bubble trap 2310. In some embodiments, a loading channel/chamber may comprise a bubble trap or purge 2310 configured to remove any bubbles which may have been trapped in the liquid during the nucleic acid purification process and/or transport between modules. The bubble trap 2310 may be coupled to a channel connecting to a jumper 503 on a first end and a loading channel 2306 on a second end. The bubble trap 2310 may comprise a chamber 2312 having a height greater than either of the channels feeding into it so as to provide a void into which the bubbles may rise as the liquid pass through the bubble trap 2310. In some embodiments, the opening 2313 of the chamber 2312 into the second channel 2315 may be restricted (e.g., have a smaller height compared to the chamber and/or first channel 2314) so as to encourage bubbles to rise rather than continue downstream into the second channel 2315.

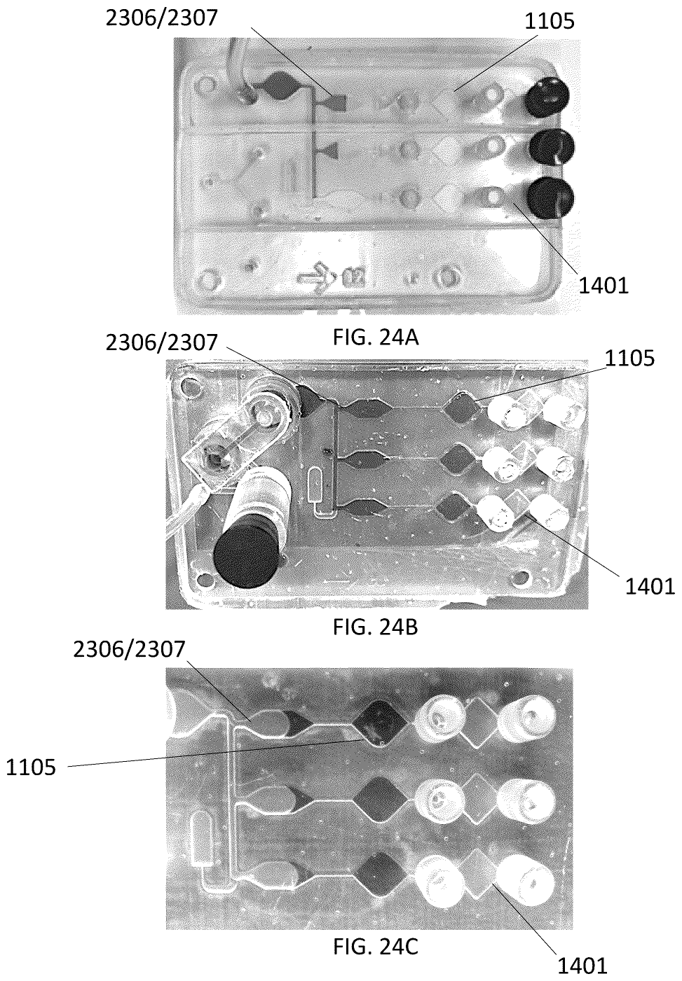

FIGS. 24A-24C show various stages of fluid movement during amplification chamber filling of an exemplary amplification region 1105 and detection region 1401. FIG. 24A shows liquid flowing through the bubble trap, into the loading channel 2306 (with gas trapped at the dead end 2309) and beginning to fill the loading chambers 2307. FIG. 24B shows liquid loading from the loading chambers into the amplification chambers 1111. FIG. 24C shows a near-completed loading of the liquid into the amplification chambers, with minimal residual liquid left behind in the loading channels.

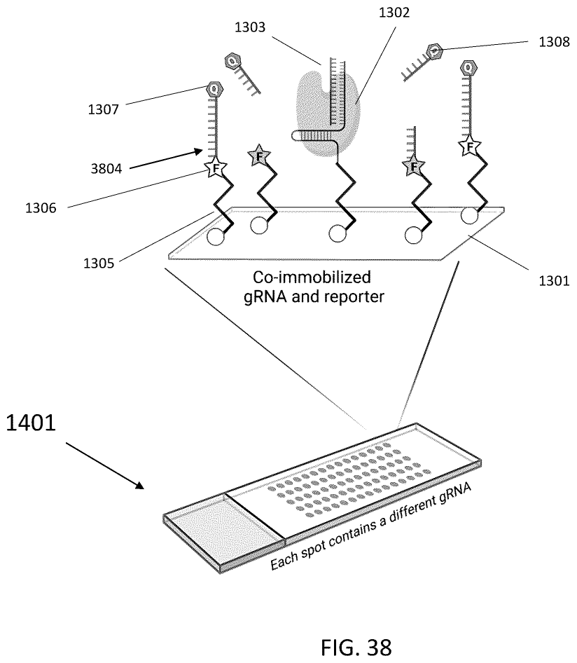

FIG. 38 depicts a surface 1301 of a detection region 1401 comprising an immobilized programmable nuclease-guide nucleic acid complex and a plurality of reporters, with one reporter having been cleaved by an activated programmable nuclease. The detection region may be substantially similar to any of the detection regions described herein. In some embodiments, one or more detection reagents may be immobilized on a surface 1031 of the detection region 1401. For example, one or more programmable nuclease 1302, one or more guide nucleic acid 1303, and/or one or more reporter 1304 may be immobilized on a surface 1301 of the detection region. In some embodiments, one or more detection reagents may be immobilized on a surface 1301 of the detection region via a linker 1305. The programmable nuclease 1032 may be complexed with a guide nucleic acid 1303 complementary to a specific target nucleic acid sequence as described herein. When a target nucleic acid is present in the sample, the programmable nuclease 1302 may be activated by binding of the guide nucleic acid 1303 (which is complexed thereto) to the target nucleic acid. Activation of the programmable nuclease 1302 may enable trans-cleavage of the reporter 1304 as described herein. In some embodiments, the reporter 1034 may comprise a detection moiety 1306 as described herein. In some embodiments, the reporter 1304 may comprise a detection moiety 1306 (e.g., a fluorophore) and a quencher moiety 1307 (e.g., a quencher) configured to generate a detectable signal when separated from one another. In some embodiments, trans-cleavage of the reporter 1304 by the activated programmable nuclease 1302 may release fluorophore of a reporter 1304 (or a quencher moiety, depending on the signal), thereby generating a signal indicative of the presence or absence of the target nucleic acid in the sample as described herein. The reporter 1304 may comprise any of the reporters described herein, and may comprise any detection moiety 1036 described herein (and/or other moieties or molecules described herein which facilitate signal detection). As shown in FIG. 38, cleavage of the reporter 1304 may release the quencher 1307 into solution, thereby allowing the fluorophore 1306 which was quenched when the reporter 1304 was intact to fluoresce. The presence of fluorescence at the detection location may therefore indicate the presence of the target nucleic acid. In another example, the reporter 1304 may not comprise a quencher 1307 and cleavage of the reporter 1304 may instead release a fluorophore into solution, thereby reducing fluorescence at the detection location. The absence of fluorescence at the detection location may therefore indicate the presence of the target nucleic acid. In some embodiments, cleavage of the reporter 1304 may result in a non-fluorescent signal as described herein. For example, cleavage of the reporter 1304 can produce a calorimetric signal, a potentiometric signal, an amperometric signal, a colorimetric signal, or a piezo-electric signal (3808).

FIGS. 39A-39B show illustrative representations of microfluidic devices comprising a plurality of chambers 2303 fluidically connected in sequence, in accordance with some embodiments. Each of the illustrated chambers 2303 comprises a well 3901, an inlet channel 3902, an outlet 3903 (also referred to as an outlet port), and a capillary valve 3904 (for simplicity, only some of each of these structures is labeled in the figures). The locations of wells 3901, capillary valves 3904, and outlets 3903 are labeled. FIGS. 39A and 39B present alternative arrangements of the capillary valves 3904 and outlets 3903 relative to the respective wells 3901. FIG. 39A shows several chambers that branch off of a common plenum, with the opening of the inlet channel 3902 to each chamber defined by a capillary valve 3904 having a smaller cross-sectional area relative to the plenum at the intersection thereof. In some embodiments, the fluid movement is laminar, due to surface tension. In exemplary embodiments, the inlet channel 3902 is 0.40 mm by 0.35 mm in width 3906 and depth 3907, respectively. In exemplary embodiments, the capillary valves 3904 are oriented such that they branch off of the main channel 3902 at an angle of about 90° or greater. In exemplary embodiments, the capillary valve 3904 dimensions are 0.30 mm by 0.20 mm in width and depth, respectively. In some embodiments, the capillary valve 3904 inlet area is much smaller than that of the inlet channel 3902 between the wells 3901. In some embodiments, the fluid fills a well 3901 of an upstream chamber by about 10% to about 100% (e.g. about 25-50%) before there is adequate pressure resulting in the fluid migrating into the capillary valve 3904 and into the next chamber 2303 downstream in the sequence. The fluid then continues filling the well 3901 of the upstream chamber while the fluid also progresses down the path from the upstream well 3901 and capillary valve 3904 to the next. In this way, fluid enters each chamber sequentially, but at any given time, one or more upstream chambers may continue to fill in parallel with one or more downstream chambers. In some embodiments, the wells 3901 have a volume of approximately 5 μL to 15 μL (e.g., about 10 μL). In some embodiments, air clears the plenum behind each well 3901 when the fluid volume input to the microfluidic device is such that the volume is equal to the amount of volume in each well 3901. In some embodiments, maintained pressure in the well-plate prohibits fluid from coming out of the well containing the fluid, initially. The outlet 3903 is connected to the well 3901 by an outlet channel 3905.

FIG. 39B shows several chambers 2303 wherein the plenum 3902 is located directly above each well 3901. In the illustrated arrangement, the inlet channel 3902 of a given chamber intersects the well 3901 at one point along the well perimeter and the capillary valve 3904 intersects at a separate point along the well perimeter (e.g., opposite one another across the well). In FIG. 39B, an outlet 3903 corresponds to an opening of the respective well 3901, which may optionally be sealed by an air-permeable membrane (not shown). In some embodiments, the back of the microfluidic device is covered with a hydrophobic membrane allowing the passage of gases, including air. In some embodiments, the hydrophobic membrane does not allow the transport of fluids at standard operating pressures. In some embodiments, fluid movement results from surface tension. In exemplary embodiments, the main channel 3902 is 0.50 mm by 0.35 mm in width and depth, respectively. In exemplary embodiments, each well 3901 is roughly 0.50 mm in diameter. In exemplary embodiments, the capillary valves 3904 are oriented such that they are directly over each well 3901. In exemplary embodiments, the capillary valve 3904 dimensions are 0.30 mm by 0.20 mm in width and depth, respectively. In some embodiments, the capillary valve inlet area is much smaller than that of the main channel 3902. In some embodiments, fluid flowing in the microfluidic device substantially fills the well 3901 before there is adequate pressure in the fluid for the fluid to begin migration into the capillary valve. The fluid then continues down the path from one well 3901 and capillary valve to the next well in sequence.

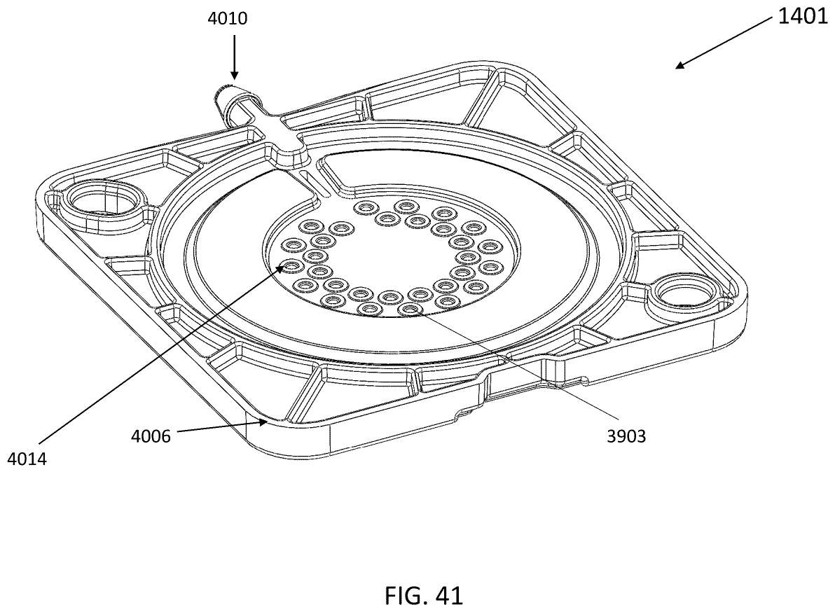

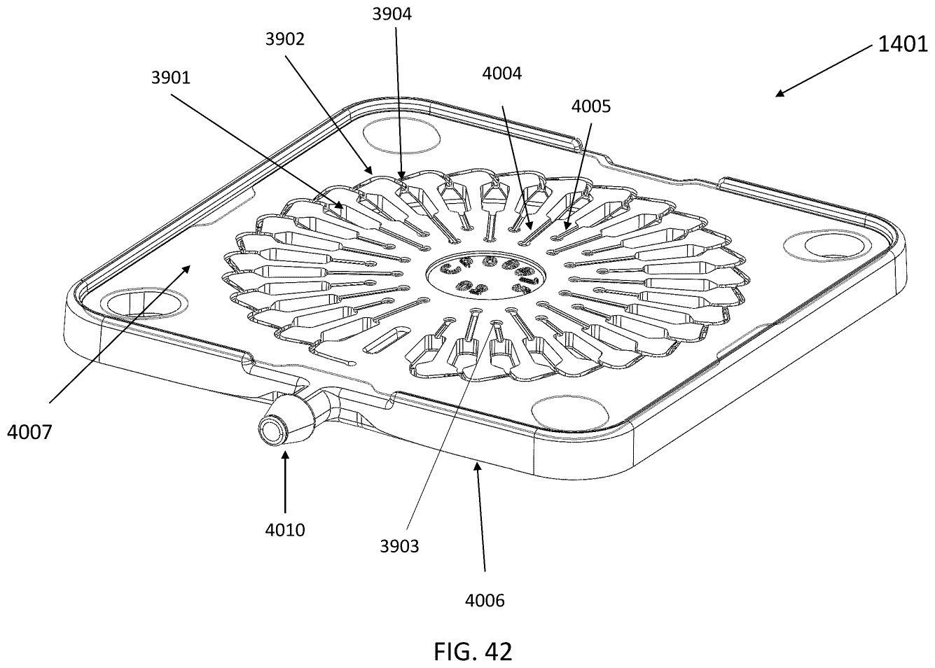

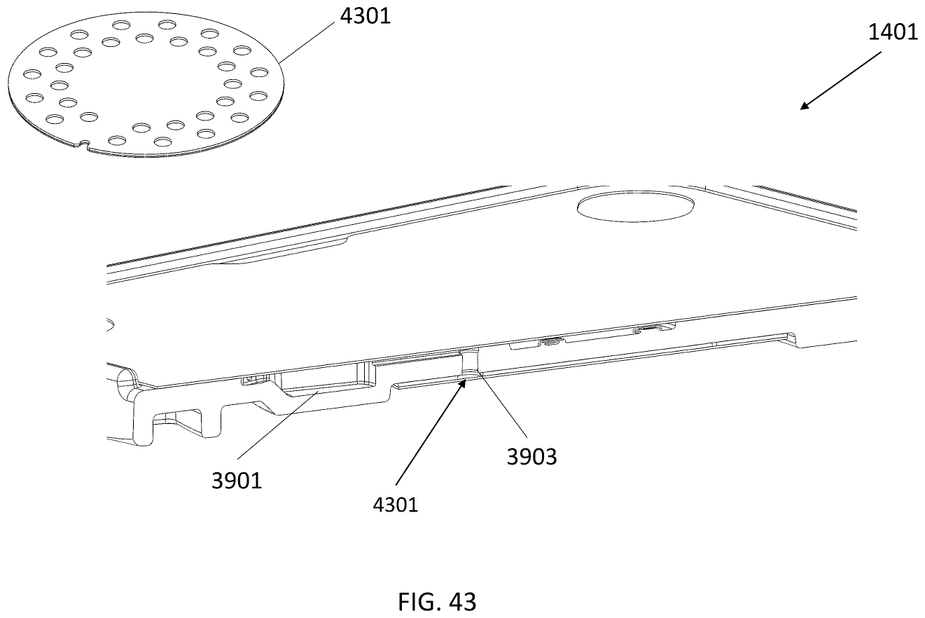

FIGS. 40A-40C depict different views of a microfluidic device in accordance with some embodiments. FIG. 40A shows a perspective view of a top surface of a detection region 1401 of the microfluidic device. FIG. 40B shows a perspective cross-sectional view of the top surface of the detection region 1401. FIG. 40C shows a plan view of the top surface of the detection region 1401. The illustrated microfluidic device comprises a plurality of chambers 2303 fluidically connected in sequence, in which each chamber of the plurality of chambers comprises a well 3901, an inlet channel 3902, an outlet 3903, and a capillary valve 3904. The arrangement of features in the device and fluid flow within the device is similar to those illustrated in FIG. 39A. The chambers branch off of a common plenum, with the opening of the inlet channel 3902 to each chamber (except the first chamber in the sequence) defined by a capillary valve 3904 having a smaller cross-sectional area relative to the plenum at the intersection thereof. The chambers are optionally arranged in a circular pattern in the device. The outlets 3903 are arranged closer to the center of the circular pattern to save space of the device, and terminate in openings at the bottom of the device that are sealed with an air-permeable, liquid-impermeable membrane 4012 (indicated in the figure as a hydrophobic membrane attached with adhesive, heat staked, laser welded, or ultrasonic welded). The base of the device may be infrared absorbent and/or opaque (4006), which may be beneficial for reducing noise when using light-based detection methods. The device may include a top lid that is infrared transmissive and/or clear (4007) (e.g., to facilitate detection of reactions in the wells 3901 when using light-based detection methods). The device may include a barb fitting inlet 4010 for loading of sample fluid. FIG. 40B illustrates the optional location 4011 of one or more detection reagents, which may be dried down or in lyophilized form within the well 3901 of the chamber prior to the introduction of a sample fluid, as described herein. FIG. 40C further illustrates a laser weld path 4013 around the channels, chambers, and outlets of the detection region 1401. The microfluidic device of FIGS. 40A-40C also illustrates that exits extending from the wells 3901 to their respective outlets 3903 may be of alternating long (4004) and short (4005) lengths, rather than all having a single uniform length, in order to maximize the use of space and minimize the overall size footprint of the detection region, which may be beneficial for manufacturing, user adoption, and/or interaction between the device and an instrument (e.g., a heater, detector, etc.). FIG. 41 depicts a perspective view of a bottom surface of the detection region 1401 of the microfluidic device, including optional features 4014 around the outlets to help ultrasonically weld an air-permeable membrane to the device. Such features may, for example, comprise raised ribs disposed abound the chamber outlets which may facilitate contact between the membrane and an ultrasonic horn for welding. FIG. 42 depicts a perspective view of a top surface of an embodiment of the microfluidic device in which a film is used to form a top surface of the chambers instead of a molded lid. FIG. 43 depicts a perspective cross-sectional view of an embodiment of the microfluidic device in which the membrane can be compounded with an adhesive backing to adhere to the device instead of using laser welding or ultrasonic welding or heat staking. The adhesive later 4301 can be cut with the same hole pattern as the chip and adhered to the membrane. In some embodiments, the membrane does not allow the transport of fluids therethrough at standard operating pressures. In some embodiments, a sample liquid may include a surfactant or other additive that reduces surface tension (e.g., TWEEN). For liquids with lower surface tension, membranes that are more hydrophobic and/or oleophobic may be preferred in order to avoid wetting. In some embodiments, the membrane comprises polytetrafluoroethylene (PTFE). The various features of FIGS. 40A-40C, 41, and 42 may be arranged in different geometries and/or provided in separate modules that are fluidically connected, or may form part of a larger device. For example, a jumper or other valve could connect the illustrated device to a sample prep module, an amplification module, or other module. Additional features and example arrangements are described herein, including in connection with various other aspects and embodiments.

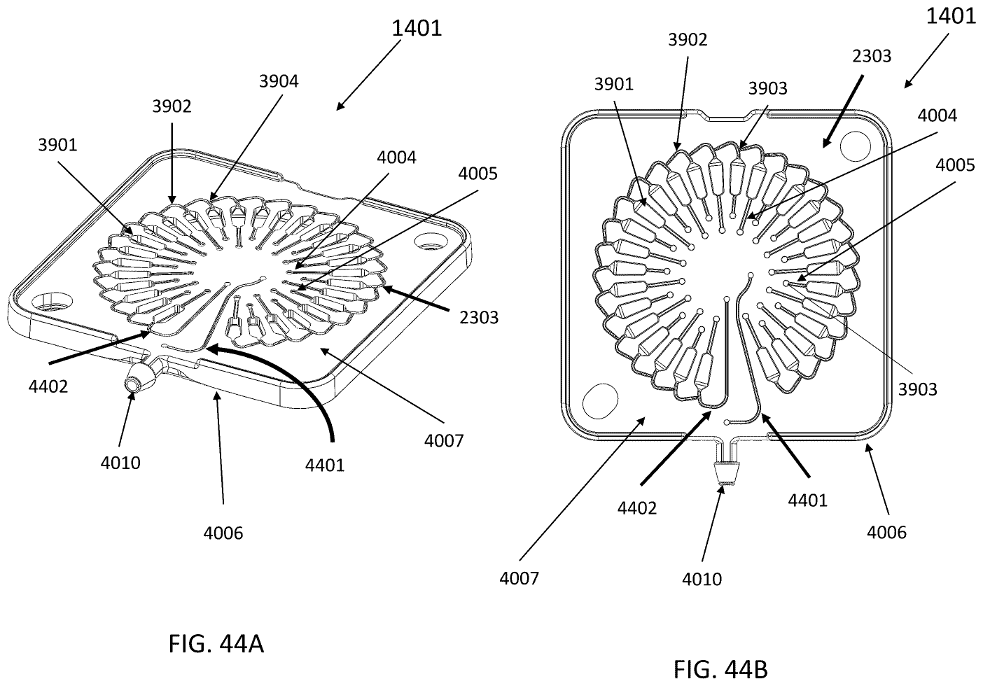

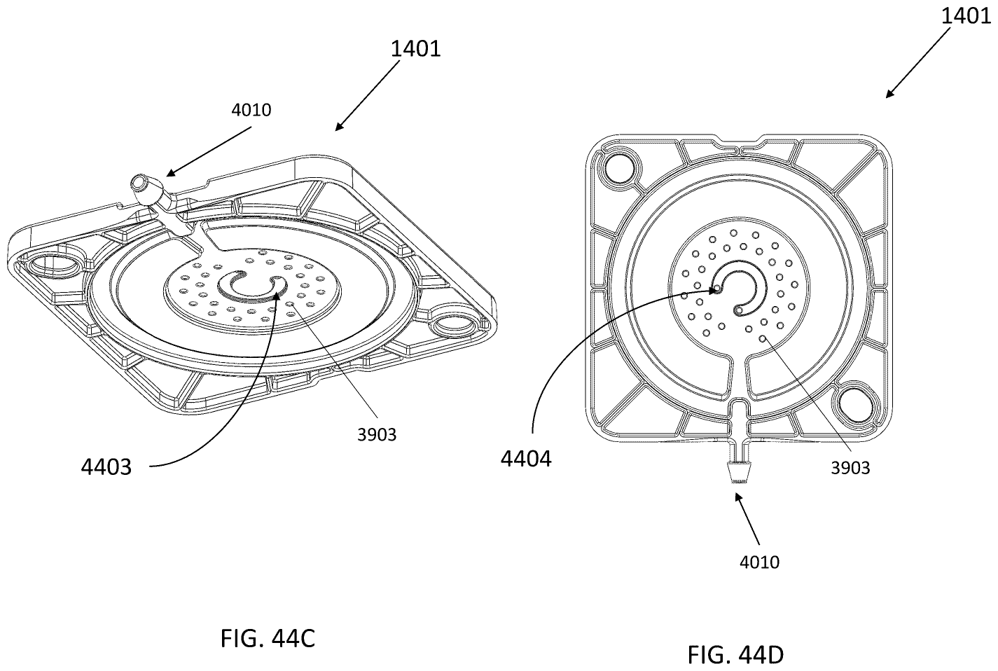

FIGS. 44A-44D depict different views of an exemplary detection region 1401 of a microfluidic device in accordance with some embodiments. FIGS. 44A-44B shows perspective and plan views of the top of the device. FIGS. 44C-44D shows perspective and plan views of the bottom of the device. The illustrated device includes a plurality of chambers 2303 fluidically connected in sequence, and connected to an upstream first inlet channel 4401 by way of a bubble purge channel 4403 (or a bubble trap) disposed therebetween. In operation, bubbles in the liquid of the first inlet channel 4401 enter the bubble purge channel 4403 and escape through a membrane 4404 that is hydrophobic and/or oleophobic and which covers the bottom surface of the bubble purge channel 4403. The liquid itself is substantially prevented from passing through the membrane and instead continues to flow along the bubble purge channel 4403 towards its exit into a second inlet channel 4402, reduced in or substantially free of bubbles. The second inlet channel 4402 feeds into the plurality of chambers 2303. Also illustrated is the location of the hydrophobic and/or oleophobic membrane 4404 (which may be the same as or different from membrane 4012 shown in FIG. 40B) that covers at least a portion of a bottom surface of the device. Non-limiting examples of membrane materials include polypropylene, polyethylene, PTFE, PTFE with polypropylene backing, polycarbonate, PVC, and PVDF. The arrangement of features in the device and fluid flow within the device is similar to those illustrated in FIG. 39A. The chambers branch off of a common plenum, with the opening of the inlet channel to each chamber (except the first chamber in the sequence) defined by a capillary valve 3904 having a smaller cross-sectional area relative to the plenum at the intersection thereof. The chambers are arranged in a circular pattern in the device. The outlets 3903 are arranged closer to the center of the circular pattern, and terminate in openings at the bottom of the device that are sealed with the membrane. The base of the device may be infrared absorbent and opaque 4006. The device may include a top lid or membrane that is infrared transmissive and clear 4007 (e.g., to facilitate detection of reactions in the wells 3901). In some embodiments, the hydrophobic or oleophobic membrane 4404 does not allow the transport of fluids at standard operating pressures. In some embodiments, fluid movement results from surface tension.

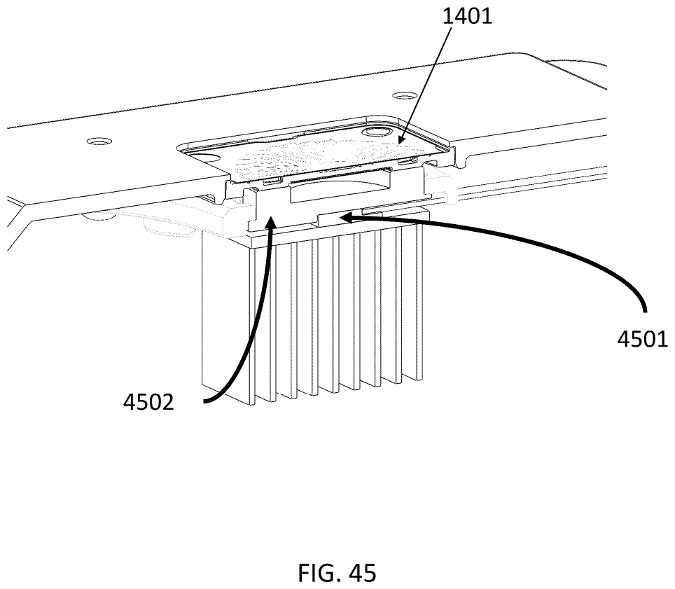

FIG. 45 depicts a system in accordance with some embodiments. The illustrated system comprises a microfluidic device in thermal communication with a heater or Peltier 4501 (e.g., thermal reservoir 1201 or TEC cooler 2801) via an optional heat spreader 4502 (e.g., comprising copper or aluminum). The heater may be square or some other suitable shape. As illustrated, the heat spreader 4502 forms a ring shape (corresponding to the annular pattern of the wells shown in FIGS. 40A-44D) that allows for control over the temperature of the wells of the detection region of the microfluidic device, without direct thermal contact with the entire device, such as a central portion of the device that may contain other elements (e.g., membrane sealed outlets and/or a bubble purge channel 4403, as shown in FIGS. 44A-44D). In some embodiments, the annular heat spreader 4502 may be replaced by or used in conjunction with an annular heater. The illustrated arrangement of the heating components is particularly useful in connection with microfluidic devices in which the chambers are arranged in a circular pattern, such as those illustrated in FIGS. 40A-44D.

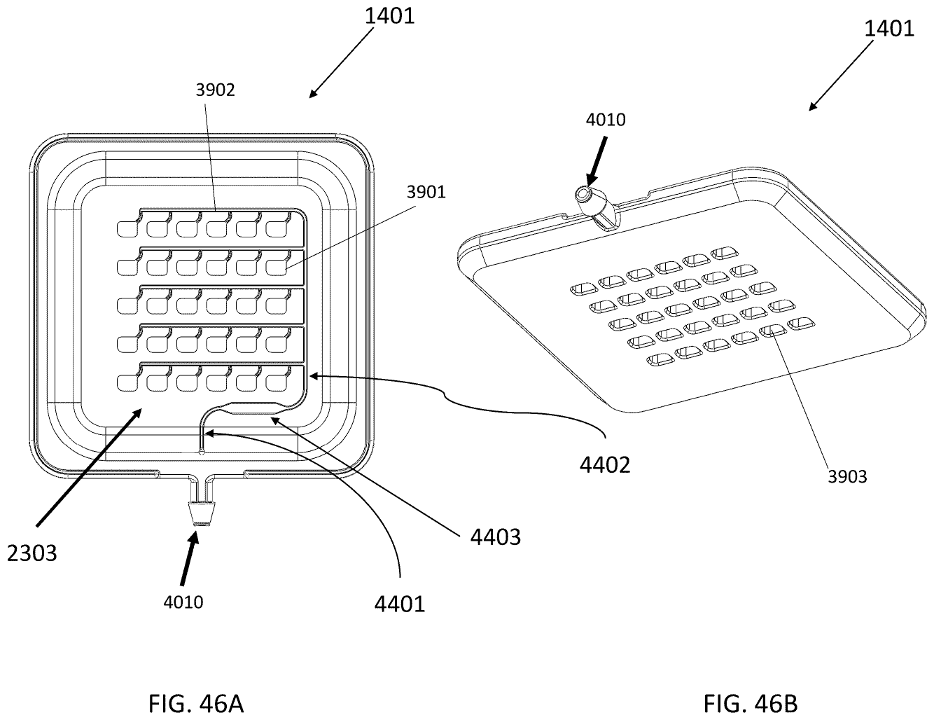

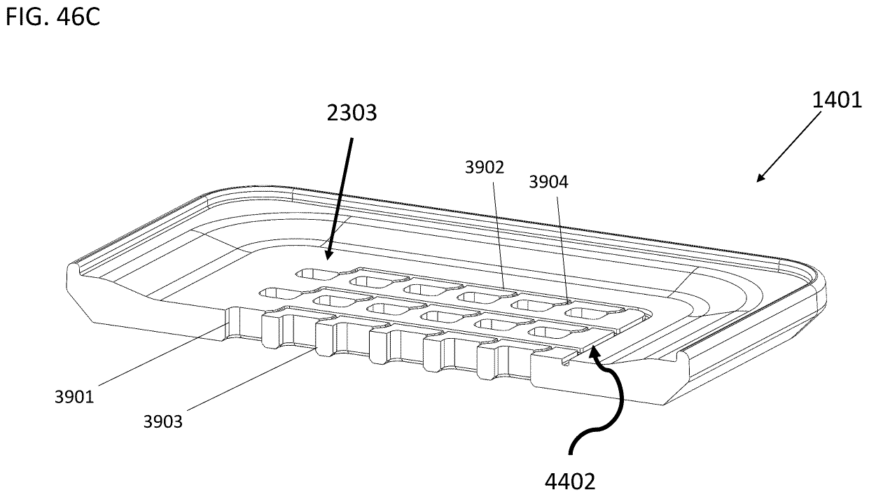

FIGS. 46A-46C depict an exemplary detection region of a microfluidic device in accordance with some embodiments. FIGS. 46A-46B show views of the top (left) and bottom (right) of the device. FIG. 46C shows a perspective view of a cross section of the device from the top. The illustrated device includes a plurality of chambers 2303 arranged in a grid. Subsets of the chambers 2303 are fluidically connected in sequence, and the subsets are all connected in parallel to branches of a common inlet channel 4402. Also illustrated is a bubble purge 4403 between the common inlet channel 4402 and a sample fluid inlet channel 4401. In operation, bubbles in the fluid entering the device float upward inside the bubble purge or trap 4403 and are retained there, whereas the fluid itself continues to flow along within the trap and exits into the common inlet channel 4402, reduced in or substantially free of bubbles. The common inlet channel 4402 feeds into the plurality of chambers 2303, and fluid collects in the wells 3901 thereof. The chambers 2303 also include outlets 3903 corresponding to an opening of the respective well at the bottom of the device, which may optionally be sealed by an air-permeable membrane (not shown). In some embodiments, the back of the microfluidic device is covered with a hydrophobic membrane allowing the passage of gases, including air. In some embodiments, the hydrophobic membrane does not allow the transport of fluids at standard operating pressures. In some embodiments, fluid movement results from surface tension.

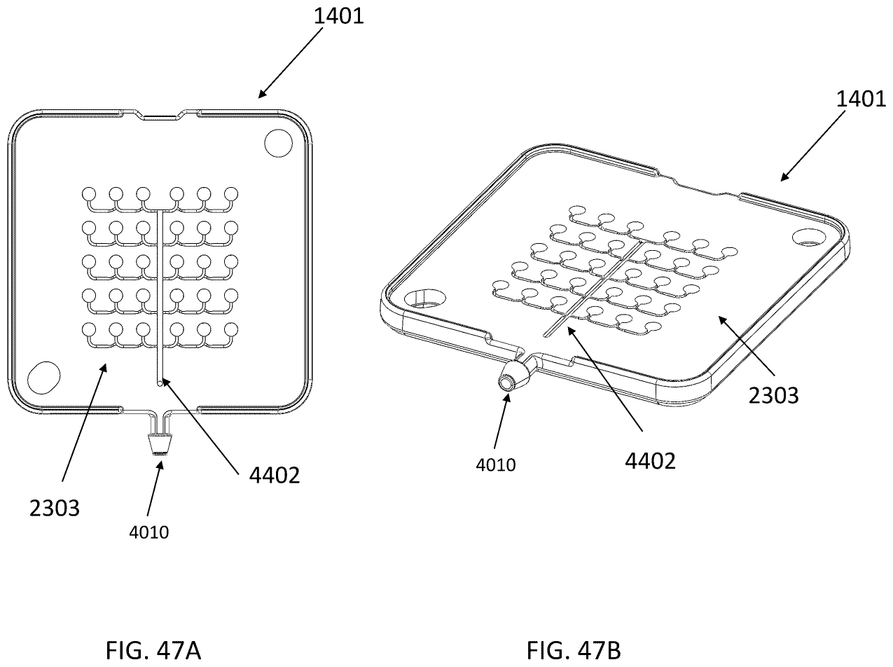

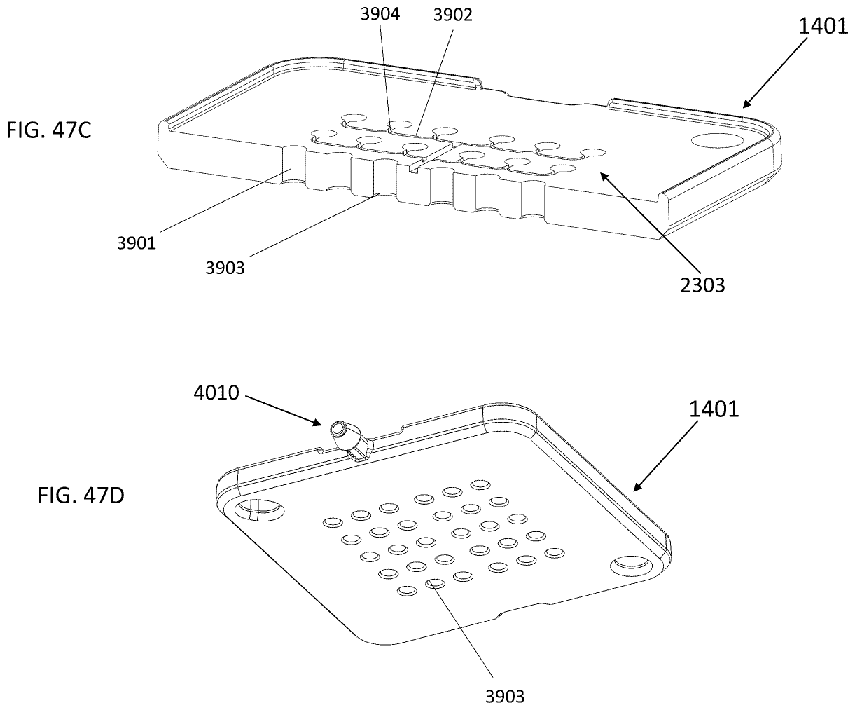

FIGS. 47A-47D depict a microfluidic device in accordance with some embodiments.

FIGS. 47A-47B show views of the top of the device. FIG. 47C shows a perspective view of a cross section of the device from the top. FIG. 47D shows a view of the bottom of the device. The illustrated device includes a plurality of chambers arranged in a grid. Subsets of the chambers are fluidically connected in sequence, and the 440 subsets are all connected in parallel to a common inlet channel 4402. Within a subset, each chamber 2303 comprises a well 3901, an inlet channel 3902, an outlet 3903, and a capillary valve 3904. The arrangement of the capillary valve provides that fluid flows into an upstream chamber up to a certain level before any substantial amount of fluid flows into the next chamber in the sequence. Upstream chambers and downstream chambers may continue to fill in parallel, but upstream chambers receive fluid first, and are the first to be completely filled. The outlets of the chambers are the bottom openings of the chambers shown in FIG. 47D, and are sealed by a membrane (not shown). The common inlet channel 4402 feeds into the plurality of chambers 2303, and fluid collects in the wells 3901 thereof. In some embodiments, the back of the microfluidic device is covered with a hydrophobic membrane allowing the passage of gases, including air. In some embodiments, the hydrophobic membrane does not allow the transport of fluids at standard operating pressures. In some embodiments, fluid movement results from surface tension. In some embodiments, the device includes a bubble trap disposed in the common channel so as to remove air from the fluid before reaching the plurality of chambers.

In some embodiments, the ratio of well width to well depth is less than 1. In some embodiments, the chambers or the wells thereof comprise a volume of approximately 0.75 μL. In some embodiments, the fluid volume input to the microfluidic device is such that the volume is equal to the amount of volume in each chamber or the wells thereof, which results in air clearing the plenum behind each well. In some embodiments, at constant pressure, the fluid in each well is substantially confined to the well, thereby limiting interference with reactions occurring in separate fluidically connected chambers.

In one aspect, the present disclosure provides a system comprising a microfluidic device comprising a plurality of chambers fluidically connected in sequence. In some embodiments, each chamber of the plurality of chambers comprises a well, an inlet channel, an outlet, and a capillary valve; the capillary valve of each chamber (i) has a cross-sectional area that is smaller than a cross-sectional area of the inlet channel of the respective chamber, and (ii) forms an entrance of the inlet channel of the next chamber in the sequence; and each outlet is air-permeable and configured to retain liquid within the respective chamber. In some embodiments, each chamber further comprises detection reagents comprising a guide nucleic acid and a reporter. In some embodiments, each guide nucleic acid (i) comprises a targeting sequence that hybridizes with a target nucleic acid of a plurality of different target nucleic acids or an amplicon thereof, and (ii) is effective to form a complex with a programmable nuclease that is activated upon binding the corresponding target nucleic acid or amplicon thereof; the guide nucleic acid of a first chamber in the plurality of chambers comprises a different targeting sequence from the guide nucleic acid of a second chamber in the plurality of chambers; and each reporter (i) comprises a cleavable nucleic acid and a detection moiety, and (ii) is configured to be cleaved to form a detectable cleavage product in response to activation of the complex in the well of the respective chamber. Non-limiting examples of guide nucleic acids, programmable nucleases, reporters, and detection moieties are described herein, including with respect to various other aspects and embodiments. In general, cleavage of the reporter in response to activation of the complex upon binding the corresponding target nucleic acid forms a detectable product. The nature of the detectable product and how it is detected may vary depending on the nature of the detection moiety. For example, the reporter may comprise a fluorescent label and a quencher, with cleavage of the reporter releasing the quencher and permitting detection of the fluorescent label upon excitation at the appropriate wavelength. As a further example, the reporter may comprise an enzyme (e.g., horseradish peroxidase) that is either in an inactive form or is physically separated from its substrate, with cleavage of the reporter releasing the enzyme to act upon its substrate, and the enzyme activity detected (e.g., as in a color change).

In some embodiments, the capillary valve has a cross-sectional area that is about 75%, 50%, 25%, 15%, or less than a cross-sectional area of the inlet channel of the respective chamber. In some embodiments, the capillary valve has a cross-sectional area that is about 50% or less than a cross sectional area of the inlet channel. In some embodiments, the respective cross-sectional areas of the capillary valve and the inlet are the cross-sectional areas of each at a point where the capillary valve and inlet intersect. In some embodiments, the respective cross-sectional areas of the capillary valve and the inlet are the cross-sectional areas of each at the point where it intersects the well.

In some embodiments, the capillary valve is oriented at an angle of about 90° or greater (e.g., about 100°, 120°, 140°, 160°, or) 180° with respect to the inlet channel of the respective chamber. In some embodiments, the capillary valve forms a junction with the inlet channel of the respective chamber (see, e.g., FIG. 39A). In some embodiments, the capillary valve and the inlet channel intersect the well of a respective chamber at separate points along a perimeter of the well (see, e.g., FIG. 39B). In some embodiments, the inlet channels comprise a width of about 0.3 mm to about 0.6 mm (e.g., about 0.3 mm, 0.35 mm, 0.4 mm, 0.45 mm, 0.5 mm, 0.55 mm, or 0.6 mm) and a depth of about 0.25 mm to about 0.45 mm (e.g., about 0.25 mm, 0.3 mm, 0.35 mm, 0.4 mm, or 0.45 mm). In some embodiments, the inlet channels comprise a width of about 0.4 mm and a depth of about 0.35 mm. In some embodiments, the inlet channels comprise a width of about 0.5 mm and a depth of about 0.35 mm. In some embodiments, the capillary valves comprise (a) a width of about 0.2 mm to about 0.4 mm (e.g., about 0.2 mm, 0.25 mm, 0.3 mm, or 0.4 mm) and a depth of about 0.1 mm to about 0.3 mm (e.g., about 0.1 mm, 0.15 mm, 0.2 mm, 0.25 mm, or 0.3 mm). In some embodiments, the capillary valves comprise a width of about 0.3 mm and a depth of about 0.2 mm.

In some embodiments, each of the wells has an internal volume of about 0.1 μL to about 50 μL, 0.5 μL to about 20 μL, or about 1 μL to about 10 μL. In some embodiments, each of the wells has an internal volume of about 0.75 μL. In some embodiments, each of the wells has an internal volume of about 10 μL.

In some embodiments, the outlet comprises an opening sized to permit displacement of air therethrough but to retain liquid within the well under an operating pressure of the microfluidic device. In instances where the outlet is configured to retain fluid based on the size of the opening, the opening will be smaller than the capillary valve, such that at the operating pressure, fluid is directed through the capillary valve, rather than the outlet (through which, air is still allowed to pass).

In some embodiments, the outlet comprises a surface comprising a hydrophobic coating. In some embodiments, the outlet comprises a surface comprising an air-permeable membrane that is hydrophobic and/or oleophobic. In some embodiments the outlet functions as a vent. In some embodiments, the outlet is open to air. In some embodiments, the outlet is connected to a pneumatic system. In some embodiments, each outlet is connected to a pressure source, which may be the same or different. In some embodiments, each outlet in the system is connected to a common pressure source. In some embodiments, the pneumatic system provides positive pressure to the microfluidic system. In some embodiments, the pneumatic system provides negative pressure to the microfluidic system.

In embodiments where the outlet comprises a surface comprising a hydrophobic membrane, any of a variety of suitable hydrophobic membranes may be used. In some embodiments, the hydrophobic membrane comprises a woven polymer (e.g., woven polypropylene or woven polyethylene). In some embodiments, the hydrophobic membrane comprises pores of about 0.1 microns to about 3 microns in size (e.g., about 0.1 microns, 0.25 microns, 0.5 microns, 0.75 microns, 1 micron, 1.5 microns, 2 microns, or 2.5 microns). In some embodiments, the hydrophobic membrane forms a bottom surface of the respective well. In some embodiments, the hydrophobic membrane comprises a woven polymer with pores of about 0.1 microns to about 2 microns in size, and forms a bottom surface of the respective well.

In embodiments where the outlet comprises a surface comprising a oleophobic membrane, any of a variety of suitable oleophobic membranes may be used. In some embodiments, the oleophobic membrane comprises a woven polymer. In some embodiments, the oleophobic membrane comprises pores of about 0.1 microns to about 3 microns in size (e.g., about 0.1 microns, 0.25 microns, 0.5 microns, 0.75 microns, 1 micron, 1.5 microns, 2 microns, or 2.5 microns). In some embodiments, the oleophobic membrane forms a bottom surface of the respective well. In some embodiments, the oleophobic membrane comprises a woven polymer with pores of about 0.1 microns to about 2 microns in size, and forms a bottom surface of the respective well.

In some embodiments, the system further comprises a sample interface configured to receive a sample, wherein the sample interface is in fluid communication with the plurality of chambers. Examples of sample interfaces are described herein, such as in connection with various other embodiments. In some embodiments, the sample interface is fluidically connected to the plurality of chambers via one or more sample preparation regions. In some embodiments, the one or more sample preparation regions comprise a lysis region configured to lyse one or more components of the sample. In some embodiments, the lysis region comprises lysis reagents. Non-limiting examples of lysis reagents include proteases (e.g., proteinase K), chaotropic agents, detergents, salts, and solutions of high osmolality, ionic strength, and pH. In some embodiments, the one or more sample preparation regions comprise an amplification region. In some embodiments, the amplification region comprises amplification reagents. Examples of amplification reagents are described herein, such as in connection with various other embodiments.

In some embodiments, the system comprises one or more heaters for use in controlling the temperature of one or more regions of the microfluidic device (e.g., a lysis region, an amplification region, and/or the plurality of chambers). Examples of heaters are disclosed herein, including heating elements that can also function as cooling elements (e.g., Peltier/thermoelectric coolers). In some embodiments, the heater is in thermal communication with a surface of the microfluidic device. In some embodiments, thermal communication is effected by integration of a heater within the microfluidic device. In some embodiments, thermal communication is effected by physical contact between the heater and the microfluidic device. In some embodiments, thermal communication is effected by placing the heater in proximity to a surface of the microfluidic device, without direct contact therewith. In some embodiments, thermal communication is effected by placing the heater in contact with a thermal conductor (e.g., a heat spreader) in direct contact with a surface of the microfluidic device In some embodiments, the heater is moveable within the system, and can be brought into thermal contact (and optionally direct contact) with the microfluidic device when needed, then separated from the microfluidic device when no longer needed. In some embodiments, the outlets vent through a first surface of the microfluidic device, and the heater is in thermal communication with a second surface of the microfluidic device, where the first surface is opposite the second surface.

The microfluidic device may comprise any suitable number of chambers. In some embodiments, all of the chambers in the microfluidic device are fluidically connected. In some embodiments, a microfluidic device comprises two or more sets of chambers, in which chambers within a set are fluidically connected in sequence, but chambers in different sets may or may not be connected. For example, two or more sets of chambers may each branch off from a common sample interface or sample preparation region. Alternatively, two or more sets of chambers in a single microfluidic device may be unconnected from each other and fed by separate sample interfaces. In some embodiments, the plurality of chambers comprises at least 10, 25, 50, 75, 100, 150, 200, 300, 400, 500, 1000 chambers fluidically connected in sequence. In some embodiments, the plurality of chambers comprises at least 10, 25, 50, or 100 chambers fluidically connected in sequence. In some embodiments, the plurality of chambers comprises at least 50 chambers fluidically connected in sequence.

In some embodiments, the detection reagents further comprise a programmable nuclease. A variety of suitable programmable nucleases are available. Non-limiting examples of programmable nucleases are provided herein, such as in connection with various other embodiments. In some embodiments, the programmable nuclease comprises a Cas protein. Non-limiting examples of Cas proteins include Cas12, Cas13, Cas14, CasPhi, and thermostable Cas proteins. In some embodiments, the detection reagents further comprise amplification reagents. Examples of amplification reagents are disclosed herein, such as in connection with various other embodiments. Amplification reagents may include one or more primers and a polymerase (e.g., a DNA polymerase). In some embodiments, the detection reagents are in a lyophilized form. In some embodiments, the guide nucleic acid and/or the reporter in each chamber are immobilized, dried, or otherwise deposited to a surface of the respective chamber. In some embodiments, immobilization is by a linkage. In some embodiments, the linkage comprises a covalent bond, a non-covalent bond, an electrostatic bond, an interaction (e.g., a covalent or noncovalent bond) between members of a binding pair (e.g., streptavidin and biotin), an amide bond, or any combination thereof. Non-limiting examples of linkages for immobilizing reagents to a surface are described herein, such as in connection with various other embodiments.

In one aspect, the present disclosure provides microfluidic devices comprising a loading channel and a plurality of chambers. In some embodiments, the loading channel comprises a first capillary valve disposed upstream of a second capillary valve disposed therein. In some embodiments, the plurality of chambers comprises a first chamber fluidically coupled to the loading channel upstream of the first capillary valve. In some embodiments, the microfluidic device comprises a second chamber fluidically coupled to the loading channel between the first capillary valve and the second capillary valve. In some embodiments, the microfluidic device comprises a third chamber fluidically coupled to the loading channel downstream of the second capillary valve. In some embodiments, each chamber of the first, second, and third chambers comprises an outlet. In some embodiments, each of the first and second capillary valves have a cross-sectional area that is smaller than a cross-sectional area of the loading channel. In some embodiments, each outlet is gas-permeable and configured to retain liquid within the respective chamber. The loading channel, plurality of chambers, and capillary valves may be sized in accordance with various embodiments described herein. The chambers may comprise one or more reagents, such as one or more detection reagents, in accordance with various embodiments described herein.

### V. CERTAIN METHODS OF USE

Any of the systems described herein (which may comprise any of the instruments and/or cartridges described herein) may be used to detect one or more target nucleic acids in a sample. In some embodiments, detecting the one or more target nucleic acids may comprise one or more of the following steps: sample collection, sample extraction, sample lysis, protein degradation, nucleic acid extraction, nucleic acid purification, nucleic acid concentration, waste removal, nucleic acid elution, nucleic acid amplification, a programmable nuclease-based detection reaction, target detection, and/or reporter detection, or any combination thereof.

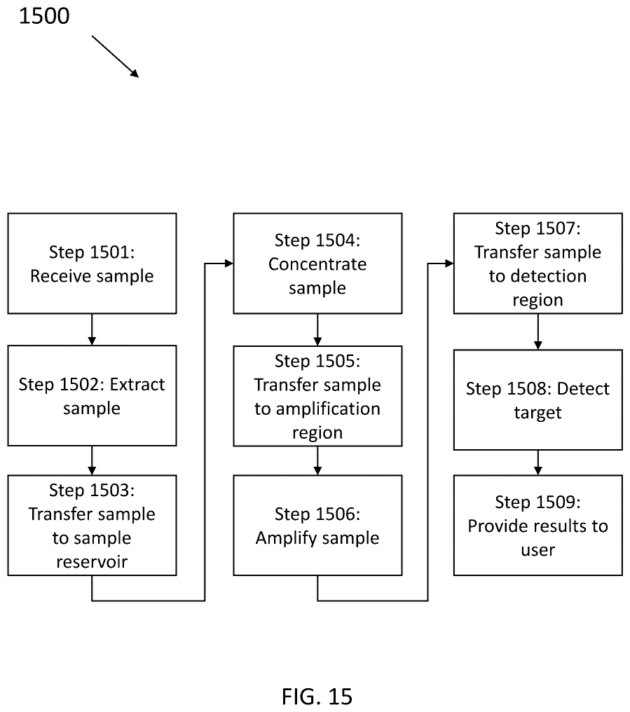

FIG. 15 shows an exemplary workflow of a method 1500 for nucleic acid analysis and detecting a target nucleic acid.

At Step 1501, the system may receive a sample. The sample may comprise any of the samples described herein. In some embodiments, the sample may comprise one or more different target nucleic acids. Receiving a sample containing a target nucleic acid may subsequently result in a “hit” for that target nucleic acid at Step 1508. In some embodiments, the sample may not comprise a target nucleic acid. In some embodiments, the sample may be collected with a sample collector (e.g., swab, tube, etc.) as described herein. In some embodiments, the sample collector may be received in a sample interface of the cartridge as described herein. In some embodiments, the sample may be directly collected at the sample interface and/or sample reservoir (e.g., without the use of a separate sample collector).

At Step 1502, In some embodiments, the sample may be extracted from the sample collector. Extracting the sample may comprise eluting at least a portion of the sample from the sample collector (e.g., when the sample collector is a swab). In some embodiments, extracting the sample may comprise scraping the sample collector against a scraper as described herein. In some embodiments, extracting the sample may comprise pipetting the sample (e.g., when the sample collector is a tube). In some embodiments, extracting the sample may comprise lysing and/or heating the sample as described herein. In some embodiments, extracting the sample may comprise inactivating one or more components in the lysis buffer as described herein. In some embodiments, extracting the sample may comprise filtering debris from the sample following elution, lysis, and/or inactivation.

At Step 1503, the sample may be transferred from the sample interface into a sample reservoir as described herein. In some embodiments, the sample reservoir may comprise a fluid chamber. In some embodiments, the sample reservoir may be part of a sample receiver module as described herein. Transferring the sample to the sample reservoir may comprise transferring the sample to the sample receiver module as described herein. In some embodiments, the sample reservoir may be part of a sample preparation or concentration module and transferring the sample to the sample reservoir may comprise transferring the sample to the sample preparation module as described herein. In some embodiments, transferring the sample may comprise transferring a fluid comprising the sample from the sample interface to the sample reservoir. In some embodiments, fluid flow between reservoirs and/or regions may be regulated by a valve (e.g., a rotary valve, jumper valve, etc.) as described herein. In some embodiments, fluid flow between reservoirs and/or regions may be driven by a positive or negative pressure source (e.g., a pump, syringe, or the like) as described herein.

At Step 1504, at least a portion of the sample may be concentrated as described. For example, the sample may comprise a plurality of nucleic acids and concentrating the sample may comprise concentrating the plurality of nucleic acids as described herein. In some embodiments, the plurality of nucleic acids may comprise one or more target nucleic acids and concentrating the sample may comprise concentrating the target nucleic acid(s).

At Step 1505, at least a portion of the sample may optionally be transferred to an amplification region of the cartridge. In some embodiments, the transferred portion of the sample may comprise the plurality of nucleic acids. In some embodiments, the transferred portion of the sample may comprise the one or more target nucleic acids. The amplification region may comprise one or more amplification chambers or channels as described herein. In some embodiments, the amplification region may be part of an amplification module as described herein. In some embodiments, the plurality of nucleic acids comprising the target nucleic acid may be transferred to the amplification module.

At Step 1506, one or more nucleic acids in the sample may optionally be amplified as described herein. For example, the one or more target nucleic acids may be amplified via an isothermal or thermal amplification reaction as described herein. In some embodiments, amplifying the sample may comprise exposing the sample to one or more amplification reagents as described herein. In some embodiments, amplifying the sample may comprise raising the temperature of the fluid containing the sample to a pre-determined temperature. In some embodiments, amplifying the sample may comprise cycling the temperature of the fluid containing the sample between a plurality of pre-determined temperatures. In some embodiments, amplifying the one or more nucleic acids in the sample may occur in an amplification region or amplification/detection region of the cartridge (e.g., in a same or different location as detection).

At Step 1507, the sample may be transferred to a detection region of the cartridge. The detection region may comprise one or more spatially separated detection locations (e.g., detection spots, microwells, chambers, etc.) as described herein. In some embodiments, the detection region may be part of a detection module and transferring the sample may comprise transferring a target nucleic acid to the detection module. In some embodiments, one or more detection reagents may be immobilized, dried, or otherwise deposited on a surface of the detection region. For example, one or more programmable nuclease, one or more guide nucleic acid, and/or one or more reporter may be immobilized, dried, or otherwise deposited on a surface of the detection region as described herein. In some embodiments, one or more of the detection reagents may be in a dried or lyophilized form prior to mixing with the sample fluid. Each programmable nuclease may be complexed with a guide nucleic acid complementary to a specific target nucleic acid sequence. In some embodiments, each detection location may comprise a different combination of detection reagents so as to provide a plurality of spatially separated multiplex-capable detection locations. For example, one or more (e.g., each) detection location may comprise a different guide nucleic acid configured to bind to a different target nucleic acid. Alternatively, or in combination, one or more (e.g., each) detection location may comprise a different programmable nuclease. Alternatively, or in combination, one or more (e.g., each) detection location may comprise a different reporter. In some embodiments, each detection location may comprise detection reagents configured to detect a different target nucleic acid of a plurality of target nucleic acids. In some embodiments, amplification and detection may occur in the same location of the detection region. For example, one or more (e.g., each) detection location may comprise a different primer configured to amplify a different target nucleic acid.

At Step 1508, if present in the sample, the one or more target nucleic acids may be detected as described herein. Detecting the target nucleic acid may comprise detecting a signal indicative of cleavage of the reporter by the programmable nuclease at the detection location. When a target nucleic acid is present in the sample, the programmable nuclease may be activated by binding of the guide nucleic acid (which is complexed thereto) to the target nucleic acid. Activation of the programmable nuclease may enable trans-cleavage of the reporter as described herein. In some embodiments, the reporter may comprise a detection moiety as described herein. In some embodiments, trans-cleavage of the reporter by the activated programmable nuclease may release the detection moiety (or a quencher moiety, depending on the signal), thereby generating a signal indicative of the presence or absence of the target nucleic acid in the sample as described herein. In some embodiments, amplification may occur simultaneously with detection (e.g., when amplification and detection occur in the same volume).

At Step 1509, the results of the detection assay may be provided to the user. As described herein, detection of a signal may indicate a presence or absence of a target nucleic acid in the sample. For example, detection of a signal may be considered a “hit” for the presence of a target nucleic acid (and the lack of a signal may indicate the absence of the target nucleic acid) or the lack of a signal may be considered a “hit” for the presence of the target nucleic acid (and detection of a signal may indicate the absence of the target nucleic acid). In some embodiments, the system may output the results to a user through a display as described herein.

Although the steps above show a method 1500 of detecting a target nucleic acid using a programmable nuclease-based detection system in accordance with embodiments, a person of ordinary skill in the art will recognize many variations based on the teachings described herein. The steps may be completed in a different order. Steps may be added or deleted. Some of the steps may comprise sub-steps. Many of the steps may be repeated as often as necessary to perform a detection assay.

For example, in many embodiments, additional target nucleic acids are detected simultaneously (e.g., in a multiplexed fashion) as described herein. Alternatively, or in combination, the method 1500 may optionally comprise coupling a first cartridge segment containing liquid reagents with a second cartridge segment containing lyophilized reagents prior to Step 1501 or Step 1502. Combining the segments may result in the first cartridge component and second cartridge component comprising an assembled cartridge. In some embodiments, the method 1500 may optionally include inactivating the sample prior to Step 1505. In some embodiments, the method 1500 may optionally include amplifying the signal produced upon cleavage of the reporter as described herein. In some embodiments, Steps 1506 and 1508 may occur in the same location and/or at the same time. In some embodiments, Steps 1505 and 1506 may be optional for assays not requiring amplification.

An exemplary method may comprise the steps of: i) receiving a swab comprising a sample containing a target nucleic acid; ii) inserting the swab into a sample interface located on a cartridge, the sample interface comprising a scraper configured to extract the sample from the swab and coupled to a sample reservoir; iii) loading the cartridge into an instrument; iv) transferring the sample from the sample interface to a sample reservoir located within the cartridge; v) lysing the sample; vi) purifying one or more target nucleic acids from the sample; vii) concentrating the one or more target nucleic acids; viii) transferring the one or more target nucleic acids to an amplification region (e.g., chamber, channel, etc.); ix) amplifying the one or more target nucleic acids; x) transferring the one or more target nucleic acids to a detection region, the detection region comprising a spatial array of detection locations (e.g., detection spots or microwells), each detection location comprising a reporter and a different programmable nuclease of a plurality of programmable nucleases, wherein each of the different programmable nucleases comprises a different guide nucleic acid complementary to a different target nucleic acid of the one or more target nucleic acids, wherein, at each detection location, the reporter and each different programmable nuclease or each different guide nucleic acid are immobilized, dried, or otherwise deposited to a surface of the detection region, wherein, at each detection location, each different programmable nuclease is configured to cleave the reporter and generate a different signal of a plurality of signals, and wherein each different signal of the plurality of signals indicates a presence or absence of each different complementary target nucleic acid at its respective detection location.

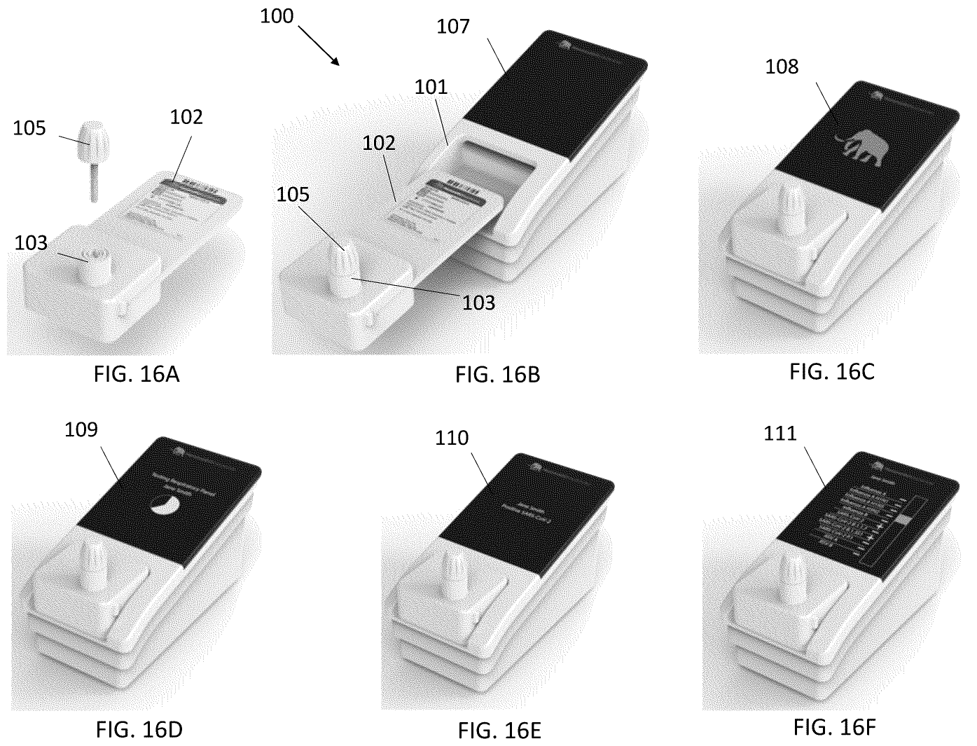

FIGS. 16A-16F show various steps of an exemplary process for using a system 100 for nucleic acid analysis. In an exemplary embodiment, a nasopharyngeal swab may be used to collect a sample from a patient. The sample collector 105 (e.g., swab) may then be inserted into a cartridge 102 as shown in FIG. 16A. The cartridge 102 may comprise any of the cartridges described herein, or any subset or combination of cartridge components, modules, regions, or reagents described herein. The cartridge 102 may then be placed into the instrument 101 as shown in FIG. 16B via interface 103. An initialization screen may be presented to the user via the display 108 to provide instructions to the user and/or receive information (e.g., patient age, patient gender, gender, nucleic acid test type, test start instructions, etc.) from the user as shown in FIG. 16C. After the test has begun, the display may present a In Progress screen 109 to provide the user with updates on the progress of the test as shown in FIG. 16D. Once the test has finished, the instrument may present important results 110 to the user via the display as shown in FIG. 16E. Alternatively, or in combination, the instrument may present the results of every test run on the multiplexed assay on an aggregate results screen 111 as shown in FIG. 16F.

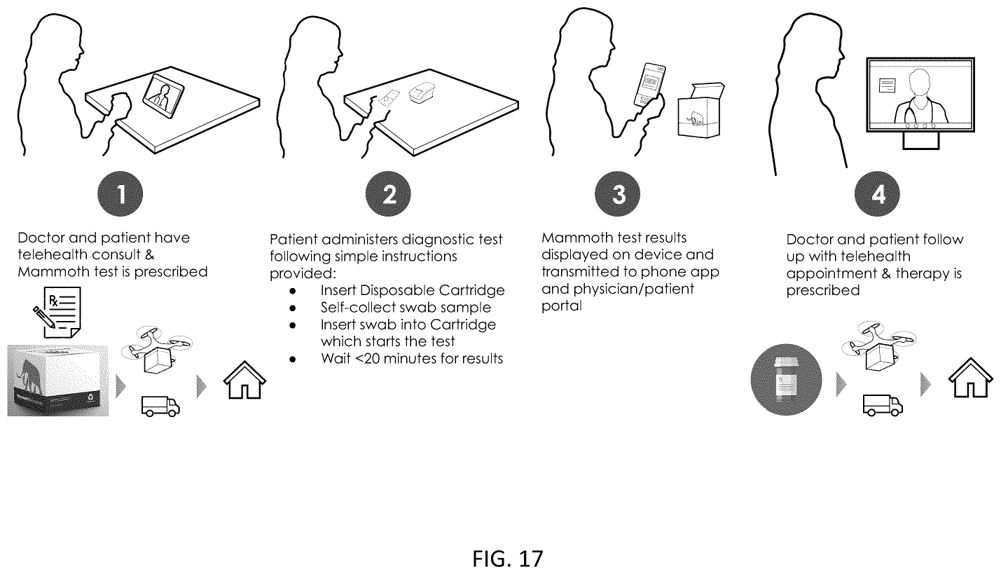

FIG. 17 shows an exemplary workflow for a patient use case of a system for nucleic acid analysis. In some embodiments, the system is may be to analyze a patient sample as depicted in FIG. 17. A health care professional (e.g., a doctor) and patient may have a health consultation (e.g., online, over the phone, or in person) during which a desired nucleic acid analysis may be prescribed. In some embodiments, a system comprising a cartridge and an instrument may be sent to the patient for at-home use. In some embodiments, sample analysis may be performed at the healthcare processional's office prior-to, during, or after an in-person consultation. In some embodiments, the patient may then administer the diagnostic test to themselves. The patient may insert the cartridge into the instrument as directed by instructions included with the system. In some embodiments, a swab or other sample collector (e.g., tube) may be used to collect a sample from the patient as described herein. For example, the patient may collect a nasopharyngeal sample using a swab provided with the system. The patient may then insert the swab into the cartridge as described herein. In some embodiments, the swab may be inserted into the cartridge prior to insertion of the cartridge into the instrument. In some embodiments, the swab may be inserted into the cartridge after insertion of the cartridge into the instrument. The sample may then be processed as described herein. In some embodiments, insertion of the swab or other sample collector into the cartridge may trigger sample processing to being. Sample processing may take a pre-determined amount of time to complete, as described herein. In some embodiments, the test results (e.g., whether the target nucleic acid was present in the sample, which target nucleic acid(s) of a plurality of target nucleic acids were present in the sample, the degree to which the target nucleic acid(s) was present in the sample, etc.) may be displayed by the instrument to the user. Alternatively, or in combination, the test results may be transmitted to a user and/or healthcare professional via a phone app. Alternatively, or in combination, the test results may be transmitted to a physician/patient portal. In some embodiments, a therapy may be prescribed to the patient by the healthcare professional based at least in part on the test results.

In some aspects, the present disclosure provides methods for detecting one or more of a plurality of different target nucleic acids in a system described herein. In some embodiments, the method comprises, (a) flowing a liquid comprising one or more of the different target nucleic acids or amplicons thereof into the plurality of chambers; (b) in one or more of the wells, forming the activated complex and cleaving the reporters; and (c) detecting the detectable cleavage products in one or more of the wells, wherein the location of a well comprising a detectable cleavage product identifies the target nucleic acid or amplicon thereof present in the well. In general, a given well at a particular location will comprise one or more guide nucleic acids of known sequence that hybridizes with a particular target nucleic acid. As such, formation of a detectable cleavage product in the given well is indicative of the presence of the corresponding target nucleic acid for that guide nucleic acid associated with the particular well at the known location. In cases where a sample comprises multiple different target nucleic acids, detectable cleavage products may form in multiple wells, identifying the presence of each of the different target nucleic acids in the sample. In some cases, a detectable product may not form in one or more, most, or even all chambers, indicating that the corresponding target nucleic acids of the respective guide nucleic acids are not present above a threshold for detection. Accordingly, in some embodiments, methods of the present disclosure provide detecting the presence, absence, or amount of a plurality of different target nucleic acids in a sample.

### VI. CERTAIN DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Terminology used herein is for the purpose of describing particular embodiments only and is not intended to be limiting of the invention. For example, as used herein, the singular forms “a”, “an”, and “the” are intended to include the plural forms as well, unless the context clearly dictates otherwise. For example, the term “a sample” includes a plurality of samples, including mixtures thereof. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, or “coupled” to another feature or element, it can be directly connected, attached, or coupled to the other feature or element or intervening features or elements may be present. In contrast, when a feature or element is referred to as being “directly connected”, “directly attached”, or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent, depending on the context.

The terms “subject,” “individual,” and “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed with or suspected of being at high risk for a disease. In some cases, the subject is not necessarily diagnosed with or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), or +/−10% of the stated value (or range of values). Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that the throughout the application, data is provided in a number of different formats, and that this data, represents endpoints and starting points, and ranges for any combination of the data points.

As used herein, the terms “treatment” and “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “thermostable” and “thermostability” refer to the stability of a composition disclosed herein at one or more temperatures, such as an elevated operating temperature for a given reaction. Stability may be assessed by the ability of the composition to perform an activity, e.g., cleaving a target nucleic acid or reporter. Improving thermostability means improving the quantity or quality of the activity at one or more temperatures.

As used herein, the terms “percent identity,” “% identity,” and “% identical” refer to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y in an alignment between the two. Generally, computer programs may be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95). For the purposes of calculating identity to the sequence, extensions, such as tags, are not included.

As used herein, a “one-pot” reaction refers to a reaction in which more than one reaction occurs in a single volume alongside a programmable nuclease-based detection (e.g., DETECTR) assay. For example, in a one-pot assay, sample preparation, reverse transcription, amplification, in vitro transcription, or any combination thereof, and programmable nuclease-based detection (e.g., DETECTR) assays are carried out in a single volume. In some embodiments, amplification and detection are carried out within a same volume or region of a device (e.g., within a detection region). Readout of the detection (e.g., DETECTR) assay may occur in the single volume or in a second volume. For example, the product of the one-pot DETECTR reaction (e.g., a cleaved detection moiety comprising an enzyme) may be transferred to another volume (e.g., a volume comprising an enzyme substrate) for signal generation and indirect detection of reporter cleavage by a sensor or detector (or by eye in the case of a colorimetric signal).

As used herein, “HotPot” refers to a one-pot reaction in which both amplification (e.g., RT-LAMP) and detection (e.g., DETECTR) reactions occur simultaneously. In many embodiments, a HotPot reaction may utilize a thermostable programmable nuclease which exhibits trans cleavage at elevated temperatures (e.g., greater than 37 C).

The terms, “amplification” and “amplifying,” as used herein, refer to a process by which a nucleic acid molecule is enzymatically copied to generate a plurality of nucleic acid molecules containing the same sequence as the original nucleic acid molecule or a distinguishable portion thereof.

The term, “complementary,” as used herein with reference to a nucleic acid refers to the characteristic of a polynucleotide having nucleotides that base pair with their Watson-Crick counterparts (C with G; or A with T/U) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “cleavage assay,” as used herein refers to an assay designed to visualize, quantitate or identify cleavage of a nucleic acid. In some cases, the cleavage activity may be cis-cleavage activity. In some cases, the cleavage activity may be trans-cleavage activity.

Assays which leverage the transcollateral cleavage properties of programmable nuclease enzymes (e.g., CRISPR-Cas enzymes) are often referred to herein as DNA endonuclease targeted CRISPR trans reporter (DETECTR) reactions. As used herein, detection of reporter cleavage (directly or indirectly) to determine the presence of a target nucleic acid sequence may be referred to as “DETECTR”.

The term, “detectable signal,” as used herein refers to a signal that can be detected using optical, fluorescent, chemiluminescent, electrochemical or other detection methods known in the art.

The term, “detecting a nucleic acid” and its grammatical equivalents, as used herein refers to detecting the presence or absence of the target nucleic acid in a sample that potentially contains the nucleic acid being detected.

The term, “effector protein,” as used herein refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. In some embodiments, the complex comprises multiple effector proteins. In some embodiments, the effector protein modifies the target nucleic acid when the complex contacts the target nucleic acid. In some embodiments, the effector protein does not modify the target nucleic acid, but it is fused to a fusion partner protein that modifies the target nucleic acid. A non-limiting example of modifying a target nucleic acid is cleaving (hydrolysis) of a phosphodiester bond. Additional examples of modifying target nucleic acids are described herein and throughout. In some embodiments, the term, “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). The effector protein may be a Cas protein (i.e., an effector protein of a CRISPR-Cas system).

The term, “guide nucleic acid,” as used herein refers to a nucleic acid comprising: a first nucleotide sequence that hybridizes to a target nucleic acid; and a second nucleotide sequence that is capable of being non-covalently bound by an effector protein. The first sequence may be referred to herein as a spacer sequence. The second sequence may be referred to herein as a repeat sequence. In some embodiments, the first sequence is located 5′ of the second nucleotide sequence. In some embodiments, the first sequence is located 3′ of the second nucleotide sequence.

The terms, “non-naturally occurring” and “engineered,” as used herein are used interchangeably and indicate the involvement of human intervention. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by human intervention.

The term, “protospacer adjacent motif (PAM),” as used herein refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The terms, “reporter” and “reporter nucleic acid,” are used interchangeably herein to refer to a non-target nucleic acid molecule that can provide a detectable signal upon cleavage by an effector protein. Examples of detectable signals and detectable moieties that generate detectable signals are provided herein.

The term, “sample,” as used herein generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term, “target nucleic acid,” as used herein refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein when used in reference to a target nucleic acid refers to a sequence of nucleotides that hybridizes to a portion (preferably an equal length portion) of a guide nucleic acid. Hybridization of the guide nucleic acid to the target sequence may bring an effector protein into contact with the target nucleic acid.

The section headings used herein are for organizational purposes only and are not to be construed as limiting the subject matter described.

### VII. ILLUSTRATIVE EMBODIMENTS

The present disclosure provides the following illustrative embodiments.

Embodiment 1. A system for detecting a target nucleic acid, the system comprising:
(a) an instrument; and
(b) a cartridge configured to interface with the instrument, the cartridge comprising:
   (i) a reagent reservoir;
   (ii) a sample interface in fluid communication with the reagent reservoir, the sample interface configured to receive a sample;
   (iii) an amplification region in fluid communication with one or more of the sample interface or the reagent reservoir and configured to amplify one or more nucleic acids in the sample; and
   (iv) a detection region in fluid communication with the amplification region, the detection region comprising:
      (1) a programmable nuclease disposed within the detection region that is complexed with a guide nucleic acid, wherein the guide nucleic acid is complementary to a target nucleic acid, or a portion thereof, of the one or more nucleic acids, wherein the programmable nuclease is configured to be activated through binding of the guide nucleic acid to the target nucleic acid; and
      (2) a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid; and wherein the detection region is configured to enable detection of the signal.

Embodiment 2. The system of embodiment 1, wherein the reagent reservoir contains one or more sample preparation reagents and one or more beads stored therein.

Embodiment 3. The system of embodiment 2, wherein the one or more sample preparation reagents comprises liquid reagents, dried reagents, lyophilized reagents, or a combination thereof.

Embodiment 4. The system of embodiment 2 or 3, wherein the one or more sample preparation reagents comprise a protein digestion reagent, a cellular digestion reagent, one or more solvents, one or more lysis reagents, or a combination thereof.

Embodiment 5. The system of any one of embodiments 2-4, wherein the one or more beads are configured to bind with the target nucleic acid.

Embodiment 6. The system of embodiment 5, wherein the one or more beads comprise a silica coating.

Embodiment 7. The system of embodiment 5 or 6, wherein the one or more beads is disposed within the reagent reservoir as i) a dry powder, ii) a mixture with a liquid, or iii) a combination thereof.

Embodiment 8. The system of embodiment 7, wherein the liquid is a sample preparation reagent.

Embodiment 9. The system of any one of embodiments 5-8, wherein the one or more beads are magnetic.

Embodiment 10. The system of any one of embodiments 5-9, wherein the one or more beads are disposed within the reagent reservoir.

Embodiment 11. The system of any one of embodiments 2-10, wherein the reagent reservoir further comprises one or more capsules configured to contain a sample preparation reagent of the one or more sample preparation reagents and the one or more beads.

Embodiment 12. The system of embodiment 11, wherein each capsule in the reagent reservoir comprises a storage volume from about 50 μL to about 1000 μL or about 50 μL to about 500 μL.

Embodiment 13. The system of any one of embodiments 11-12, wherein the reagent reservoir comprises from about 1 to about 10 capsules.

Embodiment 14. The system of any one of embodiments 11-13, wherein the reagent reservoir further comprises a silo for holding each capsule.

Embodiment 15. The system of embodiment 14, wherein each capsule within the reagent reservoir is slidably disposed within a corresponding silo.

Embodiment 16. The system of any one of embodiments 11-15, wherein each capsule within the reagent reservoir comprises a capsule chamber for holding the sample preparation reagent and/or the one or more beads therein.

Embodiment 17. The system of embodiment 16, wherein each capsule chamber within the reagent reservoir further comprises a pierceable cover disposed at an end of the capsule chamber.

Embodiment 18. The system of embodiment 17, wherein each silo of embodiment 14 or 15 comprises a piercing mechanism configured to pierce through the pierceable cover of a capsule of the one or more capsules in the reagent reservoir, wherein the capsule is translated from a closed configuration to an open configuration, so as to release the sample preparation reagent and/or one or more beads therefrom.

Embodiment 19. The system of any one of embodiments 18, wherein the piercing mechanism comprises a piercer core disposed within the silo, wherein the capsule chamber is configured to slide towards the piercer core.

Embodiment 20. The system of embodiment 18 or 19, the instrument comprises an actuator platform configured to translate the piercing mechanism.

Embodiment 21. The system of embodiment 20, wherein the actuator platform is configured to release the sample reagent and/or one or more beads from each capsule simultaneously or according to any sequence of capsules.

Embodiment 22. The system of any one of embodiments 11-21, wherein the cartridge and/or instrument is configured to transfer the sample reagent and/or one or more beads to the sample interface via one or more valves.

Embodiment 23. The system of embodiment 22, wherein the one or more valves are configured to regulate flow between the reagent reservoir and the sample interface.

Embodiment 24. The system of embodiment 23, wherein the one or more valves comprise a rotary valve, a jumper, or any combination thereof.

Embodiment 25. The system of embodiment 24, wherein at least one of the valves comprises the jumper, wherein the jumper defines a jumper channel disposed within a housing thereof and enables fluid communication between reagent reservoir and the sample interface.

Embodiment 26. The system of embodiment 25, wherein the jumper comprises 1) an initial closed configuration, wherein fluid flow between the reagent reservoir and the sample interface is prevented, and 2) an open configuration, wherein fluid flow between the reagent reservoir and the sample interface is permitted.

Embodiment 27. The system of embodiment 26, wherein a first end of the jumper is located within a first jumper silo of the reagent reservoir and a second end of the jumper is located within a second jumper silo of the sample interface.

Embodiment 28. The system of embodiment 27, wherein the first end of the jumper is slidably disposed within the first jumper silo and wherein the second end of the jumper is slidable disposed within the second jumper silo.

Embodiment 29. The system of embodiment 27 or 28, wherein translating the jumper relative to the first jumper silo and the second jumper silo from a first position to a second position moves the jumper from the initial closed configuration to the open configuration.

Embodiment 30. The system of embodiment 29, wherein translating the jumper relative to the first jumper silo and the second jumper silo from a second position to a third position moves the jumper from the open configuration to a final closed position, thereby preventing fluid flow between the reagent reservoir and the sample interface.

Embodiment 31. The system of any one of embodiments 28-30, wherein the instrument comprises an actuator platform configured to translate the jumper from the first position to the second position, and from the second position to the third position.

Embodiment 32. The system of any one of embodiments 25-31, wherein the jumper channel is configured to contain any number of the one or more sample preparation reagents therein.

Embodiment 33. The system of any one of embodiments 1-32, wherein the sample interface is configured to receive the sample as a liquid, extract the sample from a swab, or both.

Embodiment 34. The system of embodiment 33, wherein the sample interface comprises a scraper to extract the sample from the swab.

Embodiment 35. The system of embodiment 33 or 34, wherein the sample interface comprises a sample reservoir into which the sample is configured to be transferred.

Embodiment 36. The system of any one of embodiments 2-35, wherein the sample interface is configured to mix the sample with the one or more sample preparation reagents and/or the one or more beads to form a mixed sample solution.

Embodiment 37. The system of embodiment 36, wherein the sample interface is configured to mix the sample via a cartridge heater, direct mechanical actuation, generating bubbles, passive mixing via fluid introduced into the sample interface, or a combination thereof.

Embodiment 38. The system of embodiment 37, wherein the cartridge heater is further configured to heat the sample to facilitate lysing therein.

Embodiment 39. The system of any one of embodiments 2-38, wherein the instrument and/or cartridge further comprises a magnet configured to immobilize the one or more beads when adjacent the one or more beads.

Embodiment 40. The system of embodiment 39, wherein the instrument is configured to move the magnet, thereby enabling movement of the one or more beads and nucleic acid bound thereto.

Embodiment 41. The system of any one of embodiments 35-40, wherein the reagent reservoir further comprises one or more concentration reagents and/or one or more elution reagents stored therein.

Embodiment 42. The system of any one of embodiments 35-40, wherein the cartridge further comprises a sample concentration region in fluid communication with the sample interface.

Embodiment 43. The system of embodiment 42, wherein the sample concentration region comprises one or more concentration reagents and/or one or more elution reagents stored therein.

Embodiment 44. The system of any one of embodiments 42-43, wherein the cartridge and/or instrument is configured to transfer the mixed sample solution to the sample concentration region via one or more valves.

Embodiment 45. The system of embodiment 44, wherein the one or more valves are configured to regulate flow between the sample interface and the sample concentration region.

Embodiment 46. The system of embodiment 45, wherein the one or more valves comprise the rotary valve or another rotary valve, a second jumper, or any combination thereof.

Embodiment 47. The system of embodiment 46, wherein at least one concentration reagent and/or at least one elution reagent is stored within a channel defined by the second jumper.

Embodiment 48. The system of any one of embodiments 41 or 43-47, wherein the one or more concentration reagents and/or one or more elution reagents comprises liquid reagents, dried reagents, lyophilized reagents, or a combination thereof.

Embodiment 49. The system of embodiment 48, wherein the one or more concentration reagents comprises wash reagents of one or more ionic strength, an alcohol, or a combination thereof.

Embodiment 50. The system of embodiment 48 or 49, wherein the one or more elution reagents comprises a low to no salt reagent.

Embodiment 51. The system of any one of embodiments 48-50, wherein the one or more elution reagents comprises a prescribed pH that enables releasing the target nucleic acid from the one or more beads.

Embodiment 52. The system of any one of embodiments 42-51, wherein the sample concentration region further comprises one or more capsules.

Embodiment 53. The system of embodiment 52, wherein each capsule in the sample concentration region is configured to contain a concentration reagent of the one or more concentration reagents and/or an elution reagent of the one or more elution reagents.

Embodiment 54. The system of any one of embodiments 52-53, wherein each capsule in the sample concentration region comprises a storage volume from about 100 μL to about 1 mL.

Embodiment 55. The system of any one of embodiments 52-53, wherein each capsule in the sample concentration region comprises a storage volume from about 250 μL to about 750 μL.

Embodiment 56. The system of any one of embodiments 52-55, wherein the sample concentration region comprises 3-10 capsules.

Embodiment 57. The system of any one of embodiments 52-55, wherein the sample concentration region comprises 5-7 capsules.

Embodiment 58. The system of any one of embodiments 41-57, wherein the sample interface, the second jumper, and/or the sample concentration region further comprises a filter mesh configured to capture the one or more beads bound to the target nucleic acid.

Embodiment 59. The system of any one of embodiments 41-58, wherein the sample interface and/or the sample concentration region further comprises a waste region configured to receive excess fluid from the mixed sample solution and at least one concentration reagent mixed therewith, wherein the target nucleic acid is immobilized via the filter mesh and/or via a magnet located on the instrument.

Embodiment 60. The system of embodiment 59, wherein the sample interface, the reagent reservoir, and/or the sample concentration region is further configured to elute the target nucleic acid from the one or more beads by contacting the one or more elution reagents thereto, thereby forming a concentrated nucleic acid solution.

Embodiment 61. The system of any one of embodiments 1-60, wherein the amplification region is configured to amplify the target nucleic acid via an isothermal reaction, thermocycling, reverse transcription, or any combination thereof.

Embodiment 62. The system of embodiment 61, wherein isothermal reaction is Loop-mediated isothermal amplification (LAMP).

Embodiment 63. The system of embodiment 61, wherein amplification via thermal cycling comprises polymerase chain-reaction (PCR).

Embodiment 64. The system of embodiment 61, wherein the sample interface, reagent reservoir, and/or sample concentration region is configured to transfer the concentrated nucleic acid solution to the amplification region.

Embodiment 65. The system of embodiment 61, wherein the concentrated nucleic acid solution is transferred to the amplification region via one or more valves.

Embodiment 66. The system of embodiment 62, wherein the one or more valves are configured to regulate flow between the sample interface, reagent reservoir, and/or sample concentration region and the amplification region.

Embodiment 67. The system of embodiment 63, wherein the one or more valves comprise the rotary valve or another rotary valve, a third jumper, or any combination thereof.

Embodiment 68. The system of any one of embodiments 61-67, wherein the amplification region and/or the third jumper comprises one or more amplification elution reagents stored therein.

Embodiment 69. The system of embodiment 68, wherein the one or more amplification reagents comprise liquid amplification reagents, dried amplification reagents, lyophilized amplification reagents, or a combination thereof.

Embodiment 70. The system of embodiment 69, wherein the liquid amplification reagents comprise one or more activator salts.

Embodiment 71. The system of any one of embodiments 68-69, wherein the amount of liquid amplification reagents stored is about 5 μL to about 30 μL.

Embodiment 72. The system of embodiment 69, wherein the lyophilized amplification reagents comprise assay-specific primers, dNTPs, reverse transcriptase enzymes, thermostable polymerase enzymes, additional additives, or any combination thereof.

Embodiment 73. The system of embodiment 72, wherein the additional additives comprise i) excipients such trehalose and/or raffinose, ii) BSA, iii) fish gelatin, iv) magnesium, v) other salts, or vi) any combination thereof.

Embodiment 74. The system of embodiment 69, wherein the lyophilized amplification reagents are in the form of one or more pellets.

Embodiment 75. The system of any one of embodiments 61-74, wherein the amplification region comprises one or more chambers.

Embodiment 76. The system of embodiment 75, wherein the concentrated nucleic acid solution is configured to mix with the one or more amplification reagents prior to entering the one or more chambers.

Embodiment 77. The system of any one of embodiments 1-76, wherein the amplification region comprises one or more chambers.

Embodiment 78. The system of any one of embodiments 75-77, wherein instrument and/or cartridge further comprises a thermal system to provide heat to the one or more chambers.

Embodiment 79. The system of embodiment 78, wherein the thermal system comprises a heating element, a cooling element, a controller in operative communication with the heating element or cooling element, and/or a feedback monitor in operative communication with the controller, wherein the feedback monitor is configured to detect the temperature of the one or more chambers or a fluid therein.

Embodiment 80. The system of embodiment 79, wherein the thermal system is configured to control the temperature within each chamber of the one or more chambers individually.

Embodiment 81. The system of embodiment 79 or 80, wherein the heating element and/or cooling element comprises a Peltier heating and/or cooling system.

Embodiment 82. The system of any one of embodiments 79-81, wherein the heating element is configured to optically heat the one or more chambers.

Embodiment 83. The system of embodiment 82, wherein at least one chamber of the one or more chambers comprises an optically transparent material.

Embodiment 84. The system of embodiment 82, wherein at least one chamber of the one or more chambers comprises an optically transparent window.

Embodiment 85. The system of any one of embodiments 75-84, wherein the one or more chambers comprises a plastic comprising polyethylene polyimide, or any thermally conductive plastic known in the art so as to promote nucleic acid amplification.

Embodiment 86. The system of any one of embodiments 75-85, wherein the one or more chambers comprises a plastic having a thermal conductivity of about 0.1 Watts/meter*Kelvin to about 100 Watts/meter*Kelvin.

Embodiment 87. The system of any one of embodiments 75-86, wherein the one or more chambers comprises a plastic and a metallic layer to maximize heat conductivity therein.

Embodiment 88. The system of any one of embodiments 75-87, wherein the thermal system is configured to control the temperature of at least one chamber of the one or more chambers to a prescribed temperature.

Embodiment 89. The system of embodiment 88, wherein the prescribed temperature is different for at least two chambers of the one or more chambers.

Embodiment 90. The system of embodiment 88 or 89, wherein the prescribed temperature for a chamber of the one or more chambers is from about 45.0° C. to about 70° C., from about 65° C. to about 90° C., or from about 80° C. to about 100° C.

Embodiment 91. The system of any one of embodiments 88-90, wherein the thermal system is configured to control the temperature in at least one chamber of the one or more chambers to within about 0.5° C. of the prescribed temperature or within about 2° C. of the prescribed temperature.

Embodiment 92. The system of any one of embodiments 79-91, wherein the feedback monitor comprises a temperature sensor for measuring the temperature in a chamber of the one or more chambers.

Embodiment 93. The system of embodiment 92, wherein the temperature sensor comprises an infrared sensor, an integrated circuit sensor, a resistance temperature detector, and/or a thermocouple.

Embodiment 94. The system of embodiment 92, wherein the temperature sensor comprises temperature sensitive component.

Embodiment 95. The system of embodiment 94, wherein the temperature sensitive component comprises a thermistor.

Embodiment 96. The system of any one of embodiments 92-95, wherein the temperature sensor comprises a surface coating and/or packing element so as to minimize or prevent interference with an amplification reaction.

Embodiment 97. The system of any one of embodiments 75-96, wherein each chamber of the one or more chambers has an internal volume of about 10 μL to about 20 μL.

Embodiment 98. The system of any one of embodiments 75-97, wherein the amplification reagents stored on the amplification region is pre-aliquoted into separate amounts for each chamber of the one or more chambers.

Embodiment 99. The system of any one of embodiments 1-98, wherein the detection region is configured for spatially multiplexed detection of a plurality of target nucleic acids in the sample.

Embodiment 100. The system of any one of embodiments 1-99, wherein the programmable nuclease comprises a Cas protein.

Embodiment 101. The system of embodiment 100, wherein the Cas protein comprises Cas12, Cas13, Cas14, CasPhi, a thermostable Cas, or any combination thereof.

Embodiment 102. The system of any one of embodiments 1-101, wherein the detection region is configured to perform a liquid-based reaction or an immobilized array reaction.

Embodiment 103. The system of embodiment 102, wherein the detection region comprises an array having a plurality of detection spots thereon to perform an immobilized array reaction.

Embodiment 104. The system of embodiment 102, wherein each detection spot comprises a specific guide nucleic acid corresponding to a particular target nucleic acid for detection, the specific guide nucleic acid being immobilized to a surface of the detection region.

Embodiment 105. The system of embodiment 104, wherein each detection spot further comprises a reporter immobilized to the surface.

Embodiment 106. The system of embodiment 105, wherein each programmable nuclease, guide nucleic acid, and/or reporter are immobilized on a detection spot using NHS-amine chemistry, streptavidin-biotin chemistry, or a combination thereof.

Embodiment 107. The system of any one of embodiments 103-106, wherein the array comprises a microwell array, such that each detection spot corresponds to a microwell.

Embodiment 108. The system of embodiment 107, wherein each microwell is from about 150 μm to about 500 μm in diameter and from about 150 μm to about μm in depth.

Embodiment 109. The system of embodiment 107 or 108, wherein each microwell is comprises a hydrophilic material or coating on an inside surface, and/or a hydrophobic material or coating on the outside and/or surrounding the microwell.

Embodiment 110. The system of any one of embodiments 103-109, wherein the plurality of detection spots are from about 10 to about 200 detection spots.

Embodiment 111. The system of embodiment 102, wherein the detection region comprises one or more liquid detection chambers for performing a liquid-based reaction.

Embodiment 112. The system of embodiment 111, wherein the cartridge further comprises a mixing chamber between the amplification region and the detection region.

Embodiment 113. The system of embodiment 111 or 112, wherein each chamber of the one or more chambers within the amplification region is mapped to a corresponding liquid detection chamber of the one or more liquid detection chambers.

Embodiment 114. The system of any one of embodiments 111-113, wherein the thermal system or another thermal system is configured to heat each liquid detection chamber.

Embodiment 115. The system of embodiment 114, wherein the liquid detection chamber is heated from about 35° C. to about 40° C.

Embodiment 116. The system of any one of embodiments 111-115, wherein the programmable nucleic acid, guide nucleic acid, and/or reporter are immobilized within the detection region.

Embodiment 117. The system of any one of embodiments 99-116, wherein the amplification region is configured to transfer the amplified target nucleic acid to the detection region.

Embodiment 118. The system of embodiment 117, wherein the amplified target nucleic acid is transferred to the detection region via one or more valves.

Embodiment 119. The system of embodiment 117, wherein the one or more valves are configured to regulate flow between the amplification region and the detection region.

Embodiment 120. The system of embodiment 118, wherein the one or more valves comprise the rotary valve or another rotary valve, a fourth jumper, or any combination thereof.

Embodiment 121. The system of any one of embodiments 117-120, wherein the detection region and/or the fourth jumper comprises one or more detection reagents stored therein.

Embodiment 122. The system of any one of embodiments 100-121, wherein the programmable nucleic acid, guide nucleic acid, and/or reporter are provided as lyophilized detection reagents.

Embodiment 123. The system of any one of embodiments 1-122, wherein the instrument further comprises an optical sensor for detecting the presence of the target nucleic acid.

Embodiment 124. The system of embodiment 123, wherein the optical sensor comprises an image sensor or an array of discrete optical detectors.

Embodiment 125. The system of any one of embodiments 1-124, wherein the detection of the presence of the target nucleic acid is via detecting 1) fluorescence, 2) a color change, 3) a brightness change, 4) a wavelength change of a light, or 5) a combination thereof.

Embodiment 126. The system as in any one of the preceding embodiments, wherein the one or more capsules are aligned linearly or radially.

Embodiment 127. The system of any one of embodiments 1-126, wherein the interface between the instrument and cartridge enables operative communication therebetween.

Embodiment 128. The system of any one of embodiments 1-127, wherein the instrument comprises an opening to receive the cartridge.

Embodiment 129. The system of any one of embodiments 1-128, wherein the interface between the cartridge and the instrument enables alignment with 1) ports on the cartridge that facilitate movement of the sample therein, 2) the one or more capsules in the reagent reservoir, the first jumper, the second jumper, the one or more capsules in the sample concentration region, or a combination thereof, to facilitate release of contents therein, 3) the amplification region for provision of heat, 4) the detection region, for detecting the presence of the target nucleic acid, or 5) a combination thereof.

Embodiment 130. The system of any one of embodiments 1-129, wherein the instrument comprises an XYZ motorized gantry configured to operatively communicate with the cartridge.

Embodiment 131. The system of any one of embodiments 1-130, wherein the instrument comprises a pump.

Embodiment 132. The system of any one of embodiments 1-131, wherein the cartridge and/or instrument is configured to move fluid within the cartridge and different regions via positive and/or negative pressure.

Embodiment 133. The system of embodiment 132, wherein the cartridge and/or instrument further comprises a syringe to supply the positive and/or negative pressure.

Embodiment 134. The system of any one of embodiments 1-133, wherein the reagent reservoir, the sample interface, the sample concentration region, the waste region, the amplification region, and/or the detection region comprises separate modules that are coupled together and in fluid communication with each other.

Embodiment 135. The system of embodiment 134, wherein the reagent reservoir, the sample interface, the sample concentration region, the waste region, the amplification region, and/or the detection region comprises separate detachably coupled modules.

Embodiment 136. The system of any one of embodiments 1-135, wherein the sample interface is configured to be in fluid communication with a serpentine channel to enable amplification of the target nucleic acid and/or lysis of the sample.

Embodiment 137. The system of embodiment 1, wherein the instrument is configured to control fluid, temperature, and detection parameters of reactions occurring within the cartridge.

Embodiment 138. The system of embodiment 1, wherein the programmable nuclease and the reporter are immobilized to a surface of the detection region.

Embodiment 139. The system of embodiment 1, wherein the programmable nuclease and the reporter are contained within a chamber of the detection region, wherein the programmable nuclease and the reporter are configured to react in liquid phase.

Embodiment 140. The system of embodiment 1, wherein the instrument comprises an optical sensor configured to detect a detection moiety released upon cleaving of the reporter by an activated programmable nuclease.

Embodiment 141. The system of any one of embodiments 1-140, wherein the cartridge comprises two separate components coupled together.

Embodiment 142. A system for detecting a target nucleic acid, the system comprising:
(a) an instrument;
(b) a cartridge configured to interface with the instrument, the cartridge comprising: (i) a sample interface configured to receive a sample comprising one or more nucleic acids; (ii) one or more reagent capsules; and (iii) a detection region;
(c) a programmable nuclease disposed within the cartridge and that is complexed with a guide nucleic acid that is complementary to the target nucleic acid, or a portion thereof, of the one or more nucleic acids, wherein the programmable nuclease is configured to be activated through binding of the guide nucleic acid to the target nucleic acid; and
(d) a reporter disposed within the cartridge, the reporter comprising a cleavable nucleic acid and a detection moiety, wherein cleavage of the cleavable nucleic acid by the activated programmable nuclease releases the detection moiety from the cleavable nucleic acid, wherein the released detection moiety is configured to generate a signal;
   wherein the detection region is configured to detect the signal indicating the presence of the target nucleic acid.

Embodiment 143. The system of embodiment 142, wherein the sample interface comprises a scraper to extract the sample from a swab.

Embodiment 144. The system of embodiment 142, further comprising a sample preparation region configured to purify and concentrate the one or more nucleic acids.

Embodiment 145. The system of embodiment 144, wherein the sample preparation region further comprises a subset of the one or more reagent capsules, wherein the subset comprises a protein digestion reagent, a cellular digestion reagent, one or more solvents, or a combination thereof.

Embodiment 146. The system of embodiment 145, wherein the liquid capacity of each of the reagent capsules ranges from 50 μL to 500 μL in volume.

Embodiment 147. The system of embodiment 145 or 146, wherein the subset contains 4 to 6 reagent capsules.

Embodiment 148. The system of embodiment 144, wherein the sample preparation region comprises one or more beads or membranes having a silica coating, wherein the silica coating is configured to bind at least one nucleic acid of the one or more nucleic acids.

Embodiment 149. The system of embodiment 148, wherein the silica beads are magnetic silica beads.

Embodiment 150. The system of any one of embodiments 149, wherein the instrument comprises a magnet configured to immobilize and release the magnetic silica beads.

Embodiment 151. The system of embodiment 142, further comprising an amplification region.

Embodiment 152. The system of embodiment 151, further comprising amplification reagents.

Embodiment 153. The system of embodiment 152, wherein the amplification reagents are present as liquid amplification reagents and lyophilized amplification reagents.

Embodiment 154. The system of embodiment 152, wherein the liquid amplification reagents comprise one or more activator salts.

Embodiment 155. The system of embodiment 154, wherein the lyophilized amplification reagents comprise assay-specific primers, probes, dNTPs, reverse transcriptase enzymes, thermostable polymerase enzymes, additional additives, or any combination thereof.

Embodiment 156. The system of embodiment 152, wherein the lyophilized amplification reagents are in the form of one or more pellets.

Embodiment 157. The system of embodiment 142, wherein the instrument is configured to control fluid, temperature and detection parameters of reactions occurring within the cartridge.

Embodiment 158. The system of embodiment 142, wherein the detection region is configured for spatially multiplexed detection of a plurality of target nucleic acids in the sample.

Embodiment 159. The system of embodiment 142, wherein the programmable nuclease, the guide nucleic acid, or the reporter are immobilized to a surface of the detection region.

Embodiment 160. The system of embodiment 142, wherein the programmable nuclease and the reporter are contained within a chamber of the detection region, wherein the programmable nuclease and the reporter are configured to react in liquid phase.

Embodiment 161. The system of embodiment 142, wherein the instrument comprises an optical sensor configured to detect a detection moiety released upon cleaving of the reporter by an activated programmable nuclease.

Embodiment 162. A system for multiplexed detection of a plurality of target nucleic acids comprising:

(a) an instrument; and

(b) a cartridge comprising: (i) a sample interface; (ii) one or more reagent capsules; (iii) a sample preparation region; (iv) an amplification region; and (v) a detection region, the detection region comprising a plurality of detection locations, each detection location of the plurality of detection locations comprising a reporter and a programmable nuclease complexed with a guide nucleic acid that is complementary to a different target nucleic acid of a plurality of target nucleic acids;

wherein, at each detection location, the corresponding reporter and the corresponding guide nucleic acid are immobilized to a surface of the detection region;

wherein, at each detection location, the corresponding programmable nuclease is configured to cleave the reporter and generate a different signal of a plurality of signals; and

wherein each different signal of the plurality of signals indicates a presence or absence of each different target nucleic acid at its respective detection spot.

Embodiment 163. The system of embodiment 162, wherein the plurality of different locations are arranged in an array configuration.

Embodiment 164. The system of embodiment 162, wherein the plurality of different locations comprises a plurality of chambers.

Embodiment 165. The system of any one of embodiments 162-164, wherein each detection location comprises a different reporter.

Embodiment 166. A method for detecting a target nucleic acid, the method comprising the steps of:
(a) receiving a sample containing a target nucleic acid via a sample interface;
(b) concentrating the target nucleic acid;
(c) transferring the target nucleic acid to a detection region; and
(d) detecting the target nucleic acid, wherein a programmable nuclease is activated by the target nucleic acid, the activated programmable nuclease cleaving a reporter and releasing a detection moiety, thereby generating a signal indicating the presence or absence of the target nucleic acid.

Embodiment 167. The method of embodiment 166, further comprising transferring the target nucleic acid to an amplification region.

Embodiment 168. The method of embodiment 166, further comprising amplifying the target nucleic acid.

Embodiment 169. The method of embodiment 166, further comprising outputting results.

Embodiment 170. A method for detecting a target nucleic acid, the method comprising the steps of:
(a) combining a first cartridge component containing liquid reagents with a second cartridge component containing dried or lyophilized reagents, thereby reconstituting the dried or lyophilized reagents, wherein the combining results in the first cartridge component and second cartridge component comprising an assembled cartridge;
(b) receiving a sample containing a target nucleic acid via a sample interface;
(c) lysing the sample;
(d) purifying and concentrating the target nucleic acid;
(e) transferring the target nucleic acid to an amplification region;
(f) amplifying the target nucleic acid;
(g) transferring the target nucleic acid to a detection region comprising a programmable nuclease complexed to a guide nucleic acid, wherein the detection region further comprises a reporter comprising a detection moiety; and
(h) detecting the target nucleic acid, wherein the programmable nuclease is activated by binding of the target nucleic acid to the guide nucleic acid, wherein activation of the programmable nuclease cleaves a reporter, thereby releasing a detection moiety from the reporter and generating a signal indicative of a presence or absence of the target nucleic acid.

Embodiment 171. A method for detecting a target nucleic acid, the method comprising the steps of:
(a) receiving a sample containing a plurality of target nucleic acids via a sample interface;
(b) loading the cartridge into an instrument;
(c) transferring the sample to a sample preparation region located within the cartridge;
(d) lysing the sample;
(e) purifying the plurality of target nucleic acids from the sample;
(f) concentrating the plurality of target nucleic acids;
(g) transferring the plurality of target nucleic acids to an amplification region;
(h) amplifying the plurality of target nucleic acids;
(i) transferring the plurality of target nucleic acids to a detection region, the detection region comprising a plurality of detection locations, each detection location comprising a reporter and a programmable nuclease of a plurality of programmable nucleases, wherein each programmable nuclease comprises a different guide nucleic acid complementary to a different target nucleic acid of the plurality of target nucleic acids; and wherein the corresponding reporter and the corresponding programmable nuclease of each detection location are immobilized to a surface of the detection region, and wherein at each detection location, each programmable nuclease is configured to cleave the corresponding reporter, thereby generating a different signal of a plurality of signals, each different signal of the plurality of signals indicating the presence or absence of each different complementary target nucleic acid of the plurality of target nucleic acids.

Embodiment 172. A system for detecting a target nucleic acid, the system comprising a detection region comprising:
(i) a guide nucleic acid complementary to the target nucleic acid, or a portion thereof; and
(ii) a reporter immobilized to a surface of the detection region, the reporter comprising a nucleic acid and a detection moiety, wherein (a) the nucleic acid is at least 40 nucleotides in length; (b) the nucleic acid comprises a double-stranded region; or (c) a combination of (a) and (b); and
   wherein cleavage of the reporter by a programmable nuclease, activated upon hybridization to the target nucleic acid, releases the detection moiety from the nucleic acid, and wherein the release of the detection moiety is configured to generate a signal indicative of a presence of the target nucleic acid.

Embodiment 173. A system for detecting a target nucleic acid, the system comprising:
a detection region comprising at least one detection location comprising:
a programmable nuclease disposed within the detection region that is complexed with a guide nucleic acid, wherein the guide nucleic acid is complementary to a target nucleic acid, or a portion thereof, wherein the programmable nuclease is configured to be activated through binding of the guide nucleic acid to the target nucleic acid;
a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid; and
a surface comprising a hydrophobic membrane.

Embodiment 174. A system comprising a microfluidic device comprising a plurality of chambers fluidically connected in sequence, wherein:
(a) each chamber of the plurality of chambers comprises a well, an inlet channel, an outlet, and a capillary valve;
(b) the capillary valve of each chamber (i) has a cross-sectional area that is smaller than a cross-sectional area of the inlet channel of the respective chamber, and (ii) forms an entrance of the inlet channel of the next chamber in the sequence; and
(c) each outlet is air-permeable and configured to retain liquid within the respective chamber.

Embodiment 175. The system of embodiment 174, wherein each chamber further comprises detection reagents comprising a guide nucleic acid and a reporter, and further wherein:
(a) each guide nucleic acid (i) comprises a targeting sequence that hybridizes with a target nucleic acid of a plurality of different target nucleic acids or an amplicon thereof, and (ii) is effective to form a complex with a programmable nuclease that is activated upon binding the corresponding target nucleic acid or amplicon thereof;
(b) the guide nucleic acid of a first chamber in the plurality of chambers comprises a different targeting sequence from the guide nucleic acid of a second chamber in the plurality of chambers; and
(c) each reporter (i) comprises a cleavable nucleic acid and a detection moiety, and (ii) is configured to be cleaved to form a detectable cleavage product in response to activation of the complex in the well of the respective chamber.

Embodiment 176. The system of embodiment 174 or 175, wherein the capillary valve has a cross-sectional area that is 75%, 50%, or less than a cross-sectional area of the inlet channel of the respective chamber.

Embodiment 177. The system of any one of embodiments 174-176, wherein the capillary valve is oriented at an angle of 90° or greater with respect to the inlet channel of the respective chamber.

Embodiment 178. The system of embodiment 177, wherein the capillary valve forms a junction with the inlet channel of the respective chamber.

Embodiment 179. The system of any one of embodiments 174-176, wherein the capillary valve and the inlet channel intersect the well of a respective chamber at separate points along a perimeter of the well.

Embodiment 180. The system of any one of embodiments 174-179, wherein the inlet channels comprise (a) a width of about 0.3 mm to about 0.6 mm and a depth of about 0.25 mm to about 0.45 mm; (b) a width of about 0.4 mm and a depth of about 0.35 mm; or (c) a width of about 0.5 mm and a depth of about 0.35 mm.

Embodiment 181. The system of any one of embodiments 174-180, wherein the capillary valves comprise (a) a width of about 0.2 mm to about 0.4 mm and a depth of about 0.1 mm to about 0.3 mm; or (b) a width of about 0.3 mm and a depth of about 0.2 mm.

Embodiment 182. The system of any one of embodiments 174-181, wherein each of the wells has an internal volume of (a) about 0.1 μL to about 50 μL, (b) about 0.5 μL to about 20 μL, (c) about 0.75 μL, or (d) about 10 μL.

Embodiment 183. The system of any one of embodiments 174-182, wherein the outlet comprises (a) an opening sized to permit displacement of air therethrough but to retain liquid within the well under an operating pressure of the microfluidic device, (b) a surface comprising a hydrophobic coating, or (c) a surface comprising an air-permeable hydrophobic membrane.

Embodiment 184. The system of any one of embodiments 183, wherein the outlet comprises the surface comprising the air-permeable hydrophobic membrane, and further wherein the hydrophobic membrane (a) comprises woven polypropylene or woven polyethylene, (b) the hydrophobic membrane comprises pores of about 0.1 microns to about 2 microns in size, and/or (c) forms a bottom surface of the respective well.

Embodiment 185. The system of any one of embodiments 174-184, further comprising a sample interface configured to receive a sample, wherein the sample interface is in fluid communication with the plurality of chambers.

Embodiment 186. The system of any one of embodiments 174-185, wherein the sample interface is fluidically connected to the plurality of chambers via one or more sample preparation regions.

Embodiment 187. The system of embodiment 186, wherein the one or more sample preparation regions comprise a lysis region configured to lyse one or more components of the sample, optionally wherein the lysis region comprises lysis reagents.

Embodiment 188. The system of embodiment 186 or 187, wherein the one or more sample preparation regions comprise an amplification region, optionally wherein the amplification region comprises amplification reagents.

Embodiment 189. The system of any one of embodiments 186-188, wherein (a) the sample interface is fluidically connected to the plurality of chambers via a bubble purge channel, (b) the bubble purge channel is connected to a sample inlet channel at an upstream end and a sample exit channel at a downstream end; and (c) the bubble purge channel is configured to purge gas bubbles from the sample fluid.

Embodiment 190. The system of embodiment 189, wherein (a) a surface of the bubble purge channel comprises a gas-permeable membrane that is hydrophobic and/or oleophobic; and (b) the bubble purge channel is dimensioned to provide a pressure drop downstream from the bubble purge channel.

Embodiment 191. The system of any one of embodiments 174-190, wherein (a) the outlets vent through a first surface of the microfluidic device, (b) the system further comprises a heater in thermal communication with a second surface of the microfluidic device, and (c) the first surface is opposite the second surface.

Embodiment 192. The system of any one of embodiments 174-191, wherein the plurality of chambers comprises at least 10, 25, 50, or 100 chambers fluidically connected in sequence.

Embodiment 193. The system of any one of embodiments 175-192, wherein the detection reagents further comprise a programmable nuclease.

Embodiment 194. The system of embodiment 193, wherein the programmable nuclease comprises a Cas protein, optionally wherein the Cas protein is selected from a Cas12, a Cas13, a Cas14, a CasPhi, and a thermostable Cas.

Embodiment 195. The system of any one of embodiments 175-194, wherein the detection reagents further comprise amplification reagents.

Embodiment 196. The system of any one of embodiments 175-195, wherein the detection reagents are in a lyophilized form.

Embodiment 197. The system of any one of embodiments 175-196, wherein the guide nucleic acid and/or the reporter in each chamber are immobilized to a surface of the respective chamber.

Embodiment 198. A method for detecting one or more of a plurality of different target nucleic acids in a system of any one of embodiments 175-197, the method comprising:
(a) flowing a liquid comprising one or more of the different target nucleic acids or amplicons thereof into the plurality of chambers;
(b) in one or more of the wells, forming the activated complex and cleaving the reporters; and
(c) detecting the detectable cleavage products in one or more of the wells, wherein the location of a well comprising a detectable cleavage product identifies the target nucleic acid or amplicon thereof present in the well.

Embodiment 199. A microfluidic device comprising:
a loading channel comprising a first capillary valve disposed upstream of a second capillary valve disposed therein;
a first chamber fluidically coupled to the loading channel upstream of the first capillary valve;
a second chamber fluidically coupled to the loading channel between the first capillary valve and the second capillary valve; and
a third chamber fluidically coupled to the loading channel downstream of the second capillary valve, wherein:

(a) each chamber of the first, second, and third chambers comprises an outlet;
(b) each of the first and second capillary valves have a cross-sectional area that is smaller than a cross-sectional area of the loading channel; and
(c) each outlet is gas-permeable and configured to retain liquid within the respective chamber.

Embodiment 200. A cartridge for use in a system for detecting a target nucleic acid, the cartridge being configured to interface with an instrument of the system, the cartridge comprising:
a reagent reservoir;
a sample interface in fluid communication with the reagent reservoir, the sample interface configured to receive a sample;
an amplification region in fluid communication with one or more of the sample interface or the reagent reservoir and configured to amplify one or more nucleic acids in the sample; and
a detection region in fluid communication with the amplification region, the detection region comprising:
   (a) a programmable nuclease disposed within the detection region that is complexed with a guide nucleic acid, wherein the guide nucleic acid is complementary to a target nucleic acid, or a portion thereof, of the one or more nucleic acids, wherein the programmable nuclease is configured to be activated through binding of the guide nucleic acid to the target nucleic acid; and
   (b) a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid;
wherein the detection region is configured to enable detection of the signal.

Embodiment 201. An instrument for use in a system for detecting a target nucleic acid, wherein the instrument is configured to interface with a cartridge according to embodiment 200.

Embodiment 202. A cartridge for use in a system for detecting a target nucleic acid, the cartridge being configured to interface with an instrument of the system, the cartridge comprising:
a reagent reservoir;
a sample interface in fluid communication with the reagent reservoir, the sample interface configured to receive a sample;
an amplification region in fluid communication with one or more of the sample interface or the reagent reservoir and configured to amplify one or more nucleoid acids in the sample; and
a detection region in fluid communication with the amplification region, the detection region configured to generate a signal indicative of a presence of the target nucleic acid; and wherein the detection region is configured to enable detection of the signal.

Embodiment 203. A system for detecting a target nucleic acid, the system comprising a cartridge, wherein the cartridge comprises:
(a) a reagent reservoir;
(b) a sample interface in fluid communication with the reagent reservoir, wherein the sample interface is configured to receive a sample; and
(c) an amplification region in fluid communication with one or more of the sample interface or the reagent reservoir, and configured to amplify one or more nucleic acids in the sample.

Embodiment 204. The system of embodiment 203, wherein the cartridge further comprises a detection region in fluid communication with the amplification region.

Embodiment 205. The system of embodiment 203 or 204, further comprising an instrument configured to interface with the cartridge.

Embodiment 206. A cartridge for detecting a target nucleic acid, the cartridge comprising:
(a) a reagent reservoir;
(b) a programmable nuclease, wherein the programmable nuclease is configured to be activated through binding of a guide nucleic acid to a target nucleic acid;
(c) a reporter, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid;
(d) a sample interface in fluid communication with the reagent reservoir, the sample interface configured to receive a sample;
(e) a detection region in fluid communication with the sample interface, the detection region comprising:
   (i) a guide nucleic acid disposed within the detection region, wherein the guide nucleic acid is complementary to the target nucleic acid, or a portion thereof, of the one or more nucleic acids; and
   (ii) a primer disposed within the detection region, wherein the primer is designed to amplify one or more nucleic acids in the sample; and
wherein the detection region is configured to amplify the one or more nucleic acids in the sample and to enable detection of the signal.

### EXAMPLES

The following examples are included for illustrative purposes only and are not intended to limit the scope of the invention.

### Example 1: Microarray System for Programmable Nuclease-Based Multiplexed Assays

The purpose of this example is to describe a system for multiplexed detection of a plurality of target nucleic acids. The central aspect of this system is a programmable nuclease-based microarray configured for the detection of a plurality of different target nucleic acids. The system is made up of an instrument; a cartridge comprising: a sample reservoir; a sample interface; one or more reagent reservoirs comprising reagent capsules in fluid communication with the sample interface or the sample reservoir; an amplification region in fluid communication with the sample reservoir; and a detection region in fluid communication with the amplification region. A sample is collected and received in the cartridge by the sample interface fluidly coupled to a sample reservoir. The sample is extracted from the sample reservoir and eluted into the amplification region for nucleic acid amplification before being transferred to the detection region. The detection region comprises an array of detection spots, or microarray, disposed on a surface thereof. Each detection spot contains a reporter and a programmable nuclease probe, where the programmable nuclease probe contains both a programmable nuclease and a guide nucleic acid. In this example, the programmable nuclease at each detection is coupled to a guide nucleic acid that is specific toward a particular target nucleic acid sequence. The reporter and the programmable nuclease of each detection spot of the array are immobilized to the surface of the detection region. When the guide nucleic acid of the programmable nuclease selectively binds to its complementary target nucleic acid, the programmable nuclease is activated, thereby enabling trans-cleavage of a cleavable nucleic acid of the reporter. When the reporter is cleaved, a portion of the reporter comprising a detection moiety is released from the surface. The detection moiety, in this example, is a quencher. Once the quencher is released, a fluorophore still coupled to the immobilized portion of the reporter is no longer quenched and exhibits fluorescence upon excitation by an illumination source of the instrument. The excitation light from the illumination source in the instrument is transmitted through a window of the cartridge onto the array of detection spots in the detection region. Likewise, the emitted fluorescence is transmitted from the detection spot of the array in the cartridge to an optical sensor or detector in the instrument through the window of the cartridge. In this manner, each detection spot within the array is assessed and any detection spot exhibiting fluorescence signifies that the quencher has been released by an activated programmable nuclease, thus indicating that the target nucleic acid specific to the fluorescing spot is present in the sample. The detection spots are spatially encoded with known different programmable nuclease-guide complexes at each detection spot, thereby allowing for multiplexed target analysis on a single sample.

### Example 2: Thermocycling Using Thermal Reservoirs

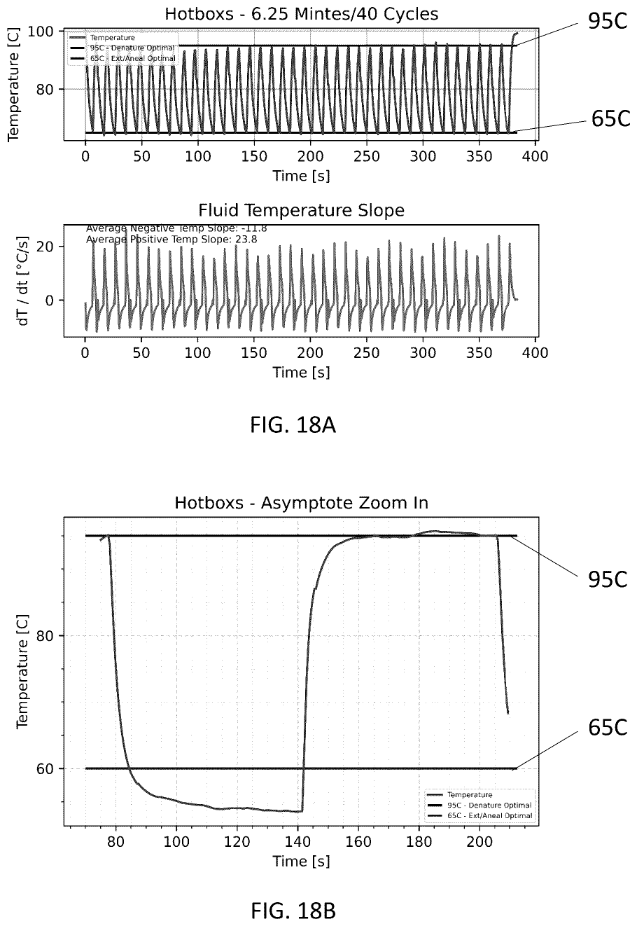

FIG. 18A shows plots of temperature over time (top) and the fluid temperature slope (bottom) generated in a fluid channel during thermocycling with the thermal reservoir setup of FIG. 12A. A first thermal reservoir was heated to 65° C. and positioned above the fluid channel. A second thermal reservoir was heated to 95° C. and positioned below the fluid channel. The first and second thermal reservoirs were alternately moved in and out of contact with the top and bottom of the fluid channel, respectively, in order to cool or heat the fluid within the channel as described herein. Forty cycles of alternating between 65° C. and 95° C. were run over a period of 6 minutes and 15 seconds. Fluid temperature was measured continuously during thermocycling with the thermal reservoirs. The thermal reservoirs were able to rapidly cycle the temperature of the fluid alternating contact of the fluid channel with the first and second thermal reservoirs.

### Example 3: Temperature Maintenance Using Thermal Reservoirs

FIG. 18B shows a plot of asymptotic temperature over time generated in a fluid channel with the thermal reservoir setup of FIG. 12A. The fluid channel was initially placed in contact with the second thermal reservoir in order to heat the fluid in the channel to 95° C. prior to temperature monitoring. Contact with the second thermal reservoir was then removed as the first thermal reservoir contacted the fluid channel. After 140 seconds, the first thermal reservoir was removed and the second thermal reservoir was contact to the fluid channel in order to heat the fluid. The thermal reservoirs were able to rapid adjust the temperature of the fluid and maintain a relatively stable fluid temperature while in contact with either of the thermal reservoirs.

### Example 4: Thermocycling Using Interzone Thermal Reservoirs

FIG. 25C shows plots of temperature over time generated in a fluid channel during thermocycling with the thermal reservoir setup of FIGS. 25A-25B. A first thermal reservoir was heated to 65° C. and positioned below a first fluid channel in a first temperature zone of the cartridge. A second thermal reservoir was heated to 95° C. and positioned below a second fluid channel in a second temperature zone of the cartridge. The fluid was pumped between first and second temperature zones in order to cool or heat the fluid within the channels as described herein. Forty cycles of alternating between 65° C. and 95° C. were run over a period of 91.7 seconds. Fluid temperature was measured continuously during thermocycling with the interzone thermal reservoirs. The interzone thermal reservoirs were able to rapidly cycle the temperature of the fluid by moving the fluid between channels in contact with the first and second thermal reservoirs.

### Example 5: Thermocycling Using Low Mass Heaters

Table 4 shows heating and cooling speeds achieved over five cycles between 65° C. and 95° C. using the cartridges shown in FIG. 26G. The temperature of the fluid was measured at the start and end of each temperature ramp and used to determine the heating and cooling speeds shown in Table 4. Table 5 shows projected cycling durations for 40 cycles based on the mean speeds determined in Table 4. Forty cycles of alternating between 65° C. and 95° C. was estimated to take a period of about 225s seconds.

TABLE 4
Minimum, Maximum and Mean Heating and Cooling Speeds from a Single Test
Run per Chip, with 5 cycles per Test Run, 65° C.-95° C.

| | Temp | Heating Speed (° C./s) | | | Cooling Speed (° C./s) | | |
|---|---|---|---|---|---|---|---|
| Chip | Sensor Used | Min. | Max. | Mean | Min. | Max. | Mean |
| MTH-1GF111 | Trace RTD | 19.687 | 32.837 | 24.950 | −8.046 | −8.536 | −8.362 |
| MTH-1GF121 | RTD2 | 13.343 | 16.512 | 14.585 | −8.054 | −8.743 | −8.443 |
| MTH-1GF211¹ | Trace RTD | 8.072 | 11.007 | 9.861 | −7.620 | −7.756 | −7.683 |
| MTH-1GF311² | Therm 1 | 6.384 | 6.669 | 6.499 | −1.760 | −1.859 | −1.822 |
| MTH-1GF321³ | RTD2 | 14.019 | 22.187 | 18.564 | −1.507 | −1.697 | −1.591 |

¹Fluid evacuated from the channel during the first cycle, due to boiling of fluid. For this configuration, the trace RTD temperature measurement is significantly lower than the actual fluid temperature.
²Test run conducted without fan during cooldown, and without fluid in channel.
³Test run conducted without fan during cooldown.

TABLE 5
Projected cycling duration, 40 cycles 60° C.-95°
C., No Hold, Based on Mean Speeds in Table 4

| | Heating | | Cooling | | Duration at |
|---|---|---|---|---|---|
| | | 60 → 95° C., | | 95 → 60° C. | 40 cycles |
| | Mean | Duration, T₁ | Mean | Duration, T₂ | {40 × (T₁ + |
| | Speed, s | {35 ÷ s} | Speed, s | {−35 ÷ s} | T₂) ÷ 60} |
| Chip | (° C./s) | (s) | (° C./s) | (s) | (min) |
| MTH- | 24.950 | 1.403 | −8.362 | 4.186 | 3.73 |
| 1GF111 | | | | | |
| MTH- | 14.585 | 2.400 | −8.443 | 4.145 | 4.36 |
| 1GF121 | | | | | |
| MTH- | 9.861 | 3.549 | −7.683 | 4.556 | 5.40 |
| 1GF211 | | | | | |
| MTH- | 6.499 | 5.385 | −1.822 | 19.210 | 16.40 |
| 1GF311 | | | | | |
| MTH- | 18.564 | 1.885 | −1.591 | 21.999 | 15.92 |
| 1GF321 | | | | | |

### Example 6: Thermocycling Using Photonics

FIG. 27B shows plots of temperature over time generated in a fluid channel during thermocycling with the photonic heater setup of FIG. 27A. Two 1465 nm laser diodes were used to heat the sample. Cooling was performed by forced air using two fans, while sensing of the liquid temperature was performed with an infrared sensing thermometer. Forty cycles of alternating between 65° C. and 95° C. were run over a period of 201 seconds. Fluid temperature was measured continuously during thermocycling with the interzone thermal reservoirs. Heating duration from 60→95° C. was about 1.83s. Cooling duration from 95→60° C. was about 4.65s. The photonic heater was able to rapidly cycle the temperature of the fluid.

### Example 7: Thermocycling Using Thermoelectric Coolers

FIGS. 28C-28D show plots of temperature over time generated in a fluid chamber (FIG. 28A) or at the surface of the heater (FIG. 28B) during thermocycling with the thermoelectric heater setup of FIGS. 28A-28B. A consumable having a single chamber was fabricated using 3D printing to provide a slab fluid volume for testing. A thermistor in the fluid was used to measure fluid temp and its relationship to top plate (heater surface) temperature. The control system, TEC drive characteristics, bubble formation (and reduction), and consumable clamping were optimized to improve speed of the assembly. Forty cycles of alternating between 65° C. and 95° C. were run over a period of 204 seconds. Fluid temperature was measured continuously during thermocycling. The TEC cooler was able to rapidly cycle the temperature of the fluid within the chamber of the consumable.

### Example 8: Designs of Improved Reporters for Immobilization on Substrates

Provided herein are methods for optimizing the cleavage of fluorescent reporters immobilized on a substrate in a system for detecting a target nucleic acid described herein. A reporter is attached to a surface via any of the chemistries described herein (e.g., amine chemistry). The immobilized reporter then can be contacted with a programmable nuclease and a guide nucleic acid in a trans cleavage reaction. FIGS. 29A-29E show the designs of various immobilized reporters. The arrow below each reporter indicates the expected signal change when the reporter is cleaved by a programmable nuclease. In some cases, cleavage of the reporter may release a fluorophore, as shown in FIGS. 29A, 29C, and 29E, thereby decreasing the fluorescence signal of the reporter. In other cases, as shown in FIGS. 29D and 29D, cleavage of the reporter may release a quencher moiety, thereby increasing the fluorescent signal of the reporter. FIGS. 29A (e.g., reporter 112) and 29B (e.g., reporter 136) depict single-stranded nucleic acid reporters that will show decreased and increased fluorescence, respectively. FIGS. 29C (e.g., reporter 204) and 29D (e.g., reporter 203) depict two exemplary optimized nucleic acid reporters that will show decreased and increased fluorescence, respectively. These reporters have increased length compared to those of FIGS. 29A and 29B and contain double-stranded regions. FIG. 29E (e.g., reporter 200) shows a single-stranded nucleic acid reporter with an increased length relative to those of FIGS. 29A-29D. Without being bound by any theory, the increased length and/or double-stranded in region in the reporters of FIGS. 29C-29E, relative to those of FIGS. 29A and 29B decrease the steric hindrance of the access of the cleavage sites on the reporter by programmable nuclease. The increased length also decreases interference of access to the cleavage site by the programmable nuclease caused, at least in part, by the surface chemistry of the substrate. FIG. 32 depicts an image of an exemplary substrate immobilized with guide nucleic acids and reporters. Each spot represents a discrete detection location on the substrate immobilized with at least one guide nucleic acid and at least one reporter.

### Example 9: Workflow for Detecting Target Nucleic Acids using Immobilized Reporters

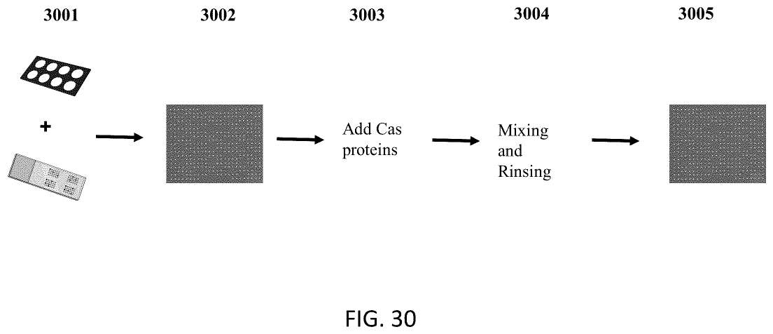

Provided herein are methods for detecting nucleic acids by cleavage of immobilized reporters and/or guide nucleic acids. FIG. 30 shows an exemplary workflow for detecting target nucleic acids using immobilized reporters. At step 3001, the reporters and/or guide nucleic acids are immobilized on a glass slide, such as by NHS-amine chemistry. At step 3002, the slide is imaged in an imaging buffer to identify the starting signal at each discrete detection position. Each spot represents one discrete detection position. At step 3003, a programmable nuclease, such as a Cas12 protein, is added onto the slide along with target nucleic acids. The programmable nuclease may also be added onto the slide with non-target nucleic acids to produce a negative control signal. The guide nucleic acid can also be added if it has not been immobilized on the slide. At step 3004, the slides are placed on a thermomixer for a period of time (e.g., 20 minutes) to allow for the programmable nuclease, the guide nucleic acid, the reporter, and the target nucleic acid to mix thoroughly. The presence of both the target nucleic acid and the guide nucleic acid that can hybridize with the target nucleic acid can trigger the trans cleavage activity of the programmable nuclease, thereby cleaving the immobilized reporter at the discrete detection position upon which a reporter is immobilized. At step 3005, the slide is imaged again in the imagining buffer to identify the changes of the signal at each discrete detection position.

### Example 10: Trans-Cleavage Reactions Successfully Cleaves Immobilized Reporters

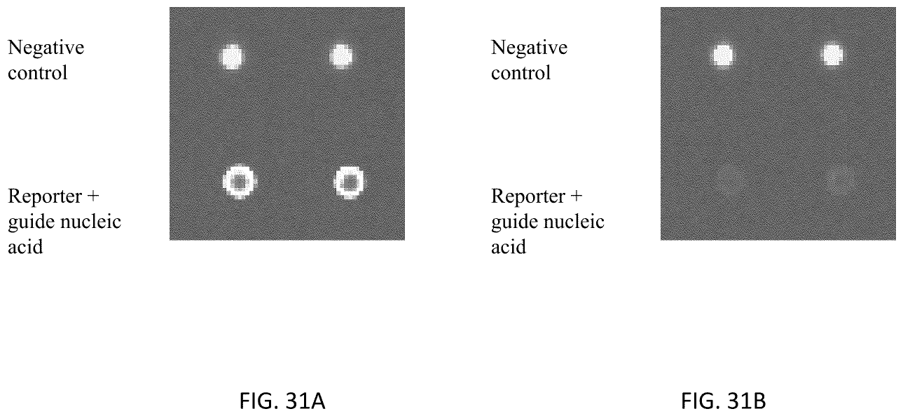

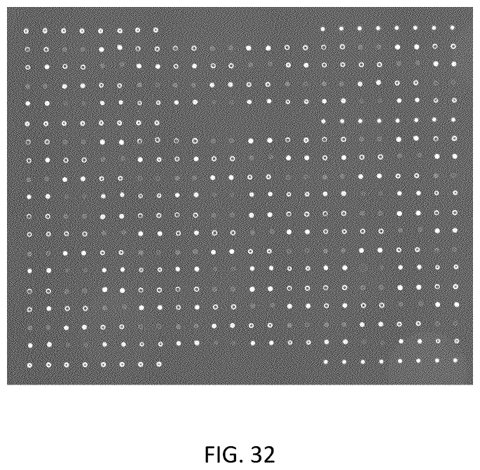

FIGS. 31A and 31B show the image of a slide on which a surface-based trans-cleavage assay was carried out thereto using the workflow of FIG. 30. FIG. 31A shows the slide before addition of the programmable nuclease and the target nucleic acid thereto. FIG. 31B shows the slide after addition of the programmable nuclease and the target nucleic acid thereto. Reporter 200 (as shown in FIG. 29C) and a guide RNA were immobilized on a glass slide (from Applied Microarrays). In the presence of a target nucleic acid and the guide nucleic acid that hybridized with the target nucleic acid, a programmable nuclease having an amino acid identical to that of SEQ ID NO: 34 was activated, triggering the trans cleavage activity and resulting in the cleavage of immobilized reporter 200, as described in Example 7. The cleavage resulted in a decrease of visible signal 20 minutes after the addition of the programmable nuclease and the target nucleic acid to the slide (the two replicates in the bottom rows). In contrast, the negative controls using uncleavable Lumiprobes did not show any changes of signals. This result suggests that the surface-based trans-cleavage assay reactions can result in visible and specific changes in the images of the array.

### Example 11: Specific and Robust Detection of Target Nucleic Acid Using Surface-Based

Trans-cleavage Assays and Optimized Immobilized Reporters

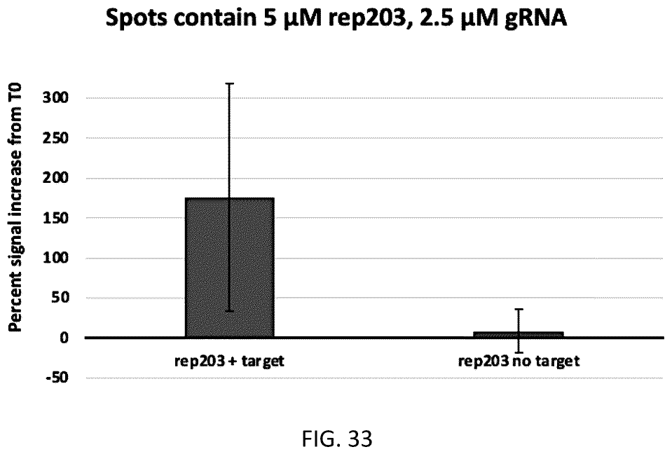

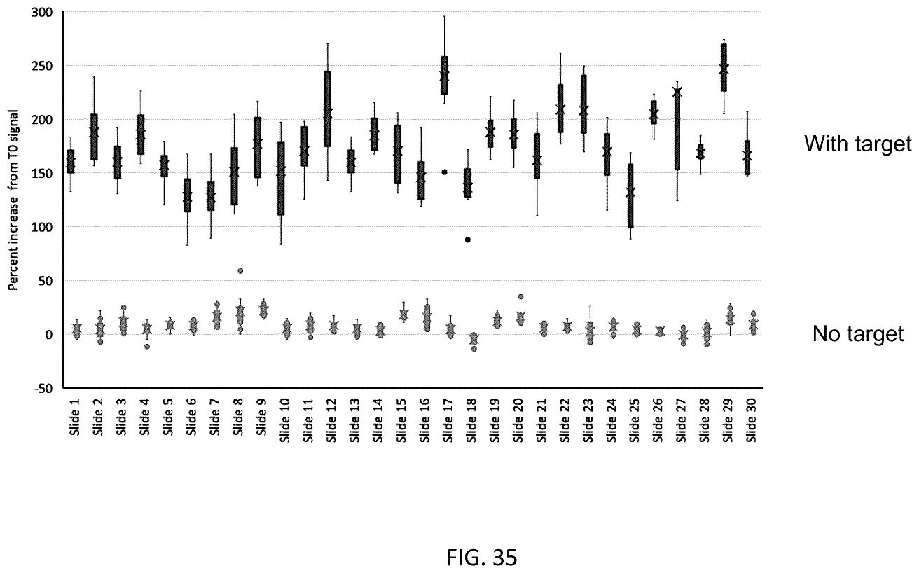

Surface-based trans-cleavage assays provided specific cleavage of immobilized reporters only in the presence of a target nucleic acid and a complementary guide nucleic acid. ˜5 μM partially double-stranded reporter 203 (as shown in FIG. 29D and described in Table 2) and ˜2.5 μM guide RNA complementary to a target nucleic acid were immobilized on a detection location of an array by NHS-amine chemistry, as described in Example 8. The array contained 10⁻¹² spots each. FIG. 33 summarizes the results of 30 arrays. FIG. 35 shows box plots of the signal increase for each array. 20 minutes after the addition of a programmable nuclease having SEQ ID NO: 34 and the target nucleic acids, the fluorescence signal increased by 150%, relative to before the programmable nuclease and the target nucleic acids were added. The increase of the signal was specific: arrays without the addition of the target nucleic acids showed minimal increase in the fluorescence signal. Therefore, surface-based trans cleavage assays and improved reporters allowed for specific detection of the target nucleic acid.

### Example 12: Trans-Cleavage Reactions Successfully Cleavage Immobilized Reporters with Multiple gRNAs

Trans-cleavage reactions were shown to successfully cleave NHS-amine bound reporter molecules in a series of multiplex assays carried out on microarray slides. The sequences of the reporters used in each of the assays described are provided in Table 2 herein. The guides used in each of the assays are provided in Table 6.

TABLE 6
Guides for Surface-Based Trans-Cleavage Assays

| Guide Description | Guide Sequence |
|---|---|
| G1 | 3′ no spacer R777 |
| G2 | 3′ C6 spacer R777 |
| G3 | 3′ C12 spacer R777 |
| G4 | 3′ C6 spacer R1763 |

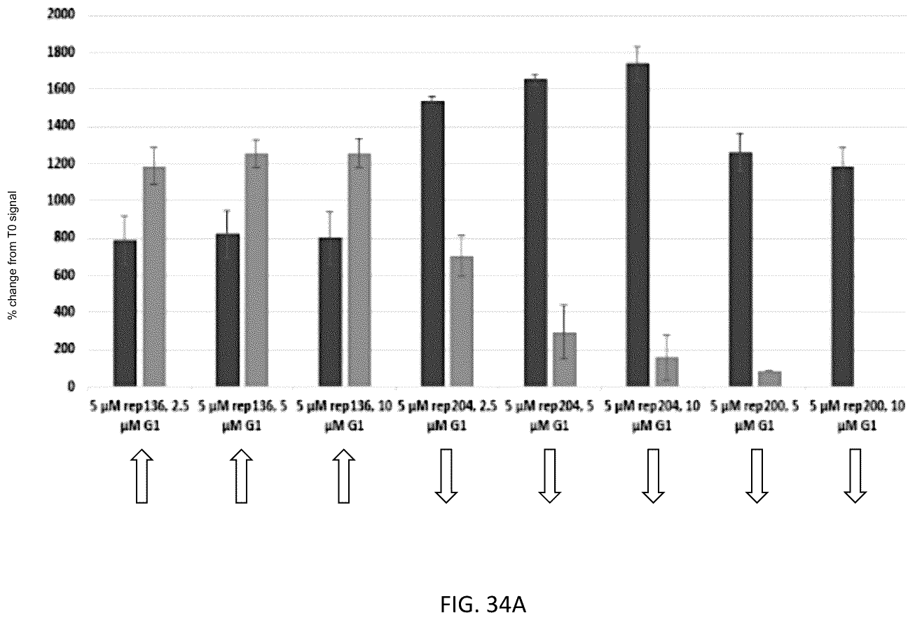

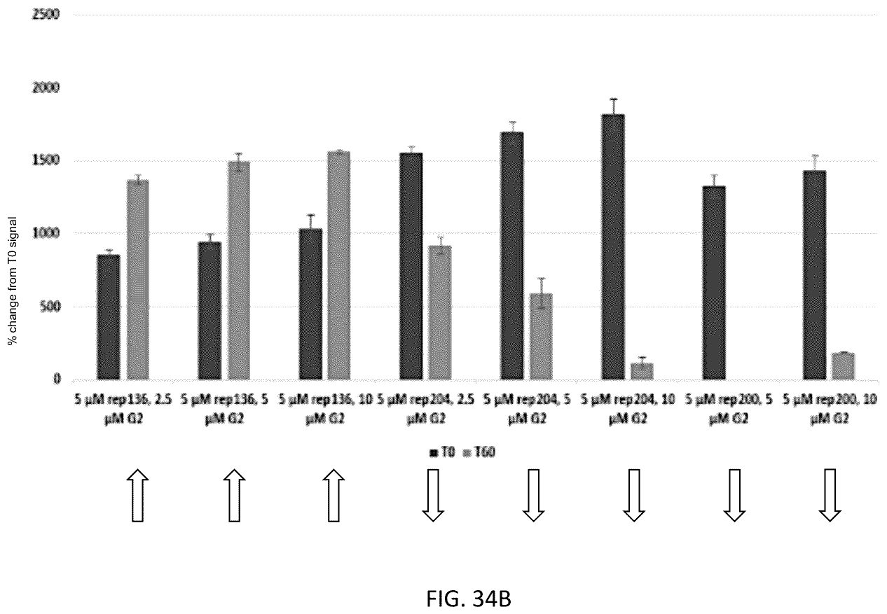

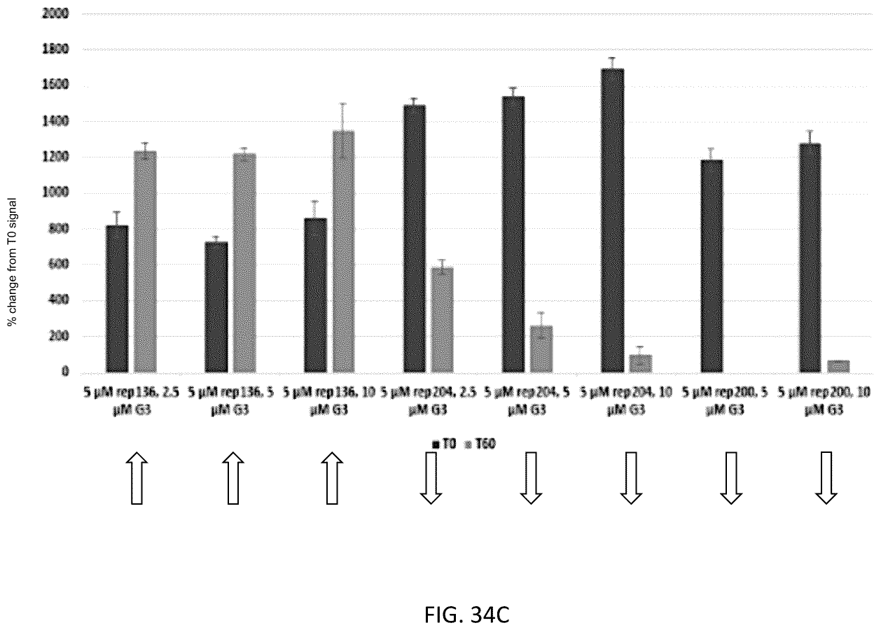

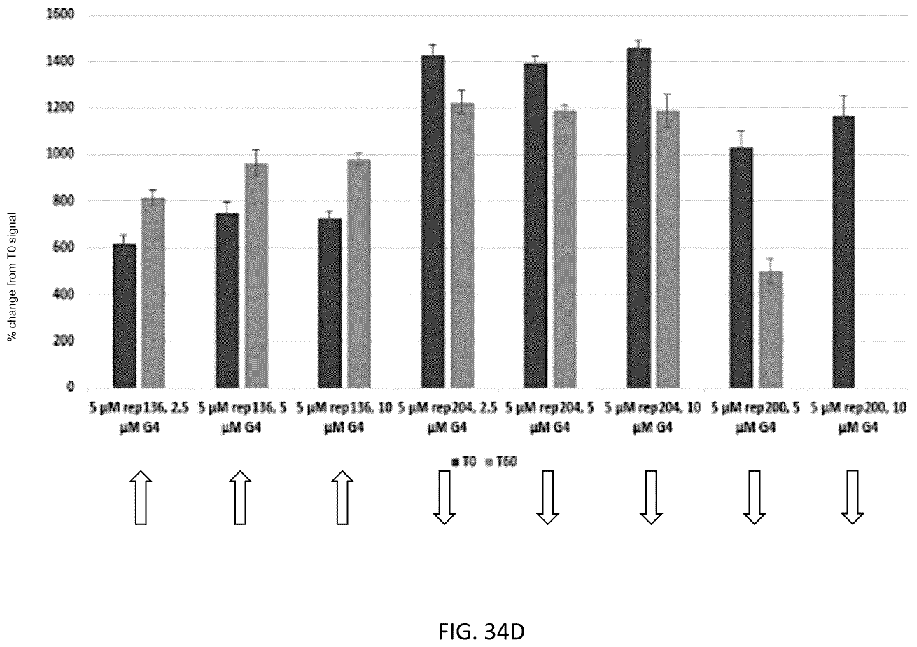

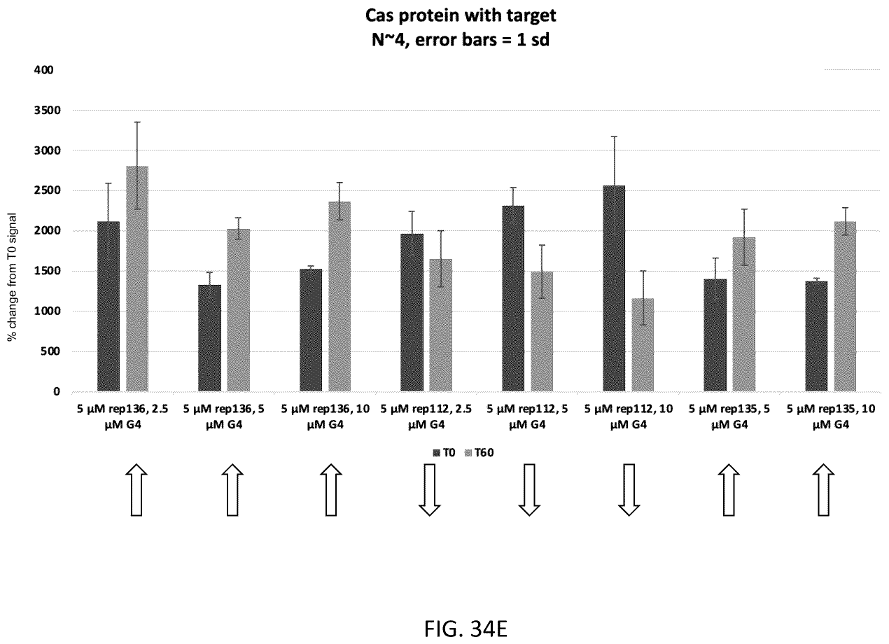

Four different assays—each assay using one of four guide nucleic acids-were carried out. For each reaction, reporters and guide nucleic acids—in a range of concentrations-were immobilized on a detection region at different discrete detection locations on a microarray slide from HD Suromodics. After immobilization, the programmable nuclease of SEQ ID NO: 34 and a buffer were added to the wells, and the plate was incubated at 55° C. on a thermomixer shaking at 500 RPM for 1 hour. The plates were imaged under the leica 100% FAM channel at 500 ms exposure before and 60 minutes after the addition of the programmable nucleases. FIGS. 34A-34D show the results from each of the four assays, with FIG. 34A showing the reactions performed with the guide G1 at varying concentrations and reporters rep 136, rep204, and rep200 at 5 μM; FIG. 34B showing the reactions performed with the guide G2 at varying concentrations and reporters rep 136, rep204, and rep200 at 5 μM; FIG. 34C showing the reactions performed with the guide G3 at varying concentrations and reporters rep 136, rep204, and rep200 at 5 μM; and FIG. 34D showing the reactions performed with the guide G4 at varying concentrations and reporters rep 136, rep204, and rep200 at 5 μM. Finally, FIG. 6E shows the reactions performed with the guide G4 at varying concentrations and reporters rep 136, rep135, and rep112 at 5 μM.

As shown in FIGS. 34A-34E, in the presence of the target nucleic acids and the guide RNAs, the fluorescence moiety (i.e., FAM) of reporter 200 (shown in FIG. 29E), 204 (shown in FIGS. 29C), and 112 (shown in FIG. 29A) was freed by the cleavage of the programmable nuclease, resulting in a significantly decreased fluorescence signal after 60 minutes of the addition of the programmable nuclease. For example, reporter 200 showed significant fluorescence signal decrease over the course of the reaction. As shown in FIGS. 34A-34E, in the presence of the target nucleic acids and the guide RNAs, the quencher moiety of reporter 136 (shown in FIG. 29B), 203 (shown in FIG. 29D), and 135 (substantially similar to FIG. 29B) was freed by the cleavage of the programmable nuclease, resulting in a significantly increased fluorescence signal after 60 minutes of the addition of the programmable nuclease.

### Example 13: Trans-Cleavage Reactions Successfully Cleavage Immobilized Reporters on Various Substrates

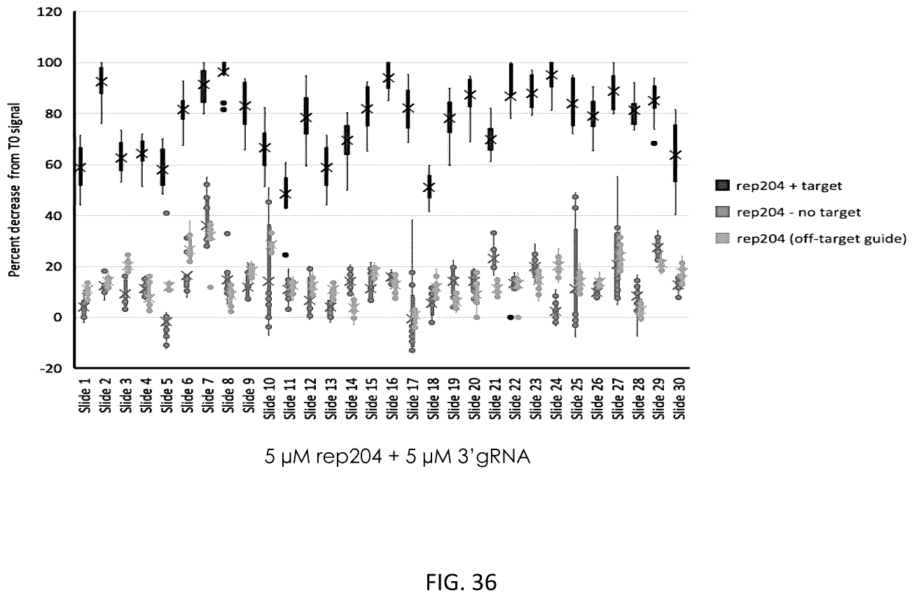

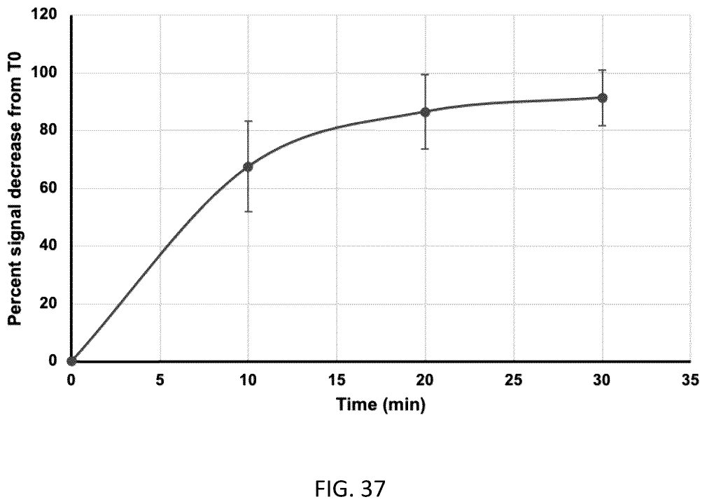

The high specificity of the immobilized reporters was reproduced in different batches of substrate. Thirty different slides, each with 10⁻¹² spots were immobilized with reporter 203 (as shown in FIG. 29C) or reporter 204 (as shown in FIG. 29D) were constructed. ˜5 μM reporter and ˜5 μM guide nucleic acids were immobilized on each discrete detection location, respectively NO: 34, significant increases of fluorescence were observed in all slides, relative to the negative controls without any target nucleic acid, as shown in FIG. 35. Furthermore, when using reporter 204, significant decreases of fluorescence were observed in all slides, relative to the negative controls without any target nucleic acid or non-complementary guide nucleic acids, as shown in FIG. 36.

To assay for when signals of immobilized reporters become detectable, the signal of reporter 204 was assayed at multiple time points after the programmable nuclease of SEQ ID NO: 34 and the target nucleic acid were added to the immobilized reporter 204 and the guide nucleic acid complementary to the target nucleic acid. ˜5 μM reporter and ˜5 μM guide nucleic acids were immobilized on each discrete detection location. As shown in FIG. 39, percentage decrease of the reporter plateaued 20 minutes after the addition of the Cas protein and the target nucleic acid. Hence, robust signals resulted from the cleavage of the immobilized reporter can be observed soon after the activation of the Cas protein.

### Example 14: Combining Immobilized Reporters with Instruments Thereof

Provided herein are methods, systems, and instruments for detecting immobilized reporters in a surface-based trans-cleavage assay and optimized immobilized reporters. For example, the surface-based trans-cleavage assay using the optimized immobilized reporters can be implemented using a microfluidic device as shown in FIG. 23A. FIG. 23A illustrates an exemplary amplification/detection module as described herein. For example, the solution comprising the target nucleic acids can be amplified in an amplification chamber in the amplification region. The amplified target nucleic acids can be transported to a detection chamber of the detection region comprising a plurality of immobilized guide RNAs. The reporters described in this disclosure are immobilized on the substrate surface of the detection chamber at discrete locations and are co-localized with the immobilized guide RNAs such that each discrete detection location comprises at least one reporter and at least one guide RNA. Improved reporters and/or guide nucleic acids described in this disclosure can be immobilized to the surface of the detection chamber as described herein. Programmable nucleases can then be added to the detection chamber. The binding of the target nucleic acid and the complementary guide nucleic acid can activate the programmable nuclease and trigger the cleavage of the immobilized reporters as described herein.

### Example 15: Detection of Target Nucleic Acids in Serially-Arranged Wells

Systems for multiplexed detection of a plurality of target nucleic acids are provided. The systems use a programmable nuclease-based microfluidic device configured for the detection of a plurality of different target nucleic acids using sequentially-arranged chambers. Exemplary arrangements of chambers connected in sequence in a microfluidic device are shown in FIGS. 39A-39B. The system includes a sample interface configured to receive a sample, wherein the sample interface is in fluid communication with the plurality of chambers. A liquid comprising one or more of the different target nucleic acids or amplicons thereof flows into the plurality of chambers. In one or more of the wells, a complex comprising a programmable nuclease and a guide nucleic acid is activated upon binding a target nucleic acid, and the activated complex cleaves the reporters to form detectable cleavage products in one or more of the wells. The detectable cleavage products are detected, and the location of a well containing the detectable cleavage product identifies the target nucleic acid or amplicon thereof present in the well. The system can include an amplification region in fluid communication with the sample reservoir, and a detection region in fluid communication with the amplification region. A portion of each well fills with fluid before the next sequential well begins to fill with fluid. A guide nucleic acid of known sequence is provided in each well. For reporters comprising a fluorescent label and a quencher, cleavage results in a detectable fluorescence in response to activation at an excitation wavelength.

Referring to FIG. 39A in particular, the arrows within the chamber indicate fluid movement. The outlet ports go through to the bottom of the chip where they are covered with a hydrophobic membrane that allows air to pass through but not liquid (except at higher pressures). Due to the fluid movement being laminar at this scale, the movement is driven mainly by surface tension. The main channel is, in this example, 0.40 mm×0.35 mm in width and depth. The capillary valves are oriented such that they branch off of the main channel at a minimum of 90°. The capillary valve dimensions in this example are 0.30 mm×0.20 mm in width and depth. The exact dimensions are not required, but in general, the capillary valve inlet area is much smaller than that of the main channel. The fluid fills the well 25-50% before the fluid has enough pressure build up to start going into the capillary valve. The fluid then continues filling the well while the remainder of the fluid progresses down the path from one well/valve to the next. In this example, each well is ˜10 μL. The design minimizes fluidic crosstalk between each well. If the fluid volume input to the chip is such that the volume is equal to the amount of volume in each well, air clears the plenum behind each well. If pressure is held, the fluid in each well does not come out of the well and interfere with the reactions occurring in a separate well. The various wells may be arranged in a microfluidic device as illustrated in FIGS. 40A-40C, in which exits extending from the wells to their respective outlets may be of alternating long and short lengths. The various features of the microfluidic device may have a width, depth, area, and ratio as provided in Table 7.

TABLE 7

| | Width (mm) | Depth (mm) | Area (mm²) | Ratio (%)* |
|---|---|---|---|---|
| Capillary valve | 0.20 | 0.30 | 0.0600 | 42.86 |
| Channel | 0.40 | 0.35 | 0.1400 | 100 |
| Exit (short) | 0.40 | 0.25 | 0.1000 | 71.43 |
| Exit (long) | 0.45 | 0.25 | 0.1125 | 80.36 |

*Ratio represents percentage of channel area

With reference to FIG. 39B, in particular, the arrows within the chamber indicate fluid movement. In this design, the outlet ports are formed at the bottom of the wells. The bottom of the microfluidic device is covered with a hydrophobic membrane that allows air to pass through but not liquid (except at higher pressures). Due to the fluid movement being laminar at this scale, the movement is driven mainly by surface tension. The main channel is, in this example, 0.50 mm×0.35 mm in width and depth. Each well is roughly 0.50 mm in diameter. The capillary valves are oriented such that they are directly over each well. The capillary valve dimensions in this example are 0.30 mm×0.20 mm in width and depth. The exact dimensions are not required, but in general, the capillary valve inlet area is much smaller than that of the main channel. The fluid substantially fills the well before the fluid has enough pressure build up to start going into the capillary valve. The fluid then continues down the path from one well/valve to the next. The aspect ratio of the well can be an important consideration for optimizing performance, with narrower wells preferred in some implementations. In this example, each well is ˜0.75 μL. The design minimizes fluidic crosstalk between each well, and reduces the fluid volume needed for analysis. If the fluid volume input to the chip is such that the volume is equal to the amount of volume in each well, air clears the plenum behind each well. If pressure is held, the fluid in each well does not come out of the well and interfere with the reactions occurring in a separate well.

A sample is inserted into the system, followed by optional lysis and amplification steps. The detection of the cleavage product is achieved by surface characteristics of fluorescence quenching. Each of the programmable nuclease probes contains both a programmable nuclease and a guide nucleic acid. In this example, the programmable nuclease at each detection is coupled to a guide nucleic acid that is specific toward a particular target nucleic acid sequence. A different guide nucleic acid resides in each chamber/well. If the desired target is present, an enzyme will be activated and resultant fluorescence/quenching will be observed. When the guide nucleic acid of the programmable nuclease selectively binds to its complementary target nucleic acid, the programmable nuclease is activated, thereby enabling trans-cleavage of a cleavable nucleic acid of the reporter. When the reporter is cleaved, a portion of the reporter is released from the surface (in this example, a quencher). Once the quencher is released, a fluorophore still coupled to the immobilized portion of the reporter is no longer quenched and exhibits fluorescence upon excitation by an illumination source of the instrument. The excitation light from the illumination source in the instrument is transmitted to an optical sensor or detector in the instrument. In this manner, each chamber/well of the microfluidic device is assessed and any detection event exhibiting fluorescence signifies that the quencher has been released by an activated programmable nuclease, thus indicating that the target nucleic acid specific to the guide nucleic acid of known sequence in the corresponding chamber is present in the sample. The chambers are spatially encoded with known different programmable nuclease-guide complexes, thereby allowing for multiplexed target analysis on a single sample.

### Example 16: Multiplex Detection of Target Nucleic Acids

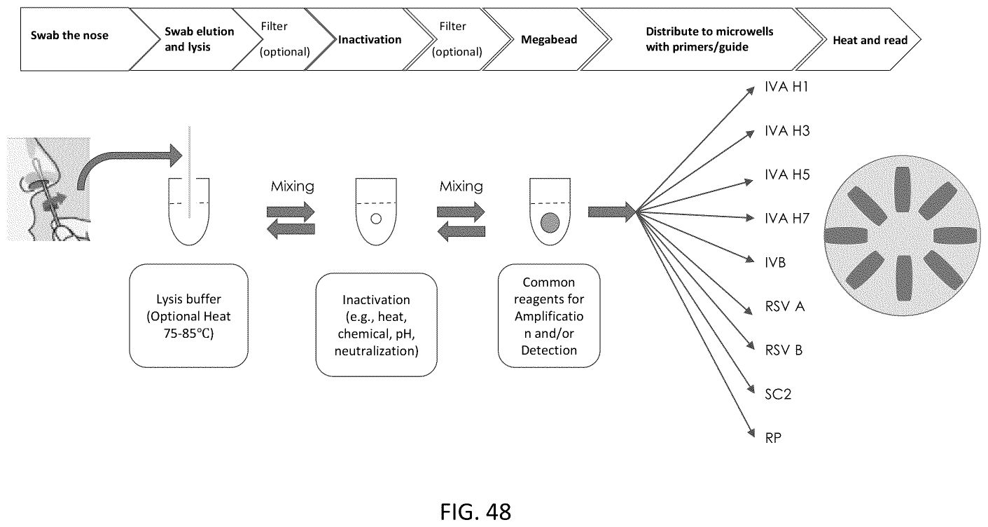

Provided herein are multiplex detection methods for determining whether any of a plurality of target nucleic acids are present in a sample. FIG. 48 depicts an exemplary high-level workflow for using a multiplex detection system in accordance with some embodiments. The illustrated process provides a multiplex method for detecting target nucleic acids from influenza virus A (IVA), influenza virus B (IVB), respiratory syncytial virus (RSV), SARS-COV-2 (SC2), and RNase P (RP). The workflow begins with a swab or other sample collector (e.g., tube) that may be used to collect a sample from the patient. For example, the patient may collect a nasopharyngeal sample using a swab provided with the system. The patient may then insert the swab into a container for swab elution and lysis. In the container, the swab may contact a lysis and elution buffer. The elution/lysis buffer is at a relatively low pH, e.g., acidic, and heated at about 75-85° C. for 1-4 min with mixing. Next, because an acidic buffer is used for the lysis step, inactivation comprises a neutralization step that raising the pH of the solution, such as to a pH of about 7. Neutralization may utilize any suitable base (e.g., potassium hydroxide or Tris), which may be provided in any suitable form (e.g., pellet or concentrated solution). A filtering step to remove debris may be included between lysis and inactivation, after inactivation, or both. Portions of the sample are then subjected to detection reactions as described herein, in which different reactions are configured to detect the presence of different target nucleic acids in the sample, such as in separate wells of a cartridge described herein. In general, the detection reaction will include a programmable nuclease and a guide nucleic acid, and may include incubation at about 58-60° C. The programmable nuclease may, for example, be immobilized to a surface of the detection region or may be reconstituted by a liquid from a dried or lyophilized form (e.g., after sample lysis and/or elution) prior to being distributed into the detection region as described herein. For example, the detection region may comprise a plurality of microwells, each of which may comprise a target-specific guide RNA and primer disposed therein (e.g., in situ dried or lyophilized within the microwell). Common reagents (e.g., amplification reagents such as a reverse transcriptase, polymerase, etc. and/or detection reagents such as a programmable nuclease, reporter, etc.) may be disposed upstream of the detection region in dried or lyophilized form (e.g., in a single lyophilized megabead or in situ dried or lyophilized within an upstream channel, chamber, jumper, or the like). Following sample preparation, the sample may be used to rehydrate the common reagents located upstream of the detection region, and the mixture may then be distributed within the detection region (e.g., into different microwells) to rehydrate the target-specific reagents disposed therein. The various chambers in FIG. 48 are illustrated as separate containers for the sake of simplicity only. The illustrated containers may be a common processing chamber to which the sample and various reagents are added and/or removed at each step, or separate chambers to which sample is transferred for each step. The illustrated containers may form part of a microfluidic device described herein, or a cartridge thereof.

In another example, the swab elution and lysis buffer may comprise a protease (e.g. Quick Extract™ buffer (ProK+Betaine)), which is incubated at a lysis temperature, then heat inactivated at 90° C. for 2 min with mixing. When a protease is included in the lysis step, neutralization may include the addition of a protease inhibitor and/or heat to inactivate the protease. A filtering step to remove debris may be included between lysis and inactivation, after inactivation, or both. Portions of the sample are then subjected to detection reactions as described herein, in which different reactions are configured to detect the presence of different target nucleic acids in the sample, such as in separate wells of a cartridge described herein. In general, the detection reaction will include a programmable nuclease and a guide nucleic acid, and may include incubation at about 58-60° C.

While preferred embodiments of the present invention have been shown and described herein, it may be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

1.-62. (canceled)

63. A system for detecting a target nucleic acid, the system comprising:
a. an instrument;
b. a cartridge configured to interface with the instrument, the cartridge comprising:
i. a sample interface configured to receive a sample comprising one or more nucleic acids;
ii. one or more reagent capsules; and
iii. a detection region;
c. a guide nucleic acid disposed within the cartridge and that is complementary to the target nucleic acid, or a portion thereof, of the one or more nucleic acids;
d. a programmable nuclease disposed within the cartridge and that is capable of being complexed with the guide nucleic acid, wherein the programmable nuclease is configured to be activated through binding of the guide nucleic acid to the target nucleic acid; and
e. a reporter disposed within the cartridge, the reporter comprising a cleavable nucleic acid and a detection moiety, wherein cleavage of the cleavable nucleic acid by the activated programmable nuclease releases the detection moiety from the cleavable nucleic acid, wherein the released detection moiety is configured to generate a signal;
wherein the detection region is configured to detect the signal indicating the presence of the target nucleic acid.

64. The system of claim 63, wherein the sample interface comprises a scraper to extract the sample from a swab.

65. The system of claim 63, further comprising a sample preparation region configured to purify and concentrate the one or more nucleic acids.

66. The system of claim 65, wherein the sample preparation region further comprises a subset of the one or more reagent capsules, wherein the subset comprises a protein digestion reagent, a cellular digestion reagent, one or more solvents, or a combination thereof.

67. The system of claim 66, wherein (a) the liquid capacity of each of the reagent capsules ranges from 50 μL to 500 μL in volume; and/or (b) the subset contains 4 to 6 reagent capsules.

68. The system of claim 65, wherein the sample preparation region comprises one or more beads or membranes having a silica coating, wherein the silica coating is configured to bind at least one nucleic acid of the one or more nucleic acids.

69. The system of claim 68, wherein the silica beads are magnetic silica beads.

70. The system of any one of claim 69, wherein the instrument comprises a magnet configured to immobilize and release the magnetic silica beads.

71. The system of claim 63, further comprising an amplification region, and optionally amplification reagents.

72. The system of claim 71, wherein the amplification reagents are present as liquid amplification reagents and lyophilized amplification reagents; and optionally wherein the liquid amplification reagents comprise one or more activator salts.

73. The system of claim 63, wherein the instrument is configured to control fluid, temperature and detection parameters of reactions occurring within the cartridge.

74. The system of claim 63, wherein:
(a) the detection region is configured for spatially multiplexed detection of a plurality of target nucleic acids in the sample;
(b) the programmable nuclease, the guide nucleic acid, or the reporter are immobilized to a surface of the detection region;
(c) the programmable nuclease and the reporter are contained within a chamber of the detection region, wherein the programmable nuclease and the reporter are configured to react in liquid phase; or
(d) the instrument comprises an optical sensor configured to detect a detection moiety released upon cleaving of the reporter by an activated programmable nuclease.

75. A system for multiplexed detection of a plurality of target nucleic acids comprising:
a. an instrument;
b. a cartridge comprising:
i. a sample interface;
ii. one or more reagent capsules;
iii. a sample preparation region;
iv. an amplification region; and
V. a detection region; the detection region comprising a plurality of detection locations, each detection location of the plurality of detection locations comprising a reporter and a programmable nuclease complexed with a guide nucleic acid that is complementary to a different target nucleic acid of a plurality of target nucleic acids,
wherein, at each detection location, the corresponding reporter and the corresponding guide nucleic acid are immobilized to a surface of the detection region,
wherein, at each detection location, the corresponding programmable nuclease is configured to cleave the reporter and generate a different signal of a plurality of signals, and
wherein each different signal of the plurality of signals indicates a presence or absence of each different target nucleic acid at its respective detection spot.

76.-81. (canceled)

82. A system for detecting a target nucleic acid, the system comprising:
a detection region comprising at least one detection location comprising:
a programmable nuclease disposed within the detection region that is complexed with a guide nucleic acid, wherein the guide nucleic acid is complementary to a target nucleic acid, or a portion thereof, wherein the programmable nuclease is configured to be activated through binding of the guide nucleic acid to the target nucleic acid;
a reporter disposed within the detection region, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid; and
a surface comprising a hydrophobic membrane.

83. A system comprising a microfluidic device comprising a plurality of chambers fluidically connected in sequence, wherein:
(a) each chamber of the plurality of chambers comprises a well, an inlet channel, an outlet, and a capillary valve;
(b) the capillary valve of each chamber (i) has a cross-sectional area that is smaller than a cross-sectional area of the inlet channel of the respective chamber, and (ii) forms an entrance of the inlet channel of the next chamber in the sequence; and
(c) each outlet is air-permeable and configured to retain liquid within the respective chamber.

84. The system of claim 83, wherein each chamber further comprises detection reagents comprising a guide nucleic acid and a reporter, and further wherein:
(a) each guide nucleic acid (i) comprises a targeting sequence that hybridizes with a target nucleic acid of a plurality of different target nucleic acids or an amplicon thereof, and (ii) is effective to form a complex with a programmable nuclease that is activated upon binding the corresponding target nucleic acid or amplicon thereof;
(b) the guide nucleic acid of a first chamber in the plurality of chambers comprises a different targeting sequence from the guide nucleic acid of a second chamber in the plurality of chambers; and
(c) each reporter (i) comprises a cleavable nucleic acid and a detection moiety, and (ii) is configured to be cleaved to form a detectable cleavage product in response to activation of the complex in the well of the respective chamber.

85.-91. (canceled)

92. The system of claim 83, wherein the outlet comprises (a) an opening sized to permit displacement of air therethrough but to retain liquid within the well under an operating pressure of the microfluidic device, (b) a surface comprising a hydrophobic coating, or (c) a surface comprising an air-permeable hydrophobic membrane.

93. The system of claim 92, wherein the outlet comprises the surface comprising the air-permeable hydrophobic membrane, and further wherein the hydrophobic membrane (a) comprises woven polypropylene or woven polyethylene, (b) the hydrophobic membrane comprises pores of about 0.1 microns to about 2 microns in size, and/or (c) forms a bottom surface of the respective well.

94.-98. (canceled)

99. The system of claim 83, wherein (a) the outlets vent through a first surface of the microfluidic device, (b) the system further comprises a heater in thermal communication with a second surface of the microfluidic device, and (c) the first surface is opposite the second surface.

100. The system of claim 83, wherein the plurality of chambers comprises at least 10 chambers fluidically connected in sequence.

101. The system of claim 84, wherein the detection reagents further comprise a programmable nuclease.

102.-104. (canceled)

105. A microfluidic device comprising:
a loading channel comprising a first capillary valve disposed upstream of a second capillary valve disposed therein;
a first chamber fluidically coupled to the loading channel upstream of the first capillary valve;
a second chamber fluidically coupled to the loading channel between the first capillary valve and the second capillary valve; and
a third chamber fluidically coupled to the loading channel downstream of the second capillary valve, wherein:
(a) each chamber of the first, second, and third chambers comprises an outlet;
(b) each of the first and second capillary valves have a cross-sectional area that is smaller than a cross-sectional area of the loading channel; and
(c) each outlet is gas-permeable and configured to retain liquid within the respective chamber.

106.-111. (canceled)

112. A cartridge for detecting a target nucleic acid, the cartridge comprising:
(a) a reagent reservoir;
(b) a programmable nuclease, wherein the programmable nuclease is configured to be activated through binding of a guide nucleic acid to a target nucleic acid;
(c) a reporter, the reporter comprising a cleavable nucleic acid and a detection moiety, wherein cleavage of the cleavable nucleic acid by the activated programmable nuclease releases the detection moiety from the cleavable nucleic acid, and wherein the released detection moiety is configured to generate a signal indicative of a presence of the target nucleic acid;
(d) a sample interface in fluid communication with the reagent reservoir, the sample interface configured to receive a sample;
(e) a detection region in fluid communication with the sample interface, the detection region comprising:
(i) a guide nucleic acid disposed within the detection region, wherein the guide nucleic acid is complementary to the target nucleic acid, or a portion thereof; and
(ii) a primer disposed within the detection region, wherein the primer is designed to amplify one or more nucleic acids in the sample; and
wherein the detection region is configured to amplify the one or more nucleic acids in the sample and to enable detection of the signal.


SEQUENCE LISTING (97 sequences)

SEQ ID NO: 1 - protein, 1307 aa, synthetic construct
  REGION 1..1307: note = Description of Artificial Sequence: Synthetic polypeptide
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 2 - protein, 492 aa, synthetic construct
  REGION 1..492: note = Description of Artificial Sequence: Synthetic polypeptide
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 3 - protein, 529 aa, synthetic construct
  REGION 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 1297 aa, synthetic construct
  REGION 1..1297: note = Description of Artificial Sequence: Synthetic polypeptide
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 5 - protein, 1145 aa, synthetic construct
  REGION 1..1145: note = Description of Artificial Sequence: Synthetic polypeptide
MCMKITKIDGISHKKYKEKGKLIKNNDTAKDIIEERFNDIEKKTKELFQKTLDFYVKNYEKCKEQNKERREKAKNYFSKVKILVDNKKITICNENTEKMEIEDFNEYDVRSGKYFNVLNKILNGENYTEEDLEVFENDLQKRTGRIKSIKNSLEENKAHFKKESINNNIIYDRVKGNNKKSLFYEYYRISSKHQEYVNNIFEAFDKLYSNSHEAMNNLFSEITKDSKDRNIRKIREAYHEILNKNKTEFGEELYKKIQDNRNNFDKLLEIEPEIKELTKSQIFYKYYIDKVNLDETSIKHCFCHLVEIEVNQLLKNYVYSKRNINKEKLENIFEYCKLKNLIKNKLVNKLNNYIRNCGKYNAYISNNDVVVNSEKISEIRTKEAFLRSIIGVSSSAYFSLRNILNTDNTQDITNKVDKEVDKLYQENKKIELEERLKLFFGNYFDINNQQEIKVFLMNIDKIISSIRHEIIHFKMETNAQNIFDFNNVNLGNTAKNIFSNEINEEKIKFKIFKQLNSANVFDYLSNKDITEYMDKVVFSFTNRNVSFVPSFTKIYNRVQDLANSLEIKKWKIPDKSEGKDAQIYLLKNIYYGKFLDEFLNEENGIFISIKDKIIELNRNQNKRTGFYKLEKFEKIEETNPKKYLEIIQSLYMINIEEIDSEGKNIFLDFIQKIFLKGFFEFIKNNYNYLLELKKIQDKKNIFDSEMSEYIAGEKTLEDIGEINEIIQDIKITEIDKILNQTDKINCFYLLLKLLNYKEITELKGNLEKYQILSKTNVYEKELMLLNIVNLDNNKVKIENFKILAEEIGKFIEKINIEEINKNKKIKTFEELRNFEKGENTGEYYNIYSDDKNIKNIRNLYNIKKYGMLDLLEKISEKTNYCIKKKDLEEYSELRKQLEDEKTNFYKIQEYLHSKYQQKPKKILLKNNKNDYEKYKKSIENIEKYVHLKNKIEFNELNLLQSLLLKILHRLVGFTSIWERDLRFRLIGEFPDELDVEDIFDHRKRYKGTGKGICKKYDRFINTHTEYKNNNKMENVKFADNNPVRNYIAHFNYLPNPKYSILKMMEKLRKLLDYDRKLKNAVMKSIKDILEEYGFKAEFIINSDKEIILNLVKSVEIIHLGKEDLKSRRNSEDLCKLVKAMLEYSK

SEQ ID NO: 6 - protein, 1271 aa, synthetic construct
  REGION 1..1271: note = Description of Artificial Sequence: Synthetic polypeptide
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 7 - protein, 1229 aa, synthetic construct
  REGION 1..1229: note = Description of Artificial Sequence: Synthetic polypeptide
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 8 - protein, 835 aa, synthetic construct
  REGION 1..835: note = Description of Artificial Sequence: Synthetic polypeptide
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 9 - protein, 1264 aa, synthetic construct
  REGION 1..1264: note = Description of Artificial Sequence: Synthetic polypeptide
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 10 - protein, 500 aa, synthetic construct
  REGION 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 11 - protein, 1261 aa, synthetic construct
  REGION 1..1261: note = Description of Artificial Sequence: Synthetic polypeptide
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 12 - protein, 478 aa, synthetic construct
  REGION 1..478: note = Description of Artificial Sequence: Synthetic polypeptide
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 13 - protein, 1229 aa, synthetic construct
  REGION 1..1229: note = Description of Artificial Sequence: Synthetic polypeptide
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 14 - protein, 1242 aa, synthetic construct
  REGION 1..1242: note = Description of Artificial Sequence: Synthetic polypeptide
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 15 - protein, 1168 aa, synthetic construct
  REGION 1..1168: note = Description of Artificial Sequence: Synthetic polypeptide
MKITKIDGILHKKYIKEGKLVKSTSEENKTDERLSELLTIRLDTYIKNPDNASEEENRIRRETLKEFFSNKVLYLKDSILYLKDRREKNQLQNKNYSEEDISEYDLKNKNSFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRIKGKTVKNKKGEEKYISGEIDKLYDNNKQNEVKKNLKMFYSYDFNMNRKKEIEDFFSNIDEAISSIRHGIVHFNLELEGKDIFTFKNIVPSQISKKMFQNEINEKKLKLKIFRQLNSANVFRYLEKYKILNYLNRTRFEFVNKNIPFVPSFTKLYSRIDDLKNSLCIYWKIPKANDNNKTKEITDAQIYLLKNIYYGEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAGKKQEFDKFLKKYEQNNNIEIPHEINEFVREIKLGKILKYTESLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGILNLLEKISDEAKYKISIEELKNYSNKKIEIEKNHTTQENLHRKYARPRKDEKFNDEDYKKYEKTIRNIQQYTHLKNKVEFNELNLLQSLLLRILHRLVGYTSIWERDLRFRLKGEFPENQYIEEIFNFDNSKNVKYKNGQIVEKYISFYKELYKDDMEKISIYSDKKVKELKKEKKDLYIRNYIAHFNYIPNAEVSLLEVLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 16 - protein, 1168 aa, synthetic construct
  REGION 1..1168: note = Description of Artificial Sequence: Synthetic polypeptide
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNSFLVLKKILLNEDINSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIRECYHKIIGRKNDKENFAKIIYEEIQNVNNIKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIAGNRQNEAFLRNIIGVSSVAYFSLRNILETENKDDITGKMRGKTRIDSKTGEEKYIPGEVDQIYYENKQNEVKNKLKMFYGYDFDMDNKKEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISREIIELNKNDKRNLKTGFYKLQKFEDIQEKTPKKYLANIQSLYMINAGNQDEEEKDTYIDFIQKIFLKGFMTYLANNGRLSLMYIGNDEQINTSLAGKKQEFDKFLKKYEQNNNIEIPHEINEFLREIKLGKILKYTESLNMFYLILKLLNHKELTNLKGSLEKYQSANKEETFSDELELINLLNLDNNRVTEDFELEANEIGKFLDFNGNKIKDRKELKKFDTKKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENQYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSVVDILKEYGFVATFKIGADKKIGIQTLESEKIVHLKNLKKKKLMTDRNSEELCKLVKVMFEYKMEEKNLKTKKCKVI

SEQ ID NO: 17 - protein, 1224 aa, synthetic construct
  REGION 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 18 - protein, 1225 aa, synthetic construct
  REGION 1..1225: note = Description of Artificial Sequence: Synthetic polypeptide
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 19 - protein, 1165 aa, synthetic construct
  REGION 1..1165: note = Description of Artificial Sequence: Synthetic polypeptide
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 20 - protein, 1194 aa, synthetic construct
  REGION 1..1194: note = Description of Artificial Sequence: Synthetic polypeptide
MKVTKIDGISHKKFEDEGKLVKFTGHFNIKNEMKERLEKLKELKLSNYIKNPENVKNKDKNKEKETKSRRENLKKYFSEIILRKKEEKYLLKKTRKFKNITEEINYDDIKKRENQQKIFDVLKELLEQRINENDKEEILNFDSVKLKEAFEEDFIKKELKIKAIEESLEKNRADYRKDYVELENEKYEDVKGQNKRSLVFEYYKNPENREKFKENIKYAFENLYTEENIKNLYSEIKEIFEKVHLKSKVRYFYQNEIIGESEFSEKDEEGISILYKQIINSVEKKEKFIEFLQKVKIKDLTRSQIFYKYFLENEELNDENIKYVFSYFVEIEVNKLLKENVYKTKKFNEGNKYRVKNIFNYDKLKNLVVYKLENKLNNYVRNCGKYNYHMENGDIATSDINMKNRQTEAFLRSILGVSSFGYFSLRNILGVNDDDFYKIEKDERKNENFILKKAKEDFTSKNIFEKVVDKSFEKKGIYQIKENLKMFYGNSFDKVDKDELKKFFVNMLEAITSVRHRIVHYNINTNSENIFDFSNIEVSKLLKNIFEKEIDTRELKLKIFRQLNSAGVFDYWESWVIKKYLENVKFEFVNKNVPFVPSFKKLYDRIDNLKGWNALKLGNNINIPKRKEAKDSQIYLLKNIYYGEFVEKFVNDNKNFEKIVKEIIEINRGAGTNKKTGFYKLEKFETLKANTPTKYLEKLQSLHKISYDKEKIEEDKDVYVDFVQKIFLKGFVNYLKKLDSLKSLNLLNLRKDETITDKKSVHDEKLKLWENSGSNLSKMPEEIYEYVKKIKISNINYNDRMSIFYLLLKLIDYRELTNLRGNLEKYESMNKNKIYSEELTIINLVNLDNNKVRTNFSLEAEDIGKFLKSSITIKNIAQLNNFSKIFADGENVIKHRSFYNIKKYGILDLLEKIVAKADLKITKEEIKKYENLQNELKRNDFYKIQEQIHRNYNQKPFSIKKIENKKDFEKYKKVIEKIQDYTQLKNKIEFNDLNLLQSLIFRILHRLAGYTSLWERDLQFKLKGEFPEDKYIDEIFNSDGNNNQKYKHGGIADKYANFLIEKKEEKSGEILNKKQRKKKIKEDLEIRNYIAHFNYLPNAEKSILEILEELRELLKHDRKLKNAVMKSIKDIFREYGFIVEFTISHTKNGKKIKVCSVKSEKIKHLKNNELITTRNSEDLCELVKIMLEHKELQK

SEQ ID NO: 21 - protein, 1159 aa, synthetic construct
  REGION 1..1159: note = Description of Artificial Sequence: Synthetic polypeptide
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 22 - protein, 1134 aa, synthetic construct
  REGION 1..1134: note = Description of Artificial Sequence: Synthetic polypeptide
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 23 - protein, 1282 aa, synthetic construct
  REGION 1..1282: note = Description of Artificial Sequence: Synthetic polypeptide
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 24 - protein, 1399 aa, synthetic construct
  REGION 1..1399: note = Description of Artificial Sequence: Synthetic polypeptide
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 25 - protein, 1313 aa, synthetic construct
  REGION 1..1313: note = Description of Artificial Sequence: Synthetic polypeptide
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 26 - protein, 1470 aa, synthetic construct
  REGION 1..1470: note = Description of Artificial Sequence: Synthetic polypeptide
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 27 - protein, 1259 aa, synthetic construct
  REGION 1..1259: note = Description of Artificial Sequence: Synthetic polypeptide
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 28 - protein, 477 aa, synthetic construct
  REGION 1..477: note = Description of Artificial Sequence: Synthetic polypeptide
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 29 - protein, 1293 aa, synthetic construct
  REGION 1..1293: note = Description of Artificial Sequence: Synthetic polypeptide
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 30 - protein, 1228 aa, synthetic construct
  REGION 1..1228: note = Description of Artificial Sequence: Synthetic polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 31 - protein, 1286 aa, synthetic construct
  REGION 1..1286: note = Description of Artificial Sequence: Synthetic polypeptide
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 32 - protein, 1272 aa, synthetic construct
  REGION 1..1272: note = Description of Artificial Sequence: Synthetic polypeptide
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 33 - protein, 477 aa, synthetic construct
  REGION 1..477: note = Description of Artificial Sequence: Synthetic polypeptide
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 34 - protein, 1281 aa, synthetic construct
  REGION 1..1281: note = Description of Artificial Sequence: Synthetic polypeptide
MLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 35 - protein, 1367 aa, synthetic construct
  REGION 1..1367: note = Description of Artificial Sequence: Synthetic polypeptide
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 36 - protein, 809 aa, synthetic construct
  REGION 1..809: note = Description of Artificial Sequence: Synthetic polypeptide
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 37 - protein, 839 aa, synthetic construct
  REGION 1..839: note = Description of Artificial Sequence: Synthetic polypeptide
LNSIEKIKKPSNRNSIPSIIISDYDENKIKEIKVKYLKLARLDKITIQDMEIRDNIVEFKKILLNGIEHTIKDNQKIEFDNYEITAYVRASKQRRDGKITQAKYVVTITDKYLRDNEKEKRFKSTERELPNDTLLMRYKQISGFDTLTSKDIYKIKRYIDFKNEMLFYFQFIEEFFSPLLPKGTNFYSLNIEQNKDKVVKYIVYRLNDDFKNQSLNQFIKKTDTIKYDFLKIQKILSDFRHALAHFDFDFIQKFFDDELDKNRFDISTISLIKTMLQEKEEKYYQEKNNYIEDSDTLTLFDEKESNFSKIHNFYIKISQKKPAFNKLINSFLSKDGVPNEELKSYLATKKIDFFEDIHSNKEYKKIYIKHKNLVVEKQKEESQEKPNGQKLKNYNDELQKLKDEMNKITKQNSLNRLEVKLRLAFGFIANEYNYNFKNFNDKFTLDVKKEQKIKVFKNSSNEKLKEYFESTFIEKRFFHFCVKFFNKKTKKEETKQKNIFNLIENETLEELVKESPLLQIITLLYLFIPKELQGEFVGFILKIYHHTKNITNDTKEDEKSIEDTQNSFSLKLKILAKNLRGLQLFNYSLSHNTLYNTKEHFFYEKGNRWQSVYKSLEISHNQDEFDIHLVIPVIKYYINLNKLIGDFEIYALLTYADKNSITEKLSDITKRDDLKFRGYYNFSTLLFKTFMINTNYEQNQKSTQYIKQTRNDIAHQNIENMLKAFENNEIFAQREEIVNYLQKEHKMQEILHYNPINDFTMKTVQYLKSLNIHSQKESKIADIHKKESLVPNDYYLIYKLKVIELLKQKVIEAIGETKDEEKIKNAIAKEEQIKKGYNK

SEQ ID NO: 38 - protein, 840 aa, synthetic construct
  REGION 1..840: note = Description of Artificial Sequence: Synthetic polypeptide
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 39 - protein, 840 aa, synthetic construct
  REGION 1..840: note = Description of Artificial Sequence: Synthetic polypeptide
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 40 - protein, 849 aa, synthetic construct
  REGION 1..849: note = Description of Artificial Sequence: Synthetic polypeptide
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 41 - protein, 822 aa, synthetic construct
  REGION 1..822: note = Description of Artificial Sequence: Synthetic polypeptide
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 42 - protein, 806 aa, synthetic construct
  REGION 1..806: note = Description of Artificial Sequence: Synthetic polypeptide
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 43 - protein, 832 aa, synthetic construct
  REGION 1..832: note = Description of Artificial Sequence: Synthetic polypeptide
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 44 - protein, 1281 aa, synthetic construct
  REGION 1..1281: note = Description of Artificial Sequence: Synthetic polypeptide
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 45 - protein, 1280 aa, synthetic construct
  REGION 1..1280: note = Description of Artificial Sequence: Synthetic polypeptide
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 46 - protein, 832 aa, synthetic construct; identical to SEQ ID NO: 43
  REGION 1..832: note = Description of Artificial Sequence: Synthetic polypeptide

SEQ ID NO: 47 - protein, 809 aa, synthetic construct; identical to SEQ ID NO: 36
  REGION 1..809: note = Description of Artificial Sequence: Synthetic polypeptide

SEQ ID NO: 48 - protein, 1285 aa, synthetic construct
  REGION 1..1285: note = Description of Artificial Sequence: Synthetic polypeptide
MEEKMLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 49 - protein, 1367 aa, synthetic construct; identical to SEQ ID NO: 35
  REGION 1..1367: note = Description of Artificial Sequence: Synthetic polypeptide

SEQ ID NO: 50 - protein, 1366 aa, synthetic construct
  REGION 1..1366: note = Description of Artificial Sequence: Synthetic polypeptide
MNTQKKEFNPKSFKDFTNLYSLNKTLRFSLTPNKKTAEILEFNKQKEVKCFSNDRKIAGAYQEIKKYLNKLHQEFIQEAMKFFAFSEEELKGFEKEYLNLLNFTDKDNFKKKNKIRNEYEQERKILTIKIATYFSKFKSEKYQSFNLANITGKKVFSILEQKYKEDKKTLKIIHIFKYKPTKDEKKEGEAVNFSTYLTGFNENRKNFYKSEDKAGQFATRTIDNLAQFIKNKKLFEDKYQKNYSKIGILDEQIKIFNLDYFNNLFLQEGLDEYNGILGNNKGEENKSNEGINQKINIFKQKEKARLKKEKENFNKSDFPLFKELYKQIGSIRKENDVYVEIKTDKELVEELNNFPKNVENYLKDIQSFYKTFFEKLQNEEYELDKIYLPKSVGTYFSYIAFSDWNKLAFIYNKRYKNEKIKIVEGGDVNVQYRSLEVLKNRIDELKDEDNLNFNKFFIDKLKFNEAKKENNWQNFWFCIEYYINSQFIGGEKNILNKEKNEYEILPFGSLKELKEKYFEAVKKYKEKMVDTESGLTDDEEKEIKETLKNYLDRIKEIERIAKYFDLKKSFEEIKQEDLDSNFYGEYQKVVDKTNELKIYQYYSEFRNYLTQNNSVEEKIKLNFNSGLLLDGWDLNKEKVKFSIIFQENGKYYLGIINKEKDKTILDKDKHPEIFTKNSDFRKMEYKLFPSPSKMLPKISFSETAKKGDEDVGWSEEIQKIKDEFAEFQEYKKKSKDNWKDEFNRGKLNKLIDYYKQVLEKHSEGYMNTYNFELKDSSKYKNLGEFNDDIARQNYKVKFVGIDKNYIDEKVANGELFLFQIYNKDFSEDKKEGSTNNLETIYFKELFSKENLENPVFKLSGGAEMFFRNKIEKKKEKKKLDKDGKPMISKKGEKVVDKRRFSENKILFHLPIEINYGKGKMPNFNKKINEYISKNPENIKIIGIDRGEKHLLYYSIIDQNGNNIESMSLNAVDEFGNFVNPEKLEEYEIDNNGKKERRWKYIVNDKEIKVTNYQRKLDELEKERQKSRQSWQNINKIKNLKKGYISFVVKKIVDLAIENNAIIILEDLNFGFKSFRQKIEKNVYQQFEKALIDKLGFVVDKQKQNQRFAPQLSAPFESFQKIGKQTGIVYYVLANNTSKVCPSCQWIKNFYLKYEKKNTIFNLQKNQKLKVFFEQEKNRFRFEYQMSKEYISVYSDVDRQRYDKTKNQNKGGYLEYKNSNQKEIIDKDGVIQKQSITLQLKELFKENHIDLEKEILKQLDNKKEKNSGYTGVYNKFIYLFNLILQIRNAISFREKDYIQCPSCHFDTRKENYLKINDGDGNGAYNIALRGLYLLKGKNGIINNLEKIKLIFSNNDYFQWAKKLKNKK

SEQ ID NO: 51 - protein, 1275 aa, synthetic construct
  REGION 1..1275: note = Description of Artificial Sequence: Synthetic polypeptide
MQNKQSFADFTNLYSLSKTLRFELKPIGQTQAMLDENKIFEVDENRKKAYDKTKPYFDRLHREFINESLSNAQLKGISEYFETFKQFRSNQNNKDLKELINKQQKFLRHQIVTLFDENGKHWATTKYAHLKIKKKNLDILFDEQVFYILKERYGSEKETQLVDKETGAVTSIFDNWKGFTGYFTKFFETRKNFYKSDGTSTALATRIIDQNLNRFFDNLETFHKIKDKIDVKEVEIFFKLKADNVFSIDFYNQCLLQNGIDKYNDFLGGQTLENGEKQKGINEIINKYRQDNKDQKLPFLKKLDKQILSEKDRFINEIESKEEFFQVLTEFYQSATVKVTIIKTLLNDFVHNTDKYKLEKIYLTKEAFNTIANKWTDETQIFEDNLDLVLKNKKITAKQDFIPLAYIKEALEVIEKDRKFFKDRYYNDPQIGFFPDQSYWEQFLAILNFEFMTHFQRVAKDKITGKKIELGYFVFEKRIKELLDSDPSLNSQSKIIIKEFADEVLHIFQMAKYFALEKKREWKGDYYQLDDQFYNHIDYGFKDQFYENAYEKIVQPYNKIRNYLTKKPYSDVKWKLNFGNPTLANGWDKNKEADNTAVILKKDGNYYLGVMKKGKNKIFSDQNKEKYKAYNSAYYEKLVYKLFPDPSKMFPKVCFSKKGLNFFQPSEEILRIYKNNEFKKGNTFSISSMQKLIAFYIDCLGLYEGWKHYEFKNIKDVRQYKENIGEFYADVAESGYKLWFEKISEEYITQKNQLGELFLFQIYNKDFAKKTTGRKNLHTIYFEELFSQTNIDNNFPFKLNGQAELFYRPKSLEKIEEKRNFKRSIVNKKRYTQNKIFFHVPITLNRTSENIGRFNVRVNNFLANNSNVNIVGVDRGEKNLAYYSIIKQNGEVLKSGSLNIINGVDYHALLTDRAQRREQERRNWQDVESIKDLKRGYISQVVHELVSLAIKYNAIIVMEDLNMRFKQIRGGIEKSTYQQLEKALIEKLNFLVNKEETDSNQAGNLLNAYQLTAPFKTFKDMGKQTGIIFYTQASYTSKIDPLTGWRPNIYLRYSNAKQAKADILMFTNIYFSEKKDRFEFTYDLEKIDDKRKDLPIKTEWTVCSNVERFSWEKSLNNNKGGYVHYPIQDSNGEESITSKLKKLFMDFGIDLTDIKTQIESLDTNKKDNANFFRKFIFYFQLICQIRNTQVNKSDDGNDFIFSPVEPFFDSRFADKFRKNLPKNGDENGAYNIARKGLIILHKISDYFVKEGSTDKISWKDLSISQTEWDNFTTDK

SEQ ID NO: 52 - protein, 1368 aa, synthetic construct
  REGION 1..1368: note = Description of Artificial Sequence: Synthetic polypeptide
MKKEKEFKSFGDFTNLYEISKTLRFELKPVENTQTMLDEADVFGKDKVIKDKYTKTKPFIDKLHREFVDESLKDVSLSGLKKYSEVLENWKKNKKDKDIVKELKKEEERLRKEVVEFFDNTAKKWANEKYKELGLKKKDIGILFEESVFDLLKEKYGEEQDSFLKEEKGDFLKNEKGEKVSIFDEWKGFVGYFTKFQETRKNFYKNDGTETALATRIIDQNLKRFCDNIDDFKKIKNKIDFSEVEKNFNKTADVFSLDFYNQCLLQKGIDSYNEFIGGKTLENGKKLKGVNELVNEYRQKNKNEKVSFLKLLDKQILSEKEKLSFGIENDEQLLVVLNSFYETAEEKTKILRTLFGDFVEHNENYDLDKTYISKVAFNTISHKWTNETHKFEELLYGAMKEDKPIGLNYDKKEDSYKFPDFIALGYLKKCLNNLDCDTKFWKEKYYENNADKKDKDKGFLTGGQNAWDQFLQIFIFEFNQLFNSEAFDNKGKEIKIGYDNFRKDFEEIINQKDFKNDENLKIAIKNFADSVLWIYQMAKYFAIEKKRGWDDDFELSEFYTNPSNGYSLFYDRAYEEIVQKYNDLRNYLTKKPYKEDKWKLNFENPTLANGFDKNKESDNSTVILRKKRKYYLGLMKKGNNKIFEDRNKAEFIRNIESGAYEKMAYKYLPDVAKMIPKCSTQLNEAKNHFRNSADDLEIKKSFSNPLKITKRIFDLNNIQYDKTNVSKKISGDNKGIKIFQKEYYKISGDFDVYKSALNDWIDFCKDFLSKYDSTKDFDFSILRKTKDYKSLDEFYVDVAKITYKISFTPVSESYIDQKNKNGELYLFEIYNQDFAKGKMGAKNLHTLYFENVFSPENISKNFPIKLNGNAELFFRPKSIESKKEKRNFVREIVNKKRYSEDKIFFHCPITLNRETGSIYRFNNYVNNFLSENNINIIGVDRGEKHLAYYSVIDKNGVKIGGGSFNEINKVDYAKKLEERAGEREQSRKDWQVVEGIKDLKKGYISQVVRELADLAIKHNAIIVLEDLNMRFKQIRGGIEKSIYQQLEKALIDKLSFLVEKGEKDPNQAGHILKAYQLAAPFTSFKDMGKQTGIVFYTQASYTSKTCPNCGFRKNNNKFYFENNIGKAQDALKKLKTFEYDSENKCFGLSYCLSDFANKEEVEKNKNKKRNNAPYSDIEKKDCFELSTKDAVRYRWHDKNTERGKTFFEGESVYEEKEEKEIGQTKRGLVKEYDISKCLIGLFEKTGLDYKQNLLDKINSGKFDGTFYKNLFNYLNLLFEIRNSISGTEIDYISCPECQFHTDKSKTIKNGDDNGSYNIARKGMIILDKIKQFKKENGSLDKMGWGELFIDLEEWDKFAQKKNNNIIDK

SEQ ID NO: 53 - protein, 1257 aa, synthetic construct
  REGION 1..1257: note = Description of Artificial Sequence: Synthetic polypeptide
MKYTDFTGIYSVSKTLRFELIPQGSTVENMKREGILNNDMHRADSYKEMKKLIDEYHKAFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFDIVAKVPEMQEHFKTIADELRSHLQVGNDIDKMFNLQFFNKVLTQSQLDVYNAVIGGKSEGNKKIQGINEYINLYNQQHKKARLPMLKLLYKQILSDRVAISWLQDEFDNDQDMLDTIEAFYNKLNSNETGVLGEGKLKQILMGLDGYNLDGVFMRNDLQLSEVSQRLCGGWNIIKDAMTSDLKRSVQKKKKETDADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDNFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHSLLEEKEAGNKKARQDWQTIESIKELKEGYLSQVIHKISMLMQRYHAIVVLEDLNGSFMRSRQKVEKQVYQKFEHMLINKLNYLVNKQYDATEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 54 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 55 - protein, 717 aa, synthetic construct
  REGION 1..717: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 56 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 57 - protein, 718 aa, synthetic construct
  REGION 1..718: note = Description of Artificial Sequence: Synthetic polypeptide
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 58 - protein, 757 aa, synthetic construct
  REGION 1..757: note = Description of Artificial Sequence: Synthetic polypeptide
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 59 - protein, 761 aa, synthetic construct
  REGION 1..761: note = Description of Artificial Sequence: Synthetic polypeptide
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 60 - protein, 708 aa, synthetic construct
  REGION 1..708: note = Description of Artificial Sequence: Synthetic polypeptide
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 61 - protein, 661 aa, synthetic construct
  REGION 1..661: note = Description of Artificial Sequence: Synthetic polypeptide
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 62 - DNA, 50 bp, synthetic construct
  misc_feature 1..50: note = Description of Artificial Sequence: Synthetic oligonucleotide
  source 1..50: mol_type = other DNA
cttacccacctctcccccaaaacaaacaccactaactcacatcacaaccc

SEQ ID NO: 63 - DNA, 59 bp, synthetic construct
  misc_feature 1..59: note = Description of Artificial Sequence: Synthetic oligonucleotide
  source 1..59: mol_type = other DNA
cttacccacctctcccccaaaacaaacaccactaactcacatcacaacccttttttttt

SEQ ID NO: 64 - DNA, 12 bp, synthetic construct
  misc_feature 1..12: note = Description of Artificial Sequence: Synthetic oligonucleotide
  source 1..12: mol_type = other DNA
tttttttttttt

SEQ ID NO: 65 - DNA, 19 bp, synthetic construct
  misc_feature 1..19: note = Description of Artificial Sequence: Synthetic oligonucleotide
  source 1..19: mol_type = other DNA
ttttttttttttttttttt

SEQ ID NO: 66 - DNA, 60 bp, synthetic construct
  misc_feature 1..60: note = Description of Artificial Sequence: Synthetic oligonucleotide
  source 1..60: mol_type = other DNA
tttttttttttttttttttttttttttttttttttttttttttttttttttttttttttt

SEQ ID NO: 67 - skipped

SEQ ID NO: 68 - DNA, 50 bp, synthetic construct
  misc_feature 1..50: note = Description of Artificial Sequence: Synthetic oligonucleotide
  source 1..50: mol_type = other DNA
gggttgtgatgtgagttagtggtgtttgttttgggggagaggtgggtaag

SEQ ID NO: 69 - DNA, 50 bp, synthetic construct; identical to SEQ ID NO: 68
  misc_feature 1..50: note = Description of Artificial Sequence: Synthetic oligonucleotide
  source 1..50: mol_type = other DNA

SEQ ID NO: 70-71 - skipped (2 entries)

SEQ ID NO: 72 - protein, 5 aa, synthetic construct
  REGION 1..5: note = Description of Artificial Sequence: Synthetic peptide
  REGION 1..5: note = See specification as filed for detailed description of substitutions and preferred embodiments
GSGGS

SEQ ID NO: 73 - protein, 6 aa, synthetic construct
  REGION 1..6: note = Description of Artificial Sequence: Synthetic peptide
  REGION 1..6: note = See specification as filed for detailed description of substitutions and preferred embodiments
GGSGGS

SEQ ID NO: 74 - protein, 4 aa, synthetic construct
  REGION 1..4: note = Description of Artificial Sequence: Synthetic peptide
  REGION 1..4: note = See specification as filed for detailed description of substitutions and preferred embodiments
GGGS

SEQ ID NO: 75 - protein, 4 aa, synthetic construct
  REGION 1..4: note = Description of Artificial Sequence: Synthetic peptide
GGSG

SEQ ID NO: 76 - protein, 5 aa, synthetic construct
  REGION 1..5: note = Description of Artificial Sequence: Synthetic peptide
GGSGG

SEQ ID NO: 77 - protein, 5 aa, synthetic construct
  REGION 1..5: note = Description of Artificial Sequence: Synthetic peptide
GSGSG

SEQ ID NO: 78 - protein, 5 aa, synthetic construct
  REGION 1..5: note = Description of Artificial Sequence: Synthetic peptide
GSGGG

SEQ ID NO: 79 - protein, 5 aa, synthetic construct
  REGION 1..5: note = Description of Artificial Sequence: Synthetic peptide
GGGSG

SEQ ID NO: 80 - protein, 5 aa, synthetic construct
  REGION 1..5: note = Description of Artificial Sequence: Synthetic peptide
GSSSG

SEQ ID NO: 81 - protein, 845 aa, synthetic construct
  REGION 1..845: note = Description of Artificial Sequence: Synthetic polypeptide
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 82 - protein, 802 aa, synthetic construct
  REGION 1..802: note = Description of Artificial Sequence: Synthetic polypeptide
MLKKPSNRYALPKVILSTVDHEKILEFKVKYEKLARLDRLVVERMHFDGESVVFDEVIANSGDLEIAYQDDHRKLLIQAAGKSYTITGKKVGGKKRKLEERISRAKIQLTLTDGQEDQHRRIRATVTEKALLEPKEDRDIYSKISDRKIKTSKEIYLVKRFLSYRSDLLFYYFFVDNFFKVGNNKQELWKIKFQNQPELIEYFRFIINDRFKNAKNDKFDNYLKNDKAIQEDLEKIQKVFEKLRHALMHYDYGFFEKLFGGEDQGFDLDIAFLDNFVKKIDKLNIDTKKEFVDDEKIKIFGEDLNLADLYKLYASISINRVGFNRVVNEMIIKDGIEKSELKRAFEKKLDKTYALDIHSDPSYKKLYNEHKRLVTEVSTYTDGNKIKEGNQKIAKLKYEMKEITKKNALVRLECKMRLAFGLIYGRYDTHEAFKNGFDTDLKRGEFAQIGSEEAIGYFNTTFEKSKPKSKEEIKKIARQIDNLSLSTLIEDDPLMKFIVLMFLFVPRELKGEFLGFWRKYYHDIHSIDSDAKSDEMPDEVSLSLKLKILTRNIRRLNLFEYSLSEKIKYSPKNTQFYTDKSPYQKVYKRLKISHNKEEFDKTLLVPLFRYYSILFKLINDFEIYSLAKANPDASSLSELTKTKHGFRGHYNFTTLMMDAHKVSQGDSKKHFGIRGEIAHINTKDLIYDPLFRKSKMAQQRNDVIDFVLKYEKEIKAVLGYDAINDFRMKVVQLRTKLKVYSDKTQTIEKLLNEVEAPDDFYVLYKVKGVEAINKYLLEIVSVTQAEEEIERKIITGNKRYNT

SEQ ID NO: 83 - protein, 1281 aa, synthetic construct
  REGION 1..1281: note = Description of Artificial Sequence: Synthetic polypeptide
MIKSYDDFTKLYSLQKTLRFELKPIGKTLEHIKKSEIIESDETLEEQYAIVKNIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPILKPLHKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYEFYNELEVYYEALLPLNSLYNKVRNYMTRKPYSVEKFKLNFYSPTLLDGWDKNKETANLSIILKKNGKYYLGIMNKENNTIFENFPKSKSNDYYEKMIYKLLPGPNKMLPKVFFSKKGLEYYKPSKEILRIYEKGEFKKDKSGNFKKESLHTLIDFYKEAIAKNEDWKIFKFKFKNTREYEDISQFYRDVEEQGYLIIFEKVDANYVDKLVEEGELYLFQIYNKDFSENKKSKGNPNLHTIYWESLFDNQNLKDVVYKLNGEAEVFYRKKSIDYPEEIYNNGHHKEELNGKFNYPIIKDRRYTQDKFLFHVPITMNFISKEEKRVNQLACEYLSTTKEDVHIIGIDRGERHLLYLSLIDKEGNIKKQLSLNTIKNENYDKEIDYRVKLDEKEKKRDEARKNWDVIENIKELKEGYMSQVIHIIAKMMAEEKAILIMEDLNIGFKRGRFKVEKQVYQKFEKMMIDKLNYLVFKNKEPLEPGGSLNAYQLTSKFDSFKKLGKQSGFIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 84 - protein, 1281 aa, synthetic construct; identical to SEQ ID NO: 83
  REGION 1..1281: note = Description of Artificial Sequence: Synthetic polypeptide

SEQ ID NO: 85 - protein, 1332 aa, synthetic construct
  REGION 1..1332: note = Description of Artificial Sequence: Synthetic polypeptide
MKSIFDNFTGLYSLSKTLRFELRPVGQTLENIKNGHFLESDKKMADDYQDVKKIIDNYHKFFIDDVLKGASFDWALLEKELTDFNKNKTDDSKVEAEQKKLREQIAKTLAGDKRFKSLTASTPNDLFNKDKDFIGWLEQSSVKEIRKDALDTFKKFSSYFKGFQENRKNVYSADDIPTAVPYRIVNDNFPKFLQNISIFKTIQEKCPQVIADVENELASYLGKEKLADIFTVQAFNKYLCQGGKENQRGIDFYNQVIGGIAEKEGGVNLRGINQFLNLYWQQHPDFAKENRRIKMVPLYKQILSDRSSLSFKIETIDTDEELKTAISEYADKLESKSNDEKKSVLDVCVELFDSIKEQNLQEIYVNRKDINNISRILTGDWSWLQSRMNLYADEVFTTKAEKTRWQKSVDGDEGENKSKGAYSLAELNRVLEYASENVAETDIRITDYFCHRNRFYYEKESGLFKQGEELVALSIKESCEDILSKRKAMNEAFANISESNSLRDNSEDIAKTKTYLDSVQDLLHRIKPLKVNGLGDPSFYAVFDSIYSALSEVISIYNKTRNYITRKAESPEKYKLNFDNPTLANGWDLNKEKDNTCVLLRKNGMYYLGIMNPKDKPKFAEKYDCGTESCYEKMIYKLLPGPNKMLPKVFFSTKGKKQYNPPENILHGYEQGKHKKGVAFDINFCHELIDWFKSAINQHEDWKKFGFKFSDTKSYKDISDFYREVTEQGYKLTFINIPESEISKMVSEGKLYLFQIYNMDFAPGANGMPNMHTLYWKNLFSEENLKDVVLKLNGEAELFYRPAGIKEPVVHAKGSYLVNRITKDGEPIPEKIHDEIYRNANGNLNNLSKEASEYKESHKVVIKQASHEIIKDRHYTEPKFLFHIPLTINFKAPSMKPTVCTAIQNENVRRFLKNNPDVNIIGLDRGERNLIYLSLINQKGEIIKQFSFNDVEREQNGQTVKVNYHEKLDQREKERDAARKSWQAIGKIAELKEGYLSAVIHQLTKLMVEYNAIVVMEDLNFGFKRGRFHVEKQVYQKFEHMLIDKLNYLVFKDRGLNEPGGALNGYQLAGQFESFQKLGKQSGMLFYVPAAYTSKIDPKTGFVSMMNFKDLTNVHKKRDFFSKFEDIHFDEATCSFVFTFDYKNFNGKAKEEMKQTKWAVYSREKRIVYFSKTKSSEDIMPTEKLRALFESDGIEYKSGNNIHDSVMAVGADLKEGAKPSKEIADFWDGLLYNFKLILQMRNSNAKTGEDYIISPVMASDGTFFDSRVEAKKGKDAKLPLDADANGAYHIALKGLSLINKINLAGEDELKKFDMKISNEDWFNFAQEKKYAE

SEQ ID NO: 86 - protein, 1345 aa, synthetic construct
  REGION 1..1345: note = Description of Artificial Sequence: Synthetic polypeptide
MTKQNKSVTQNTKRKNFGEFTNLYSLSKTLRFELKPVKATKTILEVERKDRENFKKDRKIAKNYQKLKGILNELHQEFIQDVMREFSFQEKEIKEFEERYLEALNFKEKDNYKKRTQLKNAYEKVAKKLAGKIATAFGKYNQEKYGVKFTKKNLTGENVFDILEGKYKGDKKILGIIHTFKFKPTKEEKKQGKEAVNFSTYLGGFNQNRENFYKGEMKAGQFATRTIENLIQFLKNKKLFIDKYKDNYQKIGFSQEQVEIFNLNYFNNLFLQEGLDVYNGILGAKKGEKNTENDGLNQKINLFKQKEKTRCKANGEKFNKSDYPIFKELYKQIGSIKKDNDVYVEIKSDEELVNVLQSLPEKTANTLREVQKFYENFFDKIFNDEFDLDKIYLPKSVGTHFSHLAFSDWSKLAFVFNKRWRNEKVKIKEGEDVNVQSRSLADIKKRMEEILEMDGGVSFGKTYCQKVGLEKEARTIEDVWSGFWKIIQYHINSQFIGGEKEVFDKEKKDDKTEKIQTIDDLQEEYLQATEMYRERMVESEEGLNDGEEKEIKTKLKNYLDRIKDIERIARYFDLRKHFDDIDEASKDGDFYFIYQELLQDISEAKINDHYNEIRNYLTKANVVDDKFKLNFNDGQTLSGWDLNKETEKFSLIFKRKVDGGVEYYLGIINKEKNKTIFDKKKHPEIFTENSEFEKMEYKLFPSPSKMLPKIAFTKNKEGERIKPVFLDENAGKEIAQIKKEFALFQDAKKEDKNKWSDEFDRKKLNKLIDYYKLVLEKHPEKYMQTFNFVFKSSAKYKNLGEFNDDVARQNYVTKFVSVDKDYIDQKVESGELYLFKIHNKDWNLTKAGDTKKQSKKNLHTIYFEELFSEKNIAEPVFKLSGGAEVFFRDAIEKKKQKKKKDKKGKEILEKFRFTKNKILFHVPITINYGKPSINQGQFNQKINEFIADNSRSVNILGIDRGEKHLLYYSLVDNNGKIIKSGSLNEINGVDYHEKLDKAEKERQEARKSWQKINQIKNLKAGYISQVIKKIVDLAIENNAIIVLEDLNFGFKSFRQKIEKNVYQQFEKALIDKLGFVTDKEKLNHRQAPQLSAPFESFEKMGKQTGIVFYVLATNTSKVCPQCQWKKNIFPHYSTKKSIAENLQKQYKMKMYWRENENRFEFEYKGDGDKEFSSIFSNVDRVRYDKRANNNQGGYVIYQIDSTTKEKDGRNIKEKSITNLLKELLLEKFEIDNLEGELLVKLSEKSPDVSKETIKDFFGLLNSILNIRNSMTDTEEDYIQCPACGFDTRKENKIGIKNGDDNGAYNIALRGRFLIERIKKAKKEDKKPNLTFSNNDYFQWVREFVK

SEQ ID NO: 87 - protein, 1265 aa, synthetic construct
  REGION 1..1265: note = Description of Artificial Sequence: Synthetic polypeptide
MRTTTSLDAFTNRYALSKTLRFELKPIGNTQMMLEQNNVFAKDRAIREKYEKTKPWIDLLHREFVAESLQNAQLGNLDDYYAALQNVQKITKDTNAEDKKRWKKGFEKQEKRLRKEVVALFDKAAHIWATQRYPQLKKKTKDFLFEEGVFEHVLFARYGSAPDTTVKIVTSNPETGEVIDEREESIFKGWKGFTGYFDKFFETRKNFYKDNGTATAIATRAINQNLRRFAENMQKLTDIKNNYPELLAHTDFGDFDIAHAQSLDFYARTCLLQEGIDAYNKKFVGVLKSAINEYQQKNKGVRISYPKTLDNQILGERERRLFDVIEDDRELHDVFRAFVDDGTVFAAEMRQLAQAFSAQNGTYDYTQIYISKKGFETISRKYTHDTRAWHDALADVFKAKAKKRIATTASGEKKFPAYIPVAYITQALTLVQESEDTECTWKERYASITENKTLEEGFFAIFADEFERLFVHMEATVQDTDYVVAEDKAKKLLSDGQITKNEQTTQIIKEYADALLRIYQMAKYFAVEKKSMWDDAVAIDDTFYETFKEIYGNTHSTIVASYNLLRNYLTKKPWEDVQKWKLNFENPTLLDGWDKNKEAANFGVILRDGDKFYLGIMRKGHNNIFANQHHSNFEGQGLQKMVYKFFPDPKKMFPKVCFSAKGMEFFAPSEEIVRIYKNAEFKSGDTFNVESMQKLIDFYKNALQKYDGWKIYDFKHLKDTAQYTSNIGEFYDDVAKGGYQLGWQNISKEYVEEKNANGELYLFQIKNKDWNDGATGRKNLHTLYFEYLFSEKNAAADFVFRLNGGAEVFYRPAAIESKTERRGNREVAAKKRYTQDKVFLHVPITLNRTAGDVKTSAFNDAVNRFLAGNPDINIMGIDRGEKHLAYYSIIDQNGNRIVSGSFNTIGSKDYHALLTERQGAREEARKNWQRVEQIKDLKKGYISLVVREIADLAIKHNAIIVLENLNMRFKQIRGGIEKSVYQQLEKALIEKLNFLVNKGEVDATKAGHLLRAYQLAAPFETFEKMGNQTGIIFYTTASYTSQVDPVTGWRPHVYLKYRNAQKTKEDILRIFDDIVFNDEKQRFEFAYRHNGVSWTVCSSVERHRWNRSNNAGKGGYDVFPVEGEGSITQRLQEACAQRGIDTTRNILAQIDELDESASATVSFLRDLCFYFRLICQIRNTDDGADDINAQDFLMSPVEPFFDTRNAQEQYPQNGDENGAYNIARKGIIILQKITAWGRSQDTQRRYPDTFVSQDEWDTFLTQHTT

SEQ ID NO: 88 - protein, 1259 aa, synthetic construct
  REGION 1..1259: note = Description of Artificial Sequence: Synthetic polypeptide
MLKEKQFKTFGDFTNLYELSKTLRFELRPTPETKDLLDKNKIIQTDKKIAENYQEIKKYFDKLHKKFIKEALSNTQIDFSDFCKLWEQNSKDSGKIKDLSRKLRKSIKQAFDKKGADWHKRYLEKGIKLKKKNLDILFEERVLDILKEEFKDDVDVKLFESFKGFSTYFTNFHESRKNFYKDDGTASAIATRIIDENLKRFCDNIKVKKHSKKLISELNEREAKIFEADFYNRCLLQQGIDDYNQVIGDINKKINNLRQNKIENTPTLKILYKQILGDVRRQETEQDAFIEIKNNEEVFDFLQDFIKHSDENNKYFKNLFYKFIEGKHSLDKIFLAKRFVNTISGKWFASWEVFGAELIKKFGNKKDLPDFIPFAAVKDVLQNCNIPANELFKEKIKNDEDKNIYDIFINLWKEEFDSNLKKVEESKKEVENMIAEDKVYSNKKEKRKNDNGEEIEIEIQKEKIKNYADAAMNIFRMMKYFLLEKNGKTVEGMGEDNNFYNELNIVFKGGEIDGKVYEGVKTYLYYNEFRNYLTKKPFNEEKTKLNFDCGQILSGWDKNKESEKLGVILRKDNKYYLAIINKKHNKIFDVKKNSYAYIVGDNFYEKMEYKLFPDAKRMIPKIAFAKNNKEKFGWTDEIQKIKNEYAEFQEGKKNDKNLWKDKFNKNKMEKLITYYQNCLEKGGYKDIYNFRWKSPDKYSGIGEFNDEIDRQSYCLKFVKVDFNYVFEKVKSGELYLFQIYNKDFSDKADRAQKENIHTEYFKLLFDQRNLDNVVLKLSGGAEIFYRPKTEGLPKKKDNKGNEVVRHRRYADDKYFLHLPIQLNFGRGNLSGGEFNSKINQYLSEQREIKIIGIDRGEKHLAYYSVINQDGKIEEIESLNTVNGIDYRKKLDELEKKREQERKSWQSISKIKDLKKGYISHVIKKICDLAIEHNAIIVFEDLSGGFKNSRKKIEKQIYQNLELALATKLNYLTFKDKNFGESGHYLNAYQLAPKIDNYQDIKMQTGIVFYTPAGYTSSTCPQCGFRKTLKFDYTATISKAEDLIRGSKLNIVFEKEKNRFKINYLFNPIEKKKKKIKENELFADAGAKNEFTIYSDVKRIKWHNTGTKRLEEAEGERLLENKNSRGRDKEYDINKCLTRLFRENKIDVNGDIIGQITKIKSLKLYQDLFYYLFLATLIRNNVSGSDIDYIQCPSCHFHSDGGFQKQKFNGDANGAYNIARKGILILKKIKQFAAQDKDMKNFGWKHLTVDINEWDKFTQK

SEQ ID NO: 89 - protein, 1371 aa, synthetic construct
  REGION 1..1371: note = Description of Artificial Sequence: Synthetic polypeptide
MNKNFSNFTELYTLSKTLRFELKPVAQTKENIKKGKFLESDKKKADDYKDVKKIIDNYHKFFIDDVLKNASFDWTVLEKEMSDFNKSKADDSKVEAEQKKLRDQIAKKLTSDKRFKALTASTPSDLFNKDKDFIDWFTQNSTKDINKEALETFKRFSSYFKGFQENRKNVYSAEPIPTAVPYRLVNDNFPKFLQNIALFKIIQEKCPQVISDVEKELASYLGKEKLADIFTVQAFNKYLCQGGKENQRGIDFYNNILGGIAEKEGGINLRGINQFLNLYWQQHQDFAKQNKRIKMIPLFKQILSDRSSLSFKIESINTDQELLTSITEYADKLETKSNDEKKSVLDICSDLFASIKAQNLQEIYVNRKDINSISRILTGDWSWLQSRMNVYADEVFTTKAEKTRWQKSIDGDEGENKSKGVFSLAELNSVLEYSSENVSETDVRITDFFDHRNRFYYEKESGLFKQGDELVALSIRESCEDILAKRKAMDEAFANVSENNSLRDNSEDVAKIKIYLDCVQELLHRIKPLKVNGLGDPAFYAVFDTVYNSLSEVISLYNKTRNYITKKAANPEKYKLNFDNPTLADGWDLNKEQANTSVLMRKDGMYYLGIMNPKDKPKFAEKYECGNEACYEKMIYKQFDATKQIPKCSTQVKEVKKHFQSGATDSIILNDKSKFKLDLVITKEIWFLNNHVWNGEKFVPKRESNETRPKKFQIGYYKQTGDLGGYKEALNIWISFCKTFLQSYISSSIYDYDFKESSNYDSLDEFYNYLNATCYKLSFINIPEATISQMVSEGKLYLFQIYNKDFAPGASGMPNMHTLYWKNLFSEENLKNVVLKLNGEAELFYRPAGIKEPVIHAKGSYLVNRITKDGEPIPEKIHDEIYRNANGKLESLSKEATEYKASHKVVIKEAKHDIIKDRHYTEPKFLFHVPLTINFKASGNSYINENVRRFLKNNPDVNVIGLDRGERHLIYLSLINQKGEIIKQFTFNEVERNKNGQVIKVNYHEKLDQREKVRGAARKSWQAIGKIAELKEGYLSAVIHQLTKLMVEYNAIVVMEDLNFGFKRGRFHVEKQVYQKFEHMLIDKLNYLVFKDRGLTEAGGVLNGYQLAGQFESFQKLGKQSGMLFYVPAGYTSKIDPKTGFASMSNFKDLTNVHKKRAFFSKFDDIHFDDATGSFVFTFDYKNFDGKAKEEMKRTKWSVYSKDKRIVYLSKTKSYEDVQPTEKIKASLESVGIEYMSGNNLIDSIMVIGAELKDGAKPSKEIADFWDRLLYNFKLILQMRNSNAKTGEDYIISPVMADDGTFFDSREEFKKGENAKMPVDADANGAYHIALKGLSLLKRFDAASENELKKFDMKISNVDWFKFAQEKSYAE

SEQ ID NO: 90 - protein, 1160 aa, synthetic construct
  REGION 1..1160: note = Description of Artificial Sequence: Synthetic polypeptide
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAV

SEQ ID NO: 91 - protein, 1344 aa, synthetic construct
  REGION 1..1344: note = Description of Artificial Sequence: Synthetic polypeptide
MLRRDKEVKKLYNVFNQIQVGTKPKKWNNDEKLSPEENERRAQQKNIKMKNYKWREACSKYVESSQRIINDVIFYSYRKAKNKLRYMRKNEDILKKMQEAEKLSKFSGGKLEDFVAYTLRKSLVVSKYDTQEFDSLAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKDAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPSYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNARSLNLMVGSMRSYIQYVTDIKRRAASIGNELHVSVHDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANLNFKLSN

SEQ ID NO: 92 - protein, 1285 aa, synthetic construct
  REGION 1..1285: note = Description of Artificial Sequence: Synthetic polypeptide
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 93 - DNA, 50 bp, synthetic construct; identical to SEQ ID NO: 62
  misc_feature 1..50: note = Description of Artificial Sequence: Synthetic oligonucleotide
  source 1..50: mol_type = other DNA

SEQ ID NO: 94 - DNA, 59 bp, synthetic construct; identical to SEQ ID NO: 63
  misc_feature 1..59: note = Description of Artificial Sequence: Synthetic oligonucleotide
  source 1..59: mol_type = other DNA

SEQ ID NO: 95 - DNA, 12 bp, synthetic construct
  misc_feature 1..12: note = Description of Artificial Sequence: Synthetic oligonucleotide
  source 1..12: mol_type = other DNA
tttttttttttt

SEQ ID NO: 96 - DNA, 19 bp, synthetic construct
  misc_feature 1..19: note = Description of Artificial Sequence: Synthetic oligonucleotide
  source 1..19: mol_type = other DNA
ttttttttttttttttttt

SEQ ID NO: 97 - DNA, 60 bp, synthetic construct; identical to SEQ ID NO: 66
  misc_feature 1..60: note = Description of Artificial Sequence: Synthetic oligonucleotide
  source 1..60: mol_type = other DNA